(19) United States
(12) Patent Application Publication
(10) Pub. No.: US 2024/0076370 A1
(43) Pub. Date: Mar. 7, 2024

(54) ANTIBODIES TARGETING G-PROTEIN COUPLED RECEPTOR AND METHODS OF USE

(71) Applicants: MEMORIAL SLOAN-KETTERING CANCER CENTER, New York, NY (US); EUREKA THERAPEUTICS, INC., Emeryville, CA (US)

(72) Inventors: Renier J. Brentjens, New York, NY (US); Eric L. Smith, New York, NY (US); Cheng Liu, Emeryville, CA (US)

(73) Assignees: MEMORIAL SLOAN-KETTERING CANCER CENTER, New York, NY (US); EUREKA THERAPEUTICS, INC., Emeryville, CA (US)

(21) Appl. No.: 18/145,254

(22) Filed: Dec. 22, 2022

Related U.S. Application Data
(63) Continuation of application No. 16/732,022, filed on Dec. 31, 2019, now Pat. No. 11,566,071.
Application No. 16/732,022 is a division of application No. 15/614,290, filed on Jun. 5, 2017, now Pat. No. 10,590,196.
Application No. 15/614,290 is a continuation of application No. PCT/US2015/064122, filed on Dec. 4, 2015.
(60) Provisional application No. 62/088,228, filed on Dec. 5, 2014.

(51) Int. Cl. C07K 16/28; A61K 47/68; A61P 35/00; G01N 33/68

(52) U.S. Cl. CPC C07K 16/28; A61K 47/6849; A61P 35/00; C07K 16/2809; G01N 33/6872; C07K 2317/21; C07K 2317/24; C07K 2317/31; C07K 2317/34; C07K 2317/622; C07K 2317/92; C07K 2319/21; G01N 2333/705

(57) ABSTRACT

The presently disclosed subject matter provides antibodies that bind to GPRC5D and methods of using the same.

### CROSS-REFERENCE TO RELATED APPLICATIONS

This application is a Continuation of U.S. patent application Ser. No. 16/732,022, filed Dec. 31, 2019, which is a Divisional of U.S. Pat. No. 10,590,196, filed Jun. 5, 2017, which is a Continuation of International Patent Application No. PCT/US2015/064122, filed Dec. 4, 2015, which claims priority to U.S. Provisional Application Ser. No. 62/088,228, filed Dec. 5, 2014, the contents of each of which are incorporated by reference in their entireties, and to each of which priority is claimed.

### SEQUENCE LISTING

The specification further incorporates by reference the Sequence Listing submitted herewith on Dec. 22, 2022. Pursuant to 37 C.F.R. § 1.52(e)(5), the Sequence Listing file, identified as 089333.0491.xml, is 539,543 bytes and was created on Dec. 21, 2022. The Sequence Listing electronically filed herewith, does not extend beyond the scope of the specification and thus does not contain new matter.

### FIELD OF THE INVENTION

The presently disclosed subject matter relates to human antibodies that bind to a G-protein coupled receptor (e.g., a G-protein coupled receptor family C group 5 member D (GPRC5D)), and methods of using the same.

### BACKGROUND

G protein-coupled receptors, also known as seven-transmembrane domain receptors, 7TM receptors, heptahelical receptors, serpentine receptor, and G protein-linked receptors, constitute a large protein family of receptors that sense molecules outside the cell and activate inside signal transduction pathways and, ultimately, cellular responses. GPCRs can be categorized into six classes based on sequence homology and functional similarity: Class A (Rhodopsin-like), Class B (Secretin receptor family), Class C (Metabotropic glutamate/pheromone), Class D (Fungal mating pheromone receptors), Class E (Cyclic AMP receptors), and Class F (Frizzled/Smoothened).

G-protein coupled receptor family C group 5 member D (GPRC5D) is an orphan receptor with no known ligand or function in humans. It is a member of a family of retinoic acid-inducible G-protein-coupled receptors. It is overexpressed in multiple myeloma (MM) cells and is not expressed or expressed in a significantly lower level by any other cell type, benign or malignant, as shown in FIG. 1. Several groups have identified this gene as highly differentially expressed by gene expression profiling of primary MM cells when compared to normal tissue1 or other hematologic malignancies (Frigyesi, I., et al. Robust isolation of malignant plasma cells in multiple myeloma. Blood 123, 1336-1340 (2014); Cohen, Y., Gutwein, O., Garach-Jehoshua, O., Bar-Haim, A. & Kornberg, A. GPRC5D is a promising marker for monitoring the tumor load and to target multiple myeloma cells. Hematology (Amsterdam, Netherlands) 18, 348-351 (2013); Bam, R., et al. GPRC5D Is a Cell Surface Plasma Cell Marker Whose Expression Is High In Myeloma Cells and Reduced Following Coculture With Osteoclasts. Blood 122, 3099 (2013)). It has been shown that higher mRNA expression correlates with worse overall survival (Atamaniuk, J., et al. Overexpression of G protein-coupled receptor 5D in the bone marrow is associated with poor prognosis in patients with multiple myeloma. European journal of clinical investigation 42, 953-960 (2012)). Surface staining of Bone marrow aspirates from patients with MM demonstrate plasma cell specific staining (Bam, R., et al. GPRC5D Is a Cell Surface Plasma Cell Marker Whose Expression Is High In Myeloma Cells and Reduced Following Coculture With Osteoclasts. Blood 122, 3099 (2013)). Given the significant role for GPRC5D in multiple myeloma, antibodies that recognize GPRC5D, and methods of using such agents, are desired.

### SUMMARY

The presently disclosed subject matter provides human antibodies that bind to a G-protein coupled receptor (e.g., GPRC5D), and methods of using the same.

In certain embodiments, the presently disclosed subject matter provides an isolated antibody, or an antigen-binding fragment thereof, comprising a heavy chain variable region comprising an amino acid sequence that is at least about 80%, 81%, 82%, 83%, 84%, 85%, 86%, 87%, 88%, 89%, 90%, 91%, 92%, 93%, 94%, 95%, 96%, 97%, 98% or 99% homologous to an amino acid sequence selected from the group consisting of SEQ ID NOS: 1, 5, 9, 13, 17, 21, 25, 29, 33, 37, 41, 45, 49, 53, 57, 61, 65, 69, 73, 77, 81, 85, 89, 93, 274, 286, 298, 310, 322, 334, 346 and 358, wherein the antibody or antigen-binding fragment thereof specifically binds to human GPRC5D.

In certain embodiments, the presently disclosed subject matter provides an isolated antibody, or an antigen-binding fragment thereof, comprising a light chain variable region comprising an amino acid sequence that is at least about 80%, 81%, 82%, 83%, 84%, 85%, 86%, 87%, 88%, 89%, 90%, 91%, 92%, 93%, 94%, 95%, 96%, 97%, 98% or 99% homologous to an amino acid sequence selected from the group consisting of SEQ ID NOS: 2, 6, 10, 14, 18, 22, 26, 30, 34, 38, 42, 46, 50, 54, 58, 62, 66, 70, 74, 78, 82, 86, 90, 94, 275, 287, 299, 311, 323, 335, 347 and 359, wherein the antibody or antigen-binding fragment thereof specifically binds to human GPRC5D.

In certain embodiments, the presently disclosed subject matter provides an isolated antibody, or an antigen-binding fragment thereof, comprising (a) a heavy chain variable region comprising an amino acid sequence that is at least about 80%, 81%, 82%, 83%, 84%, 85%, 86%, 87%, 88%, 89%, 90%, 91%, 92%, 93%, 94%, 95%, 96%, 97%, 98% or 99% homologous to an amino acid sequence selected from the group consisting of SEQ ID NOS: 1, 5, 9, 13, 17, 21, 25, 29, 33, 37, 41, 45, 49, 53, 57, 61, 65, 69, 73, 77, 81, 85, 89, 93, 274, 286, 298, 310, 322, 334, 346 and 358; and (b) a light chain variable region comprising an amino acid sequence that is at least about 80%, 81%, 82%, 83%, 84%, 85%, 86%, 87%, 88%, 89%, 90%, 91%, 92%, 93%, 94%, 95%, 96%, 97%, 98% or 99% homologous to an amino acid sequence selected from the group consisting of SEQ ID NOS: 2, 6, 10, 14, 18, 22, 26, 30, 34, 38, 42, 46, 50, 54, 58, 62, 66, 70, 74, 78, 82, 86, 90, 94, 275, 287, 299, 311, 323, 335, 347 and 359, wherein the antibody or antigen-binding fragment thereof specifically binds to human GPRC5D.

In certain embodiments, the presently disclosed subject matter provides an isolated antibody, or an antigen-binding fragment thereof, comprising a heavy chain variable region and a light chain variable region, wherein the heavy chain variable region and the light chain variable region are selected from the group consisting of: (i) a heavy chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:1, and a light chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:2; (ii) a heavy chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:5, and a light chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:6; (iii) a heavy chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:9, and a light chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:10; (iv) a heavy chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:13, and a light chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:14; (v) a heavy chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:17, and a light chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:18; (vi) a heavy chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:21, and a light chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:22; (vii) a heavy chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:25, and a light chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:26; (viii) a heavy chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:29, and a light chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:30; (ix) a heavy chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:33, and a light chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:34; (x) a heavy chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:37, and a light chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:38; (xi) a heavy chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:41, and a light chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:42; (xii) a heavy chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:45, and a light chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:46; (xiii) a heavy chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:49, and a light chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:50; (xiv) a heavy chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:53, and a light chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:54; (xv) a heavy chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:57, and a light chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:58; (xvi) a heavy chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:61, and a light chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:62; (xvii) a heavy chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:65, and a light chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:66; (xviii) a heavy chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:69, and a light chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:70; (xix) a heavy chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:73, and a light chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:74; (xx) a heavy chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:77, and a light chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:78; (xxi) a heavy chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:81, and a light chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:82; (xxii) a heavy chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:85, and a light chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:86; (xxiii) a heavy chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:89, and a light chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:90; (xxiv) a heavy chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:93, and a light chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:94; (xxv) a heavy chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:274, and a light chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:275; (xxvi) a heavy chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:286, and a light chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:287; (xxvii) a heavy chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:298, and a light chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:299; (xxviii) a heavy chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:310, and a light chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:311; (xxix) a heavy chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:322, and a light chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:323; (xxx) a heavy chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:334, and a light chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:335; (xxxi) a heavy chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:346, and a light chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:347; or (xxxii) a heavy chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:358, and a light chain variable region comprising amino acids having a sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the sequence set forth in SEQ ID NO:359, wherein the antibody or antigen-binding fragment thereof specifically binds to human GPRC5D.

In certain embodiments, the antibody or antigen-binding fragment comprises: (i) a heavy chain variable region comprising amino acids having a sequence set forth in SEQ ID NO:1, and a light chain variable region comprising amino acids having a sequence set forth in SEQ ID NO:2; (ii) a heavy chain variable region comprising amino acids having a sequence set forth in SEQ ID NO:5, and a light chain variable region comprising amino acids having a sequence set forth in SEQ ID NO:6; (iii) a heavy chain variable region comprising amino acids having a sequence set forth in SEQ ID NO:9, and a light chain variable region comprising amino acids having a sequence set forth in SEQ ID NO:10; (iv) a heavy chain variable region comprising amino acids having a sequence set forth in SEQ ID NO:13, and a light chain variable region comprising amino acids having a sequence set forth in SEQ ID NO:14; (v) a heavy chain variable region comprising amino acids having a sequence set forth in SEQ ID NO:17, and a light chain variable region comprising amino acids having a sequence set forth in SEQ ID NO:18; (vi) a heavy chain variable region comprising amino acids having a sequence set forth in SEQ ID NO:21, and a light chain variable region comprising amino acids having a sequence set forth in SEQ ID NO:22; (vii) a heavy chain variable region comprising amino acids having a sequence set forth in SEQ ID NO:25, and a light chain variable region comprising amino acids having a sequence set forth in SEQ ID NO:26; (viii) a heavy chain variable region comprising amino acids having a sequence set forth in SEQ ID NO:29, and a light chain variable region comprising amino acids having a sequence set forth in SEQ ID NO:30; (ix) a heavy chain variable region comprising amino acids having a sequence set forth in SEQ ID NO:33, and a light chain variable region comprising amino acids having a sequence set forth in SEQ ID NO:34; (x) a heavy chain variable region comprising amino acids having a sequence set forth in SEQ ID NO:37, and a light chain variable region comprising amino acids having a sequence set forth in SEQ ID NO:38; (xi) a heavy chain variable region comprising amino acids having a sequence set forth in SEQ ID NO:41, and a light chain variable region comprising amino acids having a sequence set forth in SEQ ID NO:42; (xii) a heavy chain variable region comprising amino acids having a sequence set forth in SEQ ID NO:45, and a light chain variable region comprising amino acids having a sequence set forth in SEQ ID NO:46; (xiii) a heavy chain variable region comprising amino acids having a sequence set forth in SEQ ID NO:49, and a light chain variable region comprising amino acids having a sequence set forth in SEQ ID NO:50; (xiv) a heavy chain variable region comprising amino acids having a sequence set forth in SEQ ID NO:53, and a light chain variable region comprising amino acids having a sequence set forth in SEQ ID NO:54; (xv) a heavy chain variable region comprising amino acids having a sequence set forth in SEQ ID NO:57, and a light chain variable region comprising amino acids having a sequence set forth in SEQ ID NO:58; (xvi) a heavy chain variable region comprising amino acids having a sequence set forth in SEQ ID NO:61, and a light chain variable region comprising amino acids having a sequence set forth in SEQ ID NO:62; (xvii) a heavy chain variable region comprising amino acids having a sequence set forth in SEQ ID NO:65, and a light chain variable region comprising amino acids having a sequence set forth in SEQ ID NO:66; (xviii) a heavy chain variable region comprising amino acids having a sequence set forth in SEQ ID NO:69, and a light chain variable region comprising amino acids having a sequence set forth in SEQ ID NO:70; (xix) a heavy chain variable region comprising amino acids having a sequence set forth in SEQ ID NO:73, and a light chain variable region comprising amino acids having a sequence set forth in SEQ ID NO:74; (xx) a heavy chain variable region comprising amino acids having a sequence set forth in SEQ ID NO:77, and a light chain variable region comprising amino acids having a sequence set forth in SEQ ID NO:78; (xxi) a heavy chain variable region comprising amino acids having a sequence set forth in SEQ ID NO:81, and a light chain variable region comprising amino acids having a sequence set forth in SEQ ID NO:82; (xxii) a heavy chain variable region comprising amino acids having a sequence set forth in SEQ ID NO:85, and a light chain variable region comprising amino acids having a sequence set forth in SEQ ID NO:86; (xxiii) a heavy chain variable region comprising amino acids having a sequence set forth in SEQ ID NO:89, and a light chain variable region comprising amino acids having a sequence set forth in SEQ ID NO:90; (xxiv) a heavy chain variable region comprising amino acids having a sequence set forth in SEQ ID NO:93, and a light chain variable region comprising amino acids having a sequence set forth in SEQ ID NO:94; (xxv) a heavy chain variable region comprising amino acids having a sequence set forth in SEQ ID NO:274, and a light chain variable region comprising amino acids having a set forth in SEQ ID NO:275; (xxvi) a heavy chain variable region comprising amino acids having a sequence set forth in SEQ ID NO:286, and a light chain variable region comprising amino acids having a sequence set forth in SEQ ID NO:287; (xxvii) a heavy chain variable region comprising amino acids having a sequence set forth in SEQ ID NO:298, and a light chain variable region comprising amino acids having a sequence set forth in SEQ ID NO:299; (xxviii) a heavy chain variable region comprising amino acids having a sequence set forth in SEQ ID NO:310, and a light chain variable region comprising amino acids having a sequence set forth in SEQ ID NO:311; (xxix) a heavy chain variable region comprising amino acids having a sequence set forth in SEQ ID NO:322, and a light chain variable region comprising amino acids having a sequence set forth in SEQ ID NO:323; (xxx) a heavy chain variable region comprising amino acids having a sequence set forth in SEQ ID NO:334, and a light chain variable region comprising amino acids having a sequence set forth in SEQ ID NO:335; (xxxi) a heavy chain variable region comprising amino acids having a sequence set forth in SEQ ID NO:346, and a light chain variable region comprising amino acids having a sequence set forth in SEQ ID NO:347; or (xxxii) a heavy chain variable region comprising amino acids having a sequence set forth in SEQ ID NO:358, and a light chain variable region comprising amino acids having a sequence set forth in SEQ ID NO:359.

In certain embodiments, the presently disclosed subject matter provides an isolated antibody, or an antigen-binding fragment thereof, comprising a heavy chain variable region comprising an amino acid sequence selected from the group consisting of SEQ ID NOS: 1, 5, 9, 13, 17, 21, 25, 29, 33, 37, 41, 45, 49, 53, 57, 61, 65, 69, 73, 77, 81, 85, 89, 93, 274, 286, 298, 310, 322, 334, 346 and 358, and conservative modifications thereof, wherein the antibody or antigen-binding fragment thereof specifically binds to human GPRC5D.

In certain embodiments, the presently disclosed subject matter provides an isolated antibody, or an antigen-binding fragment thereof, comprising a light chain variable region comprising an amino acid sequence selected from the group consisting of SEQ ID NOS: 2, 6, 10, 14, 18, 22, 26, 30, 34, 38, 42, 46, 50, 54, 58, 62, 66, 70, 74, 78, 82, 86, 90, 94, 275, 287, 299, 311, 323, 335, 347 and 359, and conservative modifications thereof, wherein the antibody or antigen-binding fragment thereof specifically binds to human GPRC5D.

In certain embodiments, the presently disclosed subject matter provides an isolated antibody, or an antigen-binding fragment thereof, comprising: a heavy chain variable region comprising an amino acid sequence selected from the group consisting of SEQ ID NOS: 1, 5, 9, 13, 17, 21, 25, 29, 33, 37, 41, 45, 49, 53, 57, 61, 65, 69, 73, 77, 81, 85, 89, 93, 274, 286, 298, 310, 322, 334, 346 and 358, and conservative modifications thereof; and a light chain variable region comprising an amino acid sequence selected from the group consisting of SEQ ID NOS: 2, 6, 10, 14, 18, 22, 26, 30, 34, 38, 42, 46, 50, 54, 58, 62, 66, 70, 74, 78, 82, 86, 90, 94, 275, 287, 299, 311, 323, 335, 347 and 359, and conservative modifications thereof, wherein the antibody or antigen-binding fragment thereof specifically binds to human GPRC5D.

The presently disclosed subject matter also provides an isolated antibody or antigen-binding fragment thereof comprising a heavy chain variable region that comprises CDR1, CDR2, and CDR3 domains; and a light chain variable region that comprises CDR1, CDR2, and CDR3 domains, wherein the heavy chain variable region and light chain variable region CDR3 domains are selected from the group consisting of:
(i) a heavy chain variable region CDR3 comprising amino acids having the sequence set forth in SEQ ID NO:126 and conservative modifications thereof; and a light chain variable region CDR3 comprising amino acids having the sequence set forth in SEQ ID NO: 129 and conservative modifications thereof;
(ii) a heavy chain variable region CDR3 comprising amino acids having the sequence set forth in SEQ ID NO: 132 and conservative modifications thereof; and a light chain variable region CDR3 comprising amino acids having the sequence set forth in SEQ ID NO: 135 and conservative modifications thereof;
(iii) a heavy chain variable region CDR3 comprising amino acids having the sequence set forth in SEQ ID NO: 138 and conservative modifications thereof; and a light chain variable region CDR3 comprising amino acids having the sequence set forth in SEQ ID NO:141 and conservative modifications thereof;
(iv) a heavy chain variable region CDR3 comprising amino acids having the sequence set forth in SEQ ID NO: 144 and conservative modifications thereof; and a light chain variable region CDR3 comprising amino acids having the sequence set forth in SEQ ID NO: 147 and conservative modifications thereof;
(v) a heavy chain variable region CDR3 comprising amino acids having the sequence set forth in SEQ ID NO:150 and conservative modifications thereof; and a light chain variable region CDR3 comprising amino acids having the sequence set forth in SEQ ID NO:153 and conservative modifications thereof;
(vi) a heavy chain variable region CDR3 comprising amino acids having the sequence set forth in SEQ ID NO: 156 and conservative modifications thereof; and a light chain variable region CDR3 comprising amino acids having the sequence set forth in SEQ ID NO: 159 and conservative modifications thereof;
(vii) a heavy chain variable region CDR3 comprising amino acids having the sequence set forth in SEQ ID NO: 162 and conservative modifications thereof; and a light chain variable region CDR3 comprising amino acids having the sequence set forth in SEQ ID NO: 165 and conservative modifications thereof;
(viii) a heavy chain variable region CDR3 comprising amino acids having the sequence set forth in SEQ ID NO: 168 and conservative modifications thereof; and a light chain variable region CDR3 comprising amino acids having the sequence set forth in SEQ ID NO:171 and conservative modifications thereof;
(ix) a heavy chain variable region CDR3 comprising amino acids having the sequence set forth in SEQ ID NO: 174 and conservative modifications thereof; and a light chain variable region CDR3 comprising amino acids having the sequence set forth in SEQ ID NO: 177 and conservative modifications thereof;
(x) a heavy chain variable region CDR3 comprising amino acids having the sequence set forth in SEQ ID NO:180 and conservative modifications thereof; and a light chain variable region CDR3 comprising amino acids having the sequence set forth in SEQ ID NO:183 and conservative modifications thereof;
(xi) a heavy chain variable region CDR3 comprising amino acids having the sequence set forth in SEQ ID NO: 186 and conservative modifications thereof; and a light chain variable region CDR3 comprising amino acids having the sequence set forth in SEQ ID NO: 189 and conservative modifications thereof;
(xii) a heavy chain variable region CDR3 comprising amino acids having the sequence set forth in SEQ ID NO: 192 and conservative modifications thereof; and a light chain variable region CDR3 comprising amino acids having the sequence set forth in SEQ ID NO:195 and conservative modifications thereof;
(xiii) a heavy chain variable region CDR3 comprising amino acids having the sequence set forth in SEQ ID NO: 198 and conservative modifications thereof; and a light chain variable region CDR3 comprising amino acids having the sequence set forth in SEQ ID NO: 201 and conservative modifications thereof;
(xiv) a heavy chain variable region CDR3 comprising amino acids having the sequence set forth in SEQ ID NO: 204 and conservative modifications thereof; and a light chain variable region CDR3 comprising amino acids having the sequence set forth in SEQ ID NO: 207 and conservative modifications thereof;
(xv) a heavy chain variable region CDR3 comprising amino acids having the sequence set forth in SEQ ID NO: 210 and conservative modifications thereof; and a light chain variable region CDR3 comprising amino acids having the sequence set forth in SEQ ID NO: 213 and conservative modifications thereof;
(xvi) a heavy chain variable region CDR3 comprising amino acids having the sequence set forth in SEQ ID NO: 216 and conservative modifications thereof; and a light chain variable region CDR3 comprising amino acids having the sequence set forth in SEQ ID NO: 219 and conservative modifications thereof;
(xvii) a heavy chain variable region CDR3 comprising amino acids having the sequence set forth in SEQ ID NO:222 and conservative modifications thereof; and a light chain variable region CDR3 comprising amino acids having the sequence set forth in SEQ ID NO:225 and conservative modifications thereof;
(xviii) a heavy chain variable region CDR3 comprising amino acids having the sequence set forth in SEQ ID NO: 228 and conservative modifications thereof; and a light chain variable region CDR3 comprising amino acids having the sequence set forth in SEQ ID NO: 231 and conservative modifications thereof;
(xix) a heavy chain variable region CDR3 comprising amino acids having the sequence set forth in SEQ ID NO: 234 and conservative modifications thereof; and a light chain variable region CDR3 comprising amino acids having the sequence set forth in SEQ ID NO:237 and conservative modifications thereof;
(xx) a heavy chain variable region CDR3 comprising amino acids having the sequence set forth in SEQ ID NO:240 and conservative modifications thereof; and a light chain variable region CDR3 comprising amino acids having the sequence set forth in SEQ ID NO: 243 and conservative modifications thereof;
(xxi) a heavy chain variable region CDR3 comprising amino acids having the sequence set forth in SEQ ID NO: 246 and conservative modifications thereof; and a light chain variable region CDR3 comprising amino acids having the sequence set forth in SEQ ID NO: 249 and conservative modifications thereof;
(xxii) a heavy chain variable region CDR3 comprising amino acids having the sequence set forth in SEQ ID NO: 252 and conservative modifications thereof; and a light chain variable region CDR3 comprising amino acids having the sequence set forth in SEQ ID NO: 255 and conservative modifications thereof;
(xxiii) a heavy chain variable region CDR3 comprising amino acids having the sequence set forth in SEQ ID NO: 258 and conservative modifications thereof; and a light chain variable region CDR3 comprising amino acids having the sequence set forth in SEQ ID NO: 261 and conservative modifications thereof;
(xxiv) a heavy chain variable region CDR3 comprising amino acids having the sequence set forth in SEQ ID NO: 264 and conservative modifications thereof; and a light chain variable region CDR3 comprising amino acids having the sequence set forth in SEQ ID NO: 267 and conservative modifications thereof;
(xxv) a heavy chain variable region CDR3 comprising amino acids having the sequence set forth in SEQ ID NO: 270 and conservative modifications thereof; and a light chain variable region CDR3 comprising amino acids having the sequence set forth in SEQ ID NO: 273 and conservative modifications thereof;
(xxvi) a heavy chain variable region CDR3 comprising amino acids having the sequence set forth in SEQ ID NO: 282 and conservative modifications thereof; and a light chain variable region CDR3 comprising amino acids having the sequence set forth in SEQ ID NO: 285 and conservative modifications thereof;
(xxvii) a heavy chain variable region CDR3 comprising amino acids having the sequence set forth in SEQ ID NO: 294 and conservative modifications thereof; and a light chain variable region CDR3 comprising amino acids having the sequence set forth in SEQ ID NO: 297 and conservative modifications thereof;
(xxviii) a heavy chain variable region CDR3 comprising amino acids having the sequence set forth in SEQ ID NO: 305 and conservative modifications thereof; and a light chain variable region CDR3 comprising amino acids having the sequence set forth in SEQ ID NO: 308 and conservative modifications thereof;
(xxix) a heavy chain variable region CDR3 comprising amino acids having the sequence set forth in SEQ ID NO: 318 and conservative modifications thereof; and a light chain variable region CDR3 comprising amino acids having the sequence set forth in SEQ ID NO: 321 and conservative modifications thereof;
(xxx) a heavy chain variable region CDR3 comprising amino acids having the sequence set forth in SEQ ID NO: 330 and conservative modifications thereof; and a light chain variable region CDR3 comprising amino acids having the sequence set forth in SEQ ID NO: 333 and conservative modifications thereof;
(xxxi) a heavy chain variable region CDR3 comprising amino acids having the sequence set forth in SEQ ID NO: 342 and conservative modifications thereof; and a light chain variable region CDR3 comprising amino acids having the sequence set forth in SEQ ID NO: 345 and conservative modifications thereof; and
(xxxii) a heavy chain variable region CDR3 comprising amino acids having the sequence set forth in SEQ ID NO: 354 and conservative modifications thereof; and a light chain variable region CDR3 comprising amino acids having the sequence set forth in SEQ ID NO: 357 and conservative modifications thereof,

wherein the antibody or antigen-binding portion thereof specifically binds to GPRC5D.

In certain embodiments, the heavy chain variable region and light chain variable region CDR2 domains the antibody or antigen-binding portion thereof are selected from the group consisting of:
(i) a heavy chain variable region CDR2 comprising amino acids having the sequence set forth in SEQ ID NO:125 and conservative modifications thereof; and a light chain variable region CDR2 comprising amino acids having the sequence set forth in SEQ ID NO: 128 and conservative modifications thereof;
(ii) a heavy chain variable region CDR2 comprising amino acids having the sequence set forth in SEQ ID NO: 131 and conservative modifications thereof; and a light chain variable region CDR2 comprising amino acids having the sequence set forth in SEQ ID NO: 134 and conservative modifications thereof;
(iii) a heavy chain variable region CDR2 comprising amino acids having the sequence set forth in SEQ ID NO: 137 and conservative modifications thereof; and a light chain variable region CDR2 comprising amino acids having the sequence set forth in SEQ ID NO: 140 and conservative modifications thereof;
(iv) a heavy chain variable region CDR2 comprising amino acids having the sequence set forth in SEQ ID NO: 143 and conservative modifications thereof; and a light chain variable region CDR2 comprising amino acids having the sequence set forth in SEQ ID NO: 146 and conservative modifications thereof;
(v) a heavy chain variable region CDR2 comprising amino acids having the sequence set forth in SEQ ID NO: 149 and conservative modifications thereof; and a light chain variable region CDR2 comprising amino acids having the sequence set forth in SEQ ID NO: 152 and conservative modifications thereof;
(vi) a heavy chain variable region CDR2 comprising amino acids having the sequence set forth in SEQ ID NO: 155 and conservative modifications thereof; and a light chain variable region CDR2 comprising amino acids having the sequence set forth in SEQ ID NO: 158 and conservative modifications thereof;
(vii) a heavy chain variable region CDR2 comprising amino acids having the sequence set forth in SEQ ID NO: 161 and conservative modifications thereof; and a light chain variable region CDR2 comprising amino acids having the sequence set forth in SEQ ID NO: 164 and conservative modifications thereof;
(viii) a heavy chain variable region CDR2 comprising amino acids having the sequence set forth in SEQ ID NO: 167 and conservative modifications thereof; and a light chain variable region CDR2 comprising amino acids having the sequence set forth in SEQ ID NO: 170 and conservative modifications thereof;
(ix) a heavy chain variable region CDR2 comprising amino acids having the sequence set forth in SEQ ID NO: 173 and conservative modifications thereof; and a light chain variable region CDR2 comprising amino acids having the sequence set forth in SEQ ID NO: 176 and conservative modifications thereof;
(x) a heavy chain variable region CDR2 comprising amino acids having the sequence set forth in SEQ ID NO:179 and conservative modifications thereof; and a light chain variable region CDR2 comprising amino acids having the sequence set forth in SEQ ID NO:182 and conservative modifications thereof;
(xi) a heavy chain variable region CDR2 comprising amino acids having the sequence set forth in SEQ ID NO: 185 and conservative modifications thereof; and a light chain variable region CDR2 comprising amino acids having the sequence set forth in SEQ ID NO: 188 and conservative modifications thereof;
(xii) a heavy chain variable region CDR2 comprising amino acids having the sequence set forth in SEQ ID NO:191 and conservative modifications thereof; and a light chain variable region CDR2 comprising amino acids having the sequence set forth in SEQ ID NO:194 and conservative modifications thereof;
(xiii) a heavy chain variable region CDR2 comprising amino acids having the sequence set forth in SEQ ID NO:197 and conservative modifications thereof; and a light chain variable region CDR2 comprising amino acids having the sequence set forth in SEQ ID NO:200 and conservative modifications thereof;
(xiv) a heavy chain variable region CDR2 comprising amino acids having the sequence set forth in SEQ ID NO: 203 and conservative modifications thereof; and a light chain variable region CDR2 comprising amino acids having the sequence set forth in SEQ ID NO: 206 and conservative modifications thereof;
(xv) a heavy chain variable region CDR2 comprising amino acids having the sequence set forth in SEQ ID NO: 209 and conservative modifications thereof; and a light chain variable region CDR2 comprising amino acids having the sequence set forth in SEQ ID NO: 212 and conservative modifications thereof;
(xvi) a heavy chain variable region CDR2 comprising amino acids having the sequence set forth in SEQ ID NO: 215 and conservative modifications thereof; and a light chain variable region CDR2 comprising amino acids having the sequence set forth in SEQ ID NO: 218 and conservative modifications thereof;
(xvii) a heavy chain variable region CDR2 comprising amino acids having the sequence set forth in SEQ ID NO: 221 and conservative modifications thereof; and a light chain variable region CDR2 comprising amino acids having the sequence set forth in SEQ ID NO: 224 and conservative modifications thereof;
(xviii) a heavy chain variable region CDR2 comprising amino acids having the sequence set forth in SEQ ID NO: 227 and conservative modifications thereof; and a light chain variable region CDR2 comprising amino acids having the sequence set forth in SEQ ID NO:230 and conservative modifications thereof;
(xix) a heavy chain variable region CDR2 comprising amino acids having the sequence set forth in SEQ ID NO:233 and conservative modifications thereof; and a light chain variable region CDR2 comprising amino acids having the sequence set forth in SEQ ID NO:236 and conservative modifications thereof;
(xx) a heavy chain variable region CDR2 comprising amino acids having the sequence set forth in SEQ ID NO:239 and conservative modifications thereof; and a light chain variable region CDR2 comprising amino acids having the sequence set forth in SEQ ID NO:242 and conservative modifications thereof;
(xxi) a heavy chain variable region CDR2 comprising amino acids having the sequence set forth in SEQ ID NO:245 and conservative modifications thereof; and a light chain variable region CDR2 comprising amino acids having the sequence set forth in SEQ ID NO:248 and conservative modifications thereof;
(xxii) a heavy chain variable region CDR2 comprising amino acids having the sequence set forth in SEQ ID NO:251 and conservative modifications thereof; and a light chain variable region CDR2 comprising amino acids having the sequence set forth in SEQ ID NO:254 and conservative modifications thereof;
(xxiii) a heavy chain variable region CDR2 comprising amino acids having the sequence set forth in SEQ ID NO:257 and conservative modifications thereof; and a light chain variable region CDR2 comprising amino acids having the sequence set forth in SEQ ID NO:260 and conservative modifications thereof;
(xxiv) a heavy chain variable region CDR2 comprising amino acids having the sequence set forth in SEQ ID NO:263 and conservative modifications thereof; and a light chain variable region CDR2 comprising amino acids having the sequence set forth in SEQ ID NO:266 and conservative modifications thereof;
(xxv) a heavy chain variable region CDR2 comprising amino acids having the sequence set forth in SEQ ID NO:269 and conservative modifications thereof; and a light chain variable region CDR2 comprising amino acids having the sequence set forth in SEQ ID NO:272 and conservative modifications thereof;
(xxvi) a heavy chain variable region CDR2 comprising amino acids having the sequence set forth in SEQ ID NO:281 and conservative modifications thereof; and a light chain variable region CDR2 comprising amino acids having the sequence set forth in SEQ ID NO:284 and conservative modifications thereof;
(xxvii) a heavy chain variable region CDR2 comprising amino acids having the sequence set forth in SEQ ID NO:293 and conservative modifications thereof; and a light chain variable region CDR2 comprising amino acids having the sequence set forth in SEQ ID NO:296 and conservative modifications thereof;
(xxviii) a heavy chain variable region CDR2 comprising amino acids having the sequence set forth in SEQ ID NO:304 and conservative modifications thereof; and a light chain variable region CDR2 comprising amino acids having the sequence set forth in SEQ ID NO:307 and conservative modifications thereof;
(xxix) a heavy chain variable region CDR2 comprising amino acids having the sequence set forth in SEQ ID NO:317 and conservative modifications thereof; and a light chain variable region CDR2 comprising amino acids having the sequence set forth in SEQ ID NO:320 and conservative modifications thereof;
(xxx) a heavy chain variable region CDR2 comprising amino acids having the sequence set forth in SEQ ID NO:329 and conservative modifications thereof; and a light chain variable region CDR2 comprising amino acids having the sequence set forth in SEQ ID NO:332 and conservative modifications thereof;
(xxxi) a heavy chain variable region CDR2 comprising amino acids having the sequence set forth in SEQ ID NO:341 and conservative modifications thereof; and a light chain variable region CDR2 comprising amino acids having the sequence set forth in SEQ ID NO:344 and conservative modifications thereof; and
(xxxii) a heavy chain variable region CDR2 comprising amino acids having the sequence set forth in SEQ ID NO:353 and conservative modifications thereof; and a light chain variable region CDR2 comprising amino acids having the sequence set forth in SEQ ID NO:356 and conservative modifications thereof, wherein the antibody or antigen-binding portion thereof specifically binds to GPRC5D.

In certain embodiments, the heavy chain variable region and light chain variable region CDR1 domains of the antibody or antigen-binding portion thereof are selected from the group consisting of:
(i) a heavy chain variable region CDR1 comprising amino acids having the sequence set forth in SEQ ID NO:124 and conservative modifications thereof; and a light chain variable region CDR1 comprising amino acids having the sequence set forth in SEQ ID NO: 127 and conservative modifications thereof;
(ii) a heavy chain variable region CDR1 comprising amino acids having the sequence set forth in SEQ ID NO:130 and conservative modifications thereof; and a light chain variable region CDR1 comprising amino acids having the sequence set forth in SEQ ID NO:133 and conservative modifications thereof;
(iii) a heavy chain variable region CDR1 comprising amino acids having the sequence set forth in SEQ ID NO:136 and conservative modifications thereof; and a light chain variable region CDR1 comprising amino acids having the sequence set forth in SEQ ID NO:139 and conservative modifications thereof;
(iv) a heavy chain variable region CDR1 comprising amino acids having the sequence set forth in SEQ ID NO:142 and conservative modifications thereof; and a light chain variable region CDR1 comprising amino acids having the sequence set forth in SEQ ID NO:145 and conservative modifications thereof;
(v) a heavy chain variable region CDR1 comprising amino acids having the sequence set forth in SEQ ID NO:148 and conservative modifications thereof; and a light chain variable region CDR1 comprising amino acids having the sequence set forth in SEQ ID NO:151 and conservative modifications thereof;
(vi) a heavy chain variable region CDR1 comprising amino acids having the sequence set forth in SEQ ID NO:154 and conservative modifications thereof; and a light chain variable region CDR1 comprising amino acids having the sequence set forth in SEQ ID NO:157 and conservative modifications thereof;
(vii) a heavy chain variable region CDR1 comprising amino acids having the sequence set forth in SEQ ID NO:160 and conservative modifications thereof; and a light chain variable region CDR1 comprising amino acids having the sequence set forth in SEQ ID NO:163 and conservative modifications thereof;
(viii) a heavy chain variable region CDR1 comprising amino acids having the sequence set forth in SEQ ID NO:166 and conservative modifications thereof; and a light chain variable region CDR1 comprising amino acids having the sequence set forth in SEQ ID NO:169 and conservative modifications thereof;
(ix) a heavy chain variable region CDR1 comprising amino acids having the sequence set forth in SEQ ID NO:172 and conservative modifications thereof; and a light chain variable region CDR1 comprising amino acids having the sequence set forth in SEQ ID NO:175 and conservative modifications thereof;
(x) a heavy chain variable region CDR1 comprising amino acids having the sequence set forth in SEQ ID NO:178 and conservative modifications thereof; and a light chain variable region CDR1 comprising amino acids having the sequence set forth in SEQ ID NO:181 and conservative modifications thereof;
(xi) a heavy chain variable region CDR1 comprising amino acids having the sequence set forth in SEQ ID NO:184 and conservative modifications thereof; and a light chain variable region CDR1 comprising amino acids having the sequence set forth in SEQ ID NO:187 and conservative modifications thereof;
(xii) a heavy chain variable region CDR1 comprising amino acids having the sequence set forth in SEQ ID NO:190 and conservative modifications thereof; and a light chain variable region CDR1 comprising amino acids having the sequence set forth in SEQ ID NO:193 and conservative modifications thereof;
(xiii) a heavy chain variable region CDR1 comprising amino acids having the sequence set forth in SEQ ID NO:196 and conservative modifications thereof; and a light chain variable region CDR1 comprising amino acids having the sequence set forth in SEQ ID NO:199 and conservative modifications thereof;
(xiv) a heavy chain variable region CDR1 comprising amino acids having the sequence set forth in SEQ ID NO:202 and conservative modifications thereof; and a light chain variable region CDR1 comprising amino acids having the sequence set forth in SEQ ID NO:205 and conservative modifications thereof;
(xv) a heavy chain variable region CDR1 comprising amino acids having the sequence set forth in SEQ ID NO:208 and conservative modifications thereof; and a light chain variable region CDR1 comprising amino acids having the sequence set forth in SEQ ID NO:211 and conservative modifications thereof;
(xvi) a heavy chain variable region CDR1 comprising amino acids having the sequence set forth in SEQ ID NO:214 and conservative modifications thereof; and a light chain variable region CDR1 comprising amino acids having the sequence set forth in SEQ ID NO:217 and conservative modifications thereof;
(xvii) a heavy chain variable region CDR1 comprising amino acids having the sequence set forth in SEQ ID NO:220 and conservative modifications thereof; and a light chain variable region CDR1 comprising amino acids having the sequence set forth in SEQ ID NO:223 and conservative modifications thereof;
(xviii) a heavy chain variable region CDR1 comprising amino acids having the sequence set forth in SEQ ID NO:226 and conservative modifications thereof; and a light chain variable region CDR1 comprising amino acids having the sequence set forth in SEQ ID NO:229 and conservative modifications thereof;
(xix) a heavy chain variable region CDR1 comprising amino acids having the sequence set forth in SEQ ID NO:232 and conservative modifications thereof; and a light chain variable region CDR1 comprising amino acids having the sequence set forth in SEQ ID NO:235 and conservative modifications thereof;
(xx) a heavy chain variable region CDR1 comprising amino acids having the sequence set forth in SEQ ID NO:238 and conservative modifications thereof; and a light chain variable region CDR1 comprising amino acids having the sequence set forth in SEQ ID NO:241 and conservative modifications thereof;
(xxi) a heavy chain variable region CDR1 comprising amino acids having the sequence set forth in SEQ ID NO:244 and conservative modifications thereof; and a light chain variable region CDR1 comprising amino acids having the sequence set forth in SEQ ID NO:247 and conservative modifications thereof;
(xxii) a heavy chain variable region CDR1 comprising amino acids having the sequence set forth in SEQ ID NO:250 and conservative modifications thereof; and a light chain variable region CDR1 comprising amino acids having the sequence set forth in SEQ ID NO:253 and conservative modifications thereof;
(xxiii) a heavy chain variable region CDR1 comprising amino acids having the sequence set forth in SEQ ID NO:256 and conservative modifications thereof; and a light chain variable region CDR1 comprising amino acids having the sequence set forth in SEQ ID NO:259 and conservative modifications thereof;
(xxiv) a heavy chain variable region CDR1 comprising amino acids having the sequence set forth in SEQ ID NO:262 and conservative modifications thereof; and a light chain variable region CDR1 comprising amino acids having the sequence set forth in SEQ ID NO:265 and conservative modifications thereof;
(xxv) a heavy chain variable region CDR1 comprising amino acids having the sequence set forth in SEQ ID NO:268 and conservative modifications thereof; and a light chain variable region CDR1 comprising amino acids having the sequence set forth in SEQ ID NO:271 and conservative modifications thereof;
(xxvi) a heavy chain variable region CDR1 comprising amino acids having the sequence set forth in SEQ ID NO:280 and conservative modifications thereof; and a light chain variable region CDR1 comprising amino acids having the sequence set forth in SEQ ID NO:283 and conservative modifications thereof;
(xxvii) a heavy chain variable region CDR1 comprising amino acids having the sequence set forth in SEQ ID NO:292 and conservative modifications thereof; and a light chain variable region CDR1 comprising amino acids having the sequence set forth in SEQ ID NO:295 and conservative modifications thereof;
(xxviii) a heavy chain variable region CDR1 comprising amino acids having the sequence set forth in SEQ ID NO:303 and conservative modifications thereof; and a light chain variable region CDR1 comprising amino acids having the sequence set forth in SEQ ID NO:306 and conservative modifications thereof;
(xxix) a heavy chain variable region CDR1 comprising amino acids having the sequence set forth in SEQ ID NO:316 and conservative modifications thereof; and a light chain variable region CDR1 comprising amino acids having the sequence set forth in SEQ ID NO:319 and conservative modifications thereof;
(xxx) a heavy chain variable region CDR1 comprising amino acids having the sequence set forth in SEQ ID NO:328 and conservative modifications thereof; and a light chain variable region CDR1 comprising amino acids having the sequence set forth in SEQ ID NO:331 and conservative modifications thereof;
(xxxi) a heavy chain variable region CDR1 comprising amino acids having the sequence set forth in SEQ ID NO:340 and conservative modifications thereof; and a light chain variable region CDR1 comprising amino acids having the sequence set forth in SEQ ID NO:343 and conservative modifications thereof; and
(xxxii) a heavy chain variable region CDR1 comprising amino acids having the sequence set forth in SEQ ID NO:352 and conservative modifications thereof; and a light chain variable region CDR1 comprising amino acids having the sequence set forth in SEQ ID NO:355 and conservative modifications thereof,

wherein the antibody or antigen-binding portion thereof specifically binds to GPRC5D.

Furthermore, the presently disclosed subject matter provides an isolated antibody, or an antigen-binding portion thereof, comprising:
(i) a heavy chain variable region CDR1 comprising amino acids having the sequence set forth in SEQ ID NO: 124; a heavy chain variable region CDR2 comprising amino acids having the sequence set forth in SEQ ID NO: 125; and a heavy chain variable region CDR3 comprising amino acids having the sequence set forth in SEQ ID NO: 126;
(ii) a heavy chain variable region CDR1 comprising amino acids having the sequence set forth in SEQ ID NO: 130; a heavy chain variable region CDR2 comprising amino acids having the sequence set forth in SEQ ID NO: 131; and a heavy chain variable region CDR3 comprising amino acids having the sequence set forth in SEQ ID NO: 132;
(iii) a heavy chain variable region CDR1 comprising amino acids having the sequence set forth in SEQ ID NO: 136; a heavy chain variable region CDR2 comprising amino acids having the sequence set forth in SEQ ID NO: 137; and a heavy chain variable region CDR3 comprising amino acids having the sequence set forth in SEQ ID NO: 138;
(iv) a heavy chain variable region CDR1 comprising amino acids having the sequence set forth in SEQ ID NO: 142; a heavy chain variable region CDR2 comprising amino acids having the sequence set forth in SEQ ID NO: 143; and a heavy chain variable region CDR3 comprising amino acids having the sequence set forth in SEQ ID NO: 144;
(v) a heavy chain variable region CDR1 comprising amino acids having the sequence set forth in SEQ ID NO: 148; a heavy chain variable region CDR2 comprising amino acids having the sequence set forth in SEQ ID NO: 149; and a heavy chain variable region CDR3 comprising amino acids having the sequence set forth in SEQ ID NO: 150;
(vi) a heavy chain variable region CDR1 comprising amino acids having the sequence set forth in SEQ ID NO: 154; a heavy chain variable region CDR2 comprising amino acids having the sequence set forth in SEQ ID NO: 155; and a heavy chain variable region CDR3 comprising amino acids having the sequence set forth in SEQ ID NO: 156;
(vii) a heavy chain variable region CDR1 comprising amino acids having the sequence set forth in SEQ ID NO: 160; a heavy chain variable region CDR2 comprising amino acids having the sequence set forth in SEQ ID NO: 161; and a heavy chain variable region CDR3 comprising amino acids having the sequence set forth in SEQ ID NO: 162;
(viii) a heavy chain variable region CDR1 comprising amino acids having the sequence set forth in SEQ ID NO: 166; a heavy chain variable region CDR2 comprising amino acids having the sequence set forth in SEQ ID NO: 167; and a heavy chain variable region CDR3 comprising amino acids having the sequence set forth in SEQ ID NO: 168;
(ix) a heavy chain variable region CDR1 comprising amino acids having the sequence set forth in SEQ ID NO: 172; a heavy chain variable region CDR2 comprising amino acids having the sequence set forth in SEQ ID NO: 173; and a heavy chain variable region CDR3 comprising amino acids having the sequence set forth in SEQ ID NO: 174;
(x) a heavy chain variable region CDR1 comprising amino acids having the sequence set forth in SEQ ID NO: 178; a heavy chain variable region CDR2 comprising amino acids having the sequence set forth in SEQ ID NO: 179; and a heavy chain variable region CDR3 comprising amino acids having the sequence set forth in SEQ ID NO: 180;
(xi) a heavy chain variable region CDR1 comprising amino acids having the sequence set forth in SEQ ID NO: 184; a heavy chain variable region CDR2 comprising amino acids having the sequence set forth in SEQ ID NO: 185; and a heavy chain variable region CDR3 comprising amino acids having the sequence set forth in SEQ ID NO: 186;
(xii) a heavy chain variable region CDR1 comprising amino acids having the sequence set forth in SEQ ID NO: 190 a heavy chain variable region CDR2 comprising amino acids having the sequence set forth in SEQ ID NO: 191; and a heavy chain variable region CDR3 comprising amino acids having the sequence set forth in SEQ ID NO: 192;
(xiii) a heavy chain variable region CDR1 comprising amino acids having the sequence set forth in SEQ ID NO: 196; a heavy chain variable region CDR2 comprising amino acids having the sequence set forth in SEQ ID NO: 197; and a heavy chain variable region CDR3 comprising amino acids having the sequence set forth in SEQ ID NO: 198;
(xiv) a heavy chain variable region CDR1 comprising amino acids having the sequence set forth in SEQ ID NO: 202; a heavy chain variable region CDR2 comprising amino acids having the sequence set forth in SEQ ID NO: 203; and a heavy chain variable region CDR3 comprising amino acids having the sequence set forth in SEQ ID NO: 204;
(xv) a heavy chain variable region CDR1 comprising amino acids having the sequence set forth in SEQ ID NO: 208; a heavy chain variable region CDR2 comprising amino acids having the sequence set forth in SEQ ID NO: 209; and a heavy chain variable region CDR3 comprising amino acids having the sequence set forth in SEQ ID NO: 210;
(xvi) a heavy chain variable region CDR1 comprising amino acids having the sequence set forth in SEQ ID NO: 214; a heavy chain variable region CDR2 comprising amino acids having the sequence set forth in SEQ ID NO: 215; and a heavy chain variable region CDR3 comprising amino acids having the sequence set forth in SEQ ID NO: 216;
(xvii) a heavy chain variable region CDR1 comprising amino acids having the sequence set forth in SEQ ID NO: 220; a heavy chain variable region CDR2 comprising amino acids having the sequence set forth in SEQ ID NO: 221; and a heavy chain variable region CDR3 comprising amino acids having the sequence set forth in SEQ ID NO: 222;
(xviii) a heavy chain variable region CDR1 comprising amino acids having the sequence set forth in SEQ ID NO: 226; a heavy chain variable region CDR2 comprising amino acids having the sequence set forth in SEQ ID NO: 227; and a heavy chain variable region CDR3 comprising amino acids having the sequence set forth in SEQ ID NO: 228;
(xix) a heavy chain variable region CDR1 comprising amino acids having the sequence set forth in SEQ ID NO: 232; a heavy chain variable region CDR2 comprising amino acids having the sequence set forth in SEQ ID NO: 233; and a heavy chain variable region CDR3 comprising amino acids having the sequence set forth in SEQ ID NO: 234;
(xx) a heavy chain variable region CDR1 comprising amino acids having the sequence set forth in SEQ ID NO: 238; a heavy chain variable region CDR2 comprising amino acids having the sequence set forth in SEQ ID NO: 239; and a heavy chain variable region CDR3 comprising amino acids having the sequence set forth in SEQ ID NO: 240;
(xxi) a heavy chain variable region CDR1 comprising amino acids having the sequence set forth in SEQ ID NO: 244; a heavy chain variable region CDR2 comprising amino acids having the sequence set forth in SEQ ID NO: 245; and a heavy chain variable region CDR3 comprising amino acids having the sequence set forth in SEQ ID NO: 246;
(xxii) a heavy chain variable region CDR1 comprising amino acids having the sequence set forth in SEQ ID NO: 250; a heavy chain variable region CDR2 comprising amino acids having the sequence set forth in SEQ ID NO: 251; and a heavy chain variable region CDR3 comprising amino acids having the sequence set forth in SEQ ID NO: 252; and
(xxiii) a heavy chain variable region CDR1 comprising amino acids having the sequence set forth in SEQ ID NO: 256; a heavy chain variable region CDR2 comprising amino acids having the sequence set forth in SEQ ID NO: 257; and a heavy chain variable region CDR3 comprising amino acids having the sequence set forth in SEQ ID NO: 258;
(xxiv) a heavy chain variable region CDR1 comprising amino acids having the sequence set forth in SEQ ID NO: 262; a heavy chain variable region CDR2 comprising amino acids having the sequence set forth in SEQ ID NO: 263; and a heavy chain variable region CDR3 comprising amino acids having the sequence set forth in SEQ ID NO: 264;
(xxv) a heavy chain variable region CDR1 comprising amino acids having the sequence set forth in SEQ ID NO: 268; a heavy chain variable region CDR2 comprising amino acids having the sequence set forth in SEQ ID NO: 269; and a heavy chain variable region CDR3 comprising amino acids having the sequence set forth in SEQ ID NO: 270;
(xxvi) a heavy chain variable region CDR1 comprising amino acids having the sequence set forth in SEQ ID NO: 280; a heavy chain variable region CDR2 comprising amino acids having the sequence set forth in SEQ ID NO: 281; and a heavy chain variable region CDR3 comprising amino acids having the sequence set forth in SEQ ID NO: 282;
(xxvii) a heavy chain variable region CDR1 comprising amino acids having the sequence set forth in SEQ ID NO: 292; a heavy chain variable region CDR2 comprising amino acids having the sequence set forth in SEQ ID NO: 293; and a heavy chain variable region CDR3 comprising amino acids having the sequence set forth in SEQ ID NO: 294;
(xxviii) a heavy chain variable region CDR1 comprising amino acids having the sequence set forth in SEQ ID NO: 303; a heavy chain variable region CDR2 comprising amino acids having the sequence set forth in SEQ ID NO: 304; and a heavy chain variable region CDR3 comprising amino acids having the sequence set forth in SEQ ID NO: 305;
(xxix) a heavy chain variable region CDR1 comprising amino acids having the sequence set forth in SEQ ID NO: 316; a heavy chain variable region CDR2 comprising amino acids having the sequence set forth in SEQ ID NO: 317; and a heavy chain variable region CDR3 comprising amino acids having the sequence set forth in SEQ ID NO: 318;
(xxx) a heavy chain variable region CDR1 comprising amino acids having the sequence set forth in SEQ ID NO: 328; a heavy chain variable region CDR2 comprising amino acids having the sequence set forth in SEQ ID NO: 329; and a heavy chain variable region CDR3 comprising amino acids having the sequence set forth in SEQ ID NO: 330;
(xxxi) a heavy chain variable region CDR1 comprising amino acids having the sequence set forth in SEQ ID NO: 340; a heavy chain variable region CDR2 comprising amino acids having the sequence set forth in SEQ ID NO: 341; and a heavy chain variable region CDR3 comprising amino acids having the sequence set forth in SEQ ID NO: 342; or
(xxxii) a heavy chain variable region CDR1 comprising amino acids having the sequence set forth in SEQ ID NO: 352; a heavy chain variable region CDR2 comprising amino acids having the sequence set forth in SEQ ID NO: 353; and a heavy chain variable region CDR3 comprising amino acids having the sequence set forth in SEQ ID NO: 354;

wherein the antibody or antigen-binding portion thereof specifically binds to GPRC5D.

Additionally, the presently disclosed subject matter provides an isolated antibody, or an antigen-binding portion thereof, comprising:
(i) a light chain variable region CDR1 comprising amino acids having the sequence set forth in SEQ ID NO: 127; a light chain variable region CDR2 comprising amino acids having the sequence set forth in SEQ ID NO:129; and a light chain variable region CDR3 comprising amino acids having the sequence set forth in SEQ ID NO: 130;
(ii) a light chain variable region CDR1 comprising amino acids having the sequence set forth in SEQ ID NO: 133; a light chain variable region CDR2 comprising amino acids having the sequence set forth in SEQ ID NO:134; and a light chain variable region CDR3 comprising SEQ ID NO: 135;
(iii) a light chain variable region CDR1 comprising amino acids having the sequence set forth in SEQ ID NO: 139; a light chain variable region CDR2 comprising amino acids having the sequence set forth in SEQ ID NO:140; and a light chain variable region CDR3 comprising amino acids having the sequence set forth in SEQ ID NO: 141;
(iv) a light chain variable region CDR1 comprising amino acids having the sequence set forth in SEQ ID NO: 145; a light chain variable region CDR2 comprising SEQ ID NO:146; and a light chain variable region CDR3 comprising amino acids having the sequence set forth in SEQ ID NO: 147;
(v) a light chain variable region CDR1 comprising amino acids having the sequence set forth in SEQ ID NO: 151; a light chain variable region CDR2 comprising amino acids having the sequence set forth in SEQ ID NO:152; and a light chain variable region CDR3 comprising amino acids having the sequence set forth in SEQ ID NO: 153;
(vi) a light chain variable region CDR1 comprising amino acids having the sequence set forth in SEQ ID NO: 157; a light chain variable region CDR2 comprising amino acids having the sequence set forth in SEQ ID NO:158; and a light chain variable region CDR3 comprising amino acids having the sequence set forth in SEQ ID NO: 159;
(vii) a light chain variable region CDR1 comprising amino acids having the sequence set forth in SEQ ID NO: 163; a light chain variable region CDR2 comprising amino acids having the sequence set forth in SEQ ID NO:164; and a light chain variable region CDR3 comprising amino acids having the sequence set forth in SEQ ID NO: 165;
(viii) a light chain variable region CDR1 comprising amino acids having the sequence set forth in SEQ ID NO: 169; a light chain variable region CDR2 comprising amino acids having the sequence set forth in SEQ ID NO:170; and a light chain variable region CDR3 comprising SEQ ID NO: 171;
(ix) a light chain variable region CDR1 comprising amino acids having the sequence set forth in SEQ ID NO: 175; a light chain variable region CDR2 comprising amino acids having the sequence set forth in SEQ ID NO:176; and a light chain variable region CDR3 comprising amino acids having the sequence set forth in SEQ ID NO: 177;
(x) a light chain variable region CDR1 comprising amino acids having the sequence set forth in SEQ ID NO: 181; a light chain variable region CDR2 comprising amino acids having the sequence set forth in SEQ ID NO:182; and a light chain variable region CDR3 comprising amino acids having the sequence set forth in SEQ ID NO: 183;
(xi) a light chain variable region CDR1 comprising amino acids having the sequence set forth in SEQ ID NO: 187; a light chain variable region CDR2 comprising amino acids having the sequence set forth in SEQ ID NO:188; and a light chain variable region CDR3 comprising amino acids having the sequence set forth in SEQ ID NO: 189;
(xii) a light chain variable region CDR1 comprising amino acids having the sequence set forth in SEQ ID NO: 193; a light chain variable region CDR2 comprising amino acids having the sequence set forth in SEQ ID NO:194; and a light chain variable region CDR3 comprising amino acids having the sequence set forth in SEQ ID NO: 195;
(xiii) a light chain variable region CDR1 comprising amino acids having the sequence set forth in SEQ ID NO: 199; a light chain variable region CDR2 comprising amino acids having the sequence set forth in SEQ ID NO:200; and a light chain variable region CDR3 comprising amino acids having the sequence set forth in SEQ ID NO: 201;
(xiv) a light chain variable region CDR1 comprising amino acids having the sequence set forth in SEQ ID NO: 205; a light chain variable region CDR2 comprising amino acids having the sequence set forth in SEQ ID NO:206; and a light chain variable region CDR3 comprising amino acids having the sequence set forth in SEQ ID NO: 207;
(xv) a light chain variable region CDR1 comprising amino acids having the sequence set forth in SEQ ID NO: 211; a light chain variable region CDR2 comprising amino acids having the sequence set forth in SEQ ID NO:212; and a light chain variable region CDR3 comprising amino acids having the sequence set forth in SEQ ID NO: 213;
(xvi) a light chain variable region CDR1 comprising amino acids having the sequence set forth in SEQ ID NO: 217; a light chain variable region CDR2 comprising amino acids having the sequence set forth in SEQ ID NO:218; and a light chain variable region CDR3 comprising amino acids having the sequence set forth in SEQ ID NO: 219;
(xvii) a light chain variable region CDR1 comprising amino acids having the sequence set forth in SEQ ID NO: 223; a light chain variable region CDR2 comprising amino acids having the sequence set forth in SEQ ID NO:224; and a light chain variable region CDR3 comprising amino acids having the sequence set forth in SEQ ID NO: 225;
(xviii) a light chain variable region CDR1 comprising amino acids having the sequence set forth in SEQ ID NO: 229; a light chain variable region CDR2 comprising amino acids having the sequence set forth in SEQ ID NO:230; and a light chain variable region CDR3 comprising amino acids having the sequence set forth in SEQ ID NO: 231;
(xix) a light chain variable region CDR1 comprising amino acids having the sequence set forth in SEQ ID NO: 235; a light chain variable region CDR2 comprising amino acids having the sequence set forth in SEQ ID NO:236; and a light chain variable region CDR3 comprising amino acids having the sequence set forth in SEQ ID NO: 237;
(xx) a light chain variable region CDR1 comprising amino acids having the sequence set forth in SEQ ID NO: 241; a light chain variable region CDR2 comprising amino acids having the sequence set forth in SEQ ID NO:242; and a light chain variable region CDR3 comprising amino acids having the sequence set forth in SEQ ID NO: 243;
(xxi) a light chain variable region CDR1 comprising amino acids having the sequence set forth in SEQ ID NO: 247; a light chain variable region CDR2 comprising amino acids having the sequence set forth in SEQ ID NO:248; and a light chain variable region CDR3 comprising amino acids having the sequence set forth in SEQ ID NO: 249;
(xxii) a light chain variable region CDR1 comprising amino acids having the sequence set forth in SEQ ID NO: 253; a light chain variable region CDR2 comprising amino acids having the sequence set forth in SEQ ID NO:254; and a light chain variable region CDR3 comprising amino acids having the sequence set forth in SEQ ID NO: 255;
(xxiii) a light chain variable region CDR1 comprising amino acids having the sequence set forth in SEQ ID NO: 259; a light chain variable region CDR2 comprising amino acids having the sequence set forth in SEQ ID NO:250; and a light chain variable region CDR3 comprising amino acids having the sequence set forth in SEQ ID NO: 261; or
(xxiv) a light chain variable region CDR1 comprising amino acids having the sequence set forth in SEQ ID NO: 265; a light chain variable region CDR2 comprising amino acids having the sequence set forth in SEQ ID NO:266; and a light chain variable region CDR3 comprising amino acids having the sequence set forth in SEQ ID NO: 267;
(xxv) a light chain variable region CDR1 comprising amino acids having the sequence set forth in SEQ ID NO: 271; a light chain variable region CDR2 comprising amino acids having the sequence set forth in SEQ ID NO:272; and a light chain variable region CDR3 comprising amino acids having the sequence set forth in SEQ ID NO: 273;
(xxvi) a light chain variable region CDR1 comprising amino acids having the sequence set forth in SEQ ID NO: 283; a light chain variable region CDR2 comprising amino acids having the sequence set forth in SEQ ID NO:284; and a light chain variable region CDR3 comprising amino acids having the sequence set forth in SEQ ID NO: 285;
(xxvii) a light chain variable region CDR1 comprising amino acids having the sequence set forth in SEQ ID NO: 295; a light chain variable region CDR2 comprising amino acids having the sequence set forth in SEQ ID NO:296; and a light chain variable region CDR3 comprising amino acids having the sequence set forth in SEQ ID NO: 297;
(xxviii) a light chain variable region CDR1 comprising amino acids having the sequence set forth in SEQ ID NO: 306; a light chain variable region CDR2 comprising amino acids having the sequence set forth in SEQ ID NO:307; and a light chain variable region CDR3 comprising amino acids having the sequence set forth in SEQ ID NO: 308;
(xxix) a light chain variable region CDR1 comprising amino acids having the sequence set forth in SEQ ID NO: 319; a light chain variable region CDR2 comprising amino acids having the sequence set forth in SEQ ID NO:320; and a light chain variable region CDR3 comprising amino acids having the sequence set forth in SEQ ID NO: 321;
(xxx) a light chain variable region CDR1 comprising amino acids having the sequence set forth in SEQ ID NO: 331; a light chain variable region CDR2 comprising amino acids having the sequence set forth in SEQ ID NO:332; and a light chain variable region CDR3 comprising amino acids having the sequence set forth in SEQ ID NO: 333;
(xxxi) a light chain variable region CDR1 comprising amino acids having the sequence set forth in SEQ ID NO: 343; a light chain variable region CDR2 comprising amino acids having the sequence set forth in SEQ ID NO:344; and a light chain variable region CDR3 comprising amino acids having the sequence set forth in SEQ ID NO: 345; or
(xxxii) a light chain variable region CDR1 comprising amino acids having the sequence set forth in SEQ ID NO: 355; a light chain variable region CDR2 comprising amino acids having the sequence set forth in SEQ ID NO:356; and a light chain variable region CDR3 comprising amino acids having the sequence set forth in SEQ ID NO: 357,

wherein the antibody or antigen-binding portion thereof specifically binds to GPRC5D.

The presently disclosed subject matter also provides an isolated antibody, or an antigen-binding portion thereof, comprising:
(i) a heavy chain variable region CDR1 comprising amino acids having the sequence set forth in SEQ ID NO: 124; a heavy chain variable region CDR2 comprising amino acids having the sequence set forth in SEQ ID NO: 125; a heavy chain variable region CDR3 comprising amino acids having the sequence set forth in SEQ ID NO: 126; a light chain variable region CDR1 comprising amino acids having the sequence set forth in SEQ ID NO: 127; a light chain variable region CDR2 comprising amino acids having the sequence set forth in SEQ ID NO:128; and a light chain variable region CDR3 comprising amino acids having the sequence set forth in SEQ ID NO: 129;
(ii) a heavy chain variable region CDR1 comprising amino acids having the sequence set forth in SEQ ID NO: 130; a heavy chain variable region CDR2 comprising amino acids having the sequence set forth in SEQ ID NO: 131; a heavy chain variable region CDR3 comprising amino acids having the sequence set forth in SEQ ID NO: 132; a light chain variable region CDR1 comprising amino acids having the sequence set forth in SEQ ID NO: 133; a light chain variable region CDR2 comprising amino acids having the sequence set forth in SEQ ID NO:134; and a light chain variable region CDR3 comprising amino acids having the sequence set forth in SEQ ID NO: 135;
(iii) a heavy chain variable region CDR1 comprising amino acids having the sequence set forth in SEQ ID NO: 136; a heavy chain variable region CDR2 comprising amino acids having the sequence set forth in SEQ ID NO: 137; a heavy chain variable region CDR3 comprising amino acids having the sequence set forth in SEQ ID NO: 138; a light chain variable region CDR1 comprising amino acids having the sequence set forth in SEQ ID NO: 139; a light chain variable region CDR2 comprising amino acids having the sequence set forth in SEQ ID NO:140; and a light chain variable region CDR3 comprising amino acids having the sequence set forth in SEQ ID NO: 141;
(iv) a heavy chain variable region CDR1 comprising amino acids having the sequence set forth in SEQ ID NO: 142; a heavy chain variable region CDR2 comprising amino acids having the sequence set forth in SEQ ID NO: 143; a heavy chain variable region CDR3 comprising amino acids having the sequence set forth in SEQ ID NO: 144; a light chain variable region CDR1 comprising amino acids having the sequence set forth in SEQ ID NO: 145; a light chain variable region CDR2 comprising amino acids having the sequence set forth in SEQ ID NO:146; and a light chain variable region CDR3 comprising amino acids having the sequence set forth in SEQ ID NO: 147;
(v) a heavy chain variable region CDR1 comprising amino acids having the sequence set forth in SEQ ID NO: 148; a heavy chain variable region CDR2 comprising amino acids having the sequence set forth in SEQ ID NO: 149; a heavy chain variable region CDR3 comprising amino acids having the sequence set forth in SEQ ID NO: 150; a light chain variable region CDR1 comprising amino acids having the sequence set forth in SEQ ID NO: 151; a light chain variable region CDR2 comprising amino acids having the sequence set forth in SEQ ID NO:152; and a light chain variable region CDR3 comprising amino acids having the sequence set forth in SEQ ID NO: 153;
(vi) a heavy chain variable region CDR1 comprising amino acids having the sequence set forth in SEQ ID NO: 154; a heavy chain variable region CDR2 comprising amino acids having the sequence set forth in SEQ ID NO: 155; a heavy chain variable region CDR3 comprising amino acids having the sequence set forth in SEQ ID NO: 156; a light chain variable region CDR1 comprising amino acids having the sequence set forth in SEQ ID NO: 157; a light chain variable region CDR2 comprising amino acids having the sequence set forth in SEQ ID NO:158; and a light chain variable region CDR3 comprising amino acids having the sequence set forth in SEQ ID NO: 159;
(vii) a heavy chain variable region CDR1 comprising amino acids having the sequence set forth in SEQ ID NO: 160; a heavy chain variable region CDR2 comprising amino acids having the sequence set forth in SEQ ID NO: 161; a heavy chain variable region CDR3 comprising amino acids having the sequence set forth in SEQ ID NO: 162; a light chain variable region CDR1 comprising amino acids having the sequence set forth in SEQ ID NO: 163; a light chain variable region CDR2 comprising amino acids having the sequence set forth in SEQ ID NO:164; and a light chain variable region CDR3 comprising amino acids having the sequence set forth in SEQ ID NO: 165;
(viii) a heavy chain variable region CDR1 comprising amino acids having the sequence set forth in SEQ ID NO: 166; a heavy chain variable region CDR2 comprising amino acids having the sequence set forth in SEQ ID NO: 167; a heavy chain variable region CDR3 comprising amino acids having the sequence set forth in SEQ ID NO: 168; a light chain variable region CDR1 comprising amino acids having the sequence set forth in SEQ ID NO: 169; a light chain variable region CDR2 comprising amino acids having the sequence set forth in SEQ ID NO:170; and a light chain variable region CDR3 comprising amino acids having the sequence set forth in SEQ ID NO: 171;
(ix) a heavy chain variable region CDR1 comprising amino acids having the sequence set forth in SEQ ID NO: 172; a heavy chain variable region CDR2 comprising amino acids having the sequence set forth in SEQ ID NO: 173; a heavy chain variable region CDR3 comprising amino acids having the sequence set forth in SEQ ID NO: 174; a light chain variable region CDR1 comprising amino acids having the sequence set forth in SEQ ID NO: 175; a light chain variable region CDR2 comprising amino acids having the sequence set forth in SEQ ID NO:176; and a light chain variable region CDR3 comprising amino acids having the sequence set forth in SEQ ID NO: 177;
(x) a heavy chain variable region CDR1 comprising amino acids having the sequence set forth in SEQ ID NO: 178; a heavy chain variable region CDR2 comprising amino acids having the sequence set forth in SEQ ID NO: 179; a heavy chain variable region CDR3 comprising amino acids having the sequence set forth in SEQ ID NO: 180; a light chain variable region CDR1 comprising amino acids having the sequence set forth in SEQ ID NO: 181; a light chain variable region CDR2 comprising amino acids having the sequence set forth in SEQ ID NO:182; and a light chain variable region CDR3 comprising amino acids having the sequence set forth in SEQ ID NO: 183;
(xi) a heavy chain variable region CDR1 comprising amino acids having the sequence set forth in SEQ ID NO: 184; a heavy chain variable region CDR2 comprising amino acids having the sequence set forth in SEQ ID NO: 185; a heavy chain variable region CDR3 comprising amino acids having the sequence set forth in SEQ ID NO: 186; a light chain variable region CDR1 comprising amino acids having the sequence set forth in SEQ ID NO: 187; a light chain variable region CDR2 comprising amino acids having the sequence set forth in SEQ ID NO:188; and a light chain variable region CDR3 comprising amino acids having the sequence set forth in SEQ ID NO: 189;
(xii) a heavy chain variable region CDR1 comprising amino acids having the sequence set forth in SEQ ID NO: 190; a heavy chain variable region CDR2 comprising amino acids having the sequence set forth in SEQ ID NO: 191; a heavy chain variable region CDR3 comprising amino acids having the sequence set forth in SEQ ID NO: 192; a light chain variable region CDR1 comprising amino acids having the sequence set forth in SEQ ID NO: 193; a light chain variable region CDR2 comprising amino acids having the sequence set forth in SEQ ID NO:194; and a light chain variable region CDR3 comprising amino acids having the sequence set forth in SEQ ID NO: 195;
(xiii) a heavy chain variable region CDR1 comprising amino acids having the sequence set forth in SEQ ID NO: 196; a heavy chain variable region CDR2 comprising amino acids having the sequence set forth in SEQ ID NO: 197; a heavy chain variable region CDR3 comprising amino acids having the sequence set forth in SEQ ID NO: 198; a light chain variable region CDR1 comprising amino acids having the sequence set forth in SEQ ID NO: 199; a light chain variable region CDR2 comprising amino acids having the sequence set forth in SEQ ID NO:200; and a light chain variable region CDR3 comprising amino acids having the sequence set forth in SEQ ID NO: 201;
(xiv) a heavy chain variable region CDR1 comprising amino acids having the sequence set forth in SEQ ID NO: 202; a heavy chain variable region CDR2 comprising amino acids having the sequence set forth in SEQ ID NO: 203; a heavy chain variable region CDR3 comprising amino acids having the sequence set forth in SEQ ID NO: 204; a light chain variable region CDR1 comprising amino acids having the sequence set forth in SEQ ID NO: 205; a light chain variable region CDR2 comprising amino acids having the sequence set forth in SEQ ID NO:206; and a light chain variable region CDR3 comprising amino acids having the sequence set forth in SEQ ID NO: 207;
(xv) a heavy chain variable region CDR1 comprising amino acids having the sequence set forth in SEQ ID NO: 208; a heavy chain variable region CDR2 comprising amino acids having the sequence set forth in SEQ ID NO: 209; a heavy chain variable region CDR3 comprising amino acids having the sequence set forth in SEQ ID NO: 210; a light chain variable region CDR1 comprising amino acids having the sequence set forth in SEQ ID NO: 211; a light chain variable region CDR2 comprising amino acids having the sequence set forth in SEQ ID NO:212; and a light chain variable region CDR3 comprising amino acids having the sequence set forth in SEQ ID NO: 213;
(xvi) a heavy chain variable region CDR1 comprising amino acids having the sequence set forth in SEQ ID NO: 214; a heavy chain variable region CDR2 comprising amino acids having the sequence set forth in SEQ ID NO: 215; a heavy chain variable region CDR3 comprising amino acids having the sequence set forth in SEQ ID NO: 216; a light chain variable region CDR1 comprising amino acids having the sequence set forth in SEQ ID NO: 217; a light chain variable region CDR2 comprising amino acids having the sequence set forth in SEQ ID NO:218; and a light chain variable region CDR3 comprising amino acids having the sequence set forth in SEQ ID NO: 219;
(xvii) a heavy chain variable region CDR1 comprising amino acids having the sequence set forth in SEQ ID NO: 220; a heavy chain variable region CDR2 comprising amino acids having the sequence set forth in SEQ ID NO: 221; a heavy chain variable region CDR3 comprising amino acids having the sequence set forth in SEQ ID NO: 222; a light chain variable region CDR1 comprising amino acids having the sequence set forth in SEQ ID NO: 223; a light chain variable region CDR2 comprising amino acids having the sequence set forth in SEQ ID NO:224; and a light chain variable region CDR3 comprising amino acids having the sequence set forth in SEQ ID NO: 225;
(xviii) a heavy chain variable region CDR1 comprising amino acids having the sequence set forth in SEQ ID NO: 226; a heavy chain variable region CDR2 comprising amino acids having the sequence set forth in SEQ ID NO: 227; a heavy chain variable region CDR3 comprising amino acids having the sequence set forth in SEQ ID NO: 228; a light chain variable region CDR1 comprising amino acids having the sequence set forth in SEQ ID NO: 229; a light chain variable region CDR2 comprising amino acids having the sequence set forth in SEQ ID NO:230; and a light chain variable region CDR3 comprising amino acids having the sequence set forth in SEQ ID NO: 231;
(xix) a heavy chain variable region CDR1 comprising amino acids having the sequence set forth in SEQ ID NO: 232; a heavy chain variable region CDR2 comprising amino acids having the sequence set forth in SEQ ID NO: 233; a heavy chain variable region CDR3 comprising amino acids having the sequence set forth in SEQ ID NO: 234; a light chain variable region CDR1 comprising amino acids having the sequence set forth in SEQ ID NO: 235; a light chain variable region CDR2 comprising amino acids having the sequence set forth in SEQ ID NO:236; and a light chain variable region CDR3 comprising amino acids having the sequence set forth in SEQ ID NO: 237;
(xx) a heavy chain variable region CDR1 comprising amino acids having the sequence set forth in SEQ ID NO: 238; a heavy chain variable region CDR2 comprising amino acids having the sequence set forth in SEQ ID NO: 239; a heavy chain variable region CDR3 comprising amino acids having the sequence set forth in SEQ ID NO: 240; a light chain variable region CDR1 comprising amino acids having the sequence set forth in SEQ ID NO: 241; a light chain variable region CDR2 comprising amino acids having the sequence set forth in SEQ ID NO:242; and a light chain variable region CDR3 comprising amino acids having the sequence set forth in SEQ ID NO: 243;
(xxi) a heavy chain variable region CDR1 comprising amino acids having the sequence set forth in SEQ ID NO: 244; a heavy chain variable region CDR2 comprising amino acids having the sequence set forth in SEQ ID NO: 245; a heavy chain variable region CDR3 comprising amino acids having the sequence set forth in SEQ ID NO: 246; a light chain variable region CDR1 comprising amino acids having the sequence set forth in SEQ ID NO: 247; a light chain variable region CDR2 comprising amino acids having the sequence set forth in SEQ ID NO:248; and a light chain variable region CDR3 comprising amino acids having the sequence set forth in SEQ ID NO: 249;
(xxii) a heavy chain variable region CDR1 comprising amino acids having the sequence set forth in SEQ ID NO: 250; a heavy chain variable region CDR2 comprising amino acids having the sequence set forth in SEQ ID NO: 251; a heavy chain variable region CDR3 comprising amino acids having the sequence set forth in SEQ ID NO: 252; a light chain variable region CDR1 comprising amino acids having the sequence set forth in SEQ ID NO: 253; a light chain variable region CDR2 comprising amino acids having the sequence set forth in SEQ ID NO:254; and a light chain variable region CDR3 comprising amino acids having the sequence set forth in SEQ ID NO: 255;
(xxiii) a heavy chain variable region CDR1 comprising amino acids having the sequence set forth in SEQ ID NO: 256; a heavy chain variable region CDR2 comprising amino acids having the sequence set forth in SEQ ID NO: 257; a heavy chain variable region CDR3 comprising amino acids having the sequence set forth in SEQ ID NO: 258; a light chain variable region CDR1 comprising amino acids having the sequence set forth in SEQ ID NO: 259; a light chain variable region CDR2 comprising amino acids having the sequence set forth in SEQ ID NO:260; and a light chain variable region CDR3 comprising amino acids having the sequence set forth in SEQ ID NO: 261;
(xxiv) a heavy chain variable region CDR1 comprising amino acids having the sequence set forth in SEQ ID NO: 262; a heavy chain variable region CDR2 comprising amino acids having the sequence set forth in SEQ ID NO: 263; a heavy chain variable region CDR3 comprising amino acids having the sequence set forth in SEQ ID NO: 264; a light chain variable region CDR1 comprising amino acids having the sequence set forth in SEQ ID NO: 265; a light chain variable region CDR2 comprising amino acids having the sequence set forth in SEQ ID NO:266; and a light chain variable region CDR3 comprising amino acids having the sequence set forth in SEQ ID NO: 267;
(xxv) a heavy chain variable region CDR1 comprising amino acids having the sequence set forth in SEQ ID NO: 268; a heavy chain variable region CDR2 comprising amino acids having the sequence set forth in SEQ ID NO: 269; a heavy chain variable region CDR3 comprising amino acids having the sequence set forth in SEQ ID NO: 270; a light chain variable region CDR1 comprising amino acids having the sequence set forth in SEQ ID NO: 271; a light chain variable region CDR2 comprising amino acids having the sequence set forth in SEQ ID NO:272; and (vi) a light chain variable region CDR3 comprising amino acids having the sequence set forth in SEQ ID NO: 273;
(xxvi) a heavy chain variable region CDR1 comprising amino acids having the sequence set forth in SEQ ID NO: 280; a heavy chain variable region CDR2 comprising amino acids having the sequence set forth in SEQ ID NO: 281; a heavy chain variable region CDR3 comprising amino acids having the sequence set forth in SEQ ID NO: 282; a light chain variable region CDR1 comprising amino acids having the sequence set forth in SEQ ID NO: 283; a light chain variable region CDR2 comprising amino acids having the sequence set forth in SEQ ID NO:284; and a light chain variable region CDR3 comprising amino acids having the sequence set forth in SEQ ID NO: 285;
(xxvii) a heavy chain variable region CDR1 comprising amino acids having the sequence set forth in SEQ ID NO: 292; a heavy chain variable region CDR2 comprising amino acids having the sequence set forth in SEQ ID NO: 293; a heavy chain variable region CDR3 comprising amino acids having the sequence set forth in SEQ ID NO: 294; a light chain variable region CDR1 comprising amino acids having the sequence set forth in SEQ ID NO: 295; a light chain variable region CDR2 comprising amino acids having the sequence set forth in SEQ ID NO:296; and a light chain variable region CDR3 comprising amino acids having the sequence set forth in SEQ ID NO: 297;
(xxviii) a heavy chain variable region CDR1 comprising amino acids having the sequence set forth in SEQ ID NO: 303; a heavy chain variable region CDR2 comprising amino acids having the sequence set forth in SEQ ID NO: 304; a heavy chain variable region CDR3 comprising amino acids having the sequence set forth in SEQ ID NO: 305; a light chain variable region CDR1 comprising amino acids having the sequence set forth in SEQ ID NO: 306; a light chain variable region CDR2 comprising amino acids having the sequence set forth in SEQ ID NO: 307; and a light chain variable region CDR3 comprising amino acids having the sequence set forth in SEQ ID NO: 308;
(xxix) a heavy chain variable region CDR1 comprising amino acids having the sequence set forth in SEQ ID NO: 316; a heavy chain variable region CDR2 comprising amino acids having the sequence set forth in SEQ ID NO: 317; a heavy chain variable region CDR3 comprising amino acids having the sequence set forth in SEQ ID NO: 318; a light chain variable region CDR1 comprising amino acids having the sequence set forth in SEQ ID NO: 319; a light chain variable region CDR2 comprising amino acids having the sequence set forth in SEQ ID NO: 320; and a light chain variable region CDR3 comprising amino acids having the sequence set forth in SEQ ID NO: 321;
(xxx) a heavy chain variable region CDR1 comprising amino acids having the sequence set forth in SEQ ID NO: 328; a heavy chain variable region CDR2 comprising amino acids having the sequence set forth in SEQ ID NO: 329; a heavy chain variable region CDR3 comprising amino acids having the sequence set forth in SEQ ID NO: 330; a light chain variable region CDR1 comprising amino acids having the sequence set forth in SEQ ID NO: 331; a light chain variable region CDR2 comprising amino acids having the sequence set forth in SEQ ID NO: 332; and a light chain variable region CDR3 comprising amino acids having the sequence set forth in SEQ ID NO: 333;
(xxxi) a heavy chain variable region CDR1 comprising amino acids having the sequence set forth in SEQ ID NO: 340; a heavy chain variable region CDR2 comprising amino acids having the sequence set forth in SEQ ID NO: 341; a heavy chain variable region CDR3 comprising amino acids having the sequence set forth in SEQ ID NO: 342; a light chain variable region CDR1 comprising amino acids having the sequence set forth in SEQ ID NO: 343; a light chain variable region CDR2 comprising amino acids having the sequence set forth in SEQ ID NO: 344; and a light chain variable region CDR3 comprising amino acids having the sequence set forth in SEQ ID NO: 345; or
(xxxii) a heavy chain variable region CDR1 comprising amino acids having the sequence set forth in SEQ ID NO: 352; a heavy chain variable region CDR2 comprising amino acids having the sequence set forth in SEQ ID NO: 353; a heavy chain variable region CDR3 comprising amino acids having the sequence set forth in SEQ ID NO: 354; a light chain variable region CDR1 comprising amino acids having the sequence set forth in SEQ ID NO: 355; a light chain variable region CDR2 comprising amino acids having the sequence set forth in SEQ ID NO: 356; and a light chain variable region CDR3 comprising amino acids having the sequence set forth in SEQ ID NO: 357.

Furthermore, the presently disclosed subject matter provides an isolated antibody, or an antigen-binding portion thereof, which cross-competes for binding to human GPRC5D with any of the disclosed antibodies. In certain embodiments, the presently disclosed subject matter provides an isolated antibody, or an antigen-binding portion thereof, which binds to the same epitope on human GPRC5D with an isolated antibody, or an antigen-binding portion thereof of any of the antibodies disclosed herein.

In certain embodiments, the presently disclosed subject matter provides an isolated antibody, or an antigen-binding portion thereof, which cross-competes for binding to human GPRC5D with a reference antibody or reference antigen-binding portion thereof comprising: (i) a heavy chain variable region comprising amino acids having a sequence set forth in SEQ ID NO:1, and a light chain variable region comprising amino acids having a sequence set forth in SEQ ID NO:2; (ii) a heavy chain variable region comprising amino acids having a sequence set forth in SEQ ID NO:5, and a light chain variable region comprising amino acids having a sequence set forth in SEQ ID NO:6; (iii) a heavy chain variable region comprising amino acids having a sequence set forth in SEQ ID NO:9, and a light chain variable region comprising amino acids having a sequence set forth in SEQ ID NO:10; (iv) a heavy chain variable region comprising amino acids having a sequence set forth in SEQ ID NO:13, and a light chain variable region comprising amino acids having a sequence set forth in SEQ ID NO:14; (v) a heavy chain variable region comprising amino acids having a sequence set forth in SEQ ID NO:17, and a light chain variable region comprising amino acids having a sequence set forth in SEQ ID NO:18; (vi) a heavy chain variable region comprising amino acids having a sequence set forth in SEQ ID NO:21, and a light chain variable region comprising amino acids having a sequence set forth in SEQ ID NO:22; (vii) a heavy chain variable region comprising amino acids having a sequence set forth in SEQ ID NO:25, and a light chain variable region comprising amino acids having a sequence set forth in SEQ ID NO:26; (viii) a heavy chain variable region comprising amino acids having a sequence set forth in SEQ ID NO:29, and a light chain variable region comprising amino acids having a sequence set forth in SEQ ID NO:30; (ix) a heavy chain variable region comprising amino acids having a sequence set forth in SEQ ID NO:33, and a light chain variable region comprising amino acids having a sequence set forth in SEQ ID NO:34; (x) a heavy chain variable region comprising amino acids having a sequence set forth in SEQ ID NO:37, and a light chain variable region comprising amino acids having a sequence set forth in SEQ ID NO:38; (xi) a heavy chain variable region comprising amino acids having a sequence set forth in SEQ ID NO:41, and a light chain variable region comprising amino acids having a sequence set forth in SEQ ID NO:42; (xii) a heavy chain variable region comprising amino acids having a sequence set forth in SEQ ID NO:45, and a light chain variable region comprising amino acids having a sequence set forth in SEQ ID NO:46; (xiii) a heavy chain variable region comprising amino acids having a sequence set forth in SEQ ID NO:49, and a light chain variable region comprising amino acids having a sequence set forth in SEQ ID NO:50; (xiv) a heavy chain variable region comprising amino acids having a sequence set forth in SEQ ID NO:53, and a light chain variable region comprising amino acids having a sequence set forth in SEQ ID NO:54; (xv) a heavy chain variable region comprising amino acids having a sequence set forth in SEQ ID NO:57, and a light chain variable region comprising amino acids having a sequence set forth in SEQ ID NO:58; (xvi) a heavy chain variable region comprising amino acids having a sequence set forth in SEQ ID NO:61, and a light chain variable region comprising amino acids having a sequence set forth in SEQ ID NO:62; (xvii) a heavy chain variable region comprising amino acids having a sequence set forth in SEQ ID NO:65, and a light chain variable region comprising amino acids having a sequence set forth in SEQ ID NO:66; (xviii) a heavy chain variable region comprising amino acids having a sequence set forth in SEQ ID NO:69, and a light chain variable region comprising amino acids having a sequence set forth in SEQ ID NO:70; (xix) a heavy chain variable region comprising amino acids having a sequence set forth in SEQ ID NO:73, and a light chain variable region comprising amino acids having a sequence set forth in SEQ ID NO:74; (xx) a heavy chain variable region comprising amino acids having a sequence set forth in SEQ ID NO:77, and a light chain variable region comprising amino acids having a sequence set forth in SEQ ID NO:78; (xxi) a heavy chain variable region comprising amino acids having a sequence set forth in SEQ ID NO:81, and a light chain variable region comprising amino acids having a sequence set forth in SEQ ID NO:82; (xxii) a heavy chain variable region comprising amino acids having a sequence set forth in SEQ ID NO:85, and a light chain variable region comprising amino acids having a sequence set forth in SEQ ID NO:86; (xxiii) a heavy chain variable region comprising amino acids having a sequence set forth in SEQ ID NO:89, and a light chain variable region comprising amino acids having a sequence set forth in SEQ ID NO:90; (xxiv) a heavy chain variable region comprising amino acids having a sequence set forth in SEQ ID NO:93, and a light chain variable region comprising amino acids having a sequence set forth in SEQ ID NO:94; (xxv) a heavy chain variable region comprising amino acids having a sequence set forth in SEQ ID NO:274, and a light chain variable region comprising amino acids having a sequence set forth in SEQ ID NO:275; (xxvi) a heavy chain variable region comprising amino acids having a sequence set forth in SEQ ID NO:286, and a light chain variable region comprising amino acids having a sequence set forth in SEQ ID NO:287; (xxvii) a heavy chain variable region comprising amino acids having a sequence set forth in SEQ ID NO:298, and a light chain variable region comprising amino acids having a sequence set forth in SEQ ID NO:299; (xviii) a heavy chain variable region comprising amino acids having a sequence set forth in SEQ ID NO:310, and a light chain variable region comprising amino acids having a sequence set forth in SEQ ID NO:311; (xxi) a heavy chain variable region comprising amino acids having a sequence set forth in SEQ ID NO:322, and a light chain variable region comprising amino acids having a sequence set forth in SEQ ID NO:323; (xx) a heavy chain variable region comprising amino acids having a sequence set forth in SEQ ID NO:334, and a light chain variable region comprising amino acids having a sequence set forth in SEQ ID NO:335; (xxi) a heavy chain variable region comprising amino acids having a sequence set forth in SEQ ID NO:346, and a light chain variable region comprising amino acids having a sequence set forth in SEQ ID NO:347; or (xxii) a heavy chain variable region comprising amino acids having a sequence set forth in SEQ ID NO:358, and a light chain variable region comprising amino acids having a sequence set forth in SEQ ID NO:359.

In addition, the presently disclosed subject matter provides an isolated antibody, or an antigen-binding portion thereof, which binds to the same epitope on human GPRC5D as a reference antibody or reference antigen-binding portion thereof comprising: (i) a heavy chain variable region comprising amino acids having a sequence set forth in SEQ ID NO:1, and a light chain variable region comprising amino acids having a sequence set forth in SEQ ID NO:2; (ii) a heavy chain variable region comprising amino acids having a sequence set forth in SEQ ID NO:5, and a light chain variable region comprising amino acids having a sequence set forth in SEQ ID NO:6; (iii) a heavy chain variable region comprising amino acids having a sequence set forth in SEQ ID NO:9, and a light chain variable region comprising amino acids having a sequence set forth in SEQ ID NO:10; (iv) a heavy chain variable region comprising amino acids having a sequence set forth in SEQ ID NO:13, and a light chain variable region comprising amino acids having a sequence set forth in SEQ ID NO:14; (v) a heavy chain variable region comprising amino acids having a sequence set forth in SEQ ID NO:17, and a light chain variable region comprising amino acids having a sequence set forth in SEQ ID NO:18; (vi) a heavy chain variable region comprising amino acids having a sequence set forth in SEQ ID NO:21, and a light chain variable region comprising amino acids having a sequence set forth in SEQ ID NO:22; (vii) a heavy chain variable region comprising amino acids having a sequence set forth in SEQ ID NO:25, and a light chain variable region comprising amino acids having a sequence set forth in SEQ ID NO:26; (viii) a heavy chain variable region comprising amino acids having a sequence set forth in SEQ ID NO:29, and a light chain variable region comprising amino acids having a sequence set forth in SEQ ID NO:30; (ix) a heavy chain variable region comprising amino acids having a sequence set forth in SEQ ID NO:33, and a light chain variable region comprising amino acids having a sequence set forth in SEQ ID NO:34; (x) a heavy chain variable region comprising amino acids having a sequence set forth in SEQ ID NO:37, and a light chain variable region comprising amino acids having a sequence set forth in SEQ ID NO:38; (xi) a heavy chain variable region comprising amino acids having a sequence set forth in SEQ ID NO:41, and a light chain variable region comprising amino acids having a sequence set forth in SEQ ID NO:42; (xii) a heavy chain variable region comprising amino acids having a sequence set forth in SEQ ID NO:45, and a light chain variable region comprising amino acids having a sequence set forth in SEQ ID NO:46; (xiii) a heavy chain variable region comprising amino acids having a sequence set forth in SEQ ID NO:49, and a light chain variable region comprising amino acids having a sequence set forth in SEQ ID NO:50; (xiv) a heavy chain variable region comprising amino acids having a sequence set forth in SEQ ID NO:53, and a light chain variable region comprising amino acids having a sequence set forth in SEQ ID NO:54; (xv) a heavy chain variable region comprising amino acids having a sequence set forth in SEQ ID NO:57, and a light chain variable region comprising amino acids having a sequence set forth in SEQ ID NO:58; (xvi) a heavy chain variable region comprising amino acids having a sequence set forth in SEQ ID NO:61, and a light chain variable region comprising amino acids having a sequence set forth in SEQ ID NO:62; (xvii) a heavy chain variable region comprising amino acids having a sequence set forth in SEQ ID NO:65, and a light chain variable region comprising amino acids having a sequence set forth in SEQ ID NO:66; (xviii) a heavy chain variable region comprising amino acids having a sequence set forth in SEQ ID NO:69, and a light chain variable region comprising amino acids having a sequence set forth in SEQ ID NO:70; (xix) a heavy chain variable region comprising amino acids having a sequence set forth in SEQ ID NO:73, and a light chain variable region comprising amino acids having a sequence set forth in SEQ ID NO:74; (xx) a heavy chain variable region comprising amino acids having a sequence set forth in SEQ ID NO:77, and a light chain variable region comprising amino acids having a sequence set forth in SEQ ID NO:78; (xxi) a heavy chain variable region comprising amino acids having a sequence set forth in SEQ ID NO:81, and a light chain variable region comprising amino acids having a sequence set forth in SEQ ID NO:82; (xxii) a heavy chain variable region comprising amino acids having a sequence set forth in SEQ ID NO:85, and a light chain variable region comprising amino acids having a sequence set forth in SEQ ID NO:86; (xxiii) a heavy chain variable region comprising amino acids having a sequence set forth in SEQ ID NO:89, and a light chain variable region comprising amino acids having a sequence set forth in SEQ ID NO:90; (xxiv) a heavy chain variable region comprising amino acids having a sequence set forth in SEQ ID NO:93, and a light chain variable region comprising amino acids having a sequence set forth in SEQ ID NO:94; (xxv) a heavy chain variable region comprising amino acids having a sequence set forth in SEQ ID NO:274, and a light chain variable region comprising amino acids having a sequence set forth in SEQ ID NO:275; (xxvi) a heavy chain variable region comprising amino acids having a sequence set forth in SEQ ID NO:286, and a light chain variable region comprising amino acids having a sequence set forth in SEQ ID NO:287; (xxvii) a heavy chain variable region comprising amino acids having a sequence set forth in SEQ ID NO:298, and a light chain variable region comprising amino acids having a sequence set forth in SEQ ID NO:299; (xviii) a heavy chain variable region comprising amino acids having a sequence set forth in SEQ ID NO:310, and a light chain variable region comprising amino acids having a sequence set forth in SEQ ID NO:311; (xxi) a heavy chain variable region comprising amino acids having a sequence set forth in SEQ ID NO:322, and a light chain variable region comprising amino acids having a sequence set forth in SEQ ID NO:323; (xx) a heavy chain variable region comprising amino acids having a sequence set forth in SEQ ID NO:334, and a light chain variable region comprising amino acids having a sequence set forth in SEQ ID NO:335; (xxi) a heavy chain variable region comprising amino acids having a sequence set forth in SEQ ID NO:346, and a light chain variable region comprising amino acids having a sequence set forth in SEQ ID NO:347; or (xxii) a heavy chain variable region comprising amino acids having a sequence set forth in SEQ ID NO:358, and a light chain variable region comprising amino acids having a sequence set forth in SEQ ID NO:359.

In certain embodiments, the antibodies of the present disclosure bind to GPRC5D comprising the amino acid sequence set forth in SEQ ID NO:97. In certain embodiments, the antibodies of the present disclosure binds to human GPRC5D with a binding affinity (K_{d}) of from about 1×10⁻⁹ M to about 1×10⁻⁸ M.

In certain embodiments, the antibodies of the present disclosure binds to one, two, three or four epitope region selected from the group consisting of an epitope region in N-terminal region comprising amino acids 1-27 of SEQ ID NO:97, an epitope region in ECL1 region comprising amino acids 85-93 of SEQ ID NO:97, an epitope region in ECL2 region comprising amino acids 145-167 of SEQ ID NO:97, and an epitope region in ECL3 region comprising amino acids 226-239 of SEQ ID NO:97. In certain embodiments, the antibodies of the present disclosure bind to an epitope region comprising amino acids 16-23 of SEQ ID NO:97. In certain embodiments, the antibodies of the present disclosure bind to an epitope region comprising amino acids 15-23 of SEQ ID NO:97. In certain embodiments, the antibodies of the present disclosure bind to an epitope region comprising amino acids 16-25 of SEQ ID NO:97. In certain embodiments, the antibodies of the present disclosure bind to an epitope region comprising amino acids 10-17 of SEQ ID NO:97. In certain embodiments, the antibodies of the present disclosure bind to an epitope region comprising amino acids 5-17 of SEQ ID NO:97. In certain embodiments, the antibodies of the present disclosure bind to an epitope region comprising amino acids 85-95 of SEQ ID NO:97. In certain embodiments, the antibodies of the present disclosure bind to an epitope region comprising amino acids 157-164 of SEQ ID NO:97. In certain embodiments, the antibodies of the present disclosure bind to an epitope region comprising amino acids 157-167 of SEQ ID NO:97. In certain embodiments, the antibodies of the present disclosure bind to an epitope region comprising amino acids 230-237 of SEQ ID NO:97. In certain embodiments, the antibodies of the present disclosure bind to an epitope region comprising amino acids 229-237 of SEQ ID NO:97. In certain embodiments, the antibodies of the present disclosure bind to an epitope region comprising amino acids 230-243 of SEQ ID NO:97. In certain embodiments, the antibodies of the present disclosure bind to an epitope region comprising amino acids 227-237 of SEQ ID NO:97.

The presently disclosed subject matter also provides an isolated antibody, or antigen-binding fragment thereof, comprising an amino acid sequence selected from the group consisting of SEQ ID NOS: 100-123, 276, 288, 300, 312, 324, 336, 348 and 360.

In certain embodiments, the antibody or antigen-binding fragment thereof comprises a human variable region framework region. In certain embodiments, the antibody or antigen-binding fragment thereof is fully human or an antigen-binding fragment thereof. In certain embodiments, the antibody or antigen-binding fragment thereof is a chimeric antibody or an antigen-binding fragment thereof. In certain embodiments, the antibody or antigen-binding portion thereof is a humanized antibody or an antigen-binding fragment thereof. In certain embodiments, the antigen-binding fragment of the antibody is an Fab, Fab′, F(ab′)₂, Fv or single chain Fv (scFv).

The presently disclosed subject matter also provides a composition comprising the antibody or antigen-binding fragment thereof disclosed herein, and a pharmaceutically acceptable carrier.

In addition, the presently disclosed subject matter provides an immunoconjugate comprising the antibody or antigen-binding fragment thereof disclosed herein, linked to a therapeutic agent. In certain embodiments, the therapeutic agent is a drug, cytotoxin, or a radioactive isotope. The presently disclosed subject matter also provides a composition comprising such immunoconjugate and a pharmaceutically acceptable carrier.

Furthermore, the presently disclosed subject matter provides a bispecific molecule comprising the antibody or antigen-binding fragment thereof disclosed herein, linked to a second functional moiety. In certain embodiments, the second functional moiety has a different binding specificity than the antibody or antigen binding fragment thereof. In certain embodiments, the second functional moiety has a binding specificity for an immune cell. In certain embodiments, the second functional moiety has a binding specificity for CD3.

The presently disclosed subject matter also provides a composition comprising such bispecific molecule and a pharmaceutically acceptable carrier.

In addition, the presently disclosed subject matter provides an isolated nucleic acid that encodes the antibody or antigen-binding fragment thereof disclosed herein, an expression vector comprising such nucleic acid molecule, and a host cell comprising such expression vector.

Furthermore, the presently disclosed subject matter provides a method for detecting GPRC5D in a whole cell or tissue. In certain embodiments, the method comprises: contacting a cell or tissue with the antibody or antigen-binding fragment thereof disclosed herein, wherein said antibody or antigen-binding fragment thereof comprises a detectable label; and determining the amount of the labeled antibody or antigen-binding fragment thereof bound to said cell or tissue by measuring the amount of detectable label associated with said cell or tissue, wherein the amount of bound antibody or antigen-binding fragment thereof indicates the amount of GPRC5D in said cell or tissue.

Furthermore, the presently disclosed subject matter provides a method of treating a tumor in a subject. In certain embodiments, the method comprises: administering an effective amount of the antibody or antigen-binding fragment thereof disclosed herein to the subject, thereby inducing death of a tumor cell in the subject. In certain embodiments, the method reduces the number of the tumor cells. In certain embodiments, the method reduces the tumor size. In certain embodiments, the method eradicates the tumor in the subject. In certain embodiments, the subject is a human.

In addition, the presently disclosed subject matter provides use of the antibody or antigen-binding fragment disclosed herein for the treatment of a tumor, and the antibody or antigen-binding fragment thereof disclosed herein for use in treating a tumor in a subject.

Furthermore, the presently disclosed subject matter provides a kit for treating a tumor, comprising the antibody or antigen-binding fragment thereof disclosed herein. In certain embodiments, the kit further comprises written instructions for using the antibody or antigen-binding fragment thereof for treating a subject having a tumor.

In certain embodiments, the tumor is multiple myeloma or Waldenstrom's Macroglobulinemia. In certain embodiments, the tumor is multiple myeloma.

### BRIEF DESCRIPTION OF THE FIGURES

The following Detailed Description, given by way of example, but not intended to limit the invention to specific embodiments described, may be understood in conjunction with the accompanying drawings.

FIG. 1 depicts the human GPRC5D expression in various tissues.

FIG. 2 illustrates the CLIPS technology. The CLIPS reaction takes place between bromo groups of the CLIPS scaffold and thiol sidechains of cysteines. The reaction is fast and specific under mild conditions. Using this elegant chemistry, native protein sequences are transformed into CLIPS constructs with a range of structures. From left to right: two different single T2 loops, T3 double loop, conjugated T2+T3 loops, stabilized beta sheet, and stabilized alpha helix (Timmerman et al., J. Mol. Recognit. 2007; 20: 283-29).

FIG. 3 illustrates combinatorial clips library screening. The target protein (left) containing a discontinuous conformational epitope is converted into a matrix library (middle). Combinatorial peptides are synthesized on a proprietary minicard and chemically converted into spatially defined CLIPS constructs (right).

FIG. 4 depicts T3 looped CLIPS™ construct.

FIGS. 5A-5D illustrates heat map technology. (i) Table of combined peptides, with two sub-sequences indicated as “Loop 1” and “Loop 2”. (ii) Data from A displayed as a matrix. (iii) Color bar indication of the heat map representation. (iv) Heat map visualization of data from A.

FIG. 6 shows intensity profiles recorded for ET150-2. Lines are drawn from the starting residue to the ending residue of a single peptide on the height at which the signal for that peptide is recorded.

FIG. 7 shows heatmap analysis of data recorded for ET150-5 under high stringency conditions.

FIG. 8 shows intensity profiles recorded for ET150-18.

FIG. 9 shows intensity profiles recorded for ET150-8.

FIG. 10 depicts schematic drawing of a GPCR containing seven transmembrane helices (TM) and 3 extracellular regions (ECLs). Colored arrows binding sites for each antibody is depicted.

FIG. 11 depicts scatterplot analysis of all data recorded for each sample. On the diagonal is the statistical data distribution.

FIG. 12 depicts FACS analysis of anti-GPRC5D antibodies.

FIG. 13 depicts FACS analysis of anti-GPRC5D antibodies.

FIG. 14 depicts the FACS analysis of anti-GPRC5D/CD3 bispecific antibodies.

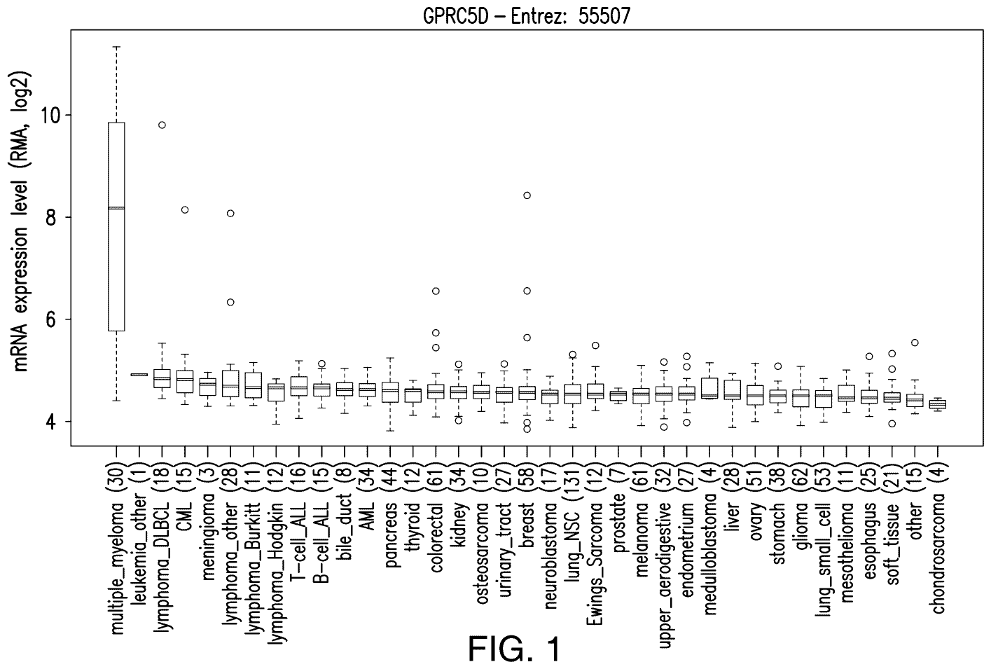

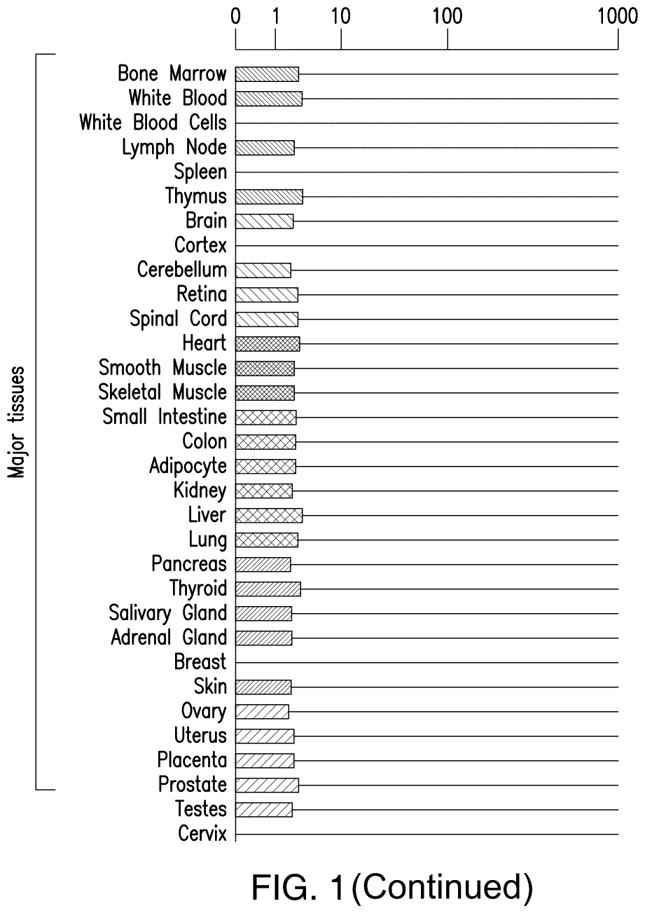

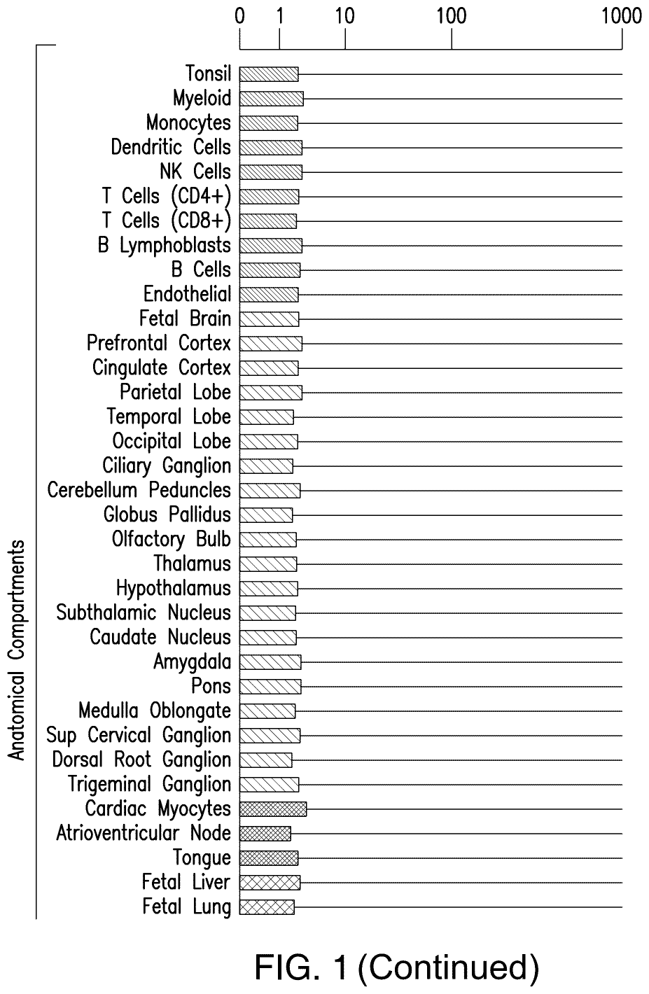

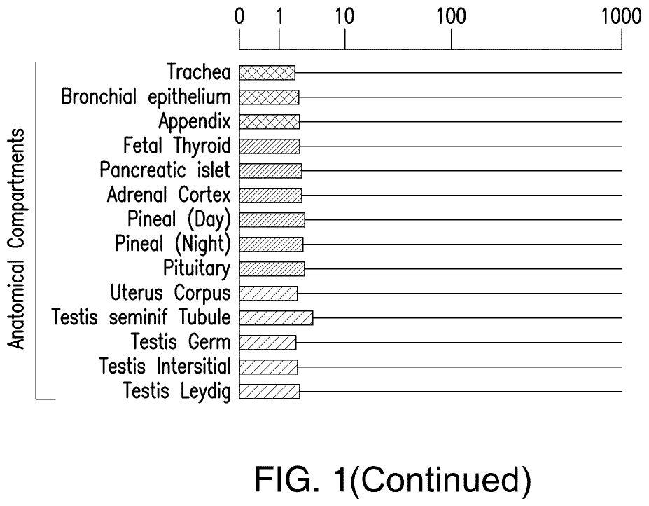

### DETAILED DESCRIPTION OF EMBODIMENTS OF THE INVENTION

All publications, patents and other references cited herein are incorporated by reference in their entirety into the present disclosure.

In practicing the presently disclosed subject matter, many conventional techniques in molecular biology, microbiology, cell biology, biochemistry, and immunology are used, which are within the skill of the art. These techniques are described in greater detail in, for example, Molecular Cloning: a Laboratory Manual 3rd edition, J. F. Sambrook and D. W. Russell, ed. Cold Spring Harbor Laboratory Press 2001; Recombinant Antibodies for Immunotherapy, Melvyn Little, ed. Cambridge University Press 2009; Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001). The contents of these references and other references containing standard protocols, widely known to and relied upon by those of skill in the art, including manufacturers' instructions are hereby incorporated by reference as part of the present disclosure.

### Definitions

In the description that follows, certain conventions will be followed as regards the usage of terminology. Generally, terms used herein are intended to be interpreted consistently with the meaning of those terms as they are known to those of skill in the art.

An “antigen-binding protein” is a protein or polypeptide that comprises an antigen-binding region or antigen-binding portion, that is, has a strong affinity to another molecule to which it binds. Antigen-binding proteins encompass antibodies, chimeric antigen receptors (CARs) and fusion proteins.

“Antibody” and “antibodies” as those terms are known in the art refer to antigen binding proteins of the immune system. The term “antibody” as referred to herein includes whole, full length antibodies having an antigen-binding region, and any fragment thereof in which the “antigen-binding portion” or “antigen-binding region” is retained, or single chains, for example, single chain variable fragment (scFv), thereof. A naturally occurring “antibody” is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (CH) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1 q) of the classical complement system.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the presently disclosed subject matter may include amino acid residues not encoded by human germline immunoglobulin sequences (e.g., mutations introduced by random or site-specific mutagenesis in vitro or by somatic mutation in vivo).

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed subject matter may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline V_{H} and V_{L} sequences, may not naturally exist within the human antibody germline repertoire in vivo.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, an antibody that “specifically binds to human GPRC5D” is intended to refer to an antibody that binds to human GPRC5D with a K_{D} of 5×10⁻⁷ M or less, 1×10⁻⁷ M or less, 5×10⁻⁸ M or less, 1×10⁻⁸ M or less, 5×10⁻⁹ M or less, 1×10⁻⁹ M or less, 5×10⁻¹⁰ M or less, or 1×10⁻¹⁰ M or less.

An “antibody that competes for binding” or “antibody that cross-competes for binding” with a reference antibody for binding to an antigen, e.g., GPRC5D, refers to an antibody that blocks binding of the reference antibody to the antigen (e.g., GPRC5D) in a competition assay by 50% or more, and conversely, the reference antibody blocks binding of the antibody to the antigen (e.g., GPRC5D) in a competition assay by 50% or more. An exemplary competition assay is described in “Antibodies”, Harlow and Lane (Cold Spring Harbor Press, Cold Spring Harbor, NY).

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen (e.g., a GPRC5D polypeptide).”

The term “antigen-binding portion” or “antigen-binding region” of an antibody, as used herein, refers to that region or portion of the antibody that binds to the antigen and which confers antigen specificity to the antibody; fragments of antigen-binding proteins, for example, antibodies includes one or more fragments of an antibody that retain the ability to specifically bind to an antigen (e.g., a GPRC5D polypeptide). It has been shown that the antigen-binding function of an antibody can be performed by fragments of a full-length antibody. Examples of antigen-binding fragments encompassed within the term “antibody fragments” of an antibody include a Fab fragment, a monovalent fragment consisting of the V_{L}, V_{H}, C_{L} and CH1 domains; a F(ab)₂ fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., 1989 Nature 341:544-546), which consists of a V_{H} domain; and an isolated complementarity determining region (CDR).

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules. These are known as single chain Fv (scFv); see e.g., Bird et al., 1988 Science 242:423-426; and Huston et al., 1988 Proc. Natl. Acad. Sci. 85:5879-5883. These antibody fragments are obtained using conventional techniques known to those of skill in the art, and the fragments are screened for utility in the same manner as are intact antibodies.

An “isolated antibody” or “isolated antigen-binding protein” is one which has been identified and separated and/or recovered from a component of its natural environment. “Synthetic antibodies” or “recombinant antibodies” are generally generated using recombinant technology or using peptide synthetic techniques known to those of skill in the art.

The terms “GPRC5D” and “G-protein coupled receptor family C group 5 member D” are used interchangeably, and include variants, isoforms, species homologs of human GPRC5D, and analogs having at least one common epitope with GPRC5D (e.g., human GPRC5D). An exemplary human GPRC5D sequence can be found under GenBank Protein Accession No: NP_061124.1.

As used herein, the term “single-chain variable fragment” or “scFv” is a fusion protein of the variable regions of the heavy (VH) and light chains (VL) of an immunoglobulin (e.g., mouse or human) covalently linked to form a VH::VL heterodimer. The heavy (VH) and light chains (VL) are either joined directly or joined by a peptide-encoding linker (e.g., 10, 15, 20, 25 amino acids), which connects the N-terminus of the VH with the C-terminus of the VL, or the C-terminus of the VH with the N-terminus of the VL. The linker is usually rich in glycine for flexibility, as well as serine or threonine for solubility. The linker can link the heavy chain variable region and the light chain variable region of the antibody or an antigen-binding fragment thereof. Non-limiting examples of linkers are disclosed in Shen et al., Anal. Chem. 80(6):1910-1917 (2008) and WO 2014/087010, the contents of which are hereby incorporated by reference in their entireties. In certain embodiments, the linker is a G4S linker (SEQ ID NO: 365).

In certain embodiments, the linker comprises amino acids having the sequence set forth in SEQ ID NO:309 as provided below:

[SEQ ID NO: 309]
GGGGSGGGGSGGGGS.

In certain embodiments, the nucleic acid sequence encoding the amino acid sequence of SEQ ID NO:309 is set forth in SEQ ID NO:364, which is provided below:

[SEQ ID NO: 364]
GGTGGAGGTGGATCAGGTGGAGGTGGATCTGGTGGAGGTGGATCT.

In one non-limiting example, the linker comprises amino acids having the sequence set forth in SEQ ID NO:98 as provided below.

[SEQ ID NO: 98]
SRGGGGSGGGGSGGGGSLEMA

In certain embodiments, the nucleic acid sequence encoding the amino acid sequence of SEQ ID NO:98 is set forth in SEQ ID NO:99, which is provided below:

[SEQ ID NO: 99]
tctagaggtggtggtggtagcggcggcggcggctctggtggtggtgga
tccctcgagatggcc

In certain embodiments, the linker comprises amino acids having the following sequence

[SEQ ID NO: 365]
GGGGS.

In certain embodiments, the linker comprises amino acids having the following sequence

[SEQ ID NO: 366]
SGGSGGS.

In certain embodiments, the linker comprises amino acids having the following sequence

[SEQ ID NO: 367]
GGGGSGGGS.

In certain embodiments, the linker comprises amino acids having the following sequence

[SEQ ID NO: 368]
GGGGSGGGGS.

In certain embodiments, the linker comprises amino acids having the following sequence

[SEQ ID NO: 369]
GGGGSGGGGSGGGGGGGS.

In certain embodiments, the linker comprises amino acids having the following sequence

[SEQ ID NO: 370]
GGGGSGGGGSGGGGSGGGGS.

In certain embodiments, the linker comprises amino acids having the following sequence

[SEQ ID NO: 371]
GGGGSGGGGSGGGGSGGGGSGGGGS.

In certain embodiments, the linker comprises amino acids having the following sequence

[SEQ ID NO: 372]
GGGGSGGGGSGGGGSGGGGSGGGGSGGGGS.

In certain embodiments, the linker comprises amino acids having the following sequence

[SEQ ID NO: 373]
GGGGSGGGGSGGGGSGGGGGGGGSGGGGSGGGGS.

In certain embodiments, the linker comprises amino acids having the following sequence

[SEQ ID NO: 374]
EPKSCDKTHTCPPCP.

In certain embodiments, the linker comprises amino acids having the following sequence

[SEQ ID NO: 375]
GGGGSGGGSEPKSCDKTHTCPPCP.

In certain embodiments, the linker comprises amino acids having the following sequence

[SEQ ID NO: 376]
ELKTPLGDTTHTCPRCPEPKSCDTPPPCPRCPEPKSCDTPPPCPRCPEPK
SCDTPP PCPRCP.

In certain embodiments, the linker comprises amino acids having the following sequence

[SEQ ID NO: 377]
GSGSGS.

In certain embodiments, the linker comprises amino acids having the following sequence

[SEQ ID NO: 378]
AAA.

Despite removal of the constant regions and the introduction of a linker, scFv proteins retain the specificity of the original immunoglobulin. Single chain Fv polypeptide antibodies can be expressed from a nucleic acid comprising VH- and VL-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27(6):455-51; Peter et al., J Cachexia Sarcopenia Muscle 2012 Aug. 12; Shieh et al., J Imunol 2009 183(4):2277-85; Giomarelli et al., Thromb Haemost 2007 97(6):955-63; Fife et al., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Bioi Chern 2003 25278(38):36740-7; Xie et al., Nat Biotech 1997 15(8):768-71; Ledbetter et al., Crit Rev Immunol 1997 17(5-6):427-55; Ho et al., BioChim Biophys Acta 2003 1638(3):257-66).

As used herein, “F(ab)” refers to a fragment of an antibody structure that binds to an antigen but is monovalent and does not have a Fc portion, for example, an antibody digested by the enzyme papain yields two F(ab) fragments and an Fc fragment (e.g., a heavy (H) chain constant region; Fc region that does not bind to an antigen).

As used herein, “F(ab)₂” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab)₂” fragment can be split into two individual Fab′ fragments.

As used herein, the term “vector” refers to any genetic element, such as a plasmid, phage, transposon, cosmid, chromosome, virus, virion, etc., which is capable of replication when associated with the proper control elements and which can transfer gene sequences into cells. Thus, the term includes cloning and expression vehicles, as well as viral vectors and plasmid vectors.

“CDRs” are defined as the complementarity determining region amino acid sequences of an antibody which are the hypervariable regions of immunoglobulin heavy and light chains. See, e. g., Kabat et al., Sequences of Proteins of Immunological Interest, 4th U. S. Department of Health and Human Services, National Institutes of Health (1987). The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise three heavy chain and three light chain CDRs or CDR regions in the variable region. CDRs provide the majority of contact residues for the binding of the antibody to the antigen or epitope.

An “isolated antibody” is one which has been separated from a component of its natural environment. In certain embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated nucleic acid” refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

An “isolated nucleic acid encoding an antibody” (including references to a specific antibody, e.g. an anti-KLB antibody) refers to one or more nucleic acid molecules encoding antibody heavy and light chains (or fragments thereof), including such nucleic acid molecule(s) in a single vector separate vectors, and such nucleic acid molecule(s) present at one or more locations in a host cell.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

An “immunoconjugate” is an antibody conjugated to one or more heterologous molecule(s), including, but not limited to, a cytotoxic agent.

An “effective amount” of an agent, e.g., an anti-GPRC5D antibody or an antigen-binding fragment thereof, a pharmaceutical comprision comprising thereof, refers to an amount effective, at dosages and for periods of time necessary, to achieve the desired therapeutic or prophylactic result, e.g., treating a tumor (e.g., multiple myeloma).

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In certain embodiments, antibodies of the presently disclosed subject matter are used to delay development of a disease or to slow the progression of a disease, e.g., a tumor (multiple myeloma).

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As described herein, any concentration range, percentage range, ratio range or integer range is to be understood to include the value of any integer within the recited range and, when appropriate, fractions thereof (such as one tenth and one hundredth of an integer), unless otherwise indicated.

Anti-GPRC5D Antibodies

The antibodies of the presently disclosed subject matter are characterized by particular functional features or properties of the antibodies. For example, the antibodies bind specifically to GPRC5D (e.g., bind to human GPRC5D and may cross-react with GPRC5D from other species, such as mouse). In certain embodiments, an antibody of the presently disclosed subject matter binds to GPRC5D with high affinity, for example with a K_{d} of 1×10⁻⁷ M or less, e.g., about about 1×10⁻⁸ M or less, about 1×10⁻⁹ M or less, or about 1×10⁻¹⁰ M or less. In certain embodiments, a presently disclosed anti-GPRC5D antibody binds to GPRC5D (e.g., human GPRC5D) with a K_{d} of from about 1×10⁻¹⁰ M to about 1×10⁻⁷ M, e.g., about from about 1×10⁻¹⁰ M to about 1×10⁻⁹ M, from 1×10⁻⁹ M to about 1×10⁻⁸ M, or from about 1×10⁻⁸ M to about 1×10⁻⁷ M. In certain embodiments, a presently disclosed anti-GPRC5D antibody binds to GPRC5D (e.g., human GPRC5D) with a K_{d} of about 1×10⁻⁸ M or less. In certain embodiments, a presently disclosed anti-GPRC5D antibody binds to GPRC5D (e.g., human GPRC5D) with a K_{d} of from about 1×10⁻⁹ M to about 1×10⁻⁸ M. In certain embodiments, a presently disclosed anti-GPRC5D antibody binds to GPRC5D (e.g., human GPRC5D) with a K_{d} of from about 1×10⁻⁹ M to about 1.5×10⁻⁹ M. In certain embodiments, a presently disclosed anti-GPRC5D antibody binds to GPRC5D (e.g., human GPRC5D) with a K_{d} of about 1.2×10⁻⁹ M. In certain embodiments, a presently disclosed anti-GPRC5D antibody binds to GPRC5D (e.g., human GPRC5D) with a K_{d} of from about 4×10⁻⁹ M to about 5×10⁻⁹ M. In certain embodiments, a presently disclosed anti-GPRC5D antibody binds to GPRC5D (e.g., human GPRC5D) with a K_{d} of about 5×10⁻⁹ M. In certain embodiments, a presently disclosed anti-GPRC5D antibody binds to GPRC5D (e.g., human GPRC5D) with a K_{d} of about 4.8×10⁻⁹ M. In certain embodiments, a presently disclosed anti-GPRC5D antibody binds to GPRC5D (e.g., human GPRC5D) with a K_{d} of from about 8×10⁻⁹ M to about 9×10⁻⁹ M. In certain embodiments, a presently disclosed anti-GPRC5D antibody binds to GPRC5D (e.g., human GPRC5D) with a K_{d} of about 8×10⁻⁹ M. In certain embodiments, a presently disclosed anti-GPRC5D antibody binds to GPRC5D (e.g., human GPRC5D) with a K_{d} of about 8.1×10⁻⁹ M.

The heavy and light chains of an antibody of the presently disclosed subject matter can be full-length (e.g., an antibody can include at least one (e.g., one or two) complete heavy chains, and at least one (e.g., one or two) complete light chains) or can include an antigen-binding portion (a Fab, F(ab′)₂, Fv or a single chain Fv fragment (“scFv”)). In certain embodiments, the antibody heavy chain constant region is chosen from, e.g IgG1, IgG2, IgG3, IgG4, IgM, IgA1, IgA2, IgD, and IgE, particularly chosen from, e.g., IgG1, IgG2, IgG3, and IgG4, more particularly, IgG1 (e.g., human IgG1). In another embodiment, the antibody light chain constant region is chosen from, e.g., kappa or lambda, particularly kappa.

1. Single-Chain Variable Fragments (scFvs)

In certain embodiments, the presently disclosed subject matter includes antibodies that have the scFv sequence fused to one or more constant domains to form an antibody with an Fc region of a human immunoglobulin to yield a bivalent protein, increasing the overall avidity and stability of the antibody. In addition, the Fc portion allows the direct conjugation of other molecules, including but not limited to fluorescent dyes, cytotoxins, radioisotopes etc. to the antibody for example, for use in antigen quantitation studies, to immobilize the antibody for affinity measurements, for targeted delivery of a therapeutic agent, to test for Fc-mediated cytotoxicity using immune effector cells and many other applications.

The results presented here highlight the specificity, sensitivity and utility of the antibodies of the invention in targeting a GPRC5D polypeptide.

The molecules of the invention are based on the identification and selection of single chain variable fragments (scFvs) using phage display, the amino acid sequence of which confers the molecules' specificity for a GPRC5D polypeptide of interest and forms the basis of all antigen binding proteins of the disclosure. The scFv, therefore, can be used to design a diverse array of “antibody” molecules, including, for example, full length antibodies, fragments thereof, such as Fab and F(ab′)₂, minibodies, fusion proteins, including scFv-Fc fusions, multivalent antibodies, that is, antibodies that have more than one specificity for the same antigen or different antigens, for example, bispecific antibodies, tribodies, etc. (see Cuesta et al., Multivalent antibodies: when design surpasses evolution. Trends in Biotechnology 28:355-362 2010).

In certain embodiments, the antigen-binding protein is a full length antibody, the heavy and light chains of an antibody of the presently disclosed subject matter can be full-length (e.g., an antibody can include at least one, and preferably two, complete heavy chains, and at least one, and preferably two, complete light chains) or can include an antigen-binding portion (a Fab, F(ab′)₂, Fv or a single chain Fv fragment (“scFv”)). In certain embodiments, the antibody heavy chain constant region is chosen from, e.g., IgG1, IgG2, IgG3, IgG4, IgM, IgA1, IgA2, IgD, and IgE. In certain embodiments, the immunoglobulin isotype is selected from IgG1, IgG2, IgG3, and IgG4, more particularly, IgG1 (e.g., human IgG1). The choice of antibody isotype can depend on the immune effector function that the antibody is designed to elicit.

In constructing a recombinant immunoglobulin, appropriate amino acid sequences for constant regions of various immunoglobulin isotypes and methods for the production of a wide array of antibodies are known to those of skill in the art.

In certain embodiments, the antibody or other antigen binding protein is an anti-GPRC5D scFv or an antigen-binding fragment thereof having an antigen-binding region that comprises the amino acid sequence of SEQ ID NO: 100 and specifically binds to a GPRC5D polypeptide (e.g., a GPRC5D polypeptide having the amino acid sequence SEQ ID NO:97 which is provided below, or fragments thereof).

[SEQ ID NO: 97]
MYKDCIESTGDYFLLCDAEGPWGIILESLAILGIVVTILLLLAFLFLMRK
IQDCSQWNVLPTQLLFLLSVLGLFGLAFAFIIELNQQTAPVRYFLFGVLF
ALCFSCLLAHASNLVKLVRGCVSFSWTTILCIAIGCSLLQIIIATEYVTL
IMTRGMMFVNIVITPCQLNVDFVVLLVYVLFLMALTFFVSKATFCGPCEN
WKQHGRLIFITVLFSIIIWVVWISMLLRGNPQFQRQPQWDDPVVCIALVT
NAWVFLLLYIVPELCILYRSCRQECPLQGNACPVTAYQHSFQVENQELSR
ARDSDGAEEDVALTSYGTPIQPQTVDPTQECFIPQAKLSPQQDAGGV

The N-terminal region of human GPRC5D has amino acids 1-27 of SEQ ID NO:97. The extracellular loop 1 (ECL1) region of human GPRC5D has amino acids 85-93 of SEQ ID NO:97. The extracellular loop 2 (ECL2) region of human GPRC5D has amino acids 145-167 of SEQ ID NO:97. The extracellular loop 3 (ECL3) region of human GPRC5D has amino acids 226-239 of SEQ ID NO:97.

In certain embodiments, the antibody or other antigen binding protein is an anti-GPRC5D scFv or an antigen-binding fragment thereof having an antigen-binding region that comprises the amino acid sequence of SEQ ID NO:100 and specifically binds to a GPRC5D polypeptide (e.g., a GPRC5D polypeptide having the amino acid sequence SEQ ID NO:97, or fragments thereof), which is designated as ET150-153 scFv (also referred to as “ET150-3 scFv”).

In certain embodiments, the anti-GPRC5D scFv antibody comprises a heavy chain variable region comprising amino acids having the sequence set forth in SEQ ID NO:1 and a light chain variable region comprising amino acids having the sequence set forth in SEQ ID NO:2, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the anti-GPRC5D scFv antibody is an scFv-Fc fusion protein or full length human IgG with V_{H} and V_{L} regions or CDRs selected from Table 1. In certain embodiments, the anti-GPRC5D scFv comprises a V_{H} comprising amino acids having the sequence set forth in SEQ ID NO:1, as shown in Table 1. In certain embodiments, the anti-GPRC5D scFv comprises a V_{L} comprising amino acids having the sequence set forth in SEQ ID NO:2, as shown in Table 1. In certain embodiments, the anti-GPRC5D scFv comprises a V_{H} comprising amino acids having the sequence set forth in SEQ ID NO:1 and a V_{L} comprising amino acids having the sequence set forth in SEQ ID NO:2, as shown in Table 1. In certain embodiments, the anti-GPRC5D scFv comprises a V H CDR1 comprising amino acids having the sequence set forth in SEQ ID NO:124 or conservative modifications thereof, a V_{H} CDR2 comprising amino acids having the sequence set forth in SEQ ID NO:125 or conservative modifications thereof, and a V_{H} CDR3 comprising amino acids having the sequence set forth in SEQ ID NO:126 or conservative modifications thereof, as shown in Table 1. In certain embodiments, the anti-GPRC5D scFv comprises a V_{L} CDR1 comprising amino acids having the sequence set forth in SEQ ID NO:127 or conservative modifications thereof, a V_{L} CDR2 comprising amino acids having the sequence set forth in SEQ ID NO:128 or conservative modifications thereof, and a V_{L} CDR3 comprising amino acids having the sequence set forth in SEQ ID NO:129 or conservative modifications thereof, as shown in Table 1. In certain embodiments, the anti-GPRC5D scFv comprises a V_{H} CDR1 comprising amino acids having the sequence set forth in SEQ ID NO:124 or conservative modifications thereof, a V_{H} CDR2 comprising amino acids having the sequence set forth in SEQ ID NO:125 or conservative modifications thereof, a V_{H} CDR3 comprising amino acids having the sequence set forth in SEQ ID NO:126 or conservative modifications thereof, a V_{L} CDR1 comprising amino acids having the sequence set forth in SEQ ID NO:127 or conservative modifications thereof, a V_{L} CDR2 comprising amino acids having the sequence set forth in SEQ ID NO:128 or conservative modifications thereof, and a V_{L} CDR3 comprising amino acids having the sequence set forth in SEQ ID NO:129 or conservative modifications thereof, as shown in Table 1. In certain embodiments, the anti-GPRC5D scFv comprises a V H CDR1 comprising amino acids having the sequence set forth in SEQ ID NO:124, a V_{H} CDR2 comprising amino acids having the sequence set forth in SEQ ID NO:125, a V_{H} CDR3 comprising amino acids having the sequence set forth in SEQ ID NO:126, a V_{L} CDR1 comprising amino acids having the sequence set forth in SEQ ID NO:127, a V_{L} CDR2 comprising amino acids having the sequence set forth in SEQ ID NO:128, and a V_{L} CDR3 comprising amino acids having the sequence set forth in SEQ ID NO:129.

TABLE 1

| Antigen | A GPRC5D polypeptide having the amino acid sequence of SEQ ID NO: 97 | | |
|---|---|---|---|
| CDRs | 1 | 2 | 3 |
| V_{H} | GYTFTSYY [SEQ ID | GYTFTSYY [SEQ ID | ARGMYRSLLFYDP |
| | NO: 124] | NO: 125] | [SEQ ID NO: 126] |
| V_{L} | RSNVGNYY [SEQ | DNN [SEQ ID NO: | GTWDGSLSAHV [SEQ |
| | ID NO: 127] | 128] | ID NO: 129] |

| | |
|---|---|
| Full V_{H} | QVQLVQSGSELKKPGASVRVSCTASGYTFTSYYMEIWVRQAPGQGLEW |
| | MGVINPNAGSTRYAQKFQGRVTMSTDTSTSTAYMDLSSLRSEDTAVYY |
| | CARGMYRSLLFYDPWGQGTLVTVSS [SEQ ID NO: 1] |
| DNA | Caggtgcagctggtgcagtctgggtctgagttgaagaagcctggggcctcagtcagagtctcctgcacggcttctg |
| | gatacaccttcaccagttactatatgcactgggtgcgacaggcccctggacaagggcttgagtggatgggagtaat |
| | caaccctaatgctggcagcacaagatacgcacagaaattccagggcagagtcaccatgagcactgacacgtcca |
| | cgagcacagcctacatggacctgagcagtctgagatctgaggacacggccgtgtattactgtgcgcgcggtatgta |
| | ccgttctctgctgttctacgatccgtggggtcaaggtactctggtgaccgtctcctca [SEQ ID NO: 3] |
| Full V_{L} | QSVLTQPPSVSAAPGQKVTIPCSGSRSNVGNYYVSWYQQLPGTAPKLLI |
| | YDNNKRPSGIPDRFSGSKSGTSATLGITGLQTGDEADYFCGTWDGSLSA |
| | HVFGTGTKVTVLG [SEQ ID NO: 2] |
| DNA | Cagtctgtgttgacgcagccgccctcagtgtctgcggccccaggacagaaggtcaccatcccctgctctggaagc |
| | cgttccaacgttgggaattattatgtgtcctggtaccagcaactcccaggaacagcccccaaactcctcatttatgac |
| | aataataagcgaccctcagggattcctgaccgattctctggctccaagtctggcacgtcagccaccctgggcatcac |
| | cggactccagactggggacgaggccgattatttctgcggaacatgggatggcagcctgagtgcccatgtatcgga |
| | actgggaccaaggtcaccgtcctaggt [SEQ ID NO: 4] |
| scFv | QSVLTQPPSVSAAPGQKVTIPCSGSRSNVGNYYVSWYQQLPGTAPKLLI |
| | YDNNKRPSGIPDRFSGSKSGTSATLGITGLQTGDEADYFCGTWDGSLSA |
| | HVFGTGTKVTVLGSRGGGGSGGGGSGGGGSLEMAQVQLVQSGSELKK |
| | PGASVRVSCTASGYTFTSYYMHWVRQAPGQGLEWMGVINPNAGSTRY |
| | AQKFQGRVTMSTDTSTSTAYIVIDLSSLRSEDTAVYYCARGMYRSLLFYD |
| | PWGQGTLVTVSS [SEQ ID NO: 100] |

In certain embodiments, the antibody or other antigen binding protein is an anti-GPRC5D scFv or an antigen-binding fragment thereof having an antigen-binding region that comprises the amino acid sequence of SEQ ID NO: 101 and specifically binds to a GPRC5D polypeptide (e.g., a GPRC5D polypeptide having the amino acid sequence SEQ ID NO:97, or fragments thereof), which is designated as ET150-166 scFv (also referred to as “ET150-16 scFv”).

In certain embodiments, the anti-GPRC5D scFv antibody comprises a heavy chain variable region comprising amino acids having the sequence set forth in SEQ ID NO:5 and a light chain variable region comprising amino acids having the sequence set forth in SEQ ID NO:6, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the anti-GPRC5D scFv antibody is an scFv-Fc fusion protein or full length human IgG with V_{H} and V_{L} regions or CDRs selected from Table 2. In certain embodiments, the anti-GPRC5D scFv comprises a V_{H} comprising amino acids having the sequence set forth in SEQ ID NO:5, as shown in Table 2. In certain embodiments, the anti-GPRC5D scFv comprises a V_{L} comprising amino acids having the sequence set forth in SEQ ID NO:6, as shown in Table 2. In certain embodiments, the anti-GPRC5D scFv comprises a V_{H} comprising amino acids having the sequence set forth in SEQ ID NO:5 and a V_{L} comprising amino acids having the sequence set forth in SEQ ID NO:6, as shown in Table 2. In certain embodiments, the anti-GPRC5D scFv comprises a V_{H} CDR1 comprising amino acids having the sequence set forth in SEQ ID NO:130 or conservative modifications thereof, a V_{H} CDR2 comprising amino acids having the sequence set forth in SEQ ID NO:131 or conservative modifications thereof, and a V_{H} CDR3 comprising amino acids having the sequence set forth in SEQ ID NO:132 or conservative modifications thereof, as shown in Table 2. In certain embodiments, the anti-GPRC5D scFv comprises a V_{L} CDR1 comprising amino acids having the sequence set forth in SEQ ID NO:133 or conservative modifications thereof, a V_{L} CDR2 comprising amino acids having the sequence set forth in SEQ ID NO:134 or conservative modifications thereof, and a V_{L} CDR3 comprising amino acids having the sequence set forth in SEQ ID NO:135 or conservative modifications thereof, as shown in Table 2. In certain embodiments, the anti-GPRC5D scFv comprises a V_{H} CDR1 comprising amino acids having the sequence set forth in SEQ ID NO:130 or conservative modifications thereof, a V H CDR2 comprising amino acids having the sequence set forth in SEQ ID NO:131 or conservative modifications thereof, a V_{H} CDR3 comprising amino acids having the sequence set forth in SEQ ID NO:132 or conservative modifications thereof, a V_{L} CDR1 comprising amino acids having the sequence set forth in SEQ ID NO:133 or conservative modifications thereof, a V_{L} CDR2 comprising amino acids having the sequence set forth in SEQ ID NO:134 or conservative modifications thereof, and a V_{L} CDR3 comprising amino acids having the sequence set forth in SEQ ID NO:135 or conservative modifications thereof, as shown in Table 2. In certain embodiments, the anti-GPRC5D scFv comprises a V_{H} CDR1 comprising amino acids having the sequence set forth in SEQ ID NO:130, a V_{H} CDR2 comprising amino acids having the sequence set forth in SEQ ID NO:131, a V_{H} CDR3 comprising amino acids having the sequence set forth in SEQ ID NO:132, a V_{L} CDR1 comprising amino acids having the sequence set forth in SEQ ID NO:133, a V_{L} CDR2 comprising amino acids having the sequence set forth in SEQ ID NO:134, and a V_{L} CDR3 comprising amino acids having the sequence set forth in SEQ ID NO:135.

TABLE 2

| Antigen | A GPRC5D polypeptide having the amino acid sequence of SEQ ID NO: 97 | | |
|---|---|---|---|
| CDRs | 1 | 2 | 3 |
| V_{H} | GFTFSNYA [SEQ ID | ISGSGNT [SEQ ID | ARGSVRYTDI [SEQ ID |
| | NO: 130] | NO: 131] | NO: 132] |
| V_{L} | SGAIAGAY [SEQ | DDN [SEQ ID | QSYDYDSSNVL [SEQ |
| | ID NO: 133] | NO: 134] | ID NO: 135] |

| | |
|---|---|
| Full V_{H} | EVQLVESGGGLVQPGGSLRLSCAASGFTFSNYAMSWVRQAPGKGLE |
| | WVSAISGSGNTYYADSVKGRFTISRDNSKNTLYLQMNSLRAEDTAVY |
| | YCARGSVRYTDIWGQGTLVTVSS [SEQ ID NO: 5] |
| DNA | Gaggtgcagctggtggagtctgggggaggcttggtacagcctggggggtccctgagactctcctgtgcagc |
| | ctctggattcacctttagcaactatgccatgagttgggtccgccaggctccagggaagggactggagtgggtct |
| | cagctattagtggtagtggtaacacatactacgcagactccgtgaagggccggttcaccatctccagagacaat |
| | tccaagaacacgctgtatctgcaaatgaacagcctgagagccgaggacacggccgtatattactgtgcgcgcg |
| | gttctgttcgttacactgatatctggggtcaaggtactctggtgaccgtctcctca [SEQ ID NO: 7] |
| Full V_{L} | NFMLTQPHSVSESPGKTVSISCTRTSGAIAGAYVQWFQQRPGSAPTTV |
| | IYDDNKRPSGVPDRFSGSIDKSSNSASLTISGLKTEDEADYYCQSYDY |
| | DSSNVLFGGGTKLTVLG [SEQ ID NO: 6] |
| DNA | Aattttatgctgactcagccccactcagtgtcggagtctccggggaagacggtaagcatctcctgcacccgca |
| | ccagtggcgccattgccggcgcctatgtgcagtggttccagcagcgcccgggcagtgcccccaccactgtga |
| | tctatgacgataacaaaagaccctctggggtccctgatcggttctctgggtccatcgacaagtcctccaactctg |
| | cctccctcaccatctctggactgaagactgaggacgaggctgactattattgtcagtatatgattatgatagcag |
| | caatgtgctattcggcggagggaccaagctgaccgtcctaggt [SEQ ID NO: 8] |
| scFv | NFMLTQPHSVSESPGKTVSISCTRTSGAIAGAYVQWFQQRPGSAPTTV |
| | IYDDNKRPSGVPDRFSGSIDKSSNSASLTISGLKTEDEADYYCQSYDY |
| | DSSNVLFGGGTKLTVLGSRGGGGSGGGGSGGGGSLEMAEVQLVESG |
| | GGLVQPGGSLRLSCAASGFTFSNYAMSWVRQAPGKGLEWVSAISGS |
| | GNTYYADSVKGRFTISRDNSKNTLYLQMNSLRAEDTAVYYCARGSV |
| | RYTDIWGQGTLVTVSS [SEQ ID NO: 101] |

In certain embodiments, the antibody or other antigen binding protein is an anti-GPRC5D scFv or an antigen-binding fragment thereof having an antigen-binding region that comprises the amino acid sequence of SEQ ID NO:102 and specifically binds to a GPRC5D polypeptide (e.g., a GPRC5D polypeptide having the amino acid sequence SEQ ID NO:97, or fragments thereof), which is designated as ET150-170 scFv (also referred to as “ET150-20 scFv”).

In certain embodiments, the anti-GPRC5D scFv antibody comprises a heavy chain variable region comprising amino acids having the sequence set forth in SEQ ID NO:9 and a light chain variable region comprising amino acids having the sequence set forth in SEQ ID NO:10, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the anti-GPRC5D scFv antibody is an scFv-Fc fusion protein or full length human IgG with V_{H} and V_{L} regions or CDRs selected from Table 3. In certain embodiments, the anti-GPRC5D scFv comprises a V_{H} comprising amino acids having the sequence set forth in SEQ ID NO:9, as shown in Table 3. In certain embodiments, the anti-GPRC5D scFv comprises a V_{L} comprising amino acids having the sequence set forth in SEQ ID NO:10, as shown in Table 3. In certain embodiments, the anti-GPRC5D scFv comprises a V_{H} comprising amino acids having the sequence set forth in SEQ ID NO:9 and a V_{L} comprising amino acids having the sequence set forth in SEQ ID NO:10, as shown in Table 3. In certain embodiments, the anti-GPRC5D scFv comprises a V H CDR1 comprising amino acids having the sequence set forth in SEQ ID NO:136 or conservative modifications thereof, a V_{H} CDR2 comprising amino acids having the sequence set forth in SEQ ID NO:137 or conservative modifications thereof, and a V_{H} CDR3 comprising amino acids having the sequence set forth in SEQ ID NO:138 or conservative modifications thereof, as shown in Table 3. In certain embodiments, the anti-GPRC5D scFv comprises a V_{L} CDR1 comprising amino acids having the sequence set forth in SEQ ID NO:139 or conservative modifications thereof, a V_{L} CDR2 comprising amino acids having the sequence set forth in SEQ ID NO:140 or conservative modifications thereof, and a V_{L} CDR3 comprising amino acids having the sequence set forth in SEQ ID NO:141 or conservative modifications thereof, as shown in Table 3. In certain embodiments, the anti-GPRC5D scFv comprises a V_{H} CDR1 comprising amino acids having the sequence set forth in SEQ ID NO:136 or conservative modifications thereof, a V_{H} CDR2 comprising amino acids having the sequence set forth in SEQ ID NO:137 or conservative modifications thereof, a V_{H} CDR3 comprising amino acids having the sequence set forth in SEQ ID NO:138 or conservative modifications thereof, a V_{L} CDR1 comprising amino acids having the sequence set forth in SEQ ID NO:139 or conservative modifications thereof, a V_{L} CDR2 comprising amino acids having the sequence set forth in SEQ ID NO:140 or conservative modifications thereof, and a V_{L} CDR3 comprising amino acids having the sequence set forth in SEQ ID NO:141 or conservative modifications thereof, as shown in Table 3. In certain embodiments, the anti-GPRC5D scFv comprises a V H CDR1 comprising amino acids having the sequence set forth in SEQ ID NO:136, a V_{H} CDR2 comprising amino acids having the sequence set forth in SEQ ID NO:137, a V_{H} CDR3 comprising amino acids having the sequence set forth in SEQ ID NO:138, a V_{L} CDR1 comprising amino acids having the sequence set forth in SEQ ID NO:139, a V_{L} CDR2 comprising amino acids having the sequence set forth in SEQ ID NO:140, and a V_{L} CDR3 comprising amino acids having the sequence set forth in SEQ ID NO:141.

TABLE 3

| Antigen | A GPRC5D polypeptide having the amino acid sequence of SEQ ID NO: 97 | | |
|---|---|---|---|
| CDRs | 1 | 2 | 3 |
| V_{H} | GFTFNNYW [SEQ | IKQDGSEK [SEQ ID | ARSMSTAV [SEQ ID |
| | ID NO: 136] | NO: 137] | NO: 138] |
| V_{L} | QSISSY [SEQ ID | AAS [SEQ ID NO: | QQSYSVPYT [SEQ ID |
| | NO: 139] | 140] | NO: 141] |

| | |
|---|---|
| Full V_{H} | EVQLVQSGGGLVQPGGSLRLSCATSGFTFNNYWMSWVRQAPGKGLE |
| | WVANIKQDGSEKYYADSVRGRFTISRDNAKNSLSLQLNNLRAEDTAV |
| | YYCARSMSTAWGYDEWGQGTLVTVSS [SEQ ID NO: 9] |
| DNA | Gaggtgcagctggtgcagtctgggggaggcttggtccagcctggggggtccctgagactctcctgtgcaacct |
| | ctggattcacctttaataactattggatgagttgggtccgccaggctccagggaaggggctggagtgggtggcc |
| | aacataaagcaagatggaagtgagaaatactacgcggactctgtgaggggccgattcaccatctccagagaca |
| | acgccaagaactcactgtctctgcaattgaacaacctgagagccgaggacacggccgtgtattactgtgcgcgc |
| | tctatgtctactgatggggttacgatgaatggggtcaaggtactctggtgaccgtctcctca [SEQ ID |
| | NO: 11] |
| Full V_{L} | DIQLTQSPSSLSASVGDRVTITCRASQSISSYLNWYQQKPGKAPKLLIY |
| | AASSLQSGVPSRFSGSGSGTDFTLTISSLQPADFATYYCQQSYSVPYTF |
| | GQGTKLEIKR [SEQ ID NO: 10] |
| DNA | Gacatccagttgacccagtctccatcctccctgtctgcatctgtcggagacagagtcaccatcacttgccgggca |
| | agtcagagcattagcagctatttaaattggtatcaacagaaaccagggaaagcccctaagctcctgatctatgctg |
| | catccagtttgcaaagtggggtcccatcaaggttcagtggcagtggatctgggacagatttcactctcaccatcag |
| | cagtctgcaacctgcagattttgcaacttactactgtcaacagagttacagtgtcccgtacacttttggccagggga |
| | ccaagctggagatcaaacgt [SEQ ID NO: 12] |
| scFv | DIQLTQSPSSLSASVGDRVTITCRASQSISSYLNWYQQKPGKAPKLLIY |
| | AASSLQSGVPSRFSGSGSGTDFTLTISSLQPADFATYYCQQSYSVPYTF |
| | GQGTKLEIKRSRGGGGSGGGGSGGGGSLEMAEVQLVQSGGGLVQPG |
| | GSLRLSCATSGFTFNNYWMSWVRQAPGKGLEWVANIKQDGSEKYYA |
| | DSVRGRFTISRDNAKNSLSLQLNNLRAEDTAVYYCARSMSTAWGYDE |
| | WGQGTLVTVSS [SEQ ID NO: 102] |

In certain embodiments, the antibody or other antigen binding protein is an anti-GPRC5D scFv or an antigen-binding fragment thereof having an antigen-binding region that comprises the amino acid sequence of SEQ ID NO: 103 and specifically binds to a GPRC5D polypeptide (e.g., a GPRC5D polypeptide having the amino acid sequence SEQ ID NO:97, or fragments thereof), which is designated as ET150-171 scFv (also referred to as “ET150-21 scFv”).

In certain embodiments, the anti-GPRC5D scFv antibody comprises a heavy chain variable region comprising amino acids having the sequence set forth in SEQ ID NO:13 and a light chain variable region comprising amino acids having the sequence set forth in SEQ ID NO:14, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the anti-GPRC5D scFv antibody is an scFv-Fc fusion protein or full length human IgG with V_{H} and V_{L} regions or CDRs selected from Table 4. In certain embodiments, the anti-GPRC5D scFv comprises a V_{H} comprising amino acids having the sequence set forth in SEQ ID NO:13, as shown in Table 4. In certain embodiments, the anti-GPRC5D scFv comprises a V_{L} comprising amino acids having the sequence set forth in SEQ ID NO:14, as shown in Table 4. In certain embodiments, the anti-GPRC5D scFv comprises a V_{H} comprising amino acids having the sequence set forth in SEQ ID NO:13 and a V_{L} comprising amino acids having the sequence set forth in SEQ ID NO:14, as shown in Table 4. In certain embodiments, the anti-GPRC5D scFv comprises a V H CDR1 comprising amino acids having the sequence set forth in SEQ ID NO:142 or conservative modifications thereof, a V_{H} CDR2 comprising amino acids having the sequence set forth in SEQ ID NO:143 or conservative modifications thereof, and a V_{H} CDR3 comprising amino acids having the sequence set forth in SEQ ID NO:144 or conservative modifications thereof, as shown in Table 4. In certain embodiments, the anti-GPRC5D scFv comprises a V_{L} CDR1 comprising amino acids having the sequence set forth in SEQ ID NO:145 or conservative modifications thereof, a V_{L} CDR2 comprising amino acids having the sequence set forth in SEQ ID NO:146 or conservative modifications thereof, and a V_{L} CDR3 comprising amino acids having the sequence set forth in SEQ ID NO:147 or conservative modifications thereof, as shown in Table 4. In certain embodiments, the anti-GPRC5D scFv comprises a V_{H} CDR1 comprising amino acids having the sequence set forth in SEQ ID NO:142 or conservative modifications thereof, a V_{H} CDR2 comprising amino acids having the sequence set forth in SEQ ID NO:143 or conservative modifications thereof, a V_{H} CDR3 comprising amino acids having the sequence set forth in SEQ ID NO:144 or conservative modifications thereof, a V_{L} CDR1 comprising amino acids having the sequence set forth in SEQ ID NO:145 or conservative modifications thereof, a V_{L} CDR2 comprising amino acids having the sequence set forth in SEQ ID NO:146 or conservative modifications thereof, and a V_{L} CDR3 comprising amino acids having the sequence set forth in SEQ ID NO:147 or conservative modifications thereof, as shown in Table 4. In certain embodiments, the anti-GPRC5D scFv comprises a V H CDR1 comprising amino acids having the sequence set forth in SEQ ID NO:142, a V_{H} CDR2 comprising amino acids having the sequence set forth in SEQ ID NO:143, a V_{H} CDR3 comprising amino acids having the sequence set forth in SEQ ID NO:144, a V_{L} CDR1 comprising amino acids having the sequence set forth in SEQ ID NO:145, a V_{L} CDR2 comprising amino acids having the sequence set forth in SEQ ID NO:146, and a V_{L} CDR3 comprising amino acids having the sequence set forth in SEQ ID NO:147.

TABLE 4

| Antigen | A GPRC5D polypeptide having the amino acid sequence of SEQ ID NO: 97 | | |
|---|---|---|---|
| CDRs | 1 | 2 | 3 |
| V_{H} | GYTFTSYY [SEQ | INPSGGST [SEQ ID | ARGSSRWGGWTGDY |
| | ID NO: 142] | NO: 143] | [SEQ ID NO: 144] |
| V_{L} | SSDVGGYNF [SEQ | DVS [SEQ ID NO: 146] | SSYTSTRTVIFAGGTKVTV |
| | ID NO: 145] | | L [SEQ ID NO: 147] |

| | |
|---|---|
| Full V_{H} | QVQLVQSGAEVKKPGASVKVSCKASGYTFTSYYMHWVRQAPGQGLEW |
| | MGIINPSGGSTRYAQKFQGRVTMTRDTSTSTVYMELSSLRSEDTAVYYC |
| | ARGSSRWGGWTGDYWGQGTLVTVSS [SEQ ID NO: 13] |
| DNA | Caggtgcagctggtgcagtctggggctgaggtgaagaagcctggggcctcagtgaaggtttcctgcaaggcatct |
| | ggatacaccttcaccagctactatatgcactgggtgcgacaggcccctggacaagggcttgagtggatgggaataat |
| | caaccctagtggtggtagcacaaggtacgcacagaagttccagggcagagtcaccatgaccagggacacgtcaac |
| | gagcacagtctacatggagctgagcagcctgagatctgaggacacggccgtgtattactgtgcgcgcggttcttctc |
| | gctggggtggttggactggtgattactggggtcaaggtactctggtgaccgtctcctca [SEQ ID NO: 15] |
| Full V_{L} | QSALTQPASVSGSPGQSITISCTGTSSDVGGYNFVSWYQQHPGKAPKVMI |
| | YDVSKRPSGISNRFSGSKSGNTASLTISGLQVEDEAEYYCSSYTSTRTVIF |
| | AGGTKVTVLG [SEQ ID NO: 14] |
| DNA | Caatctgccctgactcagcctgcctccgtgtctgggtctcctggacagtcgatcaccatctcctgcactggaaccagc |
| | agtgacgttggtggttataactttgtctcctggtaccaacagcacccaggcaaagcccccaaagtcatgatttatgatg |
| | tcagtaagcggccctcagggatttctaatcgcttctctggctccaagtctggcaacacggcctccctgaccatctctgg |
| | gctccaggttgaggacgaggctgaatattactgcagctcatatacaagcactagaactgtgatattcgccggaggga |
| | ccaaggtcaccgtcctaggt [SEQ ID NO: 16] |
| scFv | QSALTQPASVSGSPGQSITISCTGTSSDVGGYNFVSWYQQHPGKAPKVMI |
| | YDVSKRPSGISNRFSGSKSGNTASLTISGLQVEDEAEYYCSSYTSTRTVIF |
| | AGGTKVTVLG |
| | SRGGGGSGGGGSGGGGSLEMAQVQLVQSGAEVKKPGASVKVSCKASG |
| | YTFTSYYMEIWVRQAPGQGLEWMGIINPSGGSTRYAQKFQGRVTMTRDT |
| | STSTVYMELSSLRSEDTAVYYCARGSSRWGGWTGDYWGQGTLVTVSS |
| | [SEQ ID NO: 103] |

In certain embodiments, the antibody or other antigen binding protein is an anti-GPRC5D scFv or an antigen-binding fragment thereof having an antigen-binding region that comprises the amino acid sequence of SEQ ID NO: 104 and specifically binds to a GPRC5D polypeptide (e.g., a GPRC5D polypeptide having the amino acid sequence SEQ ID NO:97, or fragments thereof), which is designated as ET150-175 scFv (also referred to as “ET150-25 scFv”).

In certain embodiments, the anti-GPRC5D scFv antibody comprises a heavy chain variable region comprising amino acids having the sequence set forth in SEQ ID NO:17 and a light chain variable region comprising amino acids having the sequence set forth in SEQ ID NO:18, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the anti-GPRC5D scFv antibody is an scFv-Fc fusion protein or full length human IgG with V_{H} and V_{L} regions or CDRs selected from Table 5. In certain embodiments, the anti-GPRC5D scFv comprises a V_{H} comprising amino acids having the sequence set forth in SEQ ID NO:17, as shown in Table 5. In certain embodiments, the anti-GPRC5D scFv comprises a V_{L} comprising amino acids having the sequence set forth in SEQ ID NO:18, as shown in Table 5. In certain embodiments, the anti-GPRC5D scFv comprises a V_{H} comprising amino acids having the sequence set forth in SEQ ID NO:17 and a V_{L} comprising amino acids having the sequence set forth in SEQ ID NO:18, as shown in Table 5. In certain embodiments, the anti-GPRC5D scFv comprises a V H CDR1 comprising amino acids having the sequence set forth in SEQ ID NO:148 or conservative modifications thereof, a V_{H} CDR2 comprising amino acids having the sequence set forth in SEQ ID NO:149 or conservative modifications thereof, and a V_{H} CDR3 comprising amino acids having the sequence set forth in0 SEQ ID NO:150 or conservative modifications thereof, as shown in Table 5. In certain embodiments, the anti-GPRC5D scFv comprises a V_{L} CDR1 comprising amino acids having the sequence set forth in SEQ ID NO:151 or conservative modifications thereof, a V_{L} CDR2 comprising amino acids having the sequence set forth in SEQ ID NO:152 or conservative modifications thereof, and a V_{L} CDR3 comprising amino acids having the sequence set forth in SEQ ID NO:153 or conservative modifications thereof, as shown in Table 5. In certain embodiments, the anti-GPRC5D scFv comprises a V_{H} CDR1 comprising amino acids having the sequence set forth in SEQ ID NO:148 or conservative modifications thereof, a V_{H} CDR2 comprising amino acids having the sequence set forth in SEQ ID NO:149 or conservative modifications thereof, a V_{H} CDR3 comprising amino acids having the sequence set forth in SEQ ID NO:150 or conservative modifications thereof, a V_{L} CDR1 comprising amino acids having the sequence set forth in SEQ ID NO:151 or conservative modifications thereof, a V_{L} CDR2 comprising amino acids having the sequence set forth in SEQ ID NO:152 or conservative modifications thereof, and a V_{L} CDR3 comprising amino acids having the sequence set forth in SEQ ID NO:153 or conservative modifications thereof, as shown in Table 5. In certain embodiments, the anti-GPRC5D scFv comprises a V H CDR1 comprising amino acids having the sequence set forth in SEQ ID NO:148, a V_{H} CDR2 comprising amino acids having the sequence set forth in SEQ ID NO:149, a V_{H} CDR3 comprising amino acids having the sequence set forth in SEQ ID NO:150, a V_{L} CDR1 comprising amino acids having the sequence set forth in SEQ ID NO:151, a V_{L} CDR2 comprising amino acids having the sequence set forth in SEQ ID NO:152, and a V_{L} CDR3 comprising amino acids having the sequence set forth in SEQ ID NO:153.

TABLE 5

| Antigen | A GPRC5D polypeptide having the amino acid sequence of SEQ ID NO: 97 | | |
|---|---|---|---|
| CDRs | 1 | 2 | 3 |
| V_{H} | GSTFSSYA [SEQ ID | ISGRGRST [SEQ ID | ARYYKSKDH [SEQ |
| | NO: 148] | NO: 149] | ID NO: 150] |
| V_{L} | RSNIGTNY [SEQ ID | RNH [SEQ ID NO: 152] | AAWDDNLSGVV |
| | NO: 151] | | [SEQ ID NO: 153] |

| | |
|---|---|
| Full V_{H} | EVQLVETGGGLVQPGGSLRLSCAASGSTFSSYAMSWVRQAPGKGLE |
| | WVSAISGRGRSTYYADSVKGRFTISRDNSKNTLYLQMNSLRAEDTAV |
| | YYCARYYKSSKDHWGQGTLVTVSS [SEQ ID NO: 17] |
| DNA | Gaggtgcagctggtggagactgggggaggcttggtacagcctggggggtccctgagactctcctgtgcagcc |
| | tctggatccacctttagcagctatgccatgagctgggtccgccaggctccagggaaggggctggagtgggtctc |
| | agctattagtggtcgtggtcgtagcacatactacgcagactccgtgaagggccggttcaccatctccagagaca |
| | attccaagaacacgctgtatctgcaaatgaacagcctgagagccgaggacacggccgtatattactgtgcgcgc |
| | tactacaaatcttctaaagatcattggggtcaaggtactctggtgaccgtctcctca [SEQ ID NO: 19] |
| Full V_{L} | QSVLTQPPSLSGAPGQRVTISCSGSRSNIGTNYVSWXQQLPGTAPKLLI |
| | YRNHQWPSGVPDRFTGSKSGTSASLAISGLRSEDEADYYCAAWDDNL |
| | SGVVFGGGTKLTVLG [SEQ ID NO: 18] |
| DNA | Cagtctgtgttgacgcagccgccctcactgtctggggccccagggcagagggtcaccatctcttgttccggaag |
| | caggtccaacatcggaactaattatgtatcctggnaccagcaactcccaggaacggcccccaaactcctcatcta |
| | taggaatcatcagtggccctcaggggtccctgaccgattcactggctccaagtctggcacctcagcctccctggc |
| | catcagtgggctccggtccgaggatgaggctgattactactgtgcagcatgggatgacaatttgagtggtgtggt |
| | gttcggcggagggaccaagctgaccgtcctaggt [SEQ ID NO: 20] |
| scFv | QSVLTQPPSLSGAPGQRVTISCSGSRSNIGTNYVSWXQQLPGTAPKLLI |
| | YRNHQWPSGVPDRFTGSKSGTSASLAISGLRSEDEADYYCAAWDDNL |
| | SGVVFGGGTKLTVLGSRGGGGSGGGGSGGGGSLEMAEVQLVETGGG |
| | LVQPGGSLRLSCAASGSTFSSYAMSWVRQAPGKGLEWVSAISGRGRS |
| | TYYADSVKGRFTISRDNSKNTLYLQMNSLRAEDTAVYYCARYYKSSK |
| | DHWGQGTLVTVSS [SEQ ID NO: 104] |

In certain embodiments, the antibody or other antigen binding protein is an anti-GPRC5D scFv or an antigen-binding fragment thereof having an antigen-binding region that comprises the amino acid sequence of SEQ ID NO: 105 and specifically binds to a GPRC5D polypeptide (e.g., a GPRC5D polypeptide having the amino acid sequence SEQ ID NO:97, or fragments thereof), which is designated as ET150-154 scFv (also referred to as “ET150-4 scFv”).

In certain embodiments, the anti-GPRC5D scFv antibody comprises a heavy chain variable region comprising amino acids having the sequence set forth in SEQ ID NO:21 and a light chain variable region comprising amino acids having the sequence set forth in SEQ ID NO:22, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the anti-GPRC5D scFv antibody is an scFv-Fc fusion protein or full length human IgG with V_{H} and V_{L} regions or CDRs selected from Table 6. In certain embodiments, the anti-GPRC5D scFv comprises a V_{H} comprising amino acids having the sequence set forth in SEQ ID NO:21, as shown in Table 6. In certain embodiments, the anti-GPRC5D scFv comprises a V_{L} comprising amino acids having the sequence set forth in SEQ ID NO:22, as shown in Table 6. In certain embodiments, the anti-GPRC5D scFv comprises a V_{H} comprising amino acids having the sequence set forth in SEQ ID NO:21 and a V_{L} comprising amino acids having the sequence set forth in SEQ ID NO:22, as shown in Table 6. In certain embodiments, the anti-GPRC5D scFv comprises a V H CDR1 comprising amino acids having the sequence set forth in SEQ ID NO:154 or conservative modifications thereof, a V_{H} CDR2 comprising amino acids having the sequence set forth in SEQ ID NO:155 or conservative modifications thereof, and a V_{H} CDR3 comprising amino acids having the sequence set forth in SEQ ID NO:156 or conservative modifications thereof, as shown in Table 6. In certain embodiments, the anti-GPRC5D scFv comprises a V_{L} CDR1 comprising amino acids having the sequence set forth in SEQ ID NO:157 or conservative modifications thereof, a V_{L} CDR2 comprising amino acids having the sequence set forth in SEQ ID NO:158 or conservative modifications thereof, and a V_{L} CDR3 comprising amino acids having the sequence set forth in SEQ ID NO:159 or conservative modifications thereof, as shown in Table 6. In certain embodiments, the anti-GPRC5D scFv comprises a V_{H} CDR1 comprising amino acids having the sequence set forth in SEQ ID NO:154 or conservative modifications thereof, a V_{H} CDR2 comprising amino acids having the sequence set forth in SEQ ID NO:155 or conservative modifications thereof, a V_{H} CDR3 comprising amino acids having the sequence set forth in SEQ ID NO:156 or conservative modifications thereof, a V_{L} CDR1 comprising amino acids having the sequence set forth in SEQ ID NO:157 or conservative modifications thereof, a V_{L} CDR2 comprising amino acids having the sequence set forth in SEQ ID NO:158 or conservative modifications thereof, and a V_{L} CDR3 comprising amino acids having the sequence set forth in SEQ ID NO:159 or conservative modifications thereof, as shown in Table 6. In certain embodiments, the anti-GPRC5D scFv comprises a V H CDR1 comprising amino acids having the sequence set forth in SEQ ID NO:154, a V_{H} CDR2 comprising amino acids having the sequence set forth in SEQ ID NO:155, a V_{H} CDR3 comprising amino acids having the sequence set forth in SEQ ID NO:156, a V_{L} CDR1 comprising amino acids having the sequence set forth in SEQ ID NO:157, a V_{L} CDR2 comprising amino acids having the sequence set forth in SEQ ID NO:158, and a V_{L} CDR3 comprising amino acids having the sequence set forth in SEQ ID NO:159.

TABLE 6

| Antigen | A GPRC5D polypeptide having the amino acid sequence of SEQ ID NO: 97 | | |
|---|---|---|---|
| CDRs | 1 | 2 | 3 |
| V_{H} | AYTFTDYY [SEQ | INPKSGRT [SEQ ID | ARVYGYSRWSGFDL |
| | ID NO: 154] | NO: 155] | [SEQ ID NO: 156] |
| V_{L} | SSNIGSNY [SEQ ID | RNN [SEQ ID | AAWDDSLSGYV [SEQ |
| | NO: 157] | NO: 158] | ID NO: 159] |

| | |
|---|---|
| Full V_{H} | QVQLVQSGAEVQRPGASVRVSCKAIAYTFTDYYIEIWVRQAPGQGP |
| | EWMGWINPKSGRTQYAPKFQDRVTLARETPISTASMELRGLTSDDT |
| | AVYYCARVYGYSRWSGFDLWGQGTLVTVSS [SEQ ID NO: 21] |
| DNA | Caggtccagctggtgcagtctggggctgaggtgcagaggcctggggcctcagtgagggtctcctgcaag |
| | gctattgcgtacaccttcaccgactactatatccactgggtgcgacaggcccctggacaagggcctgagtgg |
| | atggggtggatcaaccctaaaagtggtcgcacacagtatgcaccgaagtttcaagacagggtcaccctggc |
| | cagggagacgcccatcagcacagcctccatggagctgcgcggactgacatctgacgacacggccgtgtat |
| | tactgtgcgcgcgtttacggttactctcgttggtctggtttcgatctgtggggtcaaggtactctggtgaccgtc |
| | tcctca [SEQ ID NO: 23] |
| Full V_{L} | QAVLTQPPSASGTPGQRVTISCSGSSSNIGSNYVYWYQQLPGTAPKL |
| | LIYRNNQRPSGVPDRFSGSKSGTSASLATSGLRSEDEADYYCAAWD |
| | DSLSGYVFGTGTKVTVLG [SEQ ID NO: 22] |
| DNA | Caggctgtgctgactcagccaccctcagcgtctgggacccccgggcagagggtcaccatctcttgttctgg |
| | aagcagctccaacatcggaagtaattatgtatactggtaccagcagctcccaggaacggcccccaaactcct |
| | catctataggaataatcagcggccctcaggggtccctgaccgattctctggctccaagtctggcacctcagc |
| | ctccctggccatcagtgggctccggtccgaggatgaggctgattattactgtgcagcatgggatgacagcct |
| | gagtggttatgtcttcggaactgggaccaaggtcaccgtcctaggt [SEQ ID NO: 24] |
| scFv | QAVLTQPPSASGTPGQRVTISCSGSSSNIGSNYVYWYQQLPGTAPKL |
| | LIYRNNQRPSGVPDRFSGSKSGTSASLATSGLRSEDEADYYCAAWD |
| | DSLSGYVFGTGTKVTVLGSRGGGGSGGGGSGGGGSLEMAQVQLV |
| | QSGAEVQRPGASVRVSCKAIAYTFTDYYIEWVRQAPGQGPEWMG |
| | WINPKSGRTQYAPKFQDRVTLARETPISTASMELRGLTSDDTAVYY |
| | CARVYGYSRWSGFDLWGQGTLVTVSS [SEQ ID NO: 105] |

In certain embodiments, the antibody or other antigen binding protein is an anti-GPRC5D scFv or an antigen-binding fragment thereof having an antigen-binding region that comprises the amino acid sequence of SEQ ID NO: 106 and specifically binds to a GPRC5D polypeptide (e.g., a GPRC5D polypeptide having the amino acid sequence SEQ ID NO:97, or fragments thereof), which is designated as ET150-156 scFv (also referred to as “ET150-6 scFv”).

In certain embodiments, the anti-GPRC5D scFv antibody comprises a heavy chain variable region comprising amino acids having the sequence set forth in SEQ ID NO:25 and a light chain variable region comprising amino acids having the sequence set forth in SEQ ID NO:26, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the anti-GPRC5D scFv antibody is an scFv-Fc fusion protein or full length human IgG with V_{H} and V_{L} regions or CDRs selected from Table 7. In certain embodiments, the anti-GPRC5D scFv comprises a V_{H} comprising amino acids having the sequence set forth in SEQ ID NO:25, as shown in Table 7. In certain embodiments, the anti-GPRC5D scFv comprises a V_{L} comprising amino acids having the sequence set forth in SEQ ID NO:26, as shown in Table 7. In certain embodiments, the anti-GPRC5D scFv comprises a V_{H} comprising amino acids having the sequence set forth in SEQ ID NO:25 and a V_{L} comprising amino acids having the sequence set forth in SEQ ID NO:26, as shown in Table 7. In certain embodiments, the anti-GPRC5D scFv comprises a V H CDR1 comprising amino acids having the sequence set forth in SEQ ID NO:160 or conservative modifications thereof, a V_{H} CDR2 comprising amino acids having the sequence set forth in SEQ ID NO:161 or conservative modifications thereof, and a V_{H} CDR3 comprising amino acids having the sequence set forth in SEQ ID NO:162 or conservative modifications thereof, as shown in Table 7. In certain embodiments, the anti-GPRC5D scFv comprises a V_{L} CDR1 comprising amino acids having the sequence set forth in SEQ ID NO:163 or conservative modifications thereof, a V_{L} CDR2 comprising amino acids having the sequence set forth in SEQ ID NO:164 or conservative modifications thereof, and a V_{L} CDR3 comprising amino acids having the sequence set forth in SEQ ID NO:165 or conservative modifications thereof, as shown in Table 7. In certain embodiments, the anti-GPRC5D scFv comprises a V_{H} CDR1 comprising amino acids having the sequence set forth in SEQ ID NO:160 or conservative modifications thereof, a V_{H} CDR2 comprising amino acids having the sequence set forth in SEQ ID NO:161 or conservative modifications thereof, a V_{H} CDR3 comprising amino acids having the sequence set forth in SEQ ID NO:162 or conservative modifications thereof, a V_{L} CDR1 comprising amino acids having the sequence set forth in SEQ ID NO:163 or conservative modifications thereof, a V_{L} CDR2 comprising amino acids having the sequence set forth in SEQ ID NO:164 or conservative modifications thereof, and a V_{L} CDR3 comprising amino acids having the sequence set forth in SEQ ID NO:165 or conservative modifications thereof, as shown in Table 7. In certain embodiments, the anti-GPRC5D scFv comprises a V H CDR1 comprising amino acids having the sequence set forth in SEQ ID NO:160, a V_{H} CDR2 comprising amino acids having the sequence set forth in SEQ ID NO:161, a V_{H} CDR3 comprising amino acids having the sequence set forth in SEQ ID NO:162, a V_{L} CDR1 comprising amino acids having the sequence set forth in SEQ ID NO:163, a V_{L} CDR2 comprising amino acids having the sequence set forth in SEQ ID NO:164, and a V_{L} CDR3 comprising amino acids having the sequence set forth in SEQ ID NO:165.

TABLE 7

| Antigen | A GPRC5D polypeptide having the amino acid sequence of SEQ ID NO: 97 | | |
|---|---|---|---|
| CDRs | 1 | 2 | 3 |
| V_{H} | GYTFTTYY [SEQ | INPNGGGT [SEQ ID | ARGHKVYKSHPTGGYDR |
| | ID NO: 160] | NO: 161] | [SEQ ID NO: 162] |
| V_{L} | SRDVGGYNY [SEQ | EVS [SEQ ID NO: 164] | SSYTSSSTLD [SEQ ID |
| | ID NO: 163] | | NO: 165] |

| | |
|---|---|
| Full V_{H} | QVQLVQSGAEVKQPGASVKVSCQASGYTFTTYYMEIWVRQAPGQGLE |
| | WMGIINPNGGGTFYAQKFQDRVTMTRDTSTGTVYMELSSLRSDDTAVY |
| | YCARGHKVYKSHPTGGYDRWGQGTLVTVSS [SEQ ID NO: 25] |
| DNA | Caggtgcagctggtgcaatctggggctgaggtgaagcagcctggggcctcagtgaaggtttcctgccaggcatct |
| | ggatacaccttcaccacttattatatgcactgggtgcgacaggcccctggacaagggcttgagtggatgggaataat |
| | caaccctaatggtggtggcacattctacgcacagaagttccaggacagagtcaccatgaccagggacacgtccac |
| | gggcacagtctacatggaactgagcagcctgagatctgacgacactgccgtgtattactgtgcgcgcggtcataaa |
| | gtttacaaatctcatccgactggtggttacgatcgttggggtcaaggtactctggtgaccgtctcctca [SEQ ID |
| | NO: 27] |
| Full V_{L} | QSALTQPASVSGSPGQSITISCTGTSRDVGGYNYVSWYQQYPGKAPKLM |
| | IYEVSKRPSGVSNRFSGSKSGNTASLTISGLQAEDEADYYCSSYTSSSTLD |
| | FGTGTKVTVLG [SEQ ID NO: 26] |
| DNA | Caatctgccctgactcagcctgcctccgtgtctgggtctcctggacagtcgatcaccatctcctgcactggaaccag |
| | ccgtgacgttggtggttataactatgtctcctggtaccaacagtacccaggcaaagcccccaaactcatgatttatga |
| | ggtcagtaagcggccctcaggggtttctaatcgcttctctggctccaagtctggcaacacggcctccctgaccatct |
| | ctgggctccaggctgaggacgaggctgattattactgcagctcatataccagtagcagcactttagacttcggaact |
| | gggaccaaggtcaccgtcctaggt [SEQ ID NO: 28] |
| scFv | QSALTQPASVSGSPGQSITISCTGTSRDVGGYNYVSWYQQYPGKAPKLM |
| | IYEVSKRPSGVSNRFSGSKSGNTASLTISGLQAEDEADYYCSSYTSSSTLD |
| | FGTGTKVTVLGSRGGGGSGGGGSGGGGSLEMAQVQLVQSGAEVKQPG |
| | ASVKVSCQASGYTFTTYYMHWVRQAPGQGLEWMGIINPNGGGTFYAQ |
| | KFQDRVTMTRDTSTGTVYMELSSLRSDDTAVYYCARGHKVYKSHPTG |
| | GYDRWGQGTLVTVSS [SEQ ID NO: 106] |

In certain embodiments, the antibody or other antigen binding protein is an anti-GPRC5D scFv or an antigen-binding fragment thereof having an antigen-binding region that comprises the amino acid sequence of SEQ ID NO: 107 and specifically binds to a GPRC5D polypeptide (e.g., a GPRC5D polypeptide having the amino acid sequence SEQ ID NO:97, or fragments thereof), which is designated as ET150-157 scFv (also referred to as “ET150-7 scFv”).

In certain embodiments, the anti-GPRC5D scFv antibody comprises a heavy chain variable region comprising amino acids having the sequence set forth in SEQ ID NO:29 and a light chain variable region comprising amino acids having the sequence set forth in SEQ ID NO:30, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the anti-GPRC5D scFv antibody is an scFv-Fc fusion protein or full length human IgG with V_{H} and V_{L} regions or CDRs selected from Table 8. In certain embodiments, the anti-GPRC5D scFv comprises a V_{H} comprising amino acids having the sequence set forth in SEQ ID NO:29, as shown in Table 8. In certain embodiments, the anti-GPRC5D scFv comprises a V_{L} comprising amino acids having the sequence set forth in SEQ ID NO:30, as shown in Table 8. In certain embodiments, the anti-GPRC5D scFv comprises a V_{H} comprising amino acids having the sequence set forth in SEQ ID NO:29 and a V_{L} comprising amino acids having the sequence set forth in SEQ ID NO:30, as shown in Table 8. In certain embodiments, the anti-GPRC5D scFv comprises a V H CDR1 comprising amino acids having the sequence set forth in SEQ ID NO:166 or conservative modifications thereof, a V_{H} CDR2 comprising amino acids having the sequence set forth in SEQ ID NO:167 or conservative modifications thereof, and a V_{H} CDR3 comprising amino acids having the sequence set forth in SEQ ID NO:168 or conservative modifications thereof, as shown in Table 8. In certain embodiments, the anti-GPRC5D scFv comprises a V_{L} CDR1 comprising amino acids having the sequence set forth in SEQ ID NO:169 or conservative modifications thereof, a V_{L} CDR2 comprising amino acids having the sequence set forth in SEQ ID NO:170 or conservative modifications thereof, and a V_{L} CDR3 comprising amino acids having the sequence set forth in SEQ ID NO:171 or conservative modifications thereof, as shown in Table 8. In certain embodiments, the anti-GPRC5D scFv comprises a V_{H} CDR1 comprising amino acids having the sequence set forth in SEQ ID NO:166 or conservative modifications thereof, a V_{H} CDR2 comprising amino acids having the sequence set forth in SEQ ID NO:167 or conservative modifications thereof, a V H CDR3 comprising amino acids having the sequence set forth in SEQ ID NO:168 or conservative modifications thereof, a V_{L} CDR1 comprising amino acids having the sequence set forth in SEQ ID NO:169 or conservative modifications thereof, a V_{L} CDR2 comprising amino acids having the sequence set forth in SEQ ID NO:170 or conservative modifications thereof, and a V_{L} CDR3 comprising amino acids having the sequence set forth in SEQ ID NO:171 or conservative modifications thereof, as shown in Table 8. In certain embodiments, the anti-GPRC5D scFv comprises a V H CDR1 comprising amino acids having the sequence set forth in SEQ ID NO:166, a V_{H} CDR2 comprising amino acids having the sequence set forth in SEQ ID NO:167, a V_{H} CDR3 comprising amino acids having the sequence set forth in SEQ ID NO:168, a V_{L} CDR1 comprising amino acids having the sequence set forth in SEQ ID NO:169, a V_{L} CDR2 comprising amino acids having the sequence set forth in SEQ ID NO:170, and a V_{L} CDR3 comprising amino acids having the sequence set forth in SEQ ID NO:171.

TABLE 8

| Antigen | A GPRC5D polypeptide having the amino acid sequence of SEQ ID NO: 97 | | |
|---|---|---|---|
| CDRs | 1 | 2 | 3 |
| V_{H} | GGTFSSYA [SEQ ID | IIPIFGTA [SEQ ID | ARSHVAWSLLDY [SEQ |
| | NO: 166] | NO: 167] | ID NO: 168] |
| V_{L} | SSNIGSNY [SEQ ID | RNN [SEQ ID NO: | AAWDDSLSGVV [SEQ ID |
| | NO: 169] | 170] | NO: 171] |

| | |
|---|---|
| Full V_{H} | EVQLVQSGAEVKKPGSSVKVSCKASGGTFSSYAISWVRQAPGQGLEW |
| | MGGIIPIFGTAKYAQKFQGRVTITADESTSTAYMELSSLRSEDTAVYYC |
| | ARSHVAWSLLDYWGQGTLVTVSS [SEQ ID NO: 29] |
| DNA | Gaggtccagctggtgcagtctggggctgaggtgaagaagcctgggtcctcggtgaaggtctcctgcaaggctt |
| | ctggaggcaccttcagcagctatgctatcagctgggtgcgacaggcccctggacaagggcttgagtggatggg |
| | agggattatccctatctttggtacagcaaaatatgcacagaagttccagggcagagtcacgattaccgcggacga |
| | atccacgagcacagcctacatggagctgagcagcctgagatctgaggacacggccgtgtattactgtgcgcgct |
| | ctcatgttgcttggtctctgctggattactggggtcaaggtactctggtgaccgtctcctca [SEQ ID |
| | NO: 31] |
| Full V_{L} | SYELTQPPSASGTPGQRVTISCSGSSSNIGSNYVSWYQQLPGTAPKLLIY |
| | RNNQRPSGVPDRFSGSKSGTSASLATSGLRSEDEADYYCAAWDDSLSG |
| | VVFGGGTKLTVLG [SEQ ID NO: 30] |
| DNA | Tcctatgagctgactcagccaccctcagcgtctgggacccccgggcagagggtcaccatctcttgttctggaag |
| | cagctccaacatcggaagtaattatgtatcctggtaccagcagctcccaggaacggcccccaaactcctcatcta |
| | taggaataatcagcggccctcaggggtccctgaccgattctctggctccaagtctggcacctcagcctccctggc |
| | catcagtgggctccggtccgaggatgaggctgattattactgtgcagcatgggatgacagcctgagtggtgtggt |
| | attcggcggagggaccaagctgaccgtcctaggt [SEQ ID NO: 32] |
| scFv | SYELTQPPSASGTPGQRVTISCSGSSSNIGSNYVSWYQQLPGTAPKLLIY |
| | RNNQRPSGVPDRFSGSKSGTSASLATSGLRSEDEADYYCAAWDDSLSG |
| | VVFGGGTKLTVLGSRGGGGSGGGGSGGGGSLEMAEVQLVQSGAEVK |
| | KPGSSVKVSCKASGGTFSSYAISWVRQAPGQGLEWMGGIIPIFGTAKY |
| | AQKFQGRVTITADESTSTAYMELSSLRSEDTAVYYCARSHVAWSLLD |
| | YWGQGTLVTVSS [SEQ ID NO: 107] |

In certain embodiments, the antibody or other antigen binding protein is an anti-GPRC5D scFv or an antigen-binding fragment thereof having an antigen-binding region that comprises the amino acid sequence of SEQ ID NO: 108 and specifically binds to a GPRC5D polypeptide (e.g., a GPRC5D polypeptide having the amino acid sequence SEQ ID NO:97, or fragments thereof), which is designated as ET150-159 scFv (also referred to as “ET150-9 scFv”).

In certain embodiments, the anti-GPRC5D scFv antibody comprises a heavy chain variable region comprising amino acids having the sequence set forth in SEQ ID NO:33 and a light chain variable region comprising amino acids having the sequence set forth in SEQ ID NO:34, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the anti-GPRC5D scFv antibody is an scFv-Fc fusion protein or full length human IgG with V_{H} and V_{L} regions or CDRs selected from Table 9. In certain embodiments, the anti-GPRC5D scFv comprises a V_{H} comprising amino acids having the sequence set forth in SEQ ID NO:33, as shown in Table 9. In certain embodiments, the anti-GPRC5D scFv comprises a V_{L} comprising amino acids having the sequence set forth in SEQ ID NO:34, as shown in Table 9. In certain embodiments, the anti-GPRC5D scFv comprises a V_{H} comprising amino acids having the sequence set forth in SEQ ID NO:33 and a V_{L} comprising amino acids having the sequence set forth in SEQ ID NO:34, as shown in Table 9. In certain embodiments, the anti-GPRC5D scFv comprises a V H CDR1 comprising amino acids having the sequence set forth in SEQ ID NO:172 or conservative modifications thereof, a V_{H} CDR2 comprising amino acids having the sequence set forth in SEQ ID NO:173 or conservative modifications thereof, and a V_{H} CDR3 comprising amino acids having the sequence set forth in SEQ ID NO:174 or conservative modifications thereof, as shown in Table 9. In certain embodiments, the anti-GPRC5D scFv comprises a V_{L} CDR1 comprising amino acids having the sequence set forth in SEQ ID NO:175 or conservative modifications thereof, a V_{L} CDR2 comprising amino acids having the sequence set forth in SEQ ID NO:176 or conservative modifications thereof, and a V_{L} CDR3 comprising amino acids having the sequence set forth in SEQ ID NO:177 or conservative modifications thereof, as shown in Table 9. In certain embodiments, the anti-GPRC5D scFv comprises a V_{H} CDR1 comprising amino acids having the sequence set forth in SEQ ID NO:172 or conservative modifications thereof, a V_{H} CDR2 comprising amino acids having the sequence set forth in SEQ ID NO:173 or conservative modifications thereof, a V_{H} CDR3 comprising amino acids having the sequence set forth in SEQ ID NO:174 or conservative modifications thereof, a V_{L} CDR1 comprising amino acids having the sequence set forth in SEQ ID NO:175 or conservative modifications thereof, a V_{L} CDR2 comprising amino acids having the sequence set forth in SEQ ID NO:176 or conservative modifications thereof, and a V_{L} CDR3 comprising amino acids having the sequence set forth in SEQ ID NO:177 or conservative modifications thereof, as shown in Table 9. In certain embodiments, the anti-GPRC5D scFv comprises a V_{H} CDR1 comprising amino acids having the sequence set forth in SEQ ID NO:172, a V_{H} CDR2 comprising amino acids having the sequence set forth in SEQ ID NO:173, a V_{H} CDR3 comprising amino acids having the sequence set forth in SEQ ID NO:174, a V_{L} CDR1 comprising amino acids having the sequence set forth in SEQ ID NO:175, a V_{L} CDR2 comprising amino acids having the sequence set forth in SEQ ID NO:176, and a V_{L} CDR3 comprising amino acids having the sequence set forth in SEQ ID NO:177.

TABLE 9

| Antigen | A GPRC5D polypeptide having the amino acid sequence of SEQ ID NO: 97 | | |
|---|---|---|---|
| CDRs | 1 | 2 | 3 |
| V_{H} | GGTFSSYA [SEQ ID | MNPNSGNT [SEQ ID | ARYQSYKGSQSD |
| | NO: 172] | NO: 173] | S [SEQ ID NO: 174] |
| V_{L} | SSNIGSNY[SEQ ID | RNN [SEQ ID NO: 176] | AAWDDSLSGWV |
| | NO: 175] | | [SEQ ID NO: 177] |

| | |
|---|---|
| Full V_{H} | QVQLVQSGAEVKKPGSSVKVSCKASGGTFSSYAISWVRQAPGQGL |
| | EWMGWMNPNSGNTGYAQKFQGRVTMTRNTSISTAYMELSSLRSE |
| | DTAVYYCARYQSYKGSQSDSWGQGTLVTVSS [SEQ ID NO: 33] |
| DNA | Caggtgcagctggtgcagtctggggctgaggtgaagaagcctgggtcctcagtgaaggtctcctgcaag |
| | gcttctggaggcaccttcagcagctatgctatcagctgggtgcgacaggcccctggacaagggcttgagt |
| | ggatgggatggatgaaccctaacagtggtaacacaggctatgcacagaagttccagggcagagtcaccat |
| | gaccaggaacacctccataagcacagcctacatggagctgagcagcctgagatctgaggacacggccgt |
| | gtattactgtgcgcgctaccagtatacaaaggttctcagtctgattcttggggtcaaggtactctggtgaccg |
| | tctcctca [SEQ ID NO: 35] |
| Full V_{L} | QSVLTQPPSASGTPGQRVTISCSGSSSNIGSNYVYWYQQLPGTAPK |
| | LLIYRNNQRPSGVPDRFSGSKSGTSASLAISGLRSEDEADYYCAAW |
| | DDSLSGWVFGGGTKLTVLG [SEQ ID NO: 34] |
| DNA | Cagtctgtgttgacgcagccaccctcagcgtctgggacccccgggcagagggtcaccatctcttgttctg |
| | gaagcagctccaacatcggaagtaattatgtatactggtaccagcagctcccaggaacggcccccaaact |
| | cctcatctataggaataatcageggccctcaggggtccctgaccgattctctggctccaagtctggcacctc |
| | agcctccctggccatcagtgggctccggtccgaggatgaggctgattattactgtgcagcatgggatgaca |
| | gcctgagtggttgggtgttcggcggagggaccaagctgaccgtcctaggt [SEQ ID NO: 36] |
| scFv | QSVLTQPPSASGTPGQRVTISCSGSSSNIGSNYVYWYQQLPGTAPK |
| | LLIYRNNQRPSGVPDRFSGSKSGTSASLAISGLRSEDEADYYCAAW |
| | DDSLSGWVFGGGTKLTVLGSRGGGGSGGGGSGGGGSLEMAQVQL |
| | VQSGAEVKKPGSSVKVSCKASGGTFSSYAISWVRQAPGQGLEWM |
| | GWMNPNSGNTGYAQKFQGRVTMTRNTSISTAYMELSSLRSEDTA |
| | VYYCARYQSYKGSQSDSWGQGTLVTVSS [SEQ ID NO: 108] |

In certain embodiments, the antibody or other antigen binding protein is an anti-GPRC5D scFv or an antigen-binding fragment thereof having an antigen-binding region that comprises the amino acid sequence of SEQ ID NO: 109 and specifically binds to a GPRC5D polypeptide (e.g., a GPRC5D polypeptide having the amino acid sequence SEQ ID NO:97, or fragments thereof), which is designated as as ET150-160 scFv (also referred to as “ET150-10 scFv”).

In certain embodiments, the anti-GPRC5D scFv antibody comprises a heavy chain variable region comprising amino acids having the sequence set forth in SEQ ID NO:37 and a light chain variable region comprising amino acids having the sequence set forth in SEQ ID NO:38, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the anti-GPRC5D scFv antibody is an scFv-Fc fusion protein or full length human IgG with V_{H} and V_{L} regions or CDRs selected from Table 10. In certain embodiments, the anti-GPRC5D scFv comprises a V_{H} comprising amino acids having the sequence set forth in SEQ ID NO:37, as shown in Table 10. In certain embodiments, the anti-GPRC5D scFv comprises a V_{L} comprising amino acids having the sequence set forth in SEQ ID NO:38, as shown in Table 10. In certain embodiments, the anti-GPRC5D scFv comprises a V_{H} comprising amino acids having the sequence set forth in SEQ ID NO:37 and a V_{L} comprising amino acids having the sequence set forth in SEQ ID NO:38, as shown in Table 10. In certain embodiments, the anti-GPRC5D scFv comprises a V H CDR1 comprising amino acids having the sequence set forth in SEQ ID NO:178 or conservative modifications thereof, a V_{H} CDR2 comprising amino acids having the sequence set forth in SEQ ID NO:179 or conservative modifications thereof, and a V_{H} CDR3 comprising amino acids having the sequence set forth in SEQ ID NO:180 or conservative modifications thereof, as shown in Table 10. In certain embodiments, the anti-GPRC5D scFv comprises a V_{L} CDR1 comprising amino acids having the sequence set forth in SEQ ID NO:181 or conservative modifications thereof, a V_{L} CDR2 comprising amino acids having the sequence set forth in SEQ ID NO:182 or conservative modifications thereof, and a V_{L} CDR3 comprising amino acids having the sequence set forth in SEQ ID NO:183 or conservative modifications thereof, as shown in Table 10. In certain embodiments, the anti-GPRC5D scFv comprises a V_{H} CDR1 comprising amino acids having the sequence set forth in SEQ ID NO:178 or conservative modifications thereof, a V_{H} CDR2 comprising amino acids having the sequence set forth in SEQ ID NO:179 or conservative modifications thereof, a V_{H} CDR3 comprising amino acids having the sequence set forth in SEQ ID NO:180 or conservative modifications thereof, a V_{L} CDR1 comprising amino acids having the sequence set forth in SEQ ID NO:181 or conservative modifications thereof, a V_{L} CDR2 comprising amino acids having the sequence set forth in SEQ ID NO:182 or conservative modifications thereof, and a V_{L} CDR3 comprising amino acids having the sequence set forth in SEQ ID NO:183 or conservative modifications thereof, as shown in Table 10. In certain embodiments, the anti-GPRC5D scFv comprises a V_{H} CDR1 comprising amino acids having the sequence set forth in SEQ ID NO:178, a V_{H} CDR2 comprising amino acids having the sequence set forth in SEQ ID NO:179, a V_{H} CDR3 comprising amino acids having the sequence set forth in SEQ ID NO:180, a V_{L} CDR1 comprising amino acids having the sequence set forth in SEQ ID NO:181, a V_{L} CDR2 comprising amino acids having the sequence set forth in SEQ ID NO:182, and a V_{L} CDR3 comprising amino acids having the sequence set forth in SEQ ID NO:183.

TABLE 10

| Antigen | A GPRC5D polypeptide having the amino acid sequence of SEQ ID NO: 97 | | |
|---|---|---|---|
| CDRs | 1 | 2 | 3 |
| V_{H} | GYTFTSYY[SEQ ID | INPSGGST [SEQ ID | ARGGSKKWSGEKW |
| | NO: 178] | NO: 179] | RRENFDY [SEQ ID |
| | | | NO: 180] |
| V_{L} | SSDVGGYNY [SEQ | DVS [SEQ ID NO: 182] | SSYTRSSTEV [SEQ |
| | ID NO: 181] | | ID NO: 183] |

| | |
|---|---|
| Full V_{H} | EVQLVQSGAEVKKPGASVKVSCKASGYTFTSYYMEIWVRQAPGQG |
| | LEWMGIINPSGGSTSYAQKFQGRVTMTRDTSTSTVYMELSSLRSED |
| | TAVYYCARGGSKKWSGEKWRRENFDYWGQGTLVTVSS [SEQ ID |
| | NO: 37] |
| DNA | Gaggtccagctggtacagtctggggctgaggtgaagaagcctggggcctcagtgaaggtttcctgcaagg |
| | catctggatacaccttcaccagctactatatgcactgggtgcgacaggcccctggacaagggcttgagtgga |
| | tgggaataatcaaccctagtggtggtagcacaagctacgcacagaagttccagggcagagtcaccatgacc |
| | agggacacgtccacgagcacagtctacatggagctgagcagcctgagatctgaggacacggccgtgtatt |
| | actgtgcgcgcggtggttctaaaaaatggtctggtgaaaaatggcgtcgtgaaaacttcgattactggggtca |
| | aggtactctggtgaccgtctcctca [SEQ ID NO: 39] |
| Full V_{L} | QSALTQPASVSGSPGQSITISCTGTSSDVGGYNYVSWYQQHPGKAP |
| | KLMIYDVSKRPSGVSNRFSGSKSGNTASLTISGLQAEDEADYYCSSY |
| | TRSSTEVFGGGTKLTVLG [SEQ ID NO: 38] |
| DNA | Caatctgccctgactcagcctgcctccgtgtctgggtctcctggacagtcgatcaccatctcctgcactggaa |
| | ccagcagtgacgttggtggttataactatgtctcctggtaccaacagcacccaggcaaagcccccaaactca |
| | tgatttatgatgtcagtaageggccctcaggggtttctaatcgcttctctggctccaagtctggcaacacggcc |
| | tccctgaccatctctgggctccaggctgaggacgaggctgattattactgcagctcatatacaagaagcagc |
| | actgaggtattcggcggagggaccaagctgaccgtcctaggt [SEQ ID NO: 40] |
| scFv | QSALTQPASVSGSPGQSITISCTGTSSDVGGYNYVSWYQQHPGKAP |
| | KLMIYDVSKRPSGVSNRFSGSKSGNTASLTISGLQAEDEADYYCSSY |
| | TRSSTEVFGGGTKLTVLGSRGGGGSGGGGSGGGGSLEMAEVQLVQ |
| | SGAEVKKPGASVKVSCKASGYTFTSYYMEIWVRQAPGQGLEWMGI |
| | INPSGGSTSYAQKFQGRVTMTRDTSTSTVYMELSSLRSEDTAVYYC |
| | ARGGSKKWSGEKWRRENFDYWGQGTLVTVSS [SEQ ID NO: 109] |

In certain embodiments, the antibody or other antigen binding protein is an anti-GPRC5D scFv or an antigen-binding fragment thereof having an antigen-binding region that comprises the amino acid sequence of SEQ ID NO: 110 and specifically binds to a GPRC5D polypeptide (e.g., a GPRC5D polypeptide having the amino acid sequence SEQ ID NO:97, or fragments thereof), which is designated as ET150-161 scFv (also referred to as “ET150-11 scFv”).

In certain embodiments, the anti-GPRC5D scFv antibody comprises a heavy chain variable region comprising amino acids having the sequence set forth in SEQ ID NO:41 and a light chain variable region comprising amino acids having the sequence set forth in SEQ ID NO:42, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the anti-GPRC5D scFv antibody is an scFv-Fc fusion protein or full length human IgG with V_{H} and V_{L} regions or CDRs selected from Table 11. In certain embodiments, the anti-GPRC5D scFv comprises a V_{H} comprising amino acids having the sequence set forth in SEQ ID NO:41, as shown in Table 11. In certain embodiments, the anti-GPRC5D scFv comprises a V_{L} comprising amino acids having the sequence set forth in SEQ ID NO:42, as shown in Table 11. In certain embodiments, the anti-GPRC5D scFv comprises a V_{H} comprising amino acids having the sequence set forth in SEQ ID NO:41 and a V_{L} comprising amino acids having the sequence set forth in SEQ ID NO:42, as shown in Table 11. In certain embodiments, the anti-GPRC5D scFv comprises a V_{H} CDR1 comprising amino acids having the sequence set forth in SEQ ID NO:184 or conservative modifications thereof, a V_{H} CDR2 comprising amino acids having the sequence set forth in SEQ ID NO: 185 or conservative modifications thereof, and a V_{H} CDR3 comprising amino acids having the sequence set forth in SEQ ID NO: 186 or conservative modifications thereof, as shown in Table 11. In certain embodiments, the anti-GPRC5D scFv comprises a V_{L} CDR1 comprising amino acids having the sequence set forth in SEQ ID NO: 187 or conservative modifications thereof, a V_{L} CDR2 comprising amino acids having the sequence set forth in SEQ ID NO: 188 or conservative modifications thereof, and a V_{L} CDR3 comprising amino acids having the sequence set forth in SEQ ID NO: 189 or conservative modifications thereof, as shown in Table 11. In certain embodiments, the anti-GPRC5D scFv comprises a V_{H} CDR1 comprising amino acids having the sequence set forth in SEQ ID NO: 184 or conservative modifications thereof, a V_{H} CDR2 comprising amino acids having the sequence set forth in SEQ ID NO: 185 or conservative modifications thereof, a V_{H} CDR3 comprising amino acids having the sequence set forth in SEQ ID NO: 186 or conservative modifications thereof, a V_{L} CDR1 comprising amino acids having the sequence set forth in SEQ ID NO: 187 or conservative modifications thereof, a V_{L} CDR2 comprising amino acids having the sequence set forth in SEQ ID NO: 188 or conservative modifications thereof, and a V_{L} CDR3 comprising amino acids having the sequence set forth in SEQ ID NO: 189 or conservative modifications thereof, as shown in Table 11. In certain embodiments, the anti-GPRC5D scFv comprises a V_{H} CDR1 comprising amino acids having the sequence set forth in SEQ ID NO: 184, a V_{H} CDR2 comprising amino acids having the sequence set forth in SEQ ID NO: 185, a V_{H} CDR3 comprising amino acids having the sequence set forth in SEQ ID NO: 186, a V_{L} CDR1 comprising amino acids having the sequence set forth in SEQ ID NO: 187, a V_{L} CDR2 comprising amino acids having the sequence set forth in SEQ ID NO: 188, and a V_{L} CDR3 comprising amino acids having the sequence set forth in SEQ ID NO: 189.

TABLE 11

| Antigen | A GPRC5D polypeptide having the amino acid sequence of SEQ ID NO: 97 | | |
|---|---|---|---|
| CDRs | 1 | 2 | 3 |
| V_{H} | EYTFTRHI [SEQ ID | INPGNGNT [SEQ ID NO: | ARLPDQ [SEQ ID |
| | NO: 184] | 185] | NO: 186] |
| V_{L} | SSNIGSNT [SEQ ID | RNN [SEQ ID NO: 188] | AAWDDSLSGL |
| | NO: 187] | | [SEQ ID NO: 189] |

| | |
|---|---|
| Full V_{H} | QMQLVQSGAEVKKPGASVKVSCKASEYTFTRHILHWVRQAPGQSL |
| | EWMGWINPGNGNTKYSQKFQVRVTFTRDTSASTVYMELSSLRSED |
| | TAVYYCARLPDQWGQGTLVTVSS [SEQ ID NO: 41] |
| DNA | Cagatgcagctggtgcagtctggggctgaggtgaagaagcctggggcctcagtgaaggtttcctgcaagg |
| | cttctgaatacaccttcactaggcatattctacattgggtgcgccaggctcccggacaaagccttgagtggat |
| | gggatggatcaacccaggcaatggtaatacaaaatattcacagaagttccaggtcagagtcacctttaccag |
| | ggacacatccgcgagcacagtctatatggagctgagcagcctgagatctgaagacacggccgtgtattact |
| | gtgcgcgcctgccggatcagtggggtcaaggtactctggtgaccgtctcctca [SEQ ID NO: 43] |
| Full V_{L} | SYVLTQPPSASGTPGQRVTISCSGSSSNIGSNTVNWYQQLPGTAPKL |
| | LIYRNNQRPSGVPDRFSGSKSGTSASLAISGLRSEDEADYYCAAWD |
| | DSLSGLFGTGTKVTVLG [SEQ ID NO: 42] |
| DNA | Tcctatgtgctgactcagccaccctcagcgtctgggacccccgggcagagggtcaccatctcttgttctgga |
| | agcagctccaacatcggaagtaatactgtaaactggtaccagcagctcccaggaacggcccccaaactcct |
| | catctataggaataatcageggccctcaggggtccctgaccgattctctggctccaagtctggcacctcagc |
| | ctccctggccatcagtgggctccggtccgaggatgaggctgattattactgtgcagcatgggatgacagcct |
| | gagtggtctcttcggaactgggaccaaggtcaccgtcctaggt [SEQ ID NO: 44] |
| scFv | SYVLTQPPSASGTPGQRVTISCSGSSSNIGSNTVNWYQQLPGTAPKL |
| | LIYRNNQRPSGVPDRFSGSKSGTSASLAISGLRSEDEADYYCAAWD |
| | DSLSGLFGTGTKVTVLGSRGGGGSGGGGSGGGGSLEMAQMQLVQ |
| | SGAEVKKPGASVKVSCKASEYTFTRHILHWVRQAPGQSLEWMGWI |
| | NPGNGNTKYSQKFQVRVTFTRDTSASTVYMELSSLRSEDTAVYYC |
| | ARLPDQWGQGTLVTVSS [SEQ ID NO: 110] |

In certain embodiments, the antibody or other antigen binding protein is an anti-GPRC5D scFv or an antigen-binding fragment thereof having an antigen-binding region that comprises the amino acid sequence of SEQ ID NO: 111 and specifically binds to a GPRC5D polypeptide (e.g., a GPRC5D polypeptide having the amino acid sequence SEQ ID NO:97, or fragments thereof), which is designated as ET150-162 scFv (also referred to as “ET150-12 scFv”).

In certain embodiments, the anti-GPRC5D scFv antibody comprises a heavy chain variable region comprising amino acids having the sequence set forth in SEQ ID NO:45 and a light chain variable region comprising amino acids having the sequence set forth in SEQ ID NO:46, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the anti-GPRC5D scFv antibody is an scFv-Fc fusion protein or full length human IgG with V_{H} and V_{L} regions or CDRs selected from Table 12. In certain embodiments, the anti-GPRC5D scFv comprises a V_{H} comprising amino acids having the sequence set forth in SEQ ID NO:45, as shown in Table 12. In certain embodiments, the anti-GPRC5D scFv comprises a V_{L} comprising amino acids having the sequence set forth in SEQ ID NO:46, as shown in Table 12. In certain embodiments, the anti-GPRC5D scFv comprises a V_{H} comprising amino acids having the sequence set forth in SEQ ID NO:45 and a V_{L} comprising amino acids having the sequence set forth in SEQ ID NO:46, as shown in Table 12. In certain embodiments, the anti-GPRC5D scFv comprises a V H CDR1 comprising amino acids having the sequence set forth in SEQ ID NO:190 or conservative modifications thereof, a V_{H} CDR2 comprising amino acids having the sequence set forth in SEQ ID NO: 191 or conservative modifications thereof, and a V_{H} CDR3 comprising amino acids having the sequence set forth in SEQ ID NO: 192 or conservative modifications thereof, as shown in Table 12. In certain embodiments, the anti-GPRC5D scFv comprises a V_{L} CDR1 comprising amino acids having the sequence set forth in SEQ ID NO: 193 or conservative modifications thereof, a V_{L} CDR2 comprising amino acids having the sequence set forth in SEQ ID NO: 194 or conservative modifications thereof, and a V_{L} CDR3 comprising amino acids having the sequence set forth in SEQ ID NO: 195 or conservative modifications thereof, as shown in Table 12. In certain embodiments, the anti-GPRC5D scFv comprises a V_{H} CDR1 comprising amino acids having the sequence set forth in SEQ ID NO: 190 or conservative modifications thereof, a V_{H} CDR2 comprising amino acids having the sequence set forth in SEQ ID NO: 191 or conservative modifications thereof, a V_{H} CDR3 comprising amino acids having the sequence set forth in SEQ ID NO: 192 or conservative modifications thereof, a V_{L} CDR1 comprising amino acids having the sequence set forth in SEQ ID NO: 193 or conservative modifications thereof, a V_{L} CDR2 comprising amino acids having the sequence set forth in SEQ ID NO: 194 or conservative modifications thereof, and a V_{L} CDR3 comprising amino acids having the sequence set forth in SEQ ID NO: 195 or conservative modifications thereof, as shown in Table 12. In certain embodiments, the anti-GPRC5D scFv comprises a V_{H} CDR1 comprising amino acids having the sequence set forth in SEQ ID NO: 190, a V_{H} CDR2 comprising amino acids having the sequence set forth in SEQ ID NO: 191, a V_{H} CDR3 comprising amino acids having the sequence set forth in SEQ ID NO: 192, a V_{L} CDR1 comprising amino acids having the sequence set forth in SEQ ID NO: 193, a V_{L} CDR2 comprising amino acids having the sequence set forth in SEQ ID NO: 194, and a V_{L} CDR3 comprising amino acids having the sequence set forth in SEQ ID NO: 195.

TABLE 12

| Antigen | A GPRC5D polypeptide having the amino acid sequence of SEQ ID NO: 97 | | |
|---|---|---|---|
| CDRs | 1 | 2 | 3 |
| V_{H} | GFTFGDYG [SEQ | INWNGGST [SEQ ID | ARSKQDY [SEQ ID |
| | ID NO: 190] | NO: 191] | NO: 192] |
| V_{L} | SRDAGGYNY [SEQ | EVT [SEQ ID NO: 194] | SSYGGSNNFRV |
| | ID NO: 193] | | [SEQ ID NO: 195] |

| | |
|---|---|
| Full V_{H} | EVQLVESGGGVVRPGGSLRLSCAASGFTFGDYGMSWVRQAPGKG |
| | LEWVSGINWNGGSTGYADSVKGRFTISRDNAKNSLYLQMNSLRA |
| | EDTAVYYCARSKQDYWGQGTLVTVSS [SEQ ID NO: 45] |
| DNA | Gaggtgcagctggtggagtctgggggaggtgtggtacggcctggggggtccctgagactctcctgtgca |
| | gcctctggattcacctttggtgattatggcatgagctgggtccgccaagctccagggaaggggctggagtg |
| | ggtctctggtattaattggaatggtggtagcacaggttatgcagactctgtgaagggccgattcaccatctcc |
| | agagacaacgccaagaactccctgtatctgcaaatgaacagtctgagagccgaggacacggccgtatatt |
| | actgtgcgcgctctaaacaggattactggggtcaaggtactctggtgaccgtctcctca [SEQ ID |
| | NO: 47] |
| Full V_{L} | QSALTQPPSASGSPGQSVTISCTGTSRDAGGYNYFSWYQQHPGKA |
| | PKLLIYEVTKRPSGVPDRFSGSKSGKTASLTVSGLQADDEAVYYCS |
| | SYGGSNNFRVFGGGTKLTVLG [SEQ ID NO: 46] |
| DNA | Cagtctgccctgactcagcctccctccgcgtccgggtctcctggacagtcagtcaccatctcctgcactgg |
| | aaccagcagggacgctggtggttataattatttctcctggtaccaacaacacccaggcaaagcccccaaac |
| | tcctgatttatgaggtcactaageggccctcaggggtccctgatcgcttctctggctccaagtctggcaaga |
| | cggcctccctgaccgtctctgggctccaggctgacgatgaggctgtatattactgcagctcatatggaggc |
| | agcaacaactttcgggtgttcggcggagggaccaagctgaccgtcctaggt [SEQ ID NO: 48] |
| scFv | QSALTQPPSASGSPGQSVTISCTGTSRDAGGYNYFSWYQQHPGKA |
| | PKLLIYEVTKRPSGVPDRFSGSKSGKTASLTVSGLQADDEAVYYCS |
| | SYGGSNNFRVFGGGTKLTVLGSRGGGGSGGGGSGGGGSLEMAEV |
| | QLVESGGGVVRPGGSLRLSCAASGFTFGDYGMSWVRQAPGKGLE |
| | WVSGINWNGGSTGYADSVKGRFTISRDNAKNSLYLQMNSLRAED |
| | TAVYYCARSKQDYWGQGTLVTVSS [SEQ ID NO: 111] |

In certain embodiments, the antibody or other antigen binding protein is an anti-GPRC5D scFv or an antigen-binding fragment thereof having an antigen-binding region that comprises the amino acid sequence of SEQ ID NO: 112 and specifically binds to a GPRC5D polypeptide (e.g., a GPRC5D polypeptide having the amino acid sequence SEQ ID NO:97, or fragments thereof), which is designated as ET150-163 scFv (also referred to as “ET150-13 scFv”).

In certain embodiments, the anti-GPRC5D scFv antibody comprises a heavy chain variable region comprising amino acids having the sequence set forth in SEQ ID NO:49 and a light chain variable region comprising amino acids having the sequence set forth in SEQ ID NO:50, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the anti-GPRC5D scFv antibody is an scFv-Fc fusion protein or full length human IgG with V_{H} and V_{L} regions or CDRs selected from Table 13. In certain embodiments, the anti-GPRC5D scFv comprises a V_{H} comprising amino acids having the sequence set forth in SEQ ID NO:49, as shown in Table 13. In certain embodiments, the anti-GPRC5D scFv comprises a V_{L} comprising amino acids having the sequence set forth in SEQ ID NO:50, as shown in Table 13. In certain embodiments, the anti-GPRC5D scFv comprises a V_{H} comprising amino acids having the sequence set forth in SEQ ID NO:49 and a V_{L} comprising amino acids having the sequence set forth in SEQ ID NO:50, as shown in Table 13. In certain embodiments, the anti-GPRC5D scFv comprises a V H CDR1 comprising amino acids having the sequence set forth in SEQ ID NO:196 or conservative modifications thereof, a V_{H} CDR2 comprising amino acids having the sequence set forth in SEQ ID NO: 197 or conservative modifications thereof, and a V_{H} CDR3 comprising amino acids having the sequence set forth in SEQ ID NO: 198 or conservative modifications thereof, as shown in Table 13. In certain embodiments, the anti-GPRC5D scFv comprises a V_{L} CDR1 comprising amino acids having the sequence set forth in SEQ ID NO: 199 or conservative modifications thereof, a V_{L} CDR2 comprising amino acids having the sequence set forth in SEQ ID NO:200 or conservative modifications thereof, and a V_{L} CDR3 comprising amino acids having the sequence set forth in SEQ ID NO:201 or conservative modifications thereof, as shown in Table 13. In certain embodiments, the anti-GPRC5D scFv comprises a V_{H} CDR1 comprising amino acids having the sequence set forth in SEQ ID NO: 196 or conservative modifications thereof, a V_{H} CDR2 comprising amino acids having the sequence set forth in SEQ ID NO: 197 or conservative modifications thereof, a V_{H} CDR3 comprising amino acids having the sequence set forth in SEQ ID NO: 198 or conservative modifications thereof, a V_{L} CDR1 comprising amino acids having the sequence set forth in SEQ ID NO: 199 or conservative modifications thereof, a V_{L} CDR2 comprising amino acids having the sequence set forth in SEQ ID NO:200 or conservative modifications thereof, and a V_{L} CDR3 comprising amino acids having the sequence set forth in SEQ ID NO:201 or conservative modifications thereof, as shown in Table 13. In certain embodiments, the anti-GPRC5D scFv comprises a V_{H} CDR1 comprising amino acids having the sequence set forth in SEQ ID NO: 196, a V_{H} CDR2 comprising amino acids having the sequence set forth in SEQ ID NO: 197, a V_{H} CDR3 comprising amino acids having the sequence set forth in SEQ ID NO: 198, a V_{L} CDR1 comprising amino acids having the sequence set forth in SEQ ID NO: 199, a V_{L} CDR2 comprising amino acids having the sequence set forth in SEQ ID NO:200, and a V_{L} CDR3 comprising amino acids having the sequence set forth in SEQ ID NO:201.

TABLE 13

| Antigen | A GPRC5D polypeptide having the amino acid sequence of SEQ ID NO: 97 | | |
|---|---|---|---|
| CDRs | 1 | 2 | 3 |
| V_{H} | GFSFSGTA[SEQ ID | ISSTGRST [SEQ ID NO: | ARVSFDY [SEQ ID |
| | NO: 196] | 197] | NO: 198] |
| V_{L} | SSNIGAGYD [SEQ ID | GNS [SEQ ID NO: 200] | QSYDSSLSGSYV |
| | NO: 199] | | [SEQ ID NO: 201] |

| | |
|---|---|
| Full V_{H} | EVQLVETGGNLVQPGASLRLSCAASGFSFSGTAMHWVRQAPGKGLE |
| | WVSTISSTGRSTYYRDSVKGRFTISRDNSKNTLYLQMNSLRGEDTAV |
| | YYCARVSFDYWGQGTLVTVSS [SEQ ID NO: 49] |
| DNA | Gaggtgcagctggtggagactgggggaaacttggtacagccgggggcgtccctgagactctcctgtgcagc |
| | ctctggattcagattagtggcactgccatgcactgggtccgccaggctccagggaaggggctggaatgggtc |
| | tcgactattagtagtactgggcgtagcacatactacagagactccgtgaagggccggttcaccatctccagaga |
| | caattccaagaacacgctgtatctgcaaatgaacagcctgagaggcgaggacacggccgtatattactgtgcg |
| | cgcgtttctttcgattactggggtcaaggtactctggtgaccgtctcctca [SEQ ID NO: 51] |
| Full V_{L} | QSVVTQPPSVSGAPGQRVTISCTGSSSNIGAGYDVHWYQQLPGTAPK |
| | LLIYGNSNRPSGVPDRFSGSKSGTSASLAITGLQAEDEADYYCQSYDS |
| | SLSGSYVFGTGTKLTVLG [SEQ ID NO: 50] |
| DNA | Cagtctgtcgtgacgcagccgccctcagtgtctggggccccagggcagagggtcaccatctcctgcactggg |
| | agcagctccaacatcggggcaggttatgatgtacactggtaccagcagettccaggaacagcccccaaactcc |
| | tcatctatggtaacagcaatcggccctcaggggtccctgaccgattctctggctccaagtctggcacctcagcct |
| | ccctggccatcactgggctccaggctgaggatgaggctgattattactgccagtcctatgacagcagcctgagt |
| | ggctcctacgtcttcggaactgggaccaagctgaccgtcctaggt [SEQ ID NO: 52] |
| scFv | QSVVTQPPSVSGAPGQRVTISCTGSSSNIGAGYDVHWYQQLPGTAPK |
| | LLIYGNSNRPSGVPDRFSGSKSGTSASLAITGLQAEDEADYYCQSYDS |
| | SLSGSYVFGTGTKLTVLGSRGGGGSGGGGSGGGGSLEMAEVQLVET |
| | GGNLVQPGASLRLSCAASGFSFSGTAMHWVRQAPGKGLEWVSTISST |
| | GRSTYYRDSVKGRFTISRDNSKNTLYLQMNSLRGEDTAVYYCARVSF |
| | DYWGQGTLVTVSS [SEQ ID NO: 112] |

In certain embodiments, the antibody or other antigen binding protein is an anti-GPRC5D scFv or an antigen-binding fragment thereof having an antigen-binding region that comprises the amino acid sequence of SEQ ID NO: 113 and specifically binds to a GPRC5D polypeptide (e.g., a GPRC5D polypeptide having the amino acid sequence SEQ ID NO:97, or fragments thereof), which is designated as ET150-151 scFv (also referred to as “ET150-1 scFv”).

In certain embodiments, the anti-GPRC5D scFv antibody comprises a heavy chain variable region comprising amino acids having the sequence set forth in SEQ ID NO:53 and a light chain variable region comprising amino acids having the sequence set forth in SEQ ID NO:54, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the anti-GPRC5D scFv antibody is an scFv-Fc fusion protein or full length human IgG with V_{H} and V_{L} regions or CDRs selected from Table 14. In certain embodiments, the anti-GPRC5D scFv comprises a V_{H} comprising amino acids having the sequence set forth in SEQ ID NO:53, as shown in Table 14. In certain embodiments, the anti-GPRC5D scFv comprises a V_{L} comprising amino acids having the sequence set forth in SEQ ID NO:54, as shown in Table 14. In certain embodiments, the anti-GPRC5D scFv comprises a V_{H} comprising amino acids having the sequence set forth in SEQ ID NO:53 and a V_{L} comprising amino acids having the sequence set forth in SEQ ID NO:54, as shown in Table 14. In certain embodiments, the anti-GPRC5D scFv comprises a V H CDR1 comprising amino acids having the sequence set forth in SEQ ID NO:202 or conservative modifications thereof, a V_{H} CDR2 comprising amino acids having the sequence set forth in SEQ ID NO:203 or conservative modifications thereof, and a V_{H} CDR3 comprising amino acids having the sequence set forth in SEQ ID NO: 204 or conservative modifications thereof, as shown in Table 14. In certain embodiments, the anti-GPRC5D scFv comprises a V_{L} CDR1 comprising amino acids having the sequence set forth in SEQ ID NO: 205 or conservative modifications thereof, a V_{L} CDR2 comprising amino acids having the sequence set forth in SEQ ID NO: 206 or conservative modifications thereof, and a V_{L} CDR3 comprising amino acids having the sequence set forth in SEQ ID NO: 207 or conservative modifications thereof, as shown in Table 14. In certain embodiments, the anti-GPRC5D scFv comprises a V_{H} CDR1 comprising amino acids having the sequence set forth in SEQ ID NO: 202 or conservative modifications thereof, a V_{H} CDR2 comprising amino acids having the sequence set forth in SEQ ID NO: 203 or conservative modifications thereof, a V_{H} CDR3 comprising amino acids having the sequence set forth in SEQ ID NO: 204 or conservative modifications thereof, a V_{L} CDR1 comprising amino acids having the sequence set forth in SEQ ID NO: 205 or conservative modifications thereof, a V_{L} CDR2 comprising amino acids having the sequence set forth in SEQ ID NO: 206 or conservative modifications thereof, and a V_{L} CDR3 comprising amino acids having the sequence set forth in SEQ ID NO: 207 or conservative modifications thereof, as shown in Table 14. In certain embodiments, the anti-GPRC5D scFv comprises a V_{H} CDR1 comprising amino acids having the sequence set forth in SEQ ID NO: 202, a V_{H} CDR2 comprising amino acids having the sequence set forth in SEQ ID NO: 203, a V_{H} CDR3 comprising amino acids having the sequence set forth in SEQ ID NO: 204, a V_{L} CDR1 comprising amino acids having the sequence set forth in SEQ ID NO: 205, a V_{L} CDR2 comprising amino acids having the sequence set forth in SEQ ID NO: 206, and a V_{L} CDR3 comprising amino acids having the sequence set forth in SEQ ID NO: 207.

TABLE 14

| Antigen | A GPRC5D polypeptide having the amino acid sequence of SEQ ID NO: 97 | | |
|---|---|---|---|
| CDRs | 1 | 2 | 3 |
| V_{H} | GFTFSSYA [SEQ ID | ISGRGRST [SEQ ID | ARYYHAGAFDL |
| | NO: 202] | NO: 203] | [SEQ ID NO: 204] |
| V_{L} | SSDVGGYNY [SEQ ID | DVS [SEQ ID NO: | SSYTSSSTLV [SEQ |
| | NO: 205] | 206] | ID NO: 207] |

| | |
|---|---|
| Full V_{H} | EVQLVESGGAFVQPGGSLRLSCAASGFTFSSYAMTWVRQAPGKGL |
| | EWVSTISGRGRSTFYADSVKGRFTISRDNSKNTLYLQMNSLRAEDT |
| | AVYYCARYYHAGAFDLWGQGTLVTVSS [SEQ ID NO: 53] |
| DNA | Gaggtgcagctggtggagtctgggggagcctttgtacagcctggggggtccctgagactctcctgtgcag |
| | cctctggattcacctttagcagctatgccatgacctgggtccgccaggctccagggaagggcctggaatg |
| | ggtctcgactattagtggtcgtggtcgtagcacattctacgcagactccgtgaagggccggtttaccatctcc |
| | agagacaattccaagaacacgctatatctgcaaatgaacagtctgagagccgaggacacggccgtatatt |
| | actgtgcgcgctactaccatgctggtgctttcgatctgtggggtcaaggtactctggtgaccgtctcctca |
| | [SEQ ID NO: 55] |
| Full V_{L} | QSVVTQPASVSGSPGQSITISCTGTSSDVGGYNYVSWYQQHPGKA |
| | PKLMIYDVSKRPSGVSNRFSGSKSGNTASLTISGLQAEDEADYYCS |
| | SYTSSSTLVFGGGTKLTVLG [SEQ ID NO: 54] |
| DNA | Cagtctgtcgtgacgcagcctgcctccgtgtctgggtctcctggacagtcgatcaccatctcctgcactgg |
| | aaccagcagtgacgttggtggttataactatgtctcctggtaccaacagcacccaggcaaagcccccaaac |
| | tcatgatttatgatgtcagtaagcggccctcaggggtttctaatcgcttctctggctccaagtctggcaacac |
| | ggcctccctgaccatctctgggctccaggctgaggacgaggctgattattactgcagctcatatacaagca |
| | gcagcactttggtattcggeggagggaccaagctgaccgtcctaggt [SEQ ID NO: 56] |
| scFv | QSVVTQPASVSGSPGQSITISCTGTSSDVGGYNYVSWYQQHPGKA |
| | PKLMIYDVSKRPSGVSNRFSGSKSGNTASLTISGLQAEDEADYYCS |
| | SYTSSSTLVFGGGTKLTVLGSRGGGGSGGGGSGGGGSLEMAEVQL |
| | VESGGAFVQPGGSLRLSCAASGFTFSSYAMTWVRQAPGKGLEWV |
| | STISGRGRSTFYADSVKGRFTISRDNSKNTLYLQMNSLRAEDTAVY |
| | YCARYYHAGAFDLWGQGTLVTVSS [SEQ ID NO: 113] |

In certain embodiments, the antibody or other antigen binding protein is an anti-GPRC5D scFv or an antigen-binding fragment thereof having an antigen-binding region that comprises the amino acid sequence of SEQ ID NO: 114 and specifically binds to a GPRC5D polypeptide (e.g., a GPRC5D polypeptide having the amino acid sequence SEQ ID NO:97, or fragments thereof), which is designated as ET150-152 scFv (also referred to as “ET150-2 scFv”).

In certain embodiments, the anti-GPRC5D scFv antibody comprises a heavy chain variable region comprising amino acids having the sequence set forth in SEQ ID NO:57 and a light chain variable region comprising amino acids having the sequence set forth in SEQ ID NO:58, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the anti-GPRC5D scFv antibody is an scFv-Fc fusion protein or full length human IgG with V_{H} and V_{L} regions or CDRs selected from Table 15. In certain embodiments, the anti-GPRC5D scFv comprises a V_{H} comprising amino acids having the sequence set forth in SEQ ID NO:57, as shown in Table 15. In certain embodiments, the anti-GPRC5D scFv comprises a V_{L} comprising amino acids having the sequence set forth in SEQ ID NO:58, as shown in Table 15. In certain embodiments, the anti-GPRC5D scFv comprises a V_{H} comprising amino acids having the sequence set forth in SEQ ID NO:57 and a V_{L} comprising amino acids having the sequence set forth in SEQ ID NO:58, as shown in Table 15. In certain embodiments, the anti-GPRC5D scFv comprises a V H CDR1 comprising amino acids having the sequence set forth in SEQ ID NO:208 or conservative modifications thereof, a V_{H} CDR2 comprising amino acids having the sequence set forth in SEQ ID NO: 209 or conservative modifications thereof, and a V_{H} CDR3 comprising amino acids having the sequence set forth in SEQ ID NO: 210 or conservative modifications thereof, as shown in Table 15. In certain embodiments, the anti-GPRC5D scFv comprises a V_{L} CDR1 comprising amino acids having the sequence set forth in SEQ ID NO: 211 or conservative modifications thereof, a V_{L} CDR2 comprising amino acids having the sequence set forth in SEQ ID NO: 212 or conservative modifications thereof, and a V_{L} CDR3 comprising amino acids having the sequence set forth in SEQ ID NO: 213 or conservative modifications thereof, as shown in Table 15. In certain embodiments, the anti-GPRC5D scFv comprises a V_{H} CDR1 comprising amino acids having the sequence set forth in SEQ ID NO: 208 or conservative modifications thereof, a V_{H} CDR2 comprising amino acids having the sequence set forth in SEQ ID NO: 209 or conservative modifications thereof, a V_{H} CDR3 comprising amino acids having the sequence set forth in SEQ ID NO: 210 or conservative modifications thereof, a V_{L} CDR1 comprising amino acids having the sequence set forth in SEQ ID NO: 211 or conservative modifications thereof, a V_{L} CDR2 comprising amino acids having the sequence set forth in SEQ ID NO: 212 or conservative modifications thereof, and a V_{L} CDR3 comprising amino acids having the sequence set forth in SEQ ID NO: 213 or conservative modifications thereof, as shown in Table 15. In certain embodiments, the anti-GPRC5D scFv comprises a V_{H} CDR1 comprising amino acids having the sequence set forth in SEQ ID NO: 208, a V_{H} CDR2 comprising amino acids having the sequence set forth in SEQ ID NO: 209, a V_{H} CDR3 comprising amino acids having the sequence set forth in SEQ ID NO: 210, a V_{L} CDR1 comprising amino acids having the sequence set forth in SEQ ID NO: 211, a V_{L} CDR2 comprising amino acids having the sequence set forth in SEQ ID NO: 212, and a V_{L} CDR3 comprising amino acids having the sequence set forth in SEQ ID NO: 213.

TABLE 15

| Antigen | A GPRC5D polypeptide having the amino acid sequence of SEQ ID NO: 97 | | |
|---|---|---|---|
| CDRs | 1 | 2 | 3 |
| V_{H} | GYTFNRYA [SEQ | ISAYNGNS [SEQ ID | ARMAYDS [SEQ ID |
| | ID NO: 208] | NO: 209] | NO: 210] |
| V_{L} | SNDVGAYKY [SEQ | DVF [SEQ ID NO: 212] | FSLTSSNTYV [SEQ ID |
| | ID NO: 211] | | NO: 213] |

| | |
|---|---|
| Full V_{H} | QMQLVQSGAEVKKPGASVKVSCKASGYTFNRYAITWVRQAPGQGLE |
| | WMGWISAYNGNSHYAQKLQGRVTMTTDTSTGTAYMELRRLRSDDT |
| | AVYYCARMAYDSWGQGTLVTVSS [SEQ ID NO: 57] |
| DNA | Cagatgcagctggtgcagtctggagctgaggtgaagaagcctggggcctcagtgaaggtctcctgcaaggctt |
| | ctggttacacctttaacagatatgctatcacctgggtgcgacaggcccctggacaaggccttgagtggatgggat |
| | ggatcagcgcttacaatggtaattcacactatgcacagaagctccagggcagagtcaccatgaccacagacac |
| | atccacgggcacagcctatatggagctgaggaggctgagatctgacgacacggccgtgtattactgtgcgcgc |
| | atggettacgattatggggtcaaggtactctggtgaccgtctcctca [SEQ ID NO: 59] |
| Full V_{L} | QSVLTQPASVSGSPGQSLTISCTGTSNDVGAYKYVSWYQQYPGKAPK |
| | LILYDVFKRPSGVSNRFSGSKSDNTASLTISGLQAEDEADYYCFSLTSS |
| | NTYVFGTGTKVTVLG [SEQ ID NO: 58] |
| DNA | Cagtctgtgttgacgcagcctgcctccgtgtctgggtctcctggacagtcgctcaccatctcctgcactggaacc |
| | agcaatgacgttggtgcttataagtatgtctcctggtatcaacagtacccaggcaaagcccccaaactcatacttta |
| | tgatgtattaageggccctcaggggtctctaatcgcttctctggctccaagtctgacaacacggcctccctgacc |
| | atctctgggctccaggctgaggacgaggctgattattactgcttctcacttacaagcagtaacacttatgtcttcgg |
| | aactgggaccaaggtcaccgtcctaggt [SEQ ID NO: 60] |
| scFv | QSVLTQPASVSGSPGQSLTISCTGTSNDVGAYKYVSWYQQYPGKAPK |
| | LILYDVFKRPSGVSNRFSGSKSDNTASLTISGLQAEDEADYYCFSLTSS |
| | NTYVFGTGTKVTVLGSRGGGGSGGGGSGGGGSLEMAQMQLVQSGAE |
| | VKKPGASVKVSCKASGYTFNRYAITWVRQAPGQGLEWMGWISAYNG |
| | NSHYAQKLQGRVTMTTDTSTGTAYMELRRLRSDDTAVYYCARMAY |
| | DSWGQGTLVTVSS [SEQ ID NO: 114] |

In certain embodiments, the antibody or other antigen binding protein is an anti-GPRC5D scFv or an antigen-binding fragment thereof having an antigen-binding region that comprises the amino acid sequence of SEQ ID NO: 115 and specifically binds to a GPRC5D polypeptide (e.g., a GPRC5D polypeptide having the amino acid sequence SEQ ID NO:97, or fragments thereof), which is designated as ET150-155 scFv (also referred to as “ET150-5 scFv”).

In certain embodiments, the anti-GPRC5D scFv antibody comprises a heavy chain variable region comprising amino acids having the sequence set forth in SEQ ID NO:61 and a light chain variable region comprising amino acids having the sequence set forth in SEQ ID NO:62, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the anti-GPRC5D scFv antibody is an scFv-Fc fusion protein or full length human IgG with V_{H} and V_{L} regions or CDRs selected from Table 16. In certain embodiments, the anti-GPRC5D scFv comprises a V_{H} comprising amino acids having the sequence set forth in SEQ ID NO:61, as shown in Table 16. In certain embodiments, the anti-GPRC5D scFv comprises a V_{L} comprising amino acids having the sequence set forth in SEQ ID NO:62, as shown in Table 16. In certain embodiments, the anti-GPRC5D scFv comprises a V_{H} comprising amino acids having the sequence set forth in SEQ ID NO:61 and a V_{L} comprising amino acids having the sequence set forth in SEQ ID NO:62, as shown in Table 16. In certain embodiments, the anti-GPRC5D scFv comprises a V H CDR1 comprising amino acids having the sequence set forth in SEQ ID NO:214 or conservative modifications thereof, a V_{H} CDR2 comprising amino acids having the sequence set forth in SEQ ID NO: 215 or conservative modifications thereof, and a V_{H} CDR3 comprising amino acids having the sequence set forth in SEQ ID NO: 216 or conservative modifications thereof, as shown in Table 16. In certain embodiments, the anti-GPRC5D scFv comprises a V_{L} CDR1 comprising amino acids having the sequence set forth in SEQ ID NO: 217 or conservative modifications thereof, a V_{L} CDR2 comprising amino acids having the sequence set forth in SEQ ID NO: 218 or conservative modifications thereof, and a V_{L} CDR3 comprising amino acids having the sequence set forth in SEQ ID NO: 219 or conservative modifications thereof, as shown in Table 16. In certain embodiments, the anti-GPRC5D scFv comprises a V_{H} CDR1 comprising amino acids having the sequence set forth in SEQ ID NO: 214 or conservative modifications thereof, a V_{H} CDR2 comprising amino acids having the sequence set forth in SEQ ID NO: 215 or conservative modifications thereof, a V_{H} CDR3 comprising amino acids having the sequence set forth in SEQ ID NO: 216 or conservative modifications thereof, a V_{L} CDR1 comprising amino acids having the sequence set forth in SEQ ID NO: 217 or conservative modifications thereof, a V_{L} CDR2 comprising amino acids having the sequence set forth in SEQ ID NO: 218 or conservative modifications thereof, and a V_{L} CDR3 comprising amino acids having the sequence set forth in SEQ ID NO: 219 or conservative modifications thereof, as shown in Table 16. In certain embodiments, the anti-GPRC5D scFv comprises a V_{H} CDR1 comprising amino acids having the sequence set forth in SEQ ID NO: 214, a V_{H} CDR2 comprising amino acids having the sequence set forth in SEQ ID NO: 215, a V_{H} CDR3 comprising amino acids having the sequence set forth in SEQ ID NO: 216, a V_{L} CDR1 comprising amino acids having the sequence set forth in SEQ ID NO: 217, a V_{L} CDR2 comprising amino acids having the sequence set forth in SEQ ID NO: 218, and a V_{L} CDR3 comprising amino acids having the sequence set forth in SEQ ID NO: 219.

TABLE 16

| Antigen | A GPRC5D polypeptide having the amino acid sequence of SEQ ID NO: 97 | | |
|---|---|---|---|
| CDRs | 1 | 2 | 3 |
| V_{H} | GFTFSDYY [SEQ ID | ISSSGSTI [SEQ ID NO: | ARGYGKAYDQ [SEQ |
| | NO: 214] | 215] | ID NO: 216] |
| V_{L} | RSNVGGNY [SEQ | RSN [SEQ ID NO: 218] | ATWDDSLSGFV [SEQ |
| | ID NO: 217] | | ID NO: 219] |

| | |
|---|---|
| Full V_{H} | EVQLVESGGGLVKPGGSLRLSCAASGFTFSDYYMSWIRQAPGKGLEW |
| | VSYISSSGSTIYYADSVKGRFTISRDNAKNSLYLQMNSLRAEDTAVYY |
| | CARGYGKAYDQWGQGTLVTVSS [SEQ ID NO: 61] |
| DNA | Gaggtgcagctggtggagtctgggggaggcttggtcaagcctggagggtccctgagactctcctgtgcagcct |
| | ctggattcaccttcagtgactactacatgagctggatccgccaggctccagggaaggggctggagtgggtttcat |
| | acattagtagtagtggtagtaccatatactacgcagactctgtgaagggccgattcaccatctccagggacaacg |
| | ccaagaactcactgtatctgcaaatgaacagcctgagagccgaggacacggccgtatattactgtgcgcgcggt |
| | tacggtaaagcttacgatcagtggggtcaaggtactctggtgaccgtctcctca [SEQ ID NO: 63] |
| Full V_{L} | QSVLTQPPSASGTPGQRVTISCSGSRSNVGGNYVFWYQQVPGATPKLL |
| | IYRSNQRPSGVPDRFAGSKSGSSASLATSGLRSEDEADYYCATWDDSL |
| | SGFVFGTGTKVTVLG [SEQ ID NO: 62] |
| DNA | Cagtctgtgttgactcagccaccctcagcgtctgggacccccggacagagggtcaccatctcttgttctggaag |
| | caggtccaacgtaggaggtaattatgtattttggtaccagcaagtccccggagcgacccccaaactcctcatctat |
| | aggagtaatcageggccctcgggggtccctgaccgattcgctggctccaagtctggctcctcagcctccctggc |
| | catcagtggactccggtccgaggatgaggctgattattactgtgcaacatgggatgacagcctgagtggttttgtc |
| | ttcggaactgggaccaaggtcaccgtcctaggt [SEQ ID NO: 64] |
| scFv | QSVLTQPPSASGTPGQRVTISCSGSRSNVGGNYVFWYQQVPGATPKLL |
| | IYRSNQRPSGVPDRFAGSKSGSSASLATSGLRSEDEADYYCATWDDSL |
| | SGFVFGTGTKVTVLGSRGGGGSGGGGSGGGGSLEMAEVQLVESGGG |
| | LVKPGGSLRLSCAASGFTFSDYYMSWIRQAPGKGLEWVSYISSSGSTIY |
| | YADSVKGRFTISRDNAKNSLYLQMNSLRAEDTAVYYCARGYGKAYD |
| | QWGQGTLVTVSS [SEQ ID NO: 115] |

In certain embodiments, the antibody or other antigen binding protein is an anti-GPRC5D scFv or an antigen-binding fragment thereof having an antigen-binding region that comprises the amino acid sequence of SEQ ID NO: 116 and specifically binds to a GPRC5D polypeptide (e.g., a GPRC5D polypeptide having the amino acid sequence SEQ ID NO:97, or fragments thereof), which is designated as ET150-158 scFv (also referred to as “ET150-8 scFv”).

In certain embodiments, the anti-GPRC5D scFv antibody comprises a heavy chain variable region comprising amino acids having the sequence set forth in SEQ ID NO:65 and a light chain variable region comprising amino acids having the sequence set forth in SEQ ID NO:66, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the anti-GPRC5D scFv antibody is an scFv-Fc fusion protein or full length human IgG with V_{H} and V_{L} regions or CDRs selected from Table 17. In certain embodiments, the anti-GPRC5D scFv comprises a V_{H} comprising amino acids having the sequence set forth in SEQ ID NO:65, as shown in Table 17. In certain embodiments, the anti-GPRC5D scFv comprises a V_{L} comprising amino acids having the sequence set forth in SEQ ID NO:66, as shown in Table 17. In certain embodiments, the anti-GPRC5D scFv comprises a V_{H} comprising amino acids having the sequence set forth in SEQ ID NO:65 and a V_{L} comprising amino acids having the sequence set forth in SEQ ID NO:66, as shown in Table 17. In certain embodiments, the anti-GPRC5D scFv comprises a V H CDR1 comprising amino acids having the sequence set forth in SEQ ID NO:220 or conservative modifications thereof, a V_{H} CDR2 comprising amino acids having the sequence set forth in SEQ ID NO: 221 or conservative modifications thereof, and a V_{H} CDR3 comprising amino acids having the sequence set forth in SEQ ID NO: 222 or conservative modifications thereof, as shown in Table 17. In certain embodiments, the anti-GPRC5D scFv comprises a V_{L} CDR1 comprising amino acids having the sequence set forth in SEQ ID NO: 223 or conservative modifications thereof, a V_{L} CDR2 comprising amino acids having the sequence set forth in SEQ ID NO: 224 or conservative modifications thereof, and a V_{L} CDR3 comprising amino acids having the sequence set forth in SEQ ID NO: 225 or conservative modifications thereof, as shown in Table 17. In certain embodiments, the anti-GPRC5D scFv comprises a V_{H} CDR1 comprising amino acids having the sequence set forth in SEQ ID NO: 220 or conservative modifications thereof, a V_{H} CDR2 comprising amino acids having the sequence set forth in SEQ ID NO: 221 or conservative modifications thereof, a V_{H} CDR3 comprising amino acids having the sequence set forth in SEQ ID NO: 222 or conservative modifications thereof, a V_{L} CDR1 comprising amino acids having the sequence set forth in SEQ ID NO: 223 or conservative modifications thereof, a V_{L} CDR2 comprising amino acids having the sequence set forth in SEQ ID NO: 224 or conservative modifications thereof, and a V_{L} CDR3 comprising amino acids having the sequence set forth in SEQ ID NO: 225 or conservative modifications thereof, as shown in Table 17. In certain embodiments, the anti-GPRC5D scFv comprises a V_{H} CDR1 comprising amino acids having the sequence set forth in SEQ ID NO: 220, a V_{H} CDR2 comprising amino acids having the sequence set forth in SEQ ID NO: 221, a V_{H} CDR3 comprising amino acids having the sequence set forth in SEQ ID NO: 222, a V_{L} CDR1 comprising amino acids having the sequence set forth in SEQ ID NO: 223, a V_{L} CDR2 comprising amino acids having the sequence set forth in SEQ ID NO: 224, and a V_{L} CDR3 comprising amino acids having the sequence set forth in SEQ ID NO: 225.

TABLE 17

| Antigen | A GPRC5D polypeptide having the amino acid sequence of SEQ ID NO: 97 | | |
|---|---|---|---|
| CDRs | 1 | 2 | 3 |
| V_{H} | GFTFRSHS [SEQ ID | ISSDSTYT [SEQ ID | ARSGGQWKYYDY |
| | NO: 220] | NO: 221] | [SEQ ID NO: 222] |
| V_{L} | SLRSYY [SEQ ID | GKN [SEQ ID NO: 224] | NSRDSSGNPPVV |
| | NO: 223] | | [SEQ ID NO: 225] |

| | |
|---|---|
| Full V_{H} | QVQLVESGGGLVHPGGSLRLSCAASGFTFRSHSMNWVRQAPGKGLE |
| | WVSSISSDSTYTYYADSVKGRFTISRDNAKNSLYLQMNSLRAEDTAV |
| | YYCARSGGQWKYYDYWGQGTLVTVSS [SEQ ID NO: 65] |
| DNA | Caggtgcagctggtggagtctgggggaggcctggtccaccctggggggtccctgagactctcctgtgcagcct |
| | ctggattcaccttcagaagccatagcatgaactgggtccgccaggctccagggaaggggctggagtgggtctc |
| | atccattagtagtgatagtacttacacatactacgcagactcagtgaagggccgattcaccatctccagagacaac |
| | gccaagaactcactgtatctgcaaatgaacagcctgagagccgaggacacggccgtatattactgtgcgcgctc |
| | tggtggtcagtggaaatactacgattactggggtcaaggtactctggtgaccgtctcctca [SEQ ID |
| | NO: 67] |
| Full V_{L} | SSELTQDPAVSVALGQTVRITCQGDSLRSYYASWYQQKPGQAPVLVIY |
| | GKNNRPSGIPDRFSGSSSGNTASLTITGAQAEDEADYYCNSRDSSGNPP |
| | VVFGGGTKLTVLG [SEQ ID NO: 66] |
| DNA | Tcttctgagctgactcaggaccctgctgtgtctgtggccttgggacagacagtcaggatcacatgccaaggaga |
| | cagcctcagaagctattatgcaagctggtaccagcagaagccaggacaggcccctgtacttgtcatctatggtaa |
| | aaacaaccggccctcagggatcccagaccgattctctggctccagctcaggaaacacagcttccttgaccatca |
| | ctggggctcaggeggaagatgaggctgactattactgtaactcccgggacagcagtggtaacccccctgtggta |
| | ttcggcggagggaccaagctgaccgtcctaggt [SEQ ID NO: 68] |
| scFv | SSELTQDPAVSVALGQTVRITCQGDSLRSYYASWYQQKPGQAPVLVIY |
| | GKNNRPSGIPDRFSGSSSGNTASLTITGAQAEDEADYYCNSRDSSGNPP |
| | VVFGGGTKLTVLGSRGGGGSGGGGSGGGGSLEMAQVQLVESGGGLV |
| | HPGGSLRLSCAASGFTFRSHSMNWVRQAPGKGLEWVSSISSDSTYTY |
| | YADSVKGRFTISRDNAKNSLYLQMNSLRAEDTAVYYCARSGGQWKY |
| | YDYWGQGTLVTVSS [SEQ ID NO: 116] |

In certain embodiments, the antibody or other antigen binding protein is an anti-GPRC5D scFv or an antigen-binding fragment thereof having an antigen-binding region that comprises the amino acid sequence of SEQ ID NO: 117 and specifically binds to a GPRC5D polypeptide (e.g., a GPRC5D polypeptide having the amino acid sequence SEQ ID NO:97, or fragments thereof), which is designated as ET150-168 scFv (also referred to as “ET150-18 scFv”).

In certain embodiments, the anti-GPRC5D scFv antibody comprises a heavy chain variable region comprising amino acids having the sequence set forth in SEQ ID NO:69 and a light chain variable region comprising amino acids having the sequence set forth in SEQ ID NO:70, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the anti-GPRC5D scFv antibody is an scFv-Fc fusion protein or full length human IgG with V_{H} and V_{L} regions or CDRs selected from Table 18. In certain embodiments, the anti-GPRC5D scFv comprises a V_{H} comprising amino acids having the sequence set forth in SEQ ID NO:69, as shown in Table 18. In certain embodiments, the anti-GPRC5D scFv comprises a V_{L} comprising amino acids having the sequence set forth in SEQ ID NO:70, as shown in Table 18. In certain embodiments, the anti-GPRC5D scFv comprises a V_{H} comprising amino acids having the sequence set forth in SEQ ID NO:69 and a V_{L} comprising amino acids having the sequence set forth in SEQ ID NO:70, as shown in Table 18. In certain embodiments, the anti-GPRC5D scFv comprises a V_{H} CDR1 comprising amino acids having the sequence set forth in SEQ ID NO:226 or conservative modifications thereof, a V_{H} CDR2 comprising amino acids having the sequence set forth in SEQ ID NO: 227 or conservative modifications thereof, and a V_{H} CDR3 comprising amino acids having the sequence set forth in SEQ ID NO: 228 or conservative modifications thereof, as shown in Table 18. In certain embodiments, the anti-GPRC5D scFv comprises a V_{L} CDR1 comprising amino acids having the sequence set forth in SEQ ID NO: 229 or conservative modifications thereof, a V_{L} CDR2 comprising amino acids having the sequence set forth in SEQ ID NO: 230 or conservative modifications thereof, and a V_{L} CDR3 comprising amino acids having the sequence set forth in SEQ ID NO: 231 or conservative modifications thereof, as shown in Table 18. In certain embodiments, the anti-GPRC5D scFv comprises a V_{H} CDR1 comprising amino acids having the sequence set forth in SEQ ID NO: 226 or conservative modifications thereof, a V_{H} CDR2 comprising amino acids having the sequence set forth in SEQ ID NO: 227 or conservative modifications thereof, a V_{H} CDR3 comprising amino acids having the sequence set forth in SEQ ID NO: 228 or conservative modifications thereof, a V_{L} CDR1 comprising amino acids having the sequence set forth in SEQ ID NO: 229 or conservative modifications thereof, a V_{L} CDR2 comprising amino acids having the sequence set forth in SEQ ID NO: 230 or conservative modifications thereof, and a V_{L} CDR3 comprising amino acids having the sequence set forth in SEQ ID NO: 231 or conservative modifications thereof, as shown in Table 18. In certain embodiments, the anti-GPRC5D scFv comprises a V_{H} CDR1 comprising amino acids having the sequence set forth in SEQ ID NO: 226, a V_{H} CDR2 comprising amino acids having the sequence set forth in SEQ ID NO: 227, a V_{H} CDR3 comprising amino acids having the sequence set forth in SEQ ID NO: 228, a V_{L} CDR1 comprising amino acids having the sequence set forth in SEQ ID NO: 229, a V_{L} CDR2 comprising amino acids having the sequence set forth in SEQ ID NO: 230, and a V_{L} CDR3 comprising amino acids having the sequence set forth in SEQ ID NO: 231.

TABLE 18

| Antigen | A GPRC5D polypeptide having the amino acid sequence of SEQ ID NO: 97 | | |
|---|---|---|---|
| CDRs | 1 | 2 | 3 |
| V_{H} | GFTFSNYA [SEQ ID | INGRGSST [SEQ ID NO: | ARYISRGLGDS [SEQ |
| | NO: 226] | 227] | ID NO: 228] |
| V_{L} | NSNIERNY[SEQ ID | DND [SEQ ID NO: 230] | GTWDSSLRGWV |
| | NO: 229] | | [SEQ ID NO: 231] |

| | |
|---|---|
| Full V_{H} | EVQLVESGGGLIQPGGSLRLSCAASGFTFSNYAMNWVRQAPGKGLEW |
| | VSTINGRGSSTIYADSVKGRFTISRDNSKNTLYLQMNSLRAEDTATYY |
| | CARYISRGLGDSWGQGTLVTV [SEQ ID NO: 69] |
| DNA | Gaggtgcagctggtggagtccgggggaggcttgatacagcctggggggtccctgagactctcctgtgcagcc |
| | tctggattcacctttagcaactatgccatgaactgggtccgccaggctccagggaaggggctggagtgggtctc |
| | aactattaatggtcgtggtagtagtacaatctacgcagactccgtgaagggccggttcaccatctccagagacaa |
| | ttccaagaacacgctgtatctgcaaatgaacagcctgagagccgaggacacagccacgtattactgtgcgcgct |
| | acatctctcgtggtctgggtgattcttggggtcaaggtactctggtgaccgtctcctca [SEQ ID NO: 71] |
| Full V_{L} | QSVVTQPPSMSAAPGQQVTISCSGGNSNIERNYVSWYLQLPGTAPKLV |
| | IFDNDRRPSGIPDRFSGSKSGTSATLGITGLQTGDEADYYCGTWDSSLR |
| | GWVFGGGTKLTVLG [SEQ ID NO: 70] |
| DNA | Cagtctgtcgtgacgcagccgccctcaatgtctgcggccccaggacagcaagtcaccatctcctgctctggag |
| | gcaactccaacattgagagaaattatgtatcctggtacctccagctccctggaacagcccccaaactcgtcattttt |
| | gacaatgataggcgaccctcagggattcctgaccgattctctggctccaagtctggcacgtcagccaccctggg |
| | catcaccggactccagactggggacgaggccgattattactgcggaacatgggatagcagcctgagaggttgg |
| | gtgttcggcggagggaccaagctgaccgtcctaggt [SEQ ID NO: 72] |
| scFv | QSVVTQPPSMSAAPGQQVTISCSGGNSNIERNYVSWYLQLPGTAPKLV |
| | IFDNDRRPSGIPDRFSGSKSGTSATLGITGLQTGDEADYYCGTWDSSLR |
| | GWVFGGGTKLTVLGSRGGGGSGGGGSGGGGSLEMAEVQLVESGGGL |
| | IQPGGSLRLSCAASGFTFSNYAMNWVRQAPGKGLEWVSTINGRGSSTI |
| | YADSVKGRFTISRDNSKNTLYLQMNSLRAEDTATYYCARYISRGLGDS |
| | WGQGTLVTV [SEQ ID NO: 117] |

In certain embodiments, the antibody or other antigen binding protein is an anti-GPRC5D scFv or an antigen-binding fragment thereof having an antigen-binding region that comprises the amino acid sequence of SEQ ID NO: 118 and specifically binds to a GPRC5D polypeptide (e.g., a GPRC5D polypeptide having the amino acid sequence SEQ ID NO:97, or fragments thereof), which is designated as ET150-164 scFv (also referred to as “ET150-14 scFv”).

In certain embodiments, the anti-GPRC5D scFv antibody comprises a heavy chain variable region comprising amino acids having the sequence set forth in SEQ ID NO:73 and a light chain variable region comprising amino acids having the sequence set forth in SEQ ID NO:74, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the anti-GPRC5D scFv antibody is an scFv-Fc fusion protein or full length human IgG with V_{H} and V_{L} regions or CDRs selected from Table 19. In certain embodiments, the anti-GPRC5D scFv comprises a V_{H} comprising amino acids having the sequence set forth in SEQ ID NO:73, as shown in Table 19. In certain embodiments, the anti-GPRC5D scFv comprises a V_{L} comprising amino acids having the sequence set forth in SEQ ID NO:74, as shown in Table 19. In certain embodiments, the anti-GPRC5D scFv comprises a V_{H} comprising amino acids having the sequence set forth in SEQ ID NO:73 and a V_{L} comprising amino acids having the sequence set forth in SEQ ID NO:74, as shown in Table 19. In certain embodiments, the anti-GPRC5D scFv comprises a V H CDR1 comprising amino acids having the sequence set forth in SEQ ID NO:232 or conservative modifications thereof, a V_{H} CDR2 comprising amino acids having the sequence set forth in SEQ ID NO: 233 or conservative modifications thereof, and a V_{H} CDR3 comprising amino acids having the sequence set forth in SEQ ID NO: 234 or conservative modifications thereof, as shown in Table 19. In certain embodiments, the anti-GPRC5D scFv comprises a V_{L} CDR1 comprising amino acids having the sequence set forth in SEQ ID NO: 235 or conservative modifications thereof, a V_{L} CDR2 comprising amino acids having the sequence set forth in SEQ ID NO: 236 or conservative modifications thereof, and a V_{L} CDR3 comprising amino acids having the sequence set forth in SEQ ID NO: 237 or conservative modifications thereof, as shown in Table 19. In certain embodiments, the anti-GPRC5D scFv comprises a V_{H} CDR1 comprising amino acids having the sequence set forth in SEQ ID NO: 232 or conservative modifications thereof, a V_{H} CDR2 comprising amino acids having the sequence set forth in SEQ ID NO: 233 or conservative modifications thereof, a V_{H} CDR3 comprising amino acids having the sequence set forth in SEQ ID NO: 234 or conservative modifications thereof, a V_{L} CDR1 comprising amino acids having the sequence set forth in SEQ ID NO: 235 or conservative modifications thereof, a V_{L} CDR2 comprising amino acids having the sequence set forth in SEQ ID NO: 236 or conservative modifications thereof, and a V_{L} CDR3 comprising amino acids having the sequence set forth in SEQ ID NO: 237 or conservative modifications thereof, as shown in Table 19. In certain embodiments, the anti-GPRC5D scFv comprises a V_{H} CDR1 comprising amino acids having the sequence set forth in SEQ ID NO: 232, a V_{H} CDR2 comprising amino acids having the sequence set forth in SEQ ID NO: 233, a V_{H} CDR3 comprising amino acids having the sequence set forth in SEQ ID NO: 234, a V_{L} CDR1 comprising amino acids having the sequence set forth in SEQ ID NO: 235, a V_{L} CDR2 comprising amino acids having the sequence set forth in SEQ ID NO: 236, and a V_{L} CDR3 comprising amino acids having the sequence set forth in SEQ ID NO: 237.

TABLE 19

| Antigen | A GPRC5D polypeptide having the amino acid sequence of SEQ ID NO: 97 | | |
|---|---|---|---|
| CDRs | 1 | 2 | 3 |
| V_{H} | GYTFTSYY [SEQ ID | INPSGGST [SEQ ID | ARAGMGMDT [SEQ |
| | NO: 232] | NO: 233 | ID NO: 234] |
| V_{L} | SSDVGGYNY [SEQ | EVS [SEQ ID NO: 236] | SSYAGSNTLV [SEQ |
| | ID NO: 235] | | ID NO: 237] |

| | |
|---|---|
| Full V_{H} | QMQLVQSGAEVKKPGASVKVSCKASGYTFTSYYMEIWVRQAPGQGL |
| | EWMGIINPSGGSTSYAQKFQGRVTMTRDTSTSTVYMELSSLRSEDTAV |
| | YYCARAGMGMDTWGQGTLVTVSS [SEQ ID NO: 73] |
| DNA | Cagatgcagctggtgcagtctggggctgaggtgaagaagcctggggcctcagtgaaggtttcctgcaaggcat |
| | ctggatacaccttcaccagctactatatgcactgggtgcgacaggcccctggacaagggcttgagtggatggga |
| | ataatcaaccctagtggtggtagcacaagctacgcacagaagttccagggcagagtcaccatgaccagggaca |
| | cgtccacgagcacagtctacatggagctgagcagcctgagatctgaggacacggccgtgtattactgtgcgcg |
| | cgctggtatgggtatggatacttggggtcaaggtactctggtgaccgtctcctca [SEQ ID NO: 75] |
| Full V_{L} | QSALTQPPSASGSPGQSVTISCTGTSSDVGGYNYVSWYQQHPGKAPKL |
| | MIYEVSKRPSGVPDRFSGSKSGNTASLTVSGLQAEDEADYYCSSYAGS |
| | NTLVFGGGTKLTVLG [SEQ ID NO: 74] |
| DNA | Cagtctgccctgactcagcctccctccgcgtccgggtctcctggacagtcagtcaccatctcctgcactggaac |
| | cagcagtgacgttggtggttataactatgtctcctggtaccaacagcacccaggcaaagcccccaaactcatgat |
| | ttatgaggtcagtaageggccctcaggggtccctgatcgcttctctggctccaagtctggcaacacggcctccct |
| | gaccgtctctgggctccaggctgaggatgaggctgattattactgcagctcatatgcaggcagcaacaccttggt |
| | gttcggcggagggaccaagctgaccgtcctaggt [SEQ ID NO: 76] |
| scFv | QSALTQPPSASGSPGQSVTISCTGTSSDVGGYNYVSWYQQHPGKAPKL |
| | MIYEVSKRPSGVPDRFSGSKSGNTASLTVSGLQAEDEADYYCSSYAGS |
| | NTLVFGGGTKLTVLGSRGGGGSGGGGSGGGGSLEMAQMQLVQSGAE |
| | VKKPGASVKVSCKASGYTFTSYYMHWVRQAPGQGLEWMGIINPSGG |
| | STSYAQKFQGRVTMTRDTSTSTVYMELSSLRSEDTAVYYCARAGMG |
| | MDTWGQGTLVTVSS [SEQ ID NO: 118] |

In certain embodiments, the antibody or other antigen binding protein is an anti-GPRC5D scFv or an antigen-binding fragment thereof having an antigen-binding region that comprises the amino acid sequence of SEQ ID NO: 119 and specifically binds to a GPRC5D polypeptide (e.g., a GPRC5D polypeptide having the amino acid sequence SEQ ID NO:97, or fragments thereof), which is designated as ET150-165 scFv (also referred to as “ET150-15 scFv”).

In certain embodiments, the anti-GPRC5D scFv antibody comprises a heavy chain variable region comprising amino acids having the sequence set forth in SEQ ID NO:77 and a light chain variable region comprising amino acids having the sequence set forth in SEQ ID NO:78, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the anti-GPRC5D scFv antibody is an scFv-Fc fusion protein or full length human IgG with V_{H} and V_{L} regions or CDRs selected from Table 20. In certain embodiments, the anti-GPRC5D scFv comprises a V_{H} comprising amino acids having the sequence set forth in SEQ ID NO:77, as shown in Table 20. In certain embodiments, the anti-GPRC5D scFv comprises a V_{L} comprising amino acids having the sequence set forth in SEQ ID NO:78, as shown in Table 20. In certain embodiments, the anti-GPRC5D scFv comprises a V_{H} comprising amino acids having the sequence set forth in SEQ ID NO:77 and a V_{L} comprising amino acids having the sequence set forth in SEQ ID NO:78, as shown in Table 20. In certain embodiments, the anti-GPRC5D scFv comprises a V H CDR1 comprising amino acids having the sequence set forth in SEQ ID NO:238 or conservative modifications thereof, a V_{H} CDR2 comprising amino acids having the sequence set forth in SEQ ID NO: 239 or conservative modifications thereof, and a V_{H} CDR3 comprising amino acids having the sequence set forth in SEQ ID NO: 240 or conservative modifications thereof, as shown in Table 20. In certain embodiments, the anti-GPRC5D scFv comprises a V_{L} CDR1 comprising amino acids having the sequence set forth in SEQ ID NO: 241 or conservative modifications thereof, a V_{L} CDR2 comprising amino acids having the sequence set forth in SEQ ID NO: 242 or conservative modifications thereof, and a V_{L} CDR3 comprising amino acids having the sequence set forth in SEQ ID NO: 243 or conservative modifications thereof, as shown in Table 20. In certain embodiments, the anti-GPRC5D scFv comprises a V_{H} CDR1 comprising amino acids having the sequence set forth in SEQ ID NO: 238 or conservative modifications thereof, a V_{H} CDR2 comprising amino acids having the sequence set forth in SEQ ID NO: 239 or conservative modifications thereof, a V_{H} CDR3 comprising amino acids having the sequence set forth in SEQ ID NO: 240 or conservative modifications thereof, a V_{L} CDR1 comprising amino acids having the sequence set forth in SEQ ID NO: 241 or conservative modifications thereof, a V_{L} CDR2 comprising amino acids having the sequence set forth in SEQ ID NO: 242 or conservative modifications thereof, and a V_{L} CDR3 comprising amino acids having the sequence set forth in SEQ ID NO: 243 or conservative modifications thereof, as shown in Table 20. In certain embodiments, the anti-GPRC5D scFv comprises a V_{H} CDR1 comprising amino acids having the sequence set forth in SEQ ID NO: 238, a V_{H} CDR2 comprising amino acids having the sequence set forth in SEQ ID NO: 239, a V_{H} CDR3 comprising amino acids having the sequence set forth in SEQ ID NO: 240, a V_{L} CDR1 comprising amino acids having the sequence set forth in SEQ ID NO: 241, a V_{L} CDR2 comprising amino acids having the sequence set forth in SEQ ID NO: 242, and a V_{L} CDR3 comprising amino acids having the sequence set forth in SEQ ID NO: 243.

TABLE 20

| Antigen | A GPRC5D polypeptide having the amino acid sequence of SEQ ID NO: 97 | | |
|---|---|---|---|
| CDRs | 1 | 2 | 3 |
| V_{H} | GYTFTAYS [SEQ ID | INPSSGGA [SEQ ID | ARNVGGQADD [SEQ |
| | NO: 238] | NO: 239] | ID NO: 240] |
| V_{L} | SSDIGGYNY [SEQ ID | EVN [SEQ ID NO: 242] | ASFAGRKTLV [SEQ |
| | NO: 241] | | ID NO: 243] |

| | |
|---|---|
| Full V_{H} | QVQLVQSGAEVKKPGASVKVSCRASGYTFTAYSLHWVRQAPGQGLE |
| | WMGWINPSSGGAVYAQKFQGRVTMTRDTSISTAYMELSGLRSDDTA |
| | VYYCARNVGGQADDWGQGTLVTVSS [SEQ ID NO: 77] |
| DNA | Caggtgcagctggtgcagtctggggctgaggtgaagaagcctggggcctcagtgaaggtctcctgcagggct |
| | tctggatacaccttcaccgcctactattacactgggtgcgacaggcccctggacaagggcttgagtggatggga |
| | tggatcaaccctagcagtggtggcgcagtttatgcacagaaatttcagggtagggtcaccatgaccagggacac |
| | gtccatcagcacagcctacatggagctgagtggcctgagatctgacgacacggccgtgtattactgtgcgcgca |
| | acgttggtggtcaggctgatgactggggtcaaggtactctggtgaccgtctcctca [SEQ ID NO: 79] |
| Full V_{L} | QSALTQPPSASGSPGQSVTISCTGTSSDIGGYNYVSWYQQHPGKAPKL |
| | MIYEVNKRPSGVPDRFSGSKSGNTASLTVSGLQAEDEADYYCASFAG |
| | RKTLVFGGGTKLTVLG [SEQ ID NO: 78] |
| DNA | Caatctgccctgactcagcctccctccgcgtccgggtctcctggacagtcagtcaccatctcctgcactggaacc |
| | agcagtgacattggtggttataactatgtctcctggtaccaacagcacccaggcaaagcccccaaactcatgattt |
| | atgaggtcaataageggccctcaggggtccctgatcgcttctcgggctccaagtctggcaacacggcctccctg |
| | accgtctctgggctccaggctgaggatgaggctgattattactgcgcctcatttgegggcaggaagacattggtc |
| | ttcggcggagggaccaagctgaccgtcctaggt [SEQ ID NO: 80] |
| scFv | QSALTQPPSASGSPGQSVTISCTGTSSDIGGYNYVSWYQQHPGKAPKL |
| | MIYEVNKRPSGVPDRFSGSKSGNTASLTVSGLQAEDEADYYCASFAG |
| | RKTLVFGGGTKLTVLGSRGGGGSGGGGSGGGGSLEMAQVQLVQSGA |
| | EVKKPGASVKVSCRASGYTFTAYSLHWVRQAPGQGLEWMGWINPSS |
| | GGAVYAQKFQGRVTMTRDTSISTAYMELSGLRSDDTAVYYCARNVG |
| | GQADDWGQGTLVTVSS [SEQ ID NO: 119] |

In certain embodiments, the antibody or other antigen binding protein is an anti-GPRC5D scFv or an antigen-binding fragment thereof having an antigen-binding region that comprises the amino acid sequence of SEQ ID NO: 120 and specifically binds to a GPRC5D polypeptide (e.g., a GPRC5D polypeptide having the amino acid sequence SEQ ID NO:97, or fragments thereof), which is designated as ET150-167 scFv (also referred to as “ET150-17 scFv”).

In certain embodiments, the anti-GPRC5D scFv antibody comprises a heavy chain variable region comprising amino acids having the sequence set forth in SEQ ID NO:81 and a light chain variable region comprising amino acids having the sequence set forth in SEQ ID NO:82, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the anti-GPRC5D scFv antibody is an scFv-Fc fusion protein or full length human IgG with V_{H} and V_{L} regions or CDRs selected from Table 21. In certain embodiments, the anti-GPRC5D scFv comprises a V_{H} comprising amino acids having the sequence set forth in SEQ ID NO:81, as shown in Table 21. In certain embodiments, the anti-GPRC5D scFv comprises a V_{L} comprising amino acids having the sequence set forth in SEQ ID NO:82, as shown in Table 21. In certain embodiments, the anti-GPRC5D scFv comprises a V_{H} comprising amino acids having the sequence set forth in SEQ ID NO:81 and a V_{L} comprising amino acids having the sequence set forth in SEQ ID NO:82, as shown in Table 21. In certain embodiments, the anti-GPRC5D scFv comprises a V H CDR1 comprising amino acids having the sequence set forth in SEQ ID NO:244 or conservative modifications thereof, a V_{H} CDR2 comprising amino acids having the sequence set forth in SEQ ID NO: 245 or conservative modifications thereof, and a V_{H} CDR3 comprising amino acids having the sequence set forth in SEQ ID NO: 246 or conservative modifications thereof, as shown in Table 21. In certain embodiments, the anti-GPRC5D scFv comprises a V_{L} CDR1 comprising amino acids having the sequence set forth in SEQ ID NO: 247 or conservative modifications thereof, a V_{L} CDR2 comprising amino acids having the sequence set forth in SEQ ID NO: 248 or conservative modifications thereof, and a V_{L} CDR3 comprising amino acids having the sequence set forth in SEQ ID NO: 249 or conservative modifications thereof, as shown in Table 21. In certain embodiments, the anti-GPRC5D scFv comprises a V_{H} CDR1 comprising amino acids having the sequence set forth in SEQ ID NO: 244 or conservative modifications thereof, a V_{H} CDR2 comprising amino acids having the sequence set forth in SEQ ID NO: 245 or conservative modifications thereof, a V_{H} CDR3 comprising amino acids having the sequence set forth in SEQ ID NO: 246 or conservative modifications thereof, a V_{L} CDR1 comprising amino acids having the sequence set forth in SEQ ID NO: 247 or conservative modifications thereof, a V_{L} CDR2 comprising amino acids having the sequence set forth in SEQ ID NO: 248 or conservative modifications thereof, and a V_{L} CDR3 comprising amino acids having the sequence set forth in SEQ ID NO: 249 or conservative modifications thereof, as shown in Table 21. In certain embodiments, the anti-GPRC5D scFv comprises a V_{H} CDR1 comprising amino acids having the sequence set forth in SEQ ID NO: 244, a V_{H} CDR2 comprising amino acids having the sequence set forth in SEQ ID NO: 245, a V_{H} CDR3 comprising amino acids having the sequence set forth in SEQ ID NO: 246, a V_{L} CDR1 comprising amino acids having the sequence set forth in SEQ ID NO: 247, a V_{L} CDR2 comprising amino acids having the sequence set forth in SEQ ID NO: 248, and a V_{L} CDR3 comprising amino acids having the sequence set forth in SEQ ID NO: 249.

TABLE 21

| Antigen | A GPRC5D polypeptide having the amino acid sequence of SEQ ID NO: 97 | | |
|---|---|---|---|
| CDRs | 1 | 2 | 3 |
| V_{H} | GYTFTAYS [SEQ ID | INPSSGGA [SEQ ID | ARNVGGHADD [SEQ |
| | NO: 244] | NO: 245] | ID NO: 246] |
| V_{L} | STDIGGYNY [SEQ | EVN [SEQ ID NO: 248] | ASFAGRKTLV [SEQ |
| | ID NO: 247] | | ID NO: 249] |

| | |
|---|---|
| Full V_{H} | QVQLVQSGAEVKKPGASVKVSCRASGYTFTAYSLHWVRQAPGQGLE |
| | WMGWINPSSGGAVYAQKFQGRVTMTRDTSISTAYMELSGLRSDDTA |
| | VYYCARNVGGHADDWGQGTLVTVSS [SEQ ID NO: 81] |
| DNA | Caggtgcagctggtgcagtctggggctgaggtgaaaaagcctggggcctcagtgaaagtctcctgcagggctt |
| | ctggatacaccttcaccgcctactattacactgggtgcgacaggccectggacaagggcttgagtggatgggat |
| | ggatcaaccctagcagtggtggcgcagtttatgcacagaaatttcagggtagggtcaccatgaccagggacac |
| | gtccatcagcacagcctacatggagctgagtggcctgagatctgacgacacggccgtgtattactgtgcgcgca |
| | acgttggtggtcacgctgatgactggggtcaaggtactctggtgaccgtctcctca [SEQ ID NO: 83] |
| Full V_{L} | QSALTQPPSASGSPGQSVTISCTGTSTDIGGYNYVSWYQHHPSKAPKL |
| | MIYEVNKRPSGVPDRFSGSKSGNTASLTVSGLQAEDEADYYCASFAG |
| | RKTLVFGGGTKLTVLG [SEQ ID NO: 82] |
| DNA | Caatctgccctgactcagcctccctccgcgtccgggtctcctggacagtcagtcaccatctcctgcactggaacc |
| | agcactgacattggtggttataactatgtctcctggtaccaacaccacccaagcaaagcccccaaactcatgattt |
| | atgaggtcaataageggccctcaggggtccctgatcgcttctcgggctccaagtctggcaacacggcctccctg |
| | accgtctctgggctccaggctgaggatgaggctgattattactgcgcctcatttgegggcaggaagacattggtc |
| | ttcggcggagggaccaagctgaccgtcctaggt [SEQ ID NO: 84] |
| scFv | QSALTQPPSASGSPGQSVTISCTGTSTDIGGYNYVSWYQHHPSKAPKL |
| | MIYEVNKRPSGVPDRFSGSKSGNTASLTVSGLQAEDEADYYCASFAG |
| | RKTLVFGGGTKLTVLGSRGGGGSGGGGSGGGGSLEMAQVQLVQSGA |
| | EVKKPGASVKVSCRASGYTFTAYSLHWVRQAPGQGLEWMGWINPSS |
| | GGAVYAQKFQGRVTMTRDTSISTAYMELSGLRSDDTAVYYCARNVG |
| | GHADDWGQGTLVTVSS [SEQ ID NO: 120] |

In certain embodiments, the antibody or other antigen binding protein is an anti-GPRC5D scFv or an antigen-binding fragment thereof having an antigen-binding region that comprises the amino acid sequence of SEQ ID NO: 121 and specifically binds to a GPRC5D polypeptide (e.g., a GPRC5D polypeptide having the amino acid sequence SEQ ID NO:97, or fragments thereof), which is designated as ET150-169 scFv (also referred to as “ET150-19 scFv”).

In certain embodiments, the anti-GPRC5D scFv antibody comprises a heavy chain variable region comprising amino acids having the sequence set forth in SEQ ID NO:85 and a light chain variable region comprising amino acids having the sequence set forth in SEQ ID NO:86, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the anti-GPRC5D scFv antibody is an scFv-Fc fusion protein or full length human IgG with V_{H} and V_{L} regions or CDRs selected from Table 22. In certain embodiments, the anti-GPRC5D scFv comprises a V_{H} comprising amino acids having the sequence set forth in SEQ ID NO:85, as shown in Table 22. In certain embodiments, the anti-GPRC5D scFv comprises a V_{L} comprising amino acids having the sequence set forth in SEQ ID NO:86, as shown in Table 22. In certain embodiments, the anti-GPRC5D scFv comprises a V_{H} comprising amino acids having the sequence set forth in SEQ ID NO:85 and a V_{L} comprising amino acids having the sequence set forth in SEQ ID NO:86, as shown in Table 22. In certain embodiments, the anti-GPRC5D scFv comprises a V H CDR1 comprising amino acids having the sequence set forth in SEQ ID NO:250 or conservative modifications thereof, a V_{H} CDR2 comprising amino acids having the sequence set forth in SEQ ID NO: 251 or conservative modifications thereof, and a V_{H} CDR3 comprising amino acids having the sequence set forth in SEQ ID NO: 252 or conservative modifications thereof, as shown in Table 22. In certain embodiments, the anti-GPRC5D scFv comprises a V_{L} CDR1 comprising amino acids having the sequence set forth in SEQ ID NO: 253 or conservative modifications thereof, a V_{L} CDR2 comprising amino acids having the sequence set forth in SEQ ID NO: 254 or conservative modifications thereof, and a V_{L} CDR3 comprising amino acids having the sequence set forth in SEQ ID NO: 255 or conservative modifications thereof, as shown in Table 22. In certain embodiments, the anti-GPRC5D scFv comprises a V_{H} CDR1 comprising amino acids having the sequence set forth in SEQ ID NO: 250 or conservative modifications thereof, a V_{H} CDR2 comprising amino acids having the sequence set forth in SEQ ID NO: 251 or conservative modifications thereof, a V_{H} CDR3 comprising amino acids having the sequence set forth in SEQ ID NO: 252 or conservative modifications thereof, a V_{L} CDR1 comprising amino acids having the sequence set forth in SEQ ID NO: 253 or conservative modifications thereof, a V_{L} CDR2 comprising amino acids having the sequence set forth in SEQ ID NO: 254 or conservative modifications thereof, and a V_{L} CDR3 comprising amino acids having the sequence set forth in SEQ ID NO: 255 or conservative modifications thereof, as shown in Table 22. In certain embodiments, the anti-GPRC5D scFv comprises a V_{H} CDR1 comprising amino acids having the sequence set forth in SEQ ID NO: 250, a V_{H} CDR2 comprising amino acids having the sequence set forth in SEQ ID NO: 251, a V_{H} CDR3 comprising amino acids having the sequence set forth in SEQ ID NO: 252, a V_{L} CDR1 comprising amino acids having the sequence set forth in SEQ ID NO: 253, a V_{L} CDR2 comprising amino acids having the sequence set forth in SEQ ID NO: 254, and a V_{L} CDR3 comprising amino acids having the sequence set forth in SEQ ID NO: 255.

TABLE 22

| Antigen | A GPRC5D polypeptide having the amino acid sequence of SEQ ID NO: 97 | | |
|---|---|---|---|
| CDRs | 1 | 2 | 3 |
| V_{H} | GFTFNTYG [SEQ ID | ISANNGHT [SEQ ID | ARGGYHHQMQRYYK |
| | NO: 250] | NO: 251] | ATSVYSDY [SEQ ID |
| | | | NO: 252] |
| V_{L} | SSNIGNNY [SEQ ID | DNN [SEQ ID NO: | GTWDSSLSGVV [SEQ |
| | NO: 253] | 254] | ID NO: 255] |

| | |
|---|---|
| Full V_{H} | QVQLVQSGGEVKKPGASVKVSCKASGFTFNTYGISWVRQAPGQGLE |
| | WMGWISANNGHTKSAQRFQDRVAMATDTSTSTAYMELRSLKFDDTA |
| | VYYCARGGYHHQMQRYYKATSVYSDYWGQGTLVTVSS [SEQ ID |
| | NO: 85] |
| DNA | Caggtccagctggtgcagtctggaggtgaggtgaagaagcctggggcctcagtgaaggtctcctgcaaggctt |
| | ctggtttcacctttaacacctatggcatcagttgggtgcgacaggccectggacaagggcttgagtggatgggat |
| | ggatcagcgctaacaatggtcacacaaagtctgcacagaggttccaggacagagtcgccatggccacagaca |
| | catccacgagcacggcctacatggagctgaggagcctgaaatttgacgacacggccgtgtattactgtgcgcgc |
| | ggtggttaccatcatcagatgcageggtactacaaagctacttctgtttactctgattactggggtcaaggtactctg |
| | gtgaccgtctcctca [SEQ ID NO: 87] |
| Full V_{L} | QSVVTQPPSVSAAPGQKVTISCSGSSSNIGNNYVSWYQQLPGTAPKLLI |
| | YDNNKRPSGIPDRFSGSKSGTSATLGITGLQTGDEADYYCGTWDSSLS |
| | GVVFGGGTKLTVLG [SEQ ID NO: 86] |
| DNA | Cagtctgtcgtgacgcagccgccctcagtgtctgcggccccaggacagaaggtcaccatctcctgctctggaa |
| | gcagctccaacattgggaataattatgtatcctggtaccagcaactcccaggaacagcccccaaactcctcattta |
| | tgacaataataagcgaccctcagggattcctgaccgattctctggctccaagtctggcacgtctgccaccctggg |
| | catcaccggactccagactggggacgaggccgattattactgcggaacatgggatagcagcctgagtggtgtg |
| | gtattcggcggagggaccaagctgaccgtcctaggt [SEQ ID NO: 88] |
| scFv | QSVVTQPPSVSAAPGQKVTISCSGSSSNIGNNYVSWYQQLPGTAPKLLI |
| | YDNNKRPSGIPDRFSGSKSGTSATLGITGLQTGDEADYYCGTWDSSLS |
| | GVVFGGGTKLTVLGSRGGGGSGGGGSGGGGSLEMAQVQLVQSGGEV |
| | KKPGASVKVSCKASGFTFNTYGISWVRQAPGQGLEWMGWISANNGH |
| | TKSAQRFQDRVAMATDTSTSTAYMELRSLKFDDTAVYYCARGGYHH |
| | QMQRYYKATSVYSDYWGQGTLVTVSS [SEQ ID NO: 121] |

In certain embodiments, the antibody or other antigen binding protein is an anti-GPRC5D scFv or an antigen-binding fragment thereof having an antigen-binding region that comprises the amino acid sequence of SEQ ID NO: 122 and specifically binds to a GPRC5D polypeptide (e.g., a GPRC5D polypeptide having the amino acid sequence SEQ ID NO:97, or fragments thereof), which is designated as ET150-172 scFv (also referred to as “ET150-22 scFv”).

In certain embodiments, the anti-GPRC5D scFv antibody comprises a heavy chain variable region comprising amino acids having the sequence set forth in SEQ ID NO:89 and a light chain variable region comprising amino acids having the sequence set forth in SEQ ID NO:90, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the anti-GPRC5D scFv antibody is an scFv-Fc fusion protein or full length human IgG with V_{H} and V_{L} regions or CDRs selected from Table 23. In certain embodiments, the anti-GPRC5D scFv comprises a V_{H} comprising amino acids having the sequence set forth in SEQ ID NO:89, as shown in Table 23. In certain embodiments, the anti-GPRC5D scFv comprises a V_{L} comprising amino acids having the sequence set forth in SEQ ID NO:90, as shown in Table 23. In certain embodiments, the anti-GPRC5D scFv comprises a V_{H} comprising amino acids having the sequence set forth in SEQ ID NO:89 and a V_{L} comprising amino acids having the sequence set forth in SEQ ID NO:90, as shown in Table 23. In certain embodiments, the anti-GPRC5D scFv comprises a V H CDR1 comprising amino acids having the sequence set forth in SEQ ID NO:256 or conservative modifications thereof, a V_{H} CDR2 comprising amino acids having the sequence set forth in SEQ ID NO: 257 or conservative modifications thereof, and a V_{H} CDR3 comprising amino acids having the sequence set forth in SEQ ID NO: 258 or conservative modifications thereof, as shown in Table 23. In certain embodiments, the anti-GPRC5D scFv comprises a V_{L} CDR1 comprising amino acids having the sequence set forth in SEQ ID NO: 259 or conservative modifications thereof, a V_{L} CDR2 comprising amino acids having the sequence set forth in SEQ ID NO: 260 or conservative modifications thereof, and a V_{L} CDR3 comprising amino acids having the sequence set forth in SEQ ID NO: 261 or conservative modifications thereof, as shown in Table 23. In certain embodiments, the anti-GPRC5D scFv comprises a V_{H} CDR1 comprising amino acids having the sequence set forth in SEQ ID NO: 256 or conservative modifications thereof, a V_{H} CDR2 comprising amino acids having the sequence set forth in SEQ ID NO: 257 or conservative modifications thereof, a V_{H} CDR3 comprising amino acids having the sequence set forth in SEQ ID NO: 258 or conservative modifications thereof, a V_{L} CDR1 comprising amino acids having the sequence set forth in SEQ ID NO: 259 or conservative modifications thereof, a V_{L} CDR2 comprising amino acids having the sequence set forth in SEQ ID NO: 260 or conservative modifications thereof, and a V_{L} CDR3 comprising amino acids having the sequence set forth in SEQ ID NO: 261 or conservative modifications thereof, as shown in Table 23. In certain embodiments, the anti-GPRC5D scFv comprises a V_{H} CDR1 comprising amino acids having the sequence set forth in SEQ ID NO: 256, a V_{H} CDR2 comprising amino acids having the sequence set forth in SEQ ID NO: 257, a V_{H} CDR3 comprising amino acids having the sequence set forth in SEQ ID NO: 258, a V_{L} CDR1 comprising amino acids having the sequence set forth in SEQ ID NO: 259, a V_{L} CDR2 comprising amino acids having the sequence set forth in SEQ ID NO: 260, and a V_{L} CDR3 comprising amino acids having the sequence set forth in SEQ ID NO: 261.

TABLE 23

| Antigen | A GPRC5D polypeptide having the amino acid sequence of SEQ ID NO: 97 | | |
|---|---|---|---|
| CDRs | 1 | 2 | 3 |
| V_{H} | GYTFTSYY [SEQ ID | INPSGGSS [SEQ ID | ARAGMGMDT [SEQ |
| | NO: 256] | NO: 257] | ID NO: 258] |
| V_{L} | SSDVGGYNY [SEQ ID | EVS [SEQ ID NO: 260] | SSYAGSNTLV [SEQ |
| | NO: 259] | | ID NO: 261] |

| | |
|---|---|
| Full V_{H} | QMQLVQSGAEVKKPGASVKVSCKASGYTFTSYYMHWVRQAPGQGL |
| | EWMGIINPSGGSSSYAQKFQGRVTMTRDTSTSTVYMELSSLRSEDTAV |
| | YYCARAGMGMDTWGQGTLVTVSS [SEQ ID NO: 89] |
| DNA | Cagatgcagctggtgcagtctggggctgaggtgaagaagcctggggcctcagtgaaggtttcctgcaaggcat |
| | ctggatacaccttcaccagctactatatgcactgggtgcgacaggcccctggacaagggcttgagtggatggga |
| | ataatcaaccctagtggtggtagctcaagctacgcacagaagttccagggcagagtcaccatgaccagggaca |
| | cgtccacgagcacagtctacatggagctgagcagcctgagatctgaggacacggccgtgtattactgtgcgcg |
| | cgctggtatgggtatggatacttggggtcaaggtactctggtgaccgtctcctca [SEQ ID NO: 91] |
| Full V_{L} | QSALTQPPSASGSPGQSVTISCTGTSSDVGGYNYVSWYQQHPGKAPKL |
| | MIYEVSKRPSGVPDRFSGSKSGNTASLTVSGLQAEDEADYYCSSYAGS |
| | NTLVFGGGTKLTVLG [SEQ ID NO: 90] |
| DNA | Cagtctgccctgactcagcctccctccgcgtccgggtctcctggacagtcagtcaccatctcctgcactggaac |
| | cagcagtgacgttggtggttataactatgtctcctggtaccaacagcacccaggcaaagcccccaaactcatgat |
| | ttatgaggtcagtaageggccctcaggggtccctgatcgcttctctggctccaagtctggcaacacggcctccct |
| | gaccgtctctgggctccaggctgaggatgaggctgattattactgcagctcatatgcaggcagcaacaccttggt |
| | gttcggcggagggaccaagctgaccgtcctaggt [SEQ ID NO: 92] |
| scFv | QSALTQPPSASGSPGQSVTISCTGTSSDVGGYNYVSWYQQHPGKAPKL |
| | MIYEVSKRPSGVPDRFSGSKSGNTASLTVSGLQAEDEADYYCSSYAGS |
| | NTLVFGGGTKLTVLGSRGGGGSGGGGSGGGGSLEMAQMQLVQSGAE |
| | VKKPGASVKVSCKASGYTFTSYYMHWVRQAPGQGLEWMGIINPSGG |
| | SSSYAQKFQGRVTMTRDTSTSTVYMELSSLRSEDTAVYYCARAGMG |
| | MDTWGQGTLVTVSS [SEQ ID NO: 122] |

In certain embodiments, the antibody or other antigen binding protein is an anti-GPRC5D scFv or an antigen-binding fragment thereof having an antigen-binding region that comprises the amino acid sequence of SEQ ID NO: 123 and specifically binds to a GPRC5D polypeptide (e.g., a GPRC5D polypeptide having the amino acid sequence SEQ ID NO:97, or fragments thereof), which is designated as ET150-173 scFv (also referred to as “ET150-23 scFv”).

In certain embodiments, the anti-GPRC5D scFv antibody comprises a heavy chain variable region comprising amino acids having the sequence set forth in SEQ ID NO:93 and a light chain variable region comprising amino acids having the sequence set forth in SEQ ID NO:94, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the anti-GPRC5D scFv antibody is an scFv-Fc fusion protein or full length human IgG with V_{H} and V_{L} regions or CDRs selected from Table 24. In certain embodiments, the anti-GPRC5D scFv comprises a V_{H} comprising amino acids having the sequence set forth in SEQ ID NO:93, as shown in Table 24. In certain embodiments, the anti-GPRC5D scFv comprises a V_{L} comprising amino acids having the sequence set forth in SEQ ID NO:94, as shown in Table 24. In certain embodiments, the anti-GPRC5D scFv comprises a V_{H} comprising amino acids having the sequence set forth in SEQ ID NO:93 and a V_{L} comprising amino acids having the sequence set forth in SEQ ID NO:94, as shown in Table 24. In certain embodiments, the anti-GPRC5D scFv comprises a V H CDR1 comprising amino acids having the sequence set forth in SEQ ID NO:262 or conservative modifications thereof, a V_{H} CDR2 comprising amino acids having the sequence set forth in SEQ ID NO: 263 or conservative modifications thereof, and a V_{H} CDR3 comprising amino acids having the sequence set forth in SEQ ID NO: 264 or conservative modifications thereof, as shown in Table 24. In certain embodiments, the anti-GPRC5D scFv comprises a V_{L} CDR1 comprising amino acids having the sequence set forth in SEQ ID NO: 265 or conservative modifications thereof, a V_{L} CDR2 comprising amino acids having the sequence set forth in SEQ ID NO: 266 or conservative modifications thereof, and a V_{L} CDR3 comprising amino acids having the sequence set forth in SEQ ID NO: 267 or conservative modifications thereof, as shown in Table 24. In certain embodiments, the anti-GPRC5D scFv comprises a V_{H} CDR1 comprising amino acids having the sequence set forth in SEQ ID NO: 262 or conservative modifications thereof, a V_{H} CDR2 comprising amino acids having the sequence set forth in SEQ ID NO: 263 or conservative modifications thereof, a V_{H} CDR3 comprising amino acids having the sequence set forth in SEQ ID NO: 264 or conservative modifications thereof, a V_{L} CDR1 comprising amino acids having the sequence set forth in SEQ ID NO: 265 or conservative modifications thereof, a V_{L} CDR2 comprising amino acids having the sequence set forth in SEQ ID NO: 266 or conservative modifications thereof, and a V_{L} CDR3 comprising amino acids having the sequence set forth in SEQ ID NO: 267 or conservative modifications thereof, as shown in Table 24. In certain embodiments, the anti-GPRC5D scFv comprises a V_{H} CDR1 comprising amino acids having the sequence set forth in SEQ ID NO: 262, a V_{H} CDR2 comprising amino acids having the sequence set forth in SEQ ID NO: 263, a V_{H} CDR3 comprising amino acids having the sequence set forth in SEQ ID NO: 264, a V_{L} CDR1 comprising amino acids having the sequence set forth in SEQ ID NO: 265, a V_{L} CDR2 comprising amino acids having the sequence set forth in SEQ ID NO: 266, and a V_{L} CDR3 comprising amino acids having the sequence set forth in SEQ ID NO: 267.

TABLE 24

| Antigen | A GPRC5D polypeptide having the amino acid sequence of SEQ ID NO: 97 | | |
|---|---|---|---|
| CDRs | 1 | 2 | 3 |
| V_{H} | GYTFTSYY [SEQ ID NO: 262] | INPSGGST [SEQ ID NO: 263] | ARDVISGFDS [SEQ ID NO: 264] |
| V_{L} | SSDVGGYNY [SEQ ID NO: 265] | GVS [SEQ ID NO: 266] | SSYAGVNNLM [SEQ ID NO: 267] |

| | |
|---|---|
| Full V_{H} | QVQLVQSGAEVKKPGASVKVSCKASGYTFTSYYMHWVRQAPGQGLE |
| | WMGIINPSGGSTSYAQKFQGRVTMTRDTSTSTVYMELSSLRSEDTAV |
| | YYCARDVISGFDSWGQGTLVTVSS [SEQ ID NO: 93] |
| DNA | Caggtgcagctggtgcaatctggggctgaggtgaagaagcctggggcctcagtgaaggtttcctgcaaggcat |
| | ctggatacaccttcaccagctactatatgcactgggtgcgacaggcccctggacaagggcttgagtggatggga |
| | ataatcaaccctagtggtggtagcacaagctacgcacagaagttccagggcagagtcaccatgaccagggaca |
| | cgtccacgagcacagtctacatggagctgagcagcctgagatctgaggacactgccgtgtattactgtgcgcgc |
| | gacgttatctctggtttcgattcttggggtcaaggtactctggtgaccgtctcctca [SEQ ID NO: 95] |
| Full V_{L} | QSALTQPASVSGSPGQSITISCTGTSSDVGGYNYVSWYQQSPGKAPRL |
| | MIYGVSKRPSGVPDRFSGSKSGNTASLTVSGLQAEDEADYYCSSYAG |
| | VNNLMFGGGTKLTVLG [SEQ ID NO: 94] |
| DNA | Cagtctgccctgactcagcctgcctccgtgtctgggtctcctggacagtcgatcaccatctcctgcactggaacc |
| | agcagtgacgttggtggttataactatgtctcctggtaccaacaatccccaggcaaagcccccagactcatgattt |
| | atggggtcagtaagcggccctctggggtccctgatcgcttctctggctccaagtctggcaacacggcctccctga |
| | ccgtctctgggctccaggctgaagatgaggctgattattactgcagctcatatgcaggcgtcaacaatttaatgttc |
| | ggcggagggaccaagctgaccgtcctaggt [SEQ ID NO: 96] |
| scFv | QSALTQPASVSGSPGQSITISCTGTSSDVGGYNYVSWYQQSPGKAPRL |
| | MIYGVSKRPSGVPDRFSGSKSGNTASLTVSGLQAEDEADYYCSSYAG |
| | VNNLMFGGGTKLTVLGSRGGGGSGGGGSGGGGSLEMAQVQLVQSG |
| | AEVKKPGASVKVSCKASGYTFTSYYMHWVRQAPGQGLEWMGIINPS |
| | GGSTSYAQKFQGRVTMTRDTSTSTVYMELSSLRSEDTAVYYCARDVI |
| | SGFDSWGQGTLVTVSS [SEQ ID NO: 123] |

In certain embodiments, the antibody or other antigen binding protein is an anti-GPRC5D scFv or an antigen-binding fragment thereof having an antigen-binding region that comprises the amino acid sequence of SEQ ID NO:276 and specifically binds to a GPRC5D polypeptide (e.g., a GPRC5D polypeptide having the amino acid sequence SEQ ID NO:97, or fragments thereof), which is designated as ET150-024 scFv (also referred to as “ET150-174 scFv”).

In certain embodiments, the anti-GPRC5D scFv antibody comprises a heavy chain variable region comprising amino acids having the sequence set forth in SEQ ID NO:274 and a light chain variable region comprising amino acids having the sequence set forth in SEQ ID NO:275, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the anti-GPRC5D scFv antibody is an scFv-Fc fusion protein or full length human IgG with V_{H} and V_{L} regions or CDRs selected from Table 25. In certain embodiments, the anti-GPRC5D scFv comprises a V_{H} comprising amino acids having the sequence set forth in SEQ ID NO:274, as shown in Table 25. In certain embodiments, the anti-GPRC5D scFv comprises a V_{L} comprising amino acids having the sequence set forth in SEQ ID NO:275, as shown in Table 25. In certain embodiments, the anti-GPRC5D scFv comprises a V_{H} comprising amino acids having the sequence set forth in SEQ ID NO:274 and a V_{L} comprising amino acids having the sequence set forth in SEQ ID NO:275, as shown in Table 25. In certain embodiments, the anti-GPRC5D scFv comprises a V H CDR1 comprising amino acids having the sequence set forth in SEQ ID NO:268 or conservative modifications thereof, a V_{H} CDR2 comprising amino acids having the sequence set forth in SEQ ID NO:269 or conservative modifications thereof, and a V_{H} CDR3 comprising amino acids having the sequence set forth in SEQ ID NO:270 or conservative modifications thereof, as shown in Table 8. In certain embodiments, the anti-GPRC5D scFv comprises a V_{L} CDR1 comprising amino acids having the sequence set forth in SEQ ID NO:271 or conservative modifications thereof, a V_{L} CDR2 comprising amino acids having the sequence set forth in SEQ ID NO:272 or conservative modifications thereof, and a V_{L} CDR3 comprising amino acids having the sequence set forth in SEQ ID NO:273 or conservative modifications thereof, as shown in Table 25. In certain embodiments, the anti-GPRC5D scFv comprises a V_{H} CDR1 comprising amino acids having the sequence set forth in SEQ ID NO:268 or conservative modifications thereof, a V_{H} CDR2 comprising amino acids having the sequence set forth in SEQ ID NO:269 or conservative modifications thereof, a V_{H} CDR3 comprising amino acids having the sequence set forth in SEQ ID NO:270 or conservative modifications thereof, a V_{L} CDR1 comprising amino acids having the sequence set forth in SEQ ID NO:271 or conservative modifications thereof, a V_{L} CDR2 comprising amino acids having the sequence set forth in SEQ ID NO:272 or conservative modifications thereof, and a V_{L} CDR3 comprising amino acids having the sequence set forth in SEQ ID NO:273 or conservative modifications thereof, as shown in Table 25. In certain embodiments, the anti-GPRC5D scFv comprises a V_{H} CDR1 comprising amino acids having the sequence set forth in SEQ ID NO:268, a V_{H} CDR2 comprising amino acids having the sequence set forth in SEQ ID NO:269, a V_{H} CDR3 comprising amino acids having the sequence set forth in SEQ ID NO:270, a V_{L} CDR1 comprising amino acids having the sequence set forth in SEQ ID NO:271, a V_{L} CDR2 comprising amino acids having the sequence set forth in SEQ ID NO:272, and a V_{L} CDR3 comprising amino acids having the sequence set forth in SEQ ID NO:273.

TABLE 25

| Antigen | A GPRC5D polypeptide having the amino acid sequence of SEQ ID NO: 97 | | |
|---|---|---|---|
| CDRs | 1 | 2 | 3 |
| V_{H} | GFTFGDYG [SEQ ID NO: 268] | INWNGGST [SEQ ID NO: 269] | ARSKQGY |
| | | | [SEQ ID NO: 270] |
| V_{L} | SRDAGGYNY [SEQ ID NO: 271] | EVT [SEQ ID NO: 272] | SSYGGSNNFRV |
| | | | [SEQ ID NO: 273] |

| | |
|---|---|
| Full V_{H} | EVQLVESGGGVVRPGGSLRLSCAASGFTFGDYGMSWVRQAPGKGLE |
| | WVSGINWNGGSTGYADSVKGRFTISRDNAKNSLYLQMNSLRAEDTA |
| | VYYCARSKQDYWGQGTLVTVSS [SEQ ID NO: 274] |
| DNA | GAGGTGCAGCTGGTGGAGTCTGGGGGAGGTGTGGTACGGCCTGGG |
| | GGGTCCCTGAGACTCTCCTGTGCAGCCTCTGGATTCACCTTTGGTG |
| | ATTATGGCATGAGCTGGGTCCGCCAAGCTCCAGGGAAGGGGCTGG |
| | AGTGGGTCTCTGGTATTAATTGGAATGGTGGTAGCACAGGTTATGC |
| | AGACTCTGTGAAGGGCCGATTCACCATCTCCAGAGACAACGCCAA |
| | GAACTCCCTGTATCTGCAAATGAACAGTCTGAGAGCCGAGGACAC |
| | GGCCGTATATTACTGTGCGCGCTCTAAACAGGATTACTGGGGTCAA |
| | GGTACTCTGGTGACCGTCTCCTCA [SEQ ID NO: 277] |
| Full V_{L} | MKKTAIAIAVALAGFATVAQAAELQSALTQPPSASGSPGQSVTISCTGT |
| | SRDAGGYNYFSWYQQHPGKAPKLLIYEVTKRPSGVPDRFSGSKSGKT |
| | ASLTVSGLQADDEAVYYCSSYGGSNNFRVFGGGTKLTVLG [SEQ ID |
| | NO: 275] |
| DNA | ATGAAAAAGACAGCTATCGCGATTGCAGTGGCACTGGCTGGTTTC |
| | GCTACCGTGGCCCAGGCGGCCGAGCTCCAGTCTGCCCTGACTCAGC |
| | CTCCCTCCGCGTCCGGGTCTCCTGGACAGTCAGTCACCATCTCCTG |
| | CACTGGAACCAGCAGGGACGCTGGTGGTTATAATTATTTCTCCTGG |
| | TACCAACAACACCCAGGCAAAGCCCCCAAACTCCTGATTTATGAG |
| | GTCACTAAGCGGCCCTCAGGGGTCCCTGATCGCTTCTCTGGCTCCA |
| | AGTCTGGCAAGACGGCCTCCCTGACCGTCTCTGGGCTCCAGGCTGA |
| | CGATGAGGCTGTATATTACTGCAGCTCATATGGAGGCAGCAACAA |
| | CTTTCGGGTGTTCGGCGGAGGGACCAAGCTGACCGTCCTAGGT |
| | [SEQ ID NO: 278] |
| scFv | MKKTAIAIAVALAGFATVAQAAELQSALTQPPSASGSPGQSVTISCTGT |
| | SRDAGGYNYFSWYQQHPGKAPKLLIYEVTKRPSGVPDRFSGSKSGKT |
| | ASLTVSGLQADDEAVYYCSSYGGSNNFRVFGGGTKLTVLGSRGGGGS |
| | GGGGSGGGGSLEMAEVQLVESGGGVVRPGGSLRLSCAASGFTFGDY |
| | GMSWVRQAPGKGLEWVSGINWNGGSTGYADSVKGRFTISRDNAKNS |
| | LYLQMNSLRAEDTAVYYCARSKQDYWGQGTLVTVSS [SEQ ID |
| | NO: 276] |
| DNA | ATGAAAAAGACAGCTATCGCGATTGCAGTGGCACTGGCTGGTTTC |
| | GCTACCGTGGCCCAGGCGGCCGAGCTCCAGTCTGCCCTGACTCAGC |
| | CTCCCTCCGCGTCCGGGTCTCCTGGACAGTCAGTCACCATCTCCTG |
| | CACTGGAACCAGCAGGGACGCTGGTGGTTATAATTATTTCTCCTGG |
| | TACCAACAACACCCAGGCAAAGCCCCCAAACTCCTGATTTATGAG |
| | GTCACTAAGCGGCCCTCAGGGGTCCCTGATCGCTTCTCTGGCTCCA |
| | AGTCTGGCAAGACGGCCTCCCTGACCGTCTCTGGGCTCCAGGCTGA |
| | CGATGAGGCTGTATATTACTGCAGCTCATATGGAGGCAGCAACAA |
| | CTTTCGGGTGTTCGGCGGAGGGACCAAGCTGACCGTCCTAGGTTCT |
| | AGAGGTGGTGGTGGTAGCGGCGGCGGCGGCTCTGGTGGTGGTGGA |
| | TCCCTCGAGATGGCCGAGGTGCAGCTGGTGGAGTCTGGGGGAGGT |
| | GTGGTACGGCCTGGGGGGTCCCTGAGACTCTCCTGTGCAGCCTCTG |
| | GATTCACCTTTGGTGATTATGGCATGAGCTGGGTCCGCCAAGCTCC |
| | AGGGAAGGGGCTGGAGTGGGTCTCTGGTATTAATTGGAATGGTGG |
| | TAGCACAGGTTATGCAGACTCTGTGAAGGGCCGATTCACCATCTCC |
| | AGAGACAACGCCAAGAACTCCCTGTATCTGCAAATGAACAGTCTG |
| | AGAGCCGAGGACACGGCCGTATATTACTGTGCGCGCTCTAAACAG |
| | GATTACTGGGGTCAAGGTACTCTGGTGACCGTCTCCTCA [SEQ ID |
| | NO: 279] |

In certain embodiments, the antibody or other antigen binding protein is an anti-GPRC5D scFv or an antigen-binding fragment thereof having an antigen-binding region that comprises the amino acid sequence of SEQ ID NO: 288 and specifically binds to a GPRC5D polypeptide (e.g., a GPRC5D polypeptide having the amino acid sequence SEQ ID NO:97, or fragments thereof), which is designated as ET150-026 scFv (also referred to as “ET150-176 scFv”).

In certain embodiments, the anti-GPRC5D scFv antibody comprises a heavy chain variable region comprising amino acids having the sequence set forth in SEQ ID NO:286 and a light chain variable region comprising amino acids having the sequence set forth in SEQ ID NO:287, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the anti-GPRC5D scFv antibody is an scFv-Fc fusion protein or full length human IgG with V_{H} and V_{L} regions or CDRs selected from Table 26. In certain embodiments, the anti-GPRC5D scFv comprises a V_{H} comprising amino acids having the sequence set forth in SEQ ID NO:286, as shown in Table 26. In certain embodiments, the anti-GPRC5D scFv comprises a V_{L} comprising amino acids having the sequence set forth in SEQ ID NO:287, as shown in Table 26. In certain embodiments, the anti-GPRC5D scFv comprises a V_{H} comprising amino acids having the sequence set forth in SEQ ID NO:286 and a V_{L} comprising amino acids having the sequence set forth in SEQ ID NO:287, as shown in Table 26. In certain embodiments, the anti-GPRC5D scFv comprises a V H CDR1 comprising amino acids having the sequence set forth in SEQ ID NO:280 or conservative modifications thereof, a V_{H} CDR2 comprising amino acids having the sequence set forth in SEQ ID NO:281 or conservative modifications thereof, and a V_{H} CDR3 comprising amino acids having the sequence set forth in SEQ ID NO:282 or conservative modifications thereof, as shown in Table 26. In certain embodiments, the anti-GPRC5D scFv comprises a V_{L} CDR1 comprising amino acids having the sequence set forth in SEQ ID NO:283 or conservative modifications thereof, a V_{L} CDR2 comprising amino acids having the sequence set forth in SEQ ID NO:284 or conservative modifications thereof, and a V_{L} CDR3 comprising amino acids having the sequence set forth in SEQ ID NO:285 or conservative modifications thereof, as shown in Table 26. In certain embodiments, the anti-GPRC5D scFv comprises a V_{H} CDR1 comprising amino acids having the sequence set forth in SEQ ID NO:280 or conservative modifications thereof, a V_{H} CDR2 comprising amino acids having the sequence set forth in SEQ ID NO:281 or conservative modifications thereof, a V_{H} CDR3 comprising amino acids having the sequence set forth in SEQ ID NO:282 or conservative modifications thereof, a V_{L} CDR1 comprising amino acids having the sequence set forth in SEQ ID NO:283 or conservative modifications thereof, a V_{L} CDR2 comprising amino acids having the sequence set forth in SEQ ID NO:284 or conservative modifications thereof, and a V_{L} CDR3 comprising amino acids having the sequence set forth in SEQ ID NO:285 or conservative modifications thereof, as shown in Table 26. In certain embodiments, the anti-GPRC5D scFv comprises a V_{H} CDR1 comprising amino acids having the sequence set forth in SEQ ID NO:280, a V_{H} CDR2 comprising amino acids having the sequence set forth in SEQ ID NO:281, a V_{H} CDR3 comprising amino acids having the sequence set forth in SEQ ID NO:282, a V_{L} CDR1 comprising amino acids having the sequence set forth in SEQ ID NO:283, a V_{L} CDR2 comprising amino acids having the sequence set forth in SEQ ID NO:284, and a V_{L} CDR3 comprising amino acids having the sequence set forth in SEQ ID NO:285.

TABLE 26

| Antigen | A GPRC5D polypeptide having the amino acid sequence of SEQ ID NO: 97 | | |
|---|---|---|---|
| CDRs | 1 | 2 | 3 |
| V_{H} | GFTFSNYA [SEQ ID NO: 280] | ITNSGRST [SEQ ID NO: 281] | ARVTHRRYGSTFDS |
| | | | [SEQ ID NO: 282] |
| V_{L} | SSNIGSNT [SEQ ID NO: 283] | SNN [SEQ ID NO: 284] | AAWDDSVNGYV |
| | | | [SEQ ID NO: 285] |

| | |
|---|---|
| Full V_{H} | QLQLQESGGGSVQPGGSLRLSCAASGFTFSNYAMSWVRQAPGKGLE |
| | WVSAITNSGRSTYYADSVKGRFTISRDNSKNTLSLQMSSLRAEDTAVY |
| | YCARVTHRRYGSTFDSRGQGTLVTVSS [SEQ ID NO: 286] |
| DNA | CAGCTGCAGCTGCAGGAGTCGGGGGGAGGCTCGGTACAGCCGGGG |
| | GGGTCTCTGAGACTGTCCTGTGCAGCCTCTGGATTCACCTTTAGCA |
| | ACTATGCCATGAGCTGGGTCCGCCAGGCTCCAGGGAAGGGGCTGG |
| | AGTGGGTCTCAGCTATCACTAATAGTGGTCGTAGTACATACTACGC |
| | AGACTCCGTGAAGGGCCGGTTCACCATCTCCAGAGACAATTCCAA |
| | GAACACGCTGTCTTTGCAAATGAGCAGCCTGAGAGCCGAAGACAC |
| | GGCCGTGTATTACTGTGCGCGCGTTACTCATCGTCGTTACGGTTCT |
| | ACTTTCGATTCTCGGGGTCAAGGTACTCTGGTGACCGTCTCCTCA |
| | ACTAGTGGCCAGGCCGGCCAGC [SEQ ID NO: 289] |
| Full V_{L} | MKKTAIAIAVALAGFATVAQAAELSYELTQPPSASGTPGQRVSISCSGS |
| | SSNIGSNTVNWYQQFPGTAPKLLIHSNNQRPSGVPDRFSGSKSGTSASL |
| | AISGPQSEDEADYYCAAWDDSVNGYVFGTGTKVTVLG [SEQ ID |
| | NO: 287] |
| DNA | ATGAAAAAGACAGCTATCGCGATTGCAGTGGCACTGGCTGGTTTC |
| | GCTACCGTGGCCCAGGCGGCCGAGCTCTCCTATGAGCTGACTCAGC |
| | CACCCTCAGCGTCTGGGACCCCCGGGCAGAGGGTCAGCATCTCTTG |
| | TTCTGGAAGCAGCTCCAACATCGGGAGTAATACTGTAAACTGGTAC |
| | CAACAGTTCCCCGGAACGGCCCCCAAACTCCTCATCCATAGTAATA |
| | ATCAGCGGCCCTCAGGGGTCCCTGACCGATTCTCTGGCTCCAAGTC |
| | TGGCACCTCAGCCTCCCTGGCCATCAGTGGGCCCCAGTCTGAGGAT |
| | GAGGCTGATTATTACTGTGCAGCTTGGGATGACAGTGTGAATGGTT |
| | ATGTCTTCGGAACTGGGACCAAGGTCACCGTCCTAGGT [SEQ ID |
| | NO: 290] |
| scFv | MKKTAIAIAVALAGFATVAQAAELSYELTQPPSASGTPGQRVSISCSGS |
| | SSNIGSNTVNWYQQFPGTAPKLLIHSNNQRPSGVPDRFSGSKSGTSASL |
| | AISGPQSEDEADYYCAAWDDSVNGYVFGTGTKVTVLGSRGGGGSGG |
| | GGSGGGGSLEMAQLQLQESGGGSVQPGGSLRLSCAASGFTFSNYAMS |
| | WVRQAPGKGLEWVSAITNSGRSTYYADSVKGRFTISRDNSKNTLSLQ |
| | MSSLRAEDTAVYYCARVTHRRYGSTFDSRGQGTLVTVSS [SEQ ID |
| | NO: 288] |
| DNA | ATGAAAAAGACAGCTATCGCGATTGCAGTGGCACTGGCTGGTTTC |
| | GCTACCGTGGCCCAGGCGGCCGAGCTCTCCTATGAGCTGACTCAGC |
| | CACCCTCAGCGTCTGGGACCCCCGGGCAGAGGGTCAGCATCTCTTG |
| | TTCTGGAAGCAGCTCCAACATCGGGAGTAATACTGTAAACTGGTAC |
| | CAACAGTTCCCCGGAACGGCCCCCAAACTCCTCATCCATAGTAATA |
| | ATCAGCGGCCCTCAGGGGTCCCTGACCGATTCTCTGGCTCCAAGTC |
| | TGGCACCTCAGCCTCCCTGGCCATCAGTGGGCCCCAGTCTGAGGAT |
| | GAGGCTGATTATTACTGTGCAGCTTGGGATGACAGTGTGAATGGTT |
| | ATGTCTTCGGAACTGGGACCAAGGTCACCGTCCTAGGTTCTAGAGG |
| | TGGTGGTGGTAGCGGCGGCGGCGGCTCTGGTGGTGGTGGATCCCTC |
| | GAGATGGCCCAGCTGCAGCTGCAGGAGTCGGGGGGAGGCTCGGTA |
| | CAGCCGGGGGGGTCTCTGAGACTGTCCTGTGCAGCCTCTGGATTCA |
| | CCTTTAGCAACTATGCCATGAGCTGGGTCCGCCAGGCTCCAGGGAA |
| | GGGGCTGGAGTGGGTCTCAGCTATCACTAATAGTGGTCGTAGTACA |
| | TACTACGCAGACTCCGTGAAGGGCCGGTTCACCATCTCCAGAGAC |
| | AATTCCAAGAACACGCTGTCTTTGCAAATGAGCAGCCTGAGAGCC |
| | GAAGACACGGCCGTGTATTACTGTGCGCGCGTTACTCATCGTCGTT |
| | ACGGTTCTACTTTCGATTCTCGGGGTCAAGGTACTCTGGTGACCGT |
| | CTCCTCA [SEQ ID NO: 291] |

In certain embodiments, the antibody or other antigen binding protein is an anti-GPRC5D scFv or an antigen-binding fragment thereof having an antigen-binding region that comprises the amino acid sequence of SEQ ID NO: 300 and specifically binds to a GPRC5D polypeptide (e.g., a GPRC5D polypeptide having the amino acid sequence SEQ ID NO:97, or fragments thereof), which is designated as ET150-028 scFv (also referred to as “ET150-178 scFv”).

In certain embodiments, the anti-GPRC5D scFv antibody comprises a heavy chain variable region comprising amino acids having the sequence set forth in SEQ ID NO:298 and a light chain variable region comprising amino acids having the sequence set forth in SEQ ID NO:299, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the anti-GPRC5D scFv antibody is an scFv-Fc fusion protein or full length human IgG with V_{H} and V_{L} regions or CDRs selected from Table 27. In certain embodiments, the anti-GPRC5D scFv comprises a V_{H} comprising amino acids having the sequence set forth in SEQ ID NO:298, as shown in Table 27. In certain embodiments, the anti-GPRC5D scFv comprises a V_{L} comprising amino acids having the sequence set forth in SEQ ID NO:299, as shown in Table 27. In certain embodiments, the anti-GPRC5D scFv comprises a V_{H} comprising amino acids having the sequence set forth in SEQ ID NO:298 and a V_{L} comprising amino acids having the sequence set forth in SEQ ID NO:299, as shown in Table 27. In certain embodiments, the anti-GPRC5D scFv comprises a V H CDR1 comprising amino acids having the sequence set forth in SEQ ID NO:292 or conservative modifications thereof, a V_{H} CDR2 comprising amino acids having the sequence set forth in SEQ ID NO:293 or conservative modifications thereof, and a V_{H} CDR3 comprising amino acids having the sequence set forth in SEQ ID NO:294 or conservative modifications thereof, as shown in Table 27. In certain embodiments, the anti-GPRC5D scFv comprises a V_{L} CDR1 comprising amino acids having the sequence set forth in SEQ ID NO:295 or conservative modifications thereof, a V_{L} CDR2 comprising amino acids having the sequence set forth in SEQ ID NO:296 or conservative modifications thereof, and a V_{L} CDR3 comprising amino acids having the sequence set forth in SEQ ID NO:297 or conservative modifications thereof, as shown in Table 27. In certain embodiments, the anti-GPRC5D scFv comprises a V_{H} CDR1 comprising amino acids having the sequence set forth in SEQ ID NO:292 or conservative modifications thereof, a V_{H} CDR2 comprising amino acids having the sequence set forth in SEQ ID NO:293 or conservative modifications thereof, a V_{H} CDR3 comprising amino acids having the sequence set forth in SEQ ID NO:294 or conservative modifications thereof, a V_{L} CDR1 comprising amino acids having the sequence set forth in SEQ ID NO:295 or conservative modifications thereof, a V_{L} CDR2 comprising amino acids having the sequence set forth in SEQ ID NO:296 or conservative modifications thereof, and a V_{L} CDR3 comprising amino acids having the sequence set forth in SEQ ID NO:297 or conservative modifications thereof, as shown in Table 27. In certain embodiments, the anti-GPRC5D scFv comprises a V_{H} CDR1 comprising amino acids having the sequence set forth in SEQ ID NO:292, a V_{H} CDR2 comprising amino acids having the sequence set forth in SEQ ID NO:293, a V_{H} CDR3 comprising amino acids having the sequence set forth in SEQ ID NO:294, a V_{L} CDR1 comprising amino acids having the sequence set forth in SEQ ID NO:295, a V_{L} CDR2 comprising amino acids having the sequence set forth in SEQ ID NO:296, and a V_{L} CDR3 comprising amino acids having the sequence set forth in SEQ ID NO:297.

TABLE 27

| Antigen | A GPRC5D polypeptide having the amino acid sequence | | |
|---|---|---|---|
| | of SEQ ID NO: 97 | | |
| CDRs | 1 | 2 | 3 |
| V_{H} | GGTFRSYA [SEQ ID | IIPMLDIT [SEQ ID NO: | ARTYSRSPFHMEDF |
| | NO: 292] | 293] | [SEQ ID NO: 294] |
| V_{L} | SSNIGGNT [SEQ ID | RNN [SEQ ID NO: 296] | AAWDASRQGV |
| | NO: 295] | | [SEQ ID NO: 297] |

| | |
|---|---|
| Full V_{H} | QVQLVQSGAEVKKPGSSVKVSCKASGGTFRSYAITWVRQAPGQGLE |
| | WMGRIIPMLDITNYAQKFQGRVTITADKSTSTAYMELSSLRSEDTAVY |
| | YCARTYSRSPFHMEDFWGQGTLVTVSS [SEQ ID NO: 298] |
| DNA | CAGGTGCAGCTGGTGCAGTCTGGGGCTGAGGTGAAGAAGCCTGGG |
| | TCCTCGGTGAAGGTCTCCTGCAAGGCTTCTGGAGGCACCTTCCGCA |
| | GCTATGCTATCACCTGGGTGCGACAGGCCCCTGGACAAGGGCTTG |
| | AGTGGATGGGAAGGATCATCCCTATGCTTGATATAACAAACTACG |
| | CACAGAAGTTCCAGGGCAGAGTCACGATTACCGCGGACAAATCCA |
| | CGAGCACAGCCTACATGGAGCTGAGCAGCCTGAGATCTGAGGACA |
| | CGGCCGTGTATTACTGTGCGCGCACTTACTCTCGTTCTCCGTTCCAT |
| | ATGGAAGATTTCTGGGGTCAAGGTACTCTGGTGACCGTCTCCTCA |
| | [SEQ ID NO: 300] |
| Full V_{L} | MKKTAIAIAVALAGFATVAQAAELQPVLTQPPSASGTPGQRVTISCSG |
| | SSSNIGGNTVSWYQQVPGTAPRLLIFRNNQRPPGVPDRFSGSKSGTSAS |
| | LAISGLRSEDEADYYCAAWDASRQGVFGGGTKLTVLG [SEQ ID |
| | NO: 299] |
| DNA | ATGAAAAAGACAGCTATCGCGATTGCAGTGGCACTGGCTGGTTTC |
| | GCTACCGTGGCCCAGGCGGCCGAGCTCCAGCCTGTGCTGACTCAG |
| | CCACCCTCAGCGTCTGGGACCCCCGGGCAGAGGGTCACCATCTCTT |
| | GTTCTGGAAGCAGCTCCAATATCGGAGGTAACACTGTCAGCTGGTA |
| | CCAGCAGGTCCCAGGAACGGCCCCCAGACTCCTCATTTTTAGGAAT |
| | AATCAACGGCCCCCAGGGGTCCCTGACCGATTCTCTGGCTCCAAGT |
| | CTGGCACCTCAGCCTCCCTGGCCATCAGTGGGCTCCGGTCTGAGGA |
| | TGAGGCTGATTATTACTGTGCAGCATGGGACGCCAGTCGACAAGG |
| | GGTGTTCGGCGGAGGGACCAAGCTGACCGTCCTAGGT [SEQ ID |
| | NO: 301] |
| scFv | MKKTAIAIAVALAGFATVAQAAELQPVLTQPPSASGTPGQRVTISCSG |
| | SSSNIGGNTVSWYQQVPGTAPRLLIFRNNQRPPGVPDRFSGSKSGTSAS |
| | LAISGLRSEDEADYYCAAWDASRQGVFGGGTKLTVLGSRGGGGSGG |
| | GGSGGGGSLEMAQVQLVQSGAEVKKPGSSVKVSCKASGGTFRSYAIT |
| | WVRQAPGQGLEWMGRIIPMLDITNYAQKFQGRVTITADKSTSTAYME |
| | LSSLRSEDTAVYYCARTYSRSPFHMEDFWGQGTLVTVSS [SEQ ID |
| | NO: 300] |
| DNA | ATGAAAAAGACAGCTATCGCGATTGCAGTGGCACTGGCTGGTTTC |
| | GCTACCGTGGCCCAGGCGGCCGAGCTCCAGCCTGTGCTGACTCAG |
| | CCACCCTCAGCGTCTGGGACCCCCGGGCAGAGGGTCACCATCTCTT |
| | GTTCTGGAAGCAGCTCCAATATCGGAGGTAACACTGTCAGCTGGTA |
| | CCAGCAGGTCCCAGGAACGGCCCCCAGACTCCTCATTTTTAGGAAT |
| | AATCAACGGCCCCCAGGGGTCCCTGACCGATTCTCTGGCTCCAAGT |
| | CTGGCACCTCAGCCTCCCTGGCCATCAGTGGGCTCCGGTCTGAGGA |
| | TGAGGCTGATTATTACTGTGCAGCATGGGACGCCAGTCGACAAGG |
| | GGTGTTCGGCGGAGGGACCAAGCTGACCGTCCTAGGTTCTAGAGG |
| | TGGTGGTGGTAGCGGCGGCGGCGGCTCTGGTGGTGGTGGATCCCTC |
| | GAGATGGCCCAGGTGCAGCTGGTGCAGTCTGGGGCTGAGGTGAAG |
| | AAGCCTGGGTCCTCGGTGAAGGTCTCCTGCAAGGCTTCTGGAGGCA |
| | CCTTCCGCAGCTATGCTATCACCTGGGTGCGACAGGCCCCTGGACA |
| | AGGGCTTGAGTGGATGGGAAGGATCATCCCTATGCTTGATATAAC |
| | AAACTACGCACAGAAGTTCCAGGGCAGAGTCACGATTACCGCGGA |
| | CAAATCCACGAGCACAGCCTACATGGAGCTGAGCAGCCTGAGATC |
| | TGAGGACACGGCCGTGTATTACTGTGCGCGCACTTACTCTCGTTCT |
| | CCGTTCCATATGGAAGATTTCTGGGGTCAAGGTACTCTGGTGACCG |
| | TCTCCTCA [SEQ ID NO: 302] |

In certain embodiments, the antibody or other antigen binding protein is an anti-GPRC5D scFv or an antigen-binding fragment thereof having an antigen-binding region that comprises the amino acid sequence of SEQ ID NO:312 and specifically binds to a GPRC5D polypeptide (e.g., a GPRC5D polypeptide having the amino acid sequence SEQ ID NO:97, or fragments thereof), which is designated as ET150-029 scFv (also referred to as “ET150-179 scFv”).

In certain embodiments, the anti-GPRC5D scFv antibody comprises a heavy chain variable region comprising amino acids having the sequence set forth in SEQ ID NO:310 and a light chain variable region comprising amino acids having the sequence set forth in SEQ ID NO:311, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the anti-GPRC5D scFv antibody is an scFv-Fc fusion protein or full length human IgG with V_{H} and V_{L} regions or CDRs selected from Table 28. In certain embodiments, the anti-GPRC5D scFv comprises a V_{H} comprising amino acids having the sequence set forth in SEQ ID NO:310, as shown in Table 28. In certain embodiments, the anti-GPRC5D scFv comprises a V_{L} comprising amino acids having the sequence set forth in SEQ ID NO:311, as shown in Table 28. In certain embodiments, the anti-GPRC5D scFv comprises a V_{H} comprising amino acids having the sequence set forth in SEQ ID NO:310 and a V_{L} comprising amino acids having the sequence set forth in SEQ ID NO:311, as shown in Table 28. In certain embodiments, the anti-GPRC5D scFv comprises a V H CDR1 comprising amino acids having the sequence set forth in SEQ ID NO:303 or conservative modifications thereof, a V_{H} CDR2 comprising amino acids having the sequence set forth in SEQ ID NO:304 or conservative modifications thereof, and a V_{H} CDR3 comprising amino acids having the sequence set forth in SEQ ID NO:305 or conservative modifications thereof, as shown in Table 28. In certain embodiments, the anti-GPRC5D scFv comprises a V_{L} CDR1 comprising amino acids having the sequence set forth in SEQ ID NO:306 or conservative modifications thereof, a V_{L} CDR2 comprising amino acids having the sequence set forth in SEQ ID NO:307 or conservative modifications thereof, and a V_{L} CDR3 comprising amino acids having the sequence set forth in SEQ ID NO:308 or conservative modifications thereof, as shown in Table 28. In certain embodiments, the anti-GPRC5D scFv comprises a V_{H} CDR1 comprising amino acids having the sequence set forth in SEQ ID NO:303 or conservative modifications thereof, a V_{H} CDR2 comprising amino acids having the sequence set forth in SEQ ID NO:304 or conservative modifications thereof, a V_{H} CDR3 comprising amino acids having the sequence set forth in SEQ ID NO:305 or conservative modifications thereof, a V_{L} CDR1 comprising amino acids having the sequence set forth in SEQ ID NO:306 or conservative modifications thereof, a V_{L} CDR2 comprising amino acids having the sequence set forth in SEQ ID NO:307 or conservative modifications thereof, and a V_{L} CDR3 comprising amino acids having the sequence set forth in SEQ ID NO:308 or conservative modifications thereof, as shown in Table 28. In certain embodiments, the anti-GPRC5D scFv comprises a V_{H} CDR1 comprising amino acids having the sequence set forth in SEQ ID NO:303, a V_{H} CDR2 comprising amino acids having the sequence set forth in SEQ ID NO:304, a V_{H} CDR3 comprising amino acids having the sequence set forth in SEQ ID NO:305, a V_{L} CDR1 comprising amino acids having the sequence set forth in SEQ ID NO:306, a V_{L} CDR2 comprising amino acids having the sequence set forth in SEQ ID NO:307, and a V_{L} CDR3 comprising amino acids having the sequence set forth in SEQ ID NO:308.

TABLE 28

| Antigen | A GPRC5D polypeptide having the amino acid sequence | | |
|---|---|---|---|
| | of SEQ ID NO: 97 | | |
| CDRs | 1 | 2 | 3 |
| V_{H} | GFTFSSYA[SEQ ID | ISGSGGST [SEQ ID | ARKYQDV [SEQ ID |
| | NO: 303] | NO: 304] | NO: 305] |
| V_{L} | SSNIGSNT[SEQ ID | RNN [SEQ ID NO: 307] | AAWDDSLSGRV |
| | NO: 306] | | [SEQ ID NO: 308] |

| | |
|---|---|
| Full V_{H} | EVQLVESGGGLVQPGGSLRLSCAASGFTFSSYAMSWVRQAPGKGLE |
| | WVSAISGSGGSTYYADSVKGRFTISRDNAKNTLYLQMNSLRAEDTAV |
| | YYCARKYQDVWGQGTLVTVSS [SEQ ID NO: 310] |
| DNA | GAGGTGCAGCTGGTGGAGTCTGGGGGAGGCTTGGTACAGCCTGGG |
| | GGGTCCCTGAGACTCTCCTGTGCAGCCTCTGGATTCACCTTTAGCA |
| | GCTATGCCATGAGCTGGGTCCGCCAGGCTCCAGGGAAGGGGCTGG |
| | AGTGGGTCTCAGCTATTAGTGGTAGTGGTGGTAGCACATACTACGC |
| | AGACTCCGTGAAGGGCCGGTTCACCATCTCCAGAGACAATGCCAA |
| | GAACACGCTGTATCTGCAAATGAACAGCCTGAGAGCCGAGGACAC |
| | GGCCGTATATTACTGTGCGCGCAAATACCAGGATGTTTGGGGTCAA |
| | GGTACTCTGGTGACCGTCTCCTCA [SEQ ID NO: 313] |
| Full V_{L} | MKKTAIAIAVALAGFATVAQAAELQSVLTQPPSASGTPGQRVTISCSG |
| | SSSNIGSNTVNWYQQLPGTAPKWYRNNQRPSGVPDRFSGSKSGTSAS |
| | LAISGLRSEDEADYYCAAWDDSLSGRVFGGGTKLTVLG [SEQ ID |
| | NO: 311] |
| DNA | ATGAAAAAGACAGCTATCGCGATTGCAGTGGCACTGGCTGGTTTC |
| | GCTACCGTGGCCCAGGCGGCCGAGCTCCAGTCTGTGCTGACGCAG |
| | CCGCCCTCAGCGTCTGGGACCCCCGGGCAGAGGGTCACCATCTCTT |
| | GTTCTGGAAGCAGCTCCAACATCGGAAGTAATACTGTAAACTGGT |
| | ACCAGCAGCTCCCAGGAACGGCCCCCAAACTCCTCATCTATAGGA |
| | ATAATCAGCGGCCCTCAGGGGTCCCTGACCGATTCTCTGGCTCCAA |
| | GTCTGGCACCTCAGCCTCCCTGGCCATCAGTGGGCTCCGGTCCGAG |
| | GATGAGGCTGATTATTACTGTGCAGCATGGGATGACAGCCTGAGT |
| | GGTAGGGTGTTCGGCGGAGGGACCAAGCTGACCGTCCTAGGT [SEQ |
| | ID NO: 314] |
| scFv | MKKTAIAIAVALAGFATVAQAAELQSVLTQPPSASGTPGQRVTISCSG |
| | SSSNIGSNTVNWYQQLPGTAPKWYRNNQRPSGVPDRFSGSKSGTSAS |
| | LAISGLRSEDEADYYCAAWDDSLSGRVFGGGTKLTVLGSRGGGGSGG |
| | GGSGGGGSLEMAEVQLVESGGGLVQPGGSLRLSCAASGFTFSSYAMS |
| | WVRQAPGKGLEWVSAISGSGGSTYYADSVKGRFTISRDNAKNTLYLQ |
| | MNSLRAEDTAVYYCARKYQDVWGQGTLVTVSS [SEQ ID NO: 312] |
| DNA | ATGAAAAAGACAGCTATCGCGATTGCAGTGGCACTGGCTGGTTTC |
| | GCTACCGTGGCCCAGGCGGCCGAGCTCCAGTCTGTGCTGACGCAG |
| | CCGCCCTCAGCGTCTGGGACCCCCGGGCAGAGGGTCACCATCTCTT |
| | GTTCTGGAAGCAGCTCCAACATCGGAAGTAATACTGTAAACTGGT |
| | ACCAGCAGCTCCCAGGAACGGCCCCCAAACTCCTCATCTATAGGA |
| | ATAATCAGCGGCCCTCAGGGGTCCCTGACCGATTCTCTGGCTCCAA |
| | GTCTGGCACCTCAGCCTCCCTGGCCATCAGTGGGCTCCGGTCCGAG |
| | GATGAGGCTGATTATTACTGTGCAGCATGGGATGACAGCCTGAGT |
| | GGTAGGGTGTTCGGCGGAGGGACCAAGCTGACCGTCCTAGGTTCT |
| | AGAGGTGGTGGTGGTAGCGGCGGCGGCGGCTCTGGTGGTGGTGGA |
| | TCCCTCGAGATGGCCGAGGTGCAGCTGGTGGAGTCTGGGGGAGGC |
| | TTGGTACAGCCTGGGGGGTCCCTGAGACTCTCCTGTGCAGCCTCTG |
| | GATTCACCTTTAGCAGCTATGCCATGAGCTGGGTCCGCCAGGCTCC |
| | AGGGAAGGGGCTGGAGTGGGTCTCAGCTATTAGTGGTAGTGGTGG |
| | TAGCACATACTACGCAGACTCCGTGAAGGGCCGGTTCACCATCTCC |
| | AGAGACAATGCCAAGAACACGCTGTATCTGCAAATGAACAGCCTG |
| | AGAGCCGAGGACACGGCCGTATATTACTGTGCGCGCAAATACCAG |
| | GATGTTTGGGGTCAAGGTACTCTGGTGACCGTCTCCTCA [SEQ ID |
| | NO: 315] |

In certain embodiments, the antibody or other antigen binding protein is an anti-GPRC5D scFv or an antigen-binding fragment thereof having an antigen-binding region that comprises the amino acid sequence of SEQ ID NO:324 and specifically binds to a GPRC5D polypeptide (e.g., a GPRC5D polypeptide having the amino acid sequence SEQ ID NO:97, or fragments thereof), which is designated as ET150-030 scFv (also referred to as “ET150-180 scFv”).

In certain embodiments, the anti-GPRC5D scFv antibody comprises a heavy chain variable region comprising amino acids having the sequence set forth in SEQ ID NO:322 and a light chain variable region comprising amino acids having the sequence set forth in SEQ ID NO:323, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the anti-GPRC5D scFv antibody is an scFv-Fc fusion protein or full length human IgG with V_{H} and V_{L} regions or CDRs selected from Table 29. In certain embodiments, the anti-GPRC5D scFv comprises a V_{H} comprising amino acids having the sequence set forth in SEQ ID NO:322, as shown in Table 29. In certain embodiments, the anti-GPRC5D scFv comprises a V_{L} comprising amino acids having the sequence set forth in SEQ ID NO:323, as shown in Table 29. In certain embodiments, the anti-GPRC5D scFv comprises a V_{H} comprising amino acids having the sequence set forth in SEQ ID NO:322 and a V_{L} comprising amino acids having the sequence set forth in SEQ ID NO:323, as shown in Table 29. In certain embodiments, the anti-GPRC5D scFv comprises a V H CDR1 comprising amino acids having the sequence set forth in SEQ ID NO:316 or conservative modifications thereof, a V_{H} CDR2 comprising amino acids having the sequence set forth in SEQ ID NO:317 or conservative modifications thereof, and a V_{H} CDR3 comprising amino acids having the sequence set forth in SEQ ID NO:318 or conservative modifications thereof, as shown in Table 29. In certain embodiments, the anti-GPRC5D scFv comprises a V_{L} CDR1 comprising amino acids having the sequence set forth in SEQ ID NO:319 or conservative modifications thereof, a V_{L} CDR2 comprising amino acids having the sequence set forth in SEQ ID NO:320 or conservative modifications thereof, and a V_{L} CDR3 comprising amino acids having the sequence set forth in SEQ ID NO:321 or conservative modifications thereof, as shown in Table 29. In certain embodiments, the anti-GPRC5D scFv comprises a V_{H} CDR1 comprising amino acids having the sequence set forth in SEQ ID NO:316 or conservative modifications thereof, a V_{H} CDR2 comprising amino acids having the sequence set forth in SEQ ID NO:317 or conservative modifications thereof, a V_{H} CDR3 comprising amino acids having the sequence set forth in SEQ ID NO:318 or conservative modifications thereof, a V_{L} CDR1 comprising amino acids having the sequence set forth in SEQ ID NO:319 or conservative modifications thereof, a V_{L} CDR2 comprising amino acids having the sequence set forth in SEQ ID NO:320 or conservative modifications thereof, and a V_{L} CDR3 comprising amino acids having the sequence set forth in SEQ ID NO:321 or conservative modifications thereof, as shown in Table 29. In certain embodiments, the anti-GPRC5D scFv comprises a V_{H} CDR1 comprising amino acids having the sequence set forth in SEQ ID NO:316, a V_{H} CDR2 comprising amino acids having the sequence set forth in SEQ ID NO:317, a V_{H} CDR3 comprising amino acids having the sequence set forth in SEQ ID NO:318, a V_{L} CDR1 comprising amino acids having the sequence set forth in SEQ ID NO:319, a V_{L} CDR2 comprising amino acids having the sequence set forth in SEQ ID NO:320, and a V_{L} CDR3 comprising amino acids having the sequence set forth in SEQ ID NO:321.

TABLE 29

| Antigen | A GPRC5D polypeptide having the amino acid sequence of | | |
|---|---|---|---|
| | SEQ ID NO: 97 | | |
| CDRs | 1 | 2 | 3 |
| V_{H} | GFSFSGTA [SEQ ID | ISSTGRST [SEQ ID NO: | ARPVSSMTLSIQSD |
| | NO: 316] | 317] | G [SEQ ID NO: 318] |
| V_{L} | SSNIGAGYD [SEQ ID | GNS [SEQ ID NO: 320] | QSYDSSLRGYV |
| | NO: 319] | | [SEQ ID NO: 321] |

| | |
|---|---|
| Full V_{H} | QVQLVQSGGGVVQPGRSLRLSCAASGFSFSGTAMHWVRQAPGKGLE |
| | WVSTISSTGRSTYYRDSVKGRFTISRDNSKNTLYLQMNSLRGEDTAVY |
| | YCARPVSSMTLSIQSDGWGQGTLVTVSS [SEQ ID NO: 322] |
| DNA | CAGGTGCAGCTGGTGCAGTCTGGGGGAGGCGTGGTCCAGCCTGGG |
| | AGGTCCCTGAGACTCTCCTGTGCAGCCTCTGGATTCAGCTTTAGTG |
| | GCACTGCCATGCACTGGGTCCGCCAGGCTCCAGGGAAGGGGCTGG |
| | AATGGGTCTCGACTATTAGTAGTACTGGGCGTAGCACATACTACAG |
| | AGACTCCGTGAAGGGCCGGTTCACCATCTCCAGAGACAATTCCAA |
| | GAACACGCTGTATCTGCAAATGAACAGCCTGAGAGGCGAGGACAC |
| | GGCCGTATATTACTGTGCGCGCCCGGTTTCTTCTATGACTCTGTCTA |
| | TCCAGTCTGATGGTTGGGGTCAAGGTACTCTGGTGACCGTCTCCTC |
| | A [SEQ ID NO: 325] |
| Full V_{L} | MKKTAIAIAVALAGFATVAQAAELQSVLTQPPSVSGAPGQRVTISCTG |
| | SSSNIGAGYDVHWYQQLPGRAPKLLIYGNSNRPSGVPDRFSGSKSGTS |
| | ASLAITGLQAEDEADYYCQSYDSSLRGYVFGTGTKVTVLG [SEQ ID |
| | NO: 323] |
| DNA | ATGAAAAAGACAGCTATCGCGATTGCAGTGGCACTGGCTGGTTTC |
| | GCTACCGTGGCCCAGGCGGCCGAGCTCCAGTCTGTGTTGACGCAG |
| | CCGCCCTCAGTGTCTGGGGCCCCAGGGCAGAGGGTCACCATCTCCT |
| | GCACTGGGAGCAGCTCCAACATCGGGGCAGGTTATGATGTACACT |
| | GGTACCAGCAGCTTCCAGGAAGAGCCCCCAAACTCCTCATCTATG |
| | GTAACAGCAATCGGCCCTCAGGGGTCCCTGACCGATTCTCTGGCTC |
| | CAAGTCTGGCACCTCAGCCTCCCTGGCCATCACTGGGCTCCAGGCT |
| | GAGGATGAGGCTGATTATTACTGCCAGTCCTATGACAGCAGCCTGA |
| | GAGGTTATGTCTTCGGAACTGGGACCAAGGTCACCGTCCTAGGT |
| | [SEQ ID NO: 326] |
| scFv | MKKTAIAIAVALAGFATVAQAAELQSVLTQPPSVSGAPGQRVTISCTG |
| | SSSNIGAGYDVHWYQQLPGRAPKLLIYGNSNRPSGVPDRFSGSKSGTS |
| | ASLAITGLQAEDEADYYCQSYDSSLRGYVFGTGTKVTVLGSRGGGGS |
| | GGGGSGGGGSLEMAQVQLVQSGGGVVQPGRSLRLSCAASGFSFSGTA |
| | MHWVRQAPGKGLEWVSTISSTGRSTYYRDSVKGRFTISRDNSKNTLY |
| | LQMNSLRGEDTAVYYCARPVSSMTLSIQSDGWGQGTLVTVSS [SEQ |
| | ID NO: 324] |
| DNA | ATGAAAAAGACAGCTATCGCGATTGCAGTGGCACTGGCTGGTTTC |
| | GCTACCGTGGCCCAGGCGGCCGAGCTCCAGTCTGTGTTGACGCAG |
| | CCGCCCTCAGTGTCTGGGGCCCCAGGGCAGAGGGTCACCATCTCCT |
| | GCACTGGGAGCAGCTCCAACATCGGGGCAGGTTATGATGTACACT |
| | GGTACCAGCAGCTTCCAGGAAGAGCCCCCAAACTCCTCATCTATG |
| | GTAACAGCAATCGGCCCTCAGGGGTCCCTGACCGATTCTCTGGCTC |
| | CAAGTCTGGCACCTCAGCCTCCCTGGCCATCACTGGGCTCCAGGCT |
| | GAGGATGAGGCTGATTATTACTGCCAGTCCTATGACAGCAGCCTGA |
| | GAGGTTATGTCTTCGGAACTGGGACCAAGGTCACCGTCCTAGGTTC |
| | TAGAGGTGGTGGTGGTAGCGGCGGCGGCGGCTCTGGTGGTGGTGG |
| | ATCCCTCGAGATGGCCCAGGTGCAGCTGGTGCAGTCTGGGGGAGG |
| | CGTGGTCCAGCCTGGGAGGTCCCTGAGACTCTCCTGTGCAGCCTCT |
| | GGATTCAGCTTTAGTGGCACTGCCATGCACTGGGTCCGCCAGGCTC |
| | CAGGGAAGGGGCTGGAATGGGTCTCGACTATTAGTAGTACTGGGC |
| | GTAGCACATACTACAGAGACTCCGTGAAGGGCCGGTTCACCATCTC |
| | CAGAGACAATTCCAAGAACACGCTGTATCTGCAAATGAACAGCCT |
| | GAGAGGCGAGGACACGGCCGTATATTACTGTGCGCGCCCGGTTTCT |
| | TCTATGACTCTGTCTATCCAGTCTGATGGTTGGGGTCAAGGTACTC |
| | TGGTGACCGTCTCCTCA [SEQ ID NO: 327] |

In certain embodiments, the antibody or other antigen binding protein is an anti-GPRC5D scFv or an antigen-binding fragment thereof having an antigen-binding region that comprises the amino acid sequence of SEQ ID NO:336 and specifically binds to a GPRC5D polypeptide (e.g., a GPRC5D polypeptide having the amino acid sequence SEQ ID NO:97, or fragments thereof), which is designated as ET150-031 scFv (also referred to as “ET150-181 scFv”).

In certain embodiments, the anti-GPRC5D scFv antibody comprises a heavy chain variable region comprising amino acids having the sequence set forth in SEQ ID NO:334 and a light chain variable region comprising amino acids having the sequence set forth in SEQ ID NO:335, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the anti-GPRC5D scFv antibody is an scFv-Fc fusion protein or full length human IgG with V_{H} and V_{L} regions or CDRs selected from Table 30. In certain embodiments, the anti-GPRC5D scFv comprises a V_{H} comprising amino acids having the sequence set forth in SEQ ID NO:334, as shown in Table 30. In certain embodiments, the anti-GPRC5D scFv comprises a V_{L} comprising amino acids having the sequence set forth in SEQ ID NO:335, as shown in Table 30. In certain embodiments, the anti-GPRC5D scFv comprises a V_{H} comprising amino acids having the sequence set forth in SEQ ID NO:334 and a V_{L} comprising amino acids having the sequence set forth in SEQ ID NO:335, as shown in Table 30. In certain embodiments, the anti-GPRC5D scFv comprises a V H CDR1 comprising amino acids having the sequence set forth in SEQ ID NO:328 or conservative modifications thereof, a V_{H} CDR2 comprising amino acids having the sequence set forth in SEQ ID NO:329 or conservative modifications thereof, and a V_{H} CDR3 comprising amino acids having the sequence set forth in SEQ ID NO:330 or conservative modifications thereof, as shown in Table 30. In certain embodiments, the anti-GPRC5D scFv comprises a V_{L} CDR1 comprising amino acids having the sequence set forth in SEQ ID NO:331 or conservative modifications thereof, a V_{L} CDR2 comprising amino acids having the sequence set forth in SEQ ID NO:332 or conservative modifications thereof, and a V_{L} CDR3 comprising amino acids having the sequence set forth in SEQ ID NO:333 or conservative modifications thereof, as shown in Table 30. In certain embodiments, the anti-GPRC5D scFv comprises a V_{H} CDR1 comprising amino acids having the sequence set forth in SEQ ID NO:328 or conservative modifications thereof, a V_{H} CDR2 comprising amino acids having the sequence set forth in SEQ ID NO:329 or conservative modifications thereof, a V_{H} CDR3 comprising amino acids having the sequence set forth in SEQ ID NO:330 or conservative modifications thereof, a V_{L} CDR1 comprising amino acids having the sequence set forth in SEQ ID NO:331 or conservative modifications thereof, a V_{L} CDR2 comprising amino acids having the sequence set forth in SEQ ID NO:332 or conservative modifications thereof, and a V_{L} CDR3 comprising amino acids having the sequence set forth in SEQ ID NO:333 or conservative modifications thereof, as shown in Table 30. In certain embodiments, the anti-GPRC5D scFv comprises a V_{H} CDR1 comprising amino acids having the sequence set forth in SEQ ID NO:328, a V_{H} CDR2 comprising amino acids having the sequence set forth in SEQ ID NO:329, a V_{H} CDR3 comprising amino acids having the sequence set forth in SEQ ID NO:330, a V_{L} CDR1 comprising amino acids having the sequence set forth in SEQ ID NO:331, a V_{L} CDR2 comprising amino acids having the sequence set forth in SEQ ID NO:332, and a V_{L} CDR3 comprising amino acids having the sequence set forth in SEQ ID NO:333.

TABLE 30

| Antigen | A GPRC5D polypeptide having the amino acid sequence | | |
|---|---|---|---|
| | of SEQ ID NO: 97 | | |
| CDRs | 1 | 2 | 3 |
| V_{H} | GYTFTSYY [SEQ | INPSGGST [SEQ ID | ARGQKYHSQYSRGG |
| | ID NO: 328] | NO: 329] | TGGGMTQDM [SEQ |
| | | | ID NO: 330] |
| V_{L} | SSNIGNNY [SEQ ID | DNN [SEQ ID NO: 332] | GTWDSSLRNWV |
| | NO: 331] | | [SEQ ID NO: 333] |

| | |
|---|---|
| Full V_{H} | QMQLVQSGAEVKKPGASVKVSCKASGYTFTSYYMEIWVRQAPGQGL |
| | EWMGIINPSGGSTSYAQKFQGRVTMTRDTSTSTVYMELSSLRSEDTA |
| | VYYCARGQKYHSQYSRGGTGGGMTQDMWGQGTLVTVSS [SEQ ID |
| | NO: 334] |
| DNA | CAGATGCAGCTGGTGCAGTCTGGGGCTGAGGTGAAGAAGCCTGG |
| | GGCCTCAGTGAAGGTTTCCTGCAAGGCATCTGGATACACCTTCAC |
| | CAGCTACTATATGCACTGGGTGCGACAGGCCCCTGGACAAGGGCT |
| | TGAGTGGATGGGAATAATCAACCCTAGTGGTGGTAGCACAAGCTA |
| | CGCACAAAAGTTCCAGGGCAGAGTCACCATGACCAGGGACACGT |
| | CCACGAGCACAGTCTACATGGAGCTGAGCAGCCTGAGATCTGAGG |
| | ACACGGCCGTGTATTACTGTGCGCGCGGTCAGAAATACCATTCTC |
| | AGTACTCTCGTGGTGGTACTGGTGGTGGTATGACTCAGGATATGT |
| | GGGGTCAAGGTACTCTGGTGACCGTCTCCTCA [SEQ ID NO: 337] |
| Full V_{L} | MKKTAIAIAVALAGFATVAQAAELQSVVTQPPSVSAAPGQRVTISCS |
| | GGSSNIGNNYVSWFQQLPRTAPKLLIYDNNKRPSGIPDRFSGSKSGTS |
| | AALDITVLQTGDEADYYCGTWDSSLRNWVFGGGTKLTVLG [SEQ ID |
| | NO: 335] |
| DNA | ATGAAAAAGACAGCTATCGCGATTGCAGTGGCACTGGCTGGTTTC |
| | GCTACCGTGGCCCAGGCGGCCGAGCTCCAGTCTGTCGTGACGCAG |
| | CCGCCCTCTGTGTCTGCGGCCCCAGGACAGAGGGTCACCATCTCC |
| | TGCTCTGGAGGTAGTTCCAACATTGGGAATAATTATGTTTCCTGGT |
| | TCCAACAACTCCCACGAACAGCCCCCAAACTCCTCATTTATGACA |
| | ATAATAAGCGACCCTCAGGGATTCCTGACCGATTCTCTGGCTCCA |
| | AGTCTGGCACGTCAGCCGCCCTGGACATCACCGTTCTCCAGACTG |
| | GGGACGAGGCCGATTATTACTGCGGAACTTGGGATAGCAGCCTGA |
| | GAAATTGGGTGTTCGGCGGAGGGACCAAGCTGACCGTCCTAGGT |
| | [SEQ ID NO: 338] |
| scFv | MKKTAIAIAVALAGFATVAQAAELQSVVTQPPSVSAAPGQRVTISCS |
| | GGSSNIGNNYVSWFQQLPRTAPKLLIYDNNKRPSGIPDRFSGSKSGTS |
| | AALDITVLQTGDEADYYCGTWDSSLRNWVFGGGTKLTVLGSRGGG |
| | GSGGGGSGGGGSLEMAQMQLVQSGAEVKKPGASVKVSCKASGYTF |
| | TSYYMHWVRQAPGQGLEWMGIINPSGGSTSYAQKFQGRVTMTRDTS |
| | TSTVYMELSSLRSEDTAVYYCARGQKYHSQYSRGGTGGGMTQDMW |
| | GQGTLVTVSS [SEQ ID NO: 336] |
| DNA | ATGAAAAAGACAGCTATCGCGATTGCAGTGGCACTGGCTGGTTTC |
| | GCTACCGTGGCCCAGGCGGCCGAGCTCCAGTCTGTCGTGACGCAG |
| | CCGCCCTCTGTGTCTGCGGCCCCAGGACAGAGGGTCACCATCTCC |
| | TGCTCTGGAGGTAGTTCCAACATTGGGAATAATTATGTTTCCTGGT |
| | TCCAACAACTCCCACGAACAGCCCCCAAACTCCTCATTTATGACA |
| | ATAATAAGCGACCCTCAGGGATTCCTGACCGATTCTCTGGCTCCA |
| | AGTCTGGCACGTCAGCCGCCCTGGACATCACCGTTCTCCAGACTG |
| | GGGACGAGGCCGATTATTACTGCGGAACTTGGGATAGCAGCCTGA |
| | GAAATTGGGTGTTCGGCGGAGGGACCAAGCTGACCGTCCTAGGTT |
| | CTAGAGGTGGTGGTGGTAGCGGCGGCGGCGGCTCTGGTGGTGGTG |
| | GATCCCTCGAGATGGCCCAGATGCAGCTGGTGCAGTCTGGGGCTG |
| | AGGTGAAGAAGCCTGGGGCCTCAGTGAAGGTTTCCTGCAAGGCAT |
| | CTGGATACACCTTCACCAGCTACTATATGCACTGGGTGCGACAGG |
| | CCCCTGGACAAGGGCTTGAGTGGATGGGAATAATCAACCCTAGTG |
| | GTGGTAGCACAAGCTACGCACAAAAGTTCCAGGGCAGAGTCACC |
| | ATGACCAGGGACACGTCCACGAGCACAGTCTACATGGAGCTGAG |
| | CAGCCTGAGATCTGAGGACACGGCCGTGTATTACTGTGCGCGCGG |
| | TCAGAAATACCATTCTCAGTACTCTCGTGGTGGTACTGGTGGTGGT |
| | ATGACTCAGGATATGTGGGGTCAAGGTACTCTGGTGACCGTCTCC |
| | TCA [SEQ ID NO: 339] |

In certain embodiments, the antibody or other antigen binding protein is an anti-GPRC5D scFv or an antigen-binding fragment thereof having an antigen-binding region that comprises the amino acid sequence of SEQ ID NO:348 and specifically binds to a GPRC5D polypeptide (e.g., a GPRC5D polypeptide having the amino acid sequence SEQ ID NO:97, or fragments thereof), which is designated as ET150-032 scFv (also referred to as “ET150-182 scFv”).

In certain embodiments, the anti-GPRC5D scFv antibody comprises a heavy chain variable region comprising amino acids having the sequence set forth in SEQ ID NO:346 and a light chain variable region comprising amino acids having the sequence set forth in SEQ ID NO:347, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the anti-GPRC5D scFv antibody is an scFv-Fc fusion protein or full length human IgG with V_{H} and V_{L} regions or CDRs selected from Table 31. In certain embodiments, the anti-GPRC5D scFv comprises a V_{H} comprising amino acids having the sequence set forth in SEQ ID NO:346, as shown in Table 31. In certain embodiments, the anti-GPRC5D scFv comprises a V_{L} comprising amino acids having the sequence set forth in SEQ ID NO:347, as shown in Table 31. In certain embodiments, the anti-GPRC5D scFv comprises a V_{H} comprising amino acids having the sequence set forth in SEQ ID NO:346 and a V_{L} comprising amino acids having the sequence set forth in SEQ ID NO:347, as shown in Table 31. In certain embodiments, the anti-GPRC5D scFv comprises a V H CDR1 comprising amino acids having the sequence set forth in SEQ ID NO:340 or conservative modifications thereof, a V_{H} CDR2 comprising amino acids having the sequence set forth in SEQ ID NO:341 or conservative modifications thereof, and a V_{H} CDR3 comprising amino acids having the sequence set forth in SEQ ID NO:342 or conservative modifications thereof, as shown in Table 31. In certain embodiments, the anti-GPRC5D scFv comprises a V_{L} CDR1 comprising amino acids having the sequence set forth in SEQ ID NO:343 or conservative modifications thereof, a V_{L} CDR2 comprising amino acids having the sequence set forth in SEQ ID NO:344 or conservative modifications thereof, and a V_{L} CDR3 comprising amino acids having the sequence set forth in SEQ ID NO:345 or conservative modifications thereof, as shown in Table 31. In certain embodiments, the anti-GPRC5D scFv comprises a V_{H} CDR1 comprising amino acids having the sequence set forth in SEQ ID NO:340 or conservative modifications thereof, a V_{H} CDR2 comprising amino acids having the sequence set forth in SEQ ID NO:341 or conservative modifications thereof, a V_{H} CDR3 comprising amino acids having the sequence set forth in SEQ ID NO:342 or conservative modifications thereof, a V_{L} CDR1 comprising amino acids having the sequence set forth in SEQ ID NO:343 or conservative modifications thereof, a V_{L} CDR2 comprising amino acids having the sequence set forth in SEQ ID NO:344 or conservative modifications thereof, and a V_{L} CDR3 comprising amino acids having the sequence set forth in SEQ ID NO:345 or conservative modifications thereof, as shown in Table 31. In certain embodiments, the anti-GPRC5D scFv comprises a V_{H} CDR1 comprising amino acids having the sequence set forth in SEQ ID NO:340, a V_{H} CDR2 comprising amino acids having the sequence set forth in SEQ ID NO:341, a V_{H} CDR3 comprising amino acids having the sequence set forth in SEQ ID NO:342, a V_{L} CDR1 comprising amino acids having the sequence set forth in SEQ ID NO:343, a V_{L} CDR2 comprising amino acids having the sequence set forth in SEQ ID NO:344, and a V_{L} CDR3 comprising amino acids having the sequence set forth in SEQ ID NO:345.

TABLE 31

| Antigen | A GPRC5D polypeptide having the amino acid sequence | | |
|---|---|---|---|
| | of SEQ ID NO: 97 | | |
| CDRs | 1 | 2 | 3 |
| V_{H} | GYTFSRYY [SEQ ID | MNPNSGNT [SEQ ID | ARGRYHVIDY [SEQ |
| | NO: 340] | NO: 341] | ID NO: 342] |
| V_{L} | SSDVGGYNH [SEQ | EVT [SEQ ID NO: 344] | SSYAGSAHWV |
| | ID NO: 343] | | [SEQ ID NO: 345] |

| | |
|---|---|
| Full V_{H} | EVQLVQSGAEVKKPGASVKVSCKASGYTFSRYYIHWVRQAPGQGLE |
| | WMGWMNPNSGNTGYAQKFQGRVTMTRNTSISTAYMELSSLRSEDTA |
| | VYYCARGRYHVIDYWGQGTLVTVSS [SEQ ID NO: 346] |
| DNA | GAGGTCCAGCTGGTGCAGTCTGGGGCTGAGGTGAAGAAGCCTGGG |
| | GCCTCAGTGAAGGTTTCCTGCAAGGCATCTGGATACACCTTCAGCA |
| | GGTACTATATACACTGGGTGCGACAGGCCCCTGGACAAGGGCTTG |
| | AGTGGATGGGATGGATGAACCCTAACAGTGGTAACACAGGCTATG |
| | CACAGAAGTTCCAGGGCAGAGTCACCATGACCAGGAACACCTCCA |
| | TAAGCACAGCCTACATGGAGCTGAGCAGCCTGAGATCTGAGGACA |
| | CGGCCGTGTATTACTGTGCGCGCGGTCGTTACCATGTTATCGATTA |
| | CTGGGGTCAAGGTACTCTGGTGACCGTCTCCTCA [SEQ ID NO: 349] |
| Full V_{L} | MKKTAIAIAVALAGFATVAQAAELQSVLTQPPSASGSPGQSLTISCTGT |
| | SSDVGGYNHVSWYQQYPGKAPKLMIYEVTKRPSGVPDRFSGSKSGNT |
| | ASLTVSGLQAEDEADYYCSSYAGSAHWVFGGGTKLTVLG [SEQ ID |
| | NO: 347] |
| DNA | ATGAAAAAGACAGCTATCGCGATTGCAGTGGCACTGGCTGGTTTC |
| | GCTACCGTGGCCCAGGCGGCCGAGCTCCAGTCTGTGTTGACTCAGC |
| | CACCCTCCGCGTCCGGGTCTCCTGGACAGTCACTCACCATCTCCTG |
| | CACTGGAACCAGCAGTGACGTTGGTGGTTATAACCATGTCTCCTGG |
| | TACCAACAGTACCCAGGCAAAGCCCCCAAACTCATGATTTATGAG |
| | GTCACTAAGCGGCCCTCAGGGGTCCCTGATCGCTTCTCTGGCTCCA |
| | AGTCTGGCAACACGGCCTCCCTGACCGTCTCTGGGCTCCAGGCTGA |
| | GGATGAGGCTGATTATTACTGCAGCTCATATGCAGGCAGCGCCCAT |
| | TGGGTGTTCGGCGGAGGGACCAAGCTGACCGTCCTAGGT [SEQ ID |
| | NO: 350] |
| scFv | MKKTAIAIAVALAGFATVAQAAELQSVLTQPPSASGSPGQSLTISCTGT |
| | SSDVGGYNHVSWYQQYPGKAPKLMIYEVTKRPSGVPDRFSGSKSGNT |
| | ASLTVSGLQAEDEADYYCSSYAGSAHWVFGGGTKLTVLGSRGGGGS |
| | GGGGSGGGGSLEMAEVQLVQSGAEVKKPGASVKVSCKASGYTFSRY |
| | YIHWVRQAPGQGLEWMGWMNPNSGNTGYAQKFQGRVTMTRNTSIS |
| | TAYMELSSLRSEDTAVYYCARGRYHVIDYWGQGTLVTVSS [SEQ ID |
| | NO: 348] |
| DNA | ATGAAAAAGACAGCTATCGCGATTGCAGTGGCACTGGCTGGTTTC |
| | GCTACCGTGGCCCAGGCGGCCGAGCTCCAGTCTGTGTTGACTCAGC |
| | CACCCTCCGCGTCCGGGTCTCCTGGACAGTCACTCACCATCTCCTG |
| | CACTGGAACCAGCAGTGACGTTGGTGGTTATAACCATGTCTCCTGG |
| | TACCAACAGTACCCAGGCAAAGCCCCCAAACTCATGATTTATGAG |
| | GTCACTAAGCGGCCCTCAGGGGTCCCTGATCGCTTCTCTGGCTCCA |
| | AGTCTGGCAACACGGCCTCCCTGACCGTCTCTGGGCTCCAGGCTGA |
| | GGATGAGGCTGATTATTACTGCAGCTCATATGCAGGCAGCGCCCAT |
| | TGGGTGTTCGGCGGAGGGACCAAGCTGACCGTCCTAGGTTCTAGA |
| | GGTGGTGGTGGTAGCGGCGGCGGCGGCTCTGGTGGTGGTGGATCC |
| | CTCGAGATGGCCGAGGTCCAGCTGGTGCAGTCTGGGGCTGAGGTG |
| | AAGAAGCCTGGGGCCTCAGTGAAGGTTTCCTGCAAGGCATCTGGA |
| | TACACCTTCAGCAGGTACTATATACACTGGGTGCGACAGGCCCCTG |
| | GACAAGGGCTTGAGTGGATGGGATGGATGAACCCTAACAGTGGTA |
| | ACACAGGCTATGCACAGAAGTTCCAGGGCAGAGTCACCATGACCA |
| | GGAACACCTCCATAAGCACAGCCTACATGGAGCTGAGCAGCCTGA |
| | GATCTGAGGACACGGCCGTGTATTACTGTGCGCGCGGTCGTTACCA |
| | TGTTATCGATTACTGGGGTCAAGGTACTCTGGTGACCGTCTCCTCA |
| | [SEQ ID NO: 351] |

In certain embodiments, the antibody or other antigen binding protein is an anti-GPRC5D scFv or an antigen-binding fragment thereof having an antigen-binding region that comprises the amino acid sequence of SEQ ID NO:360 and specifically binds to a GPRC5D polypeptide (e.g., a GPRC5D polypeptide having the amino acid sequence SEQ ID NO:97, or fragments thereof), which is designated as ET150-033 scFv (also referred to as “ET150-183 scFv”).

In certain embodiments, the anti-GPRC5D scFv antibody comprises a heavy chain variable region comprising amino acids having the sequence set forth in SEQ ID NO:358 and a light chain variable region comprising amino acids having the sequence set forth in SEQ ID NO:359, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the anti-GPRC5D scFv antibody is an scFv-Fc fusion protein or full length human IgG with V_{H} and V_{L} regions or CDRs selected from Table 32. In certain embodiments, the anti-GPRC5D scFv comprises a V_{H} comprising amino acids having the sequence set forth in SEQ ID NO:358, as shown in Table 32. In certain embodiments, the anti-GPRC5D scFv comprises a V_{L} comprising amino acids having the sequence set forth in SEQ ID NO:359, as shown in Table 32. In certain embodiments, the anti-GPRC5D scFv comprises a V_{H} comprising amino acids having the sequence set forth in SEQ ID NO:358 and a V_{L} comprising amino acids having the sequence set forth in SEQ ID NO:359, as shown in Table 32. In certain embodiments, the anti-GPRC5D scFv comprises a V H CDR1 comprising amino acids having the sequence set forth in SEQ ID NO:352 or conservative modifications thereof, a V_{H} CDR2 comprising amino acids having the sequence set forth in SEQ ID NO:353 or conservative modifications thereof, and a V_{H} CDR3 comprising amino acids having the sequence set forth in SEQ ID NO:354 or conservative modifications thereof, as shown in Table 32. In certain embodiments, the anti-GPRC5D scFv comprises a V_{L} CDR1 comprising amino acids having the sequence set forth in SEQ ID NO:355 or conservative modifications thereof, a V_{L} CDR2 comprising amino acids having the sequence set forth in SEQ ID NO:356 or conservative modifications thereof, and a V_{L} CDR3 comprising amino acids having the sequence set forth in SEQ ID NO:357 or conservative modifications thereof, as shown in Table 32. In certain embodiments, the anti-GPRC5D scFv comprises a V_{H} CDR1 comprising amino acids having the sequence set forth in SEQ ID NO:352 or conservative modifications thereof, a V_{H} CDR2 comprising amino acids having the sequence set forth in SEQ ID NO:353 or conservative modifications thereof, a V_{H} CDR3 comprising amino acids having the sequence set forth in SEQ ID NO:354 or conservative modifications thereof, a V_{L} CDR1 comprising amino acids having the sequence set forth in SEQ ID NO:355 or conservative modifications thereof, a V_{L} CDR2 comprising amino acids having the sequence set forth in SEQ ID NO:356 or conservative modifications thereof, and a V_{L} CDR3 comprising amino acids having the sequence set forth in SEQ ID NO:357 or conservative modifications thereof, as shown in Table 32. In certain embodiments, the anti-GPRC5D scFv comprises a V_{H} CDR1 comprising amino acids having the sequence set forth in SEQ ID NO:352, a V_{H} CDR2 comprising amino acids having the sequence set forth in SEQ ID NO:353, a V_{H} CDR3 comprising amino acids having the sequence set forth in SEQ ID NO:354, a V_{L} CDR1 comprising amino acids having the sequence set forth in SEQ ID NO:355, a V_{L} CDR2 comprising amino acids having the sequence set forth in SEQ ID NO:356, and a V_{L} CDR3 comprising amino acids having the sequence set forth in SEQ ID NO:357.

TABLE 32

| Antigen | A GPRC5D polypeptide having the amino acid sequence | | |
|---|---|---|---|
| | of SEQ ID NO: 97 | | |
| CDRs | 1 | 2 | 3 |
| V_{H} | GYTFNTYY [SEQ ID | INPNNGGT [SEQ ID | ARSYDY [SEQ ID NO: |
| | NO: 352] | NO: 353] | 354] |
| V_{L} | SSNIGSNY [SEQ ID | RNN [SEQ ID NO: 356] | AAWDDSLSGRV [SEQ |
| | NO: 355] | | ID NO: 357] |

| | |
|---|---|
| Full V_{H} | QLQLVQSGAEVKKPGSSVKVSCKASGYTFNTYYLHWVRQAPGQGLE |
| | WMGRINPNNGGTNYAQKFQGRVTMTRDTSINTAYMELSRLRSDDTA |
| | VYYCARSYDYWGQGTLVTVSS [SEQ ID NO: 358] |
| DNA | CAGCTGCAGCTGGTGCAATCTGGGGCTGAGGTGAAGAAGCCTGGG |
| | TCCTCGGTGAAGGTCTCCTGCAAGGCTTCTGGATACACCTTCAACA |
| | CCTACTATCTGCACTGGGTACGACAGGCCCCTGGACAAGGGCTTGA |
| | GTGGATGGGACGGATCAACCCTAACAATGGTGGCACAAACTATGC |
| | ACAGAAGTTTCAGGGCAGGGTCACCATGACCAGGGACACGTCCAT |
| | CAACACAGCCTACATGGAGCTGAGCAGGCTGAGATCTGACGACAC |
| | GGCCGTGTATTACTGTGCGCGCTCTTACGATTACTGGGGTCAAGGT |
| | ACTCTGGTGACCGTCTCCTCA [SEQ ID NO: 361] |
| Full V_{L} | MKKTAIAIAVALAGFATVAQAAELQAVLTQPPSASGTPGQRVTISCSG |
| | SSSNIGSNYVYWYQQLPGTAPKLLIYRNNQRPSGVPDRFSGSKSGTSA |
| | SLAISGLRSEDEADYYCAAWDDSLSGRVFGTGTKVTVLG [SEQ ID |
| | NO: 359] |
| DNA | ATGAAAAAGACAGCTATCGCGATTGCAGTGGCACTGGCTGGTTTC |
| | GCTACCGTGGCCCAGGCGGCCGAGCTCCAGGCTGTGCTGACTCAG |
| | CCACCCTCAGCGTCTGGGACCCCCGGGCAGAGGGTCACCATCTCTT |
| | GTTCTGGAAGCAGCTCCAACATCGGAAGTAATTATGTATACTGGTA |
| | CCAGCAGCTCCCAGGAACGGCCCCCAAACTCCTCATCTATAGGAA |
| | TAATCAGCGGCCCTCAGGGGTCCCTGACCGATTCTCTGGCTCCAAG |
| | TCTGGCACCTCAGCCTCCCTGGCCATCAGTGGGCTCCGGTCCGAGG |
| | ATGAGGCTGATTATTACTGTGCAGCATGGGATGACAGCCTGAGTG |
| | GTCGGGTCTTCGGAACTGGGACCAAGGTCACCGTCCTAGGT [SEQ |
| | ID NO: 362] |
| scFv | MKKTAIAIAVALAGFATVAQAAELQAVLTQPPSASGTPGQRVTISCSG |
| | SSSNIGSNYVYWYQQLPGTAPKWYRNNQRPSGVPDRFSGSKSGTSA |
| | SLAISGLRSEDEADYYCAAWDDSLSGRVFGTGTKVTVLGSRGGGGSG |
| | GGGSGGGGSLEMAQLQLVQSGAEVKKPGSSVKVSCKASGYTFNTYY |
| | LHWVRQAPGQGLEWMGRINPNNGGTNYAQKFQGRVTMTRDTSINTA |
| | YMELSRLRSDDTAVYYCARSYDYWGQGTLVTVSS [SEQ ID NO: 360] |
| DNA | ATGAAAAAGACAGCTATCGCGATTGCAGTGGCACTGGCTGGTTTC |
| | GCTACCGTGGCCCAGGCGGCCGAGCTCCAGGCTGTGCTGACTCAG |
| | CCACCCTCAGCGTCTGGGACCCCCGGGCAGAGGGTCACCATCTCTT |
| | GTTCTGGAAGCAGCTCCAACATCGGAAGTAATTATGTATACTGGTA |
| | CCAGCAGCTCCCAGGAACGGCCCCCAAACTCCTCATCTATAGGAA |
| | TAATCAGCGGCCCTCAGGGGTCCCTGACCGATTCTCTGGCTCCAAG |
| | TCTGGCACCTCAGCCTCCCTGGCCATCAGTGGGCTCCGGTCCGAGG |
| | ATGAGGCTGATTATTACTGTGCAGCATGGGATGACAGCCTGAGTG |
| | GTCGGGTCTTCGGAACTGGGACCAAGGTCACCGTCCTAGGTTCTAG |
| | AGGTGGTGGTGGTAGCGGCGGCGGCGGCTCTGGTGGTGGTGGATC |
| | CCTCGAGATGGCCCAGCTGCAGCTGGTGCAATCTGGGGCTGAGGT |
| | GAAGAAGCCTGGGTCCTCGGTGAAGGTCTCCTGCAAGGCTTCTGG |
| | ATACACCTTCAACACCTACTATCTGCACTGGGTACGACAGGCCCCT |
| | GGACAAGGGCTTGAGTGGATGGGACGGATCAACCCTAACAATGGT |
| | GGCACAAACTATGCACAGAAGTTTCAGGGCAGGGTCACCATGACC |
| | AGGGACACGTCCATCAACACAGCCTACATGGAGCTGAGCAGGCTG |
| | AGATCTGACGACACGGCCGTGTATTACTGTGCGCGCTCTTACGATT |
| | ACTGGGGTCAAGGTACTCTGGTGACCGTCTCCTCA [SEQ ID NO: 363] |

The presently disclosed subject matter further provides anti-GPRC5D scFv antibodies comprising a heavy chain variable region, a light chain variable region, a linker peptide between the heavy chain variable region and the light chain variable region, and an His-tag and an HA-tag. In certain embodiments, the amino acid sequence of the His-tag and HA-tag comprises the amino acid sequence of SEQ ID NO:379, which is provided below:

[SEQ ID NO: 379]
TSGQAGQHHHHHHGAYPYDVPDYAS

The nucleotide sequence encoding SEQ ID NO: 379 is SEQ ID NO: 380, which is provided below:

[SEQ ID NO: 380]
ACTAGTGGCCAGGCCGGCCAGCACCATCACCATCACCATGGCGCATACCC
GTACGACGTTCCGGACTACGCTTCT

2. Monoclonal Antibodies

The presently disclosed subject matter provides human antibodies (e.g., human monoclonal antibodies) that specifically bind to GPRC5D (e.g., human GPRC5D) and were isolated and structurally characterized as described in Example 2. The V_{H} amino acid sequences of human anti-GPRC5D antibodies ET150-153, ET150-166, ET150-170, ET150-171, ET150-175, ET150-154, ET150-156, ET150-157, ET150-159, ET150-160, ET150-161, ET150-162, ET150-163, ET150-151, ET150-152, ET150-155, ET150-158, ET150-168, ET150-165, ET150-167, ET150-169, ET150-172, ET150-173, ET150-024, ET150-026, ET150-028, ET150-029, ET150-030, ET150-031, ET150-032 and ET150-033 are shown in SEQ ID NOs: 1, 5, 9, 13, 17, 21, 25, 29, 33, 37, 41, 45, 49, 53, 57, 61, 65, 69, 73, 77, 81, 85, 89, 93, 274, 286, 298, 310, 322, 334, 346 and 358, respectively. The V L amino acid sequences of ET150-153, ET150-166, ET150-170, ET150-171, ET150-175, ET150-154, ET150-156, ET150-157, ET150-159, ET150-160, ET150-161, ET150-162, ET150-163, ET150-151, ET150-152, ET150-155, ET150-158, ET150-168, ET150-165, ET150-167, ET150-169, ET150-172, ET150-173, ET150-024, ET150-026, ET150-028, ET150-029, ET150-030, ET150-031, ET150-032 and ET150-033 are shown in SEQ ID NOs: 2, 6, 10, 14, 18, 22, 26, 30, 34, 38, 42, 46, 50, 54, 58, 62, 66, 70, 74, 78, 82, 86, 90, 94, 275, 287, 299, 311, 323, 335, 347 and 359, respectively.

Given that each of ET150-153, ET150-166, ET150-170, ET150-171, ET150-175, ET150-154, ET150-156, ET150-157, ET150-159, ET150-160, ET150-161, ET150-162, ET150-163, ET150-151, ET150-152, ET150-155, ET150-158, ET150-168, ET150-165, ET150-167, ET150-169, ET150-172, ET150-173, ET150-024, ET150-026, ET150-028, ET150-029, ET150-030, ET150-031, ET150-032 and ET150-033 antibodies can bind to GPRC5D, the V_{H} and V_{L} sequences can be “mixed and matched” to create other anti-GPRC5D binding molecules. GPRC5D binding of such “mixed and matched” antibodies can be tested using the binding assays known in the art, including for example, ELISAs, Western blots, RIAs, Biacore® analysis. Preferably, when V_{H} and V_{L} chains are mixed and matched, a V_{H} sequence from a particular V_{H}/V_{L} pairing is replaced with a structurally similar V_{H} sequence. Likewise, a V_{L} sequence from a particular V_{H}/V_{L} pairing is replaced with a structurally similar V_{L} sequence.

In certain embodiments, the presently disclosed subject matter provides an isolated antibody, or antigen-binding portion thereof comprising: (i) a heavy chain variable region comprising an amino acid sequence selected from the group consisting of SEQ ID NOs: 1, 5, 9, 13, 17, 21, 25, 29, 33, 37, 41, 45, 49, 53, 57, 61, 65, 69, 73, 77, 81, 85, 89, 93, 274, 286, 298, 310, 322, 334, 346 and 358; and (ii) a light chain variable region comprising an amino acid sequence selected from the group consisting of SEQ ID NOs: 2, 6, 10, 14, 18, 22, 26, 30, 34, 38, 42, 46, 50, 54, 58, 62, 66, 70, 74, 78, 82, 86, 90, 94, 275, 287, 299, 311, 323, 335, 347 and 359; wherein the antibody specifically binds GPRC5D, e.g., human GPRC5D.

Preferred heavy and light chain combinations include:
(i) a heavy chain variable region comprising amino acids having a sequence set forth in SEQ ID NO:1, and a light chain variable region comprising amino acids having a sequence set forth in SEQ ID NO:2; or
(ii) a heavy chain variable region comprising amino acids having a sequence set forth in SEQ ID NO:5, and a light chain variable region comprising amino acids having a sequence set forth in SEQ ID NO:6;
(iii) a heavy chain variable region comprising amino acids having a sequence set forth in SEQ ID NO:9, and a light chain variable region comprising amino acids having a sequence set forth in SEQ ID NO:10;
(iv) a heavy chain variable region comprising amino acids having a sequence set forth in SEQ ID NO:13, and a light chain variable region comprising amino acids having a sequence set forth in SEQ ID NO:14;
(v) a heavy chain variable region comprising amino acids having a sequence set forth in SEQ ID NO:17, and a light chain variable region comprising amino acids having a sequence set forth in SEQ ID NO:18;
(vi) a heavy chain variable region comprising amino acids having a sequence set forth in SEQ ID NO:21, and a light chain variable region comprising amino acids having a sequence set forth in SEQ ID NO:22;
(vii) a heavy chain variable region comprising amino acids having a sequence set forth in SEQ ID NO:25, and a light chain variable region comprising amino acids having a sequence set forth in SEQ ID NO:26;
(viii) a heavy chain variable region comprising amino acids having a sequence set forth in SEQ ID NO:29, and a light chain variable region comprising amino acids having a sequence set forth in SEQ ID NO:30;
(ix) a heavy chain variable region comprising amino acids having a sequence set forth in SEQ ID NO:33, and a light chain variable region comprising amino acids having a sequence set forth in SEQ ID NO:34;
(x) a heavy chain variable region comprising amino acids having a sequence set forth in SEQ ID NO:37, and a light chain variable region comprising amino acids having a sequence set forth in SEQ ID NO:38;
(xi) a heavy chain variable region comprising amino acids having a sequence set forth in SEQ ID NO:41, and a light chain variable region comprising amino acids having a sequence set forth in SEQ ID NO:42;
(xii) a heavy chain variable region comprising amino acids having a sequence set forth in SEQ ID NO:45, and a light chain variable region comprising amino acids having a sequence set forth in SEQ ID NO:46;
(xiii) a heavy chain variable region comprising amino acids having a sequence set forth in SEQ ID NO:49, and a light chain variable region comprising amino acids having a sequence set forth in SEQ ID NO:50;
(xiv) a heavy chain variable region comprising amino acids having a sequence set forth in SEQ ID NO:53, and a light chain variable region comprising amino acids having a sequence set forth in SEQ ID NO:54;
(xv) a heavy chain variable region comprising amino acids having a sequence set forth in SEQ ID NO:57, and a light chain variable region comprising amino acids having a sequence set forth in SEQ ID NO:58;
(xvi) a heavy chain variable region comprising amino acids having a sequence set forth in SEQ ID NO:61, and a light chain variable region comprising amino acids having a sequence set forth in SEQ ID NO:62;
(xvii) a heavy chain variable region comprising amino acids having a sequence set forth in SEQ ID NO:65, and a light chain variable region comprising amino acids having a sequence set forth in SEQ ID NO:66;
(xviii) a heavy chain variable region comprising amino acids having a sequence set forth in SEQ ID NO:69, and a light chain variable region comprising amino acids having a sequence set forth in SEQ ID NO:70;
(xix) a heavy chain variable region comprising amino acids having a sequence set forth in SEQ ID NO:73, and a light chain variable region comprising amino acids having a sequence set forth in SEQ ID NO:74;
(xx) a heavy chain variable region comprising amino acids having a sequence set forth in SEQ ID NO:77, and a light chain variable region comprising amino acids having a sequence set forth in SEQ ID NO:78;
(xxi) a heavy chain variable region comprising amino acids having a sequence set forth in SEQ ID NO:81, and a light chain variable region comprising amino acids having a sequence set forth in SEQ ID NO:82;
(xxii) a heavy chain variable region comprising amino acids having a sequence set forth in SEQ ID NO:85, and a light chain variable region comprising amino acids having a sequence set forth in SEQ ID NO:86;
(xxiii) a heavy chain variable region comprising amino acids having a sequence set forth in SEQ ID NO:89, and a light chain variable region comprising amino acids having a sequence set forth in SEQ ID NO:90;
(x) a heavy chain variable region comprising amino acids having a sequence set forth in SEQ ID NO:93, and a light chain variable region comprising amino acids having a sequence set forth in SEQ ID NO:94.
(xvi) a heavy chain variable region comprising amino acids having a sequence set forth in SEQ ID NO:274, and a light chain variable region comprising amino acids having a sequence set forth in SEQ ID NO:275;
(xvii) a heavy chain variable region comprising amino acids having a sequence set forth in SEQ ID NO:286, and a light chain variable region comprising amino acids having a sequence set forth in SEQ ID NO:287;
(xviii) a heavy chain variable region comprising amino acids having a sequence set forth in SEQ ID NO:298, and a light chain variable region comprising amino acids having a sequence set forth in SEQ ID NO:299;
(xix) a heavy chain variable region comprising amino acids having a sequence set forth in SEQ ID NO:310, and a light chain variable region comprising amino acids having a sequence set forth in SEQ ID NO:311;
(xx) a heavy chain variable region comprising amino acids having a sequence set forth in SEQ ID NO:322, and a light chain variable region comprising amino acids having a sequence set forth in SEQ ID NO:323;
(xxi) a heavy chain variable region comprising amino acids having a sequence set forth in SEQ ID NO:334, and a light chain variable region comprising amino acids having a sequence set forth in SEQ ID NO:335;
(xxii) a heavy chain variable region comprising amino acids having a sequence set forth in SEQ ID NO:346, and a light chain variable region comprising amino acids having a sequence set forth in SEQ ID NO:347; or
(xxiii) a heavy chain variable region comprising amino acids having a sequence set forth in SEQ ID NO:358, and a light chain variable region comprising amino acids having a sequence set forth in SEQ ID NO:359.

In certain embodiments, the presently disclosed subject matter provides antibodies that comprise the heavy chain and light chain CDR1s, CDR2s and CDR3s of ET150-153, ET150-166, ET150-170, ET150-171, ET150-175, ET150-154, ET150-156, ET150-157, ET150-159, ET150-160, ET150-161, ET150-162, ET150-163, ET150-151, ET150-152, ET150-155, ET150-158, ET150-168, ET150-165, ET150-167, ET150-169, ET150-172, ET150-173, ET150-024, ET150-026, ET150-028, ET150-029, ET150-030, ET150-031, ET150-032 and ET150-033 antibodies. The amino acid sequences of the V H CDR1s of ET150-153, ET150-166, ET150-170, ET150-171, ET150-175, ET150-154, ET150-156, ET150-157, ET150-159, ET150-160, ET150-161, ET150-162, ET150-163, ET150-151, ET150-152, ET150-155, ET150-158, ET150-168, ET150-165, ET150-167, ET150-169, ET150-172, ET150-173, ET150-024, ET150-026, ET150-028, ET150-029, ET150-030, ET150-031, ET150-032 and ET150-033 are shown in SEQ ID NOs: 124, 130, 136, 142, 148, 154, 160, 166, 172, 178, 184, 190, 196, 202, 208, 214, 220, 226, 232, 238, 244, 250, 256, 262, 268, 280, 292, 303, 316, 328, 340 and 352, respectively. The amino acid sequences of the V H CDR2s of ET150-153, ET150-166, ET150-170, ET150-171, ET150-175, ET150-154, ET150-156, ET150-157, ET150-159, ET150-160, ET150-161, ET150-162, ET150-163, ET150-151, ET150-152, ET150-155, ET150-158, ET150-168, ET150-165, ET150-167, ET150-169, ET150-172, ET150-173, ET150-024, ET150-026, ET150-028, ET150-029, ET150-030, ET150-031, ET150-032 and ET150-033 antibodies are shown in SEQ ID NOs: 125, 131, 137, 143, 149, 155, 161, 167, 173, 179, 185, 191, 197, 203, 209, 215, 221, 227, 233, 239, 245, 251, 257, 263, 269, 281, 293, 304, 317, 329, 341 and 353, respectively. The amino acid sequences of the V H CDR3s of ET150-153, ET150-166, ET150-170, ET150-171, ET150-175, ET150-154, ET150-156, ET150-157, ET150-159, ET150-160, ET150-161, ET150-162, ET150-163, ET150-151, ET150-152, ET150-155, ET150-158, ET150-168, ET150-165, ET150-167, ET150-169, ET150-172, ET150-173, ET150-024, ET150-026, ET150-028, ET150-029, ET150-030, ET150-031, ET150-032 and ET150-033 are shown in SEQ ID NOs: 126, 132, 138, 144, 150, 156, 162, 168, 174, 180, 186, 192, 198, 204, 210, 216, 222, 228, 234, 240, 246, 252, 258, 264, 270, 282, 294, 305, 318, 330, 342 and 354, respectively.

The amino acid sequences of the V_{L} CDR1s of 1 ET150-153, ET150-166, ET150-170, ET150-171, ET150-175, ET150-154, ET150-156, ET150-157, ET150-159, ET150-160, ET150-161, ET150-162, ET150-163, ET150-151, ET150-152, ET150-155, ET150-158, ET150-168, ET150-165, ET150-167, ET150-169, ET150-172, ET150-173, ET150-024, ET150-026, ET150-028, ET150-029, ET150-030, ET150-031, ET150-032 and ET150-033 are shown in SEQ ID NOs: 127, 133, 139, 145, 151, 157, 163, 169, 175, 181, 187, 193, 199, 205, 211, 217, 223, 229, 235, 241, 247, 253, 259, 265, 271, 283, 295, 306, 319, 331, 343 and 355, respectively. The amino acid sequences of the V_{L} CDR2s of ET150-153, ET150-166, ET150-170, ET150-171, ET150-175, ET150-154, ET150-156, ET150-157, ET150-159, ET150-160, ET150-161, ET150-162, ET150-163, ET150-151, ET150-152, ET150-155, ET150-158, ET150-168, ET150-165, ET150-167, ET150-169, ET150-172, ET150-173, ET150-024, ET150-026, ET150-028, ET150-029, ET150-030, ET150-031, ET150-032 and ET150-033 are shown in SEQ ID NOs: 128, 134, 140, 146, 152, 158, 164, 170, 176, 182, 188, 194, 200, 206, 212, 218, 224, 230, 236, 242, 248, 254, 260, 266, 272, 284, 296, 307, 320, 332, 344 and 356, respectively. The amino acid sequences of the V_{L} CDR3s of ET150-153, ET150-166, ET150-170, ET150-171, ET150-175, ET150-154, ET150-156, ET150-157, ET150-159, ET150-160, ET150-161, ET150-162, ET150-163, ET150-151, ET150-152, ET150-155, ET150-158, ET150-168, ET150-165, ET150-167, ET150-169, ET150-172, ET150-173, ET150-024, ET150-026, ET150-028, ET150-029, ET150-030, ET150-031, ET150-032 and ET150-033 are shown in SEQ ID NOs: 129, 135, 141, 147, 153, 159, 165, 171, 177, 183, 189, 195, 201, 207, 213, 219, 225, 231, 237, 243, 249, 255, 261, 267, 273, 285, 297, 308, 321, 333, 345 and 357, respectively. The CDR regions are delineated using the Kabat system (Kabat, E. A., et al. (1991) Sequences of Proteins of Immunological Interest, Fifth Edition, U.S. Department of Health and Human Services, NIH Publication No. 91-3242).

Given that each of these antibodies can bind to GPRC5D and that antigen-binding specificity is provided primarily by the CDR1, CDR2, and CDR3 regions, the V_{H} CDR1, CDR2, and CDR3 sequences and V_{L} CDR1, CDR2, and CDR3 sequences can be “mixed and matched” (i.e., CDRs from different antibodies can be mixed and match, although each antibody must contain a V_{H} CDR1, CDR2, and CDR3 and a V_{L} CDR1, CDR2, and CDR3) to create other anti-GPRC5D binding molecules. GPRC5D binding of such “mixed and matched” antibodies can be tested using the binding assays described above. When V_{H} CDR sequences are mixed and matched, the CDR1, CDR2 and/or CDR3 sequence from a particular V_{H} sequence is replaced with a structurally similar CDR sequence(s). Likewise, when V_{L} CDR sequences are mixed and matched, the CDR1, CDR2 and/or CDR3 sequence from a particular V_{L} sequence preferably is replaced with a structurally similar CDR sequence(s). It will be readily apparent to the ordinarily skilled artisan that novel V_{H} and V_{L} sequences can be created by substituting one or more V_{H} and/or V_{L} CDR region sequences with structurally similar sequences from the CDR sequences of the antibodies disclosed herein ET150-153, ET150-166, ET150-170, ET150-171, ET150-175, ET150-154, ET150-156, ET150-157, ET150-159, ET150-160, ET150-161, ET150-162, ET150-163, ET150-151, ET150-152, ET150-155, ET150-158, ET150-168, ET150-165, ET150-167, ET150-169, ET150-172, ET150-173, ET150-024, ET150-026, ET150-028, ET150-029, ET150-030, ET150-031, ET150-032 and ET150-033.

In certain embodiments, the presently disclosed subject matter provides an isolated antibody, or antigen-binding portion thereof comprising: (ix) a heavy chain variable region CDR1 comprising an amino acid sequence selected from the group consisting of SEQ ID NOs: 124, 130, 136, 142, 148, 154, 160, 166, 172, 178, 184, 190, 196, 202, 208, 214, 220, 226, 232, 238, 244, 250, 256, 262, 268, 280, 292, 303, 316, 328, 340 and 352; (ii) a heavy chain variable region CDR2 comprising an amino acid sequence selected from the group consisting of SEQ ID NOs: 125, 131, 137, 143, 149, 155, 161, 167, 173, 179, 185, 191, 197, 203, 209, 215, 221, 227, 233, 239, 245, 251, 257, 263, 269, 281, 293, 304, 317, 329, 341 and 353; (iii) a heavy chain variable region CDR3 comprising an amino acid sequence selected from the group consisting of SEQ ID NOs: 126, 132, 138, 144, 150, 156, 162, 168, 174, 180, 186, 192, 198, 204, 210, 216, 222, 228, 234, 240, 246, 252, 258, 264, 270, 282, 294, 305, 318, 330, 342 and 354; (iv) a light chain variable region CDR1 comprising an amino acid sequence selected from the group consisting of SEQ ID NOs: 127, 133, 139, 145, 151, 157, 163, 169, 175, 181, 187, 193, 199, 205, 211, 217, 223, 229, 235, 241, 247, 253, 259, 265, 271, 283, 295, 306, 319, 331, 343 and 355; (v) a light chain variable region CDR2 comprising an amino acid sequence selected from the group consisting of SEQ ID NOs: 128, 134, 140, 146, 152, 158, 164, 170, 176, 182, 188, 194, 200, 206, 212, 218, 224, 230, 236, 242, 248, 254, 260, 266, 272, 284, 296, 307, 320, 332, 344 and 356; and (f) a light chain variable region CDR3 comprising an amino acid sequence selected from the group consisting of SEQ ID NOs: 129, 135, 141, 147, 153, 159, 165, 171, 177, 183, 189, 195, 201, 207, 213, 219, 225, 231, 237, 243, 249, 255, 261, 267, 273, 285, 297, 308, 321, 333, 345 and 357; wherein the antibody specifically binds GPRC5D, e.g., human GPRC5D.

In certain embodiments, the antibody comprises:
(i) a heavy chain variable region CDR1 comprising amino acids having the sequence set forth in SEQ ID NO: 124;
(ii) a heavy chain variable region CDR2 comprising amino acids having the sequence set forth in SEQ ID NO: 125;
(iii) a heavy chain variable region CDR3 comprising amino acids having the sequence set forth in SEQ ID NO: 126;
(iv) a light chain variable region CDR1 comprising amino acids having the sequence set forth in SEQ ID NO: 127;
(v) a light chain variable region CDR2 comprising amino acids having the sequence set forth in SEQ ID NO:128; and
(vi) a light chain variable region CDR3 comprising amino acids having the sequence set forth in SEQ ID NO: 129.

In certain embodiments, the antibody comprises:
(i) a heavy chain variable region CDR1 comprising amino acids having the sequence set forth in SEQ ID NO: 130;
(ii) a heavy chain variable region CDR2 comprising amino acids having the sequence set forth in SEQ ID NO: 131;
(iii) a heavy chain variable region CDR3 comprising amino acids having the sequence set forth in SEQ ID NO: 132;
(iv) a light chain variable region CDR1 comprising amino acids having the sequence set forth in SEQ ID NO: 133;
(v) a light chain variable region CDR2 comprising amino acids having the sequence set forth in SEQ ID NO:134; and
(vi) a light chain variable region CDR3 comprising amino acids having the sequence set forth in SEQ ID NO: 135.

In certain embodiments, the antibody comprises:
(i) a heavy chain variable region CDR1 comprising amino acids having the sequence set forth in SEQ ID NO: 136;
(ii) a heavy chain variable region CDR2 comprising amino acids having the sequence set forth in SEQ ID NO: 137;
(iii) a heavy chain variable region CDR3 comprising amino acids having the sequence set forth in SEQ ID NO: 138;
(iv) a light chain variable region CDR1 comprising amino acids having the sequence set forth in SEQ ID NO: 139;
(v) a light chain variable region CDR2 comprising amino acids having the sequence set forth in SEQ ID NO:140; and
(vi) a light chain variable region CDR3 comprising amino acids having the sequence set forth in SEQ ID NO: 141.

In certain embodiments, the antibody comprises:
(i) a heavy chain variable region CDR1 comprising amino acids having the sequence set forth in SEQ ID NO: 142;
(ii) a heavy chain variable region CDR2 comprising amino acids having the sequence set forth in SEQ ID NO: 143;
(iii) a heavy chain variable region CDR3 comprising amino acids having the sequence set forth in SEQ ID NO: 144;
(iv) a light chain variable region CDR1 comprising amino acids having the sequence set forth in SEQ ID NO: 145;
(v) a light chain variable region CDR2 comprising amino acids having the sequence set forth in SEQ ID NO:146; and
(vi) a light chain variable region CDR3 comprising amino acids having the sequence set forth in SEQ ID NO: 147.

In certain embodiments, the antibody comprises:
(i) a heavy chain variable region CDR1 comprising amino acids having the sequence set forth in SEQ ID NO: 148;
(ii) a heavy chain variable region CDR2 comprising amino acids having the sequence set forth in SEQ ID NO: 149;
(iii) a heavy chain variable region CDR3 comprising amino acids having the sequence set forth in SEQ ID NO: 150;
(iv) a light chain variable region CDR1 comprising amino acids having the sequence set forth in SEQ ID NO: 151;
(v) a light chain variable region CDR2 comprising amino acids having the sequence set forth in SEQ ID NO:152; and
(vi) a light chain variable region CDR3 comprising amino acids having the sequence set forth in SEQ ID NO: 153.

In certain embodiments, the antibody comprises:
(i) a heavy chain variable region CDR1 comprising amino acids having the sequence set forth in SEQ ID NO: 154;
(ii) a heavy chain variable region CDR2 comprising amino acids having the sequence set forth in SEQ ID NO: 155;
(iii) a heavy chain variable region CDR3 comprising amino acids having the sequence set forth in SEQ ID NO: 156;
(iv) a light chain variable region CDR1 comprising amino acids having the sequence set forth in SEQ ID NO: 157;
(v) a light chain variable region CDR2 comprising amino acids having the sequence set forth in SEQ ID NO:158; and
(vi) a light chain variable region CDR3 comprising amino acids having the sequence set forth in SEQ ID NO: 159.

In certain embodiments, the antibody comprises:
(i) a heavy chain variable region CDR1 comprising amino acids having the sequence set forth in SEQ ID NO: 160;
(ii) a heavy chain variable region CDR2 comprising amino acids having the sequence set forth in SEQ ID NO: 161;
(iii) a heavy chain variable region CDR3 comprising amino acids having the sequence set forth in SEQ ID NO: 162;
(iv) a light chain variable region CDR1 comprising amino acids having the sequence set forth in SEQ ID NO: 163;
(v) a light chain variable region CDR2 comprising amino acids having the sequence set forth in SEQ ID NO:164; and
(vi) a light chain variable region CDR3 comprising amino acids having the sequence set forth in SEQ ID NO: 165.

In certain embodiments, the antibody comprises:
(i) a heavy chain variable region CDR1 comprising amino acids having the sequence set forth in SEQ ID NO: 166;
(ii) a heavy chain variable region CDR2 comprising amino acids having the sequence set forth in SEQ ID NO: 167;
(iii) a heavy chain variable region CDR3 comprising amino acids having the sequence set forth in SEQ ID NO: 168;
(iv) a light chain variable region CDR1 comprising amino acids having the sequence set forth in SEQ ID NO: 169;
(v) a light chain variable region CDR2 comprising amino acids having the sequence set forth in SEQ ID NO:170; and
(vi) a light chain variable region CDR3 comprising amino acids having the sequence set forth in SEQ ID NO: 171.

In certain embodiments, the antibody comprises:
(i) a heavy chain variable region CDR1 comprising amino acids having the sequence set forth in SEQ ID NO: 172;
(ii) a heavy chain variable region CDR2 comprising amino acids having the sequence set forth in SEQ ID NO: 173;
(iii) a heavy chain variable region CDR3 comprising amino acids having the sequence set forth in SEQ ID NO: 174;
(iv) a light chain variable region CDR1 comprising amino acids having the sequence set forth in SEQ ID NO: 175;
(v) a light chain variable region CDR2 comprising amino acids having the sequence set forth in SEQ ID NO:176; and
(vi) a light chain variable region CDR3 comprising amino acids having the sequence set forth in SEQ ID NO: 177.

In certain embodiments, the antibody comprises:
(i) a heavy chain variable region CDR1 comprising amino acids having the sequence set forth in SEQ ID NO: 178;
(ii) a heavy chain variable region CDR2 comprising amino acids having the sequence set forth in SEQ ID NO: 179;
(iii) a heavy chain variable region CDR3 comprising amino acids having the sequence set forth in SEQ ID NO: 180;
(iv) a light chain variable region CDR1 comprising amino acids having the sequence set forth in SEQ ID NO: 181;
(v) a light chain variable region CDR2 comprising amino acids having the sequence set forth in SEQ ID NO:182; and
(vi) a light chain variable region CDR3 comprising amino acids having the sequence set forth in SEQ ID NO: 183.

In certain embodiments, the antibody comprises:
(i) a heavy chain variable region CDR1 comprising amino acids having the sequence set forth in SEQ ID NO: 184;
(ii) a heavy chain variable region CDR2 comprising amino acids having the sequence set forth in SEQ ID NO: 185;
(iii) a heavy chain variable region CDR3 comprising amino acids having the sequence set forth in SEQ ID NO: 186;
(iv) a light chain variable region CDR1 comprising amino acids having the sequence set forth in SEQ ID NO: 187;
(v) a light chain variable region CDR2 comprising amino acids having the sequence set forth in SEQ ID NO:188; and
(vi) a light chain variable region CDR3 comprising amino acids having the sequence set forth in SEQ ID NO: 189.

In certain embodiments, the antibody comprises:
(i) a heavy chain variable region CDR1 comprising amino acids having the sequence set forth in SEQ ID NO: 190;
(ii) a heavy chain variable region CDR2 comprising amino acids having the sequence set forth in SEQ ID NO: 191;
(iii) a heavy chain variable region CDR3 comprising amino acids having the sequence set forth in SEQ ID NO: 192;
(iv) a light chain variable region CDR1 comprising amino acids having the sequence set forth in SEQ ID NO: 193;
(v) a light chain variable region CDR2 comprising amino acids having the sequence set forth in SEQ ID NO:194; and
(vi) a light chain variable region CDR3 comprising amino acids having the sequence set forth in SEQ ID NO: 195.

In certain embodiments, the antibody comprises:
(i) a heavy chain variable region CDR1 comprising amino acids having the sequence set forth in SEQ ID NO: 196;
(ii) a heavy chain variable region CDR2 comprising amino acids having the sequence set forth in SEQ ID NO: 197;
(iii) a heavy chain variable region CDR3 comprising amino acids having the sequence set forth in SEQ ID NO: 198;
(iv) a light chain variable region CDR1 comprising amino acids having the sequence set forth in SEQ ID NO: 199;
(xxii) a light chain variable region CDR2 comprising amino acids having the sequence set forth in SEQ ID NO:200; and
(vi) a light chain variable region CDR3 comprising amino acids having the sequence set forth in SEQ ID NO: 201.

In certain embodiments, the antibody comprises:
(i) a heavy chain variable region CDR1 comprising amino acids having the sequence set forth in SEQ ID NO: 202;
(ii) a heavy chain variable region CDR2 comprising amino acids having the sequence set forth in SEQ ID NO: 203;
(iii) a heavy chain variable region CDR3 comprising amino acids having the sequence set forth in SEQ ID NO: 204;
(iv) a light chain variable region CDR1 comprising amino acids having the sequence set forth in SEQ ID NO: 205;
(xxii) a light chain variable region CDR2 comprising amino acids having the sequence set forth in SEQ ID NO:206; and
(vi) a light chain variable region CDR3 comprising amino acids having the sequence set forth in SEQ ID NO: 207.

In certain embodiments, the antibody comprises:
(i) a heavy chain variable region CDR1 comprising amino acids having the sequence set forth in SEQ ID NO: 208;
(ii) a heavy chain variable region CDR2 comprising amino acids having the sequence set forth in SEQ ID NO: 209;
(iii) a heavy chain variable region CDR3 comprising amino acids having the sequence set forth in SEQ ID NO: 210;
(iv) a light chain variable region CDR1 comprising amino acids having the sequence set forth in SEQ ID NO: 211;
(xxii) a light chain variable region CDR2 comprising amino acids having the sequence set forth in SEQ ID NO:212; and
(vi) a light chain variable region CDR3 comprising amino acids having the sequence set forth in SEQ ID NO: 213.

In certain embodiments, the antibody comprises:
(i) a heavy chain variable region CDR1 comprising amino acids having the sequence set forth in SEQ ID NO: 214;
(ii) a heavy chain variable region CDR2 comprising amino acids having the sequence set forth in SEQ ID NO: 215;
(iii) a heavy chain variable region CDR3 comprising amino acids having the sequence set forth in SEQ ID NO: 216;
(iv) a light chain variable region CDR1 comprising amino acids having the sequence set forth in SEQ ID NO: 217;
(xxii) a light chain variable region CDR2 comprising amino acids having the sequence set forth in SEQ ID NO:218; and
(vi) a light chain variable region CDR3 comprising amino acids having the sequence set forth in SEQ ID NO: 219.

In certain embodiments, the antibody comprises:
(i) a heavy chain variable region CDR1 comprising amino acids having the sequence set forth in SEQ ID NO: 220;
(ii) a heavy chain variable region CDR2 comprising amino acids having the sequence set forth in SEQ ID NO: 221;
(iii) a heavy chain variable region CDR3 comprising amino acids having the sequence set forth in SEQ ID NO: 222;
(iv) a light chain variable region CDR1 comprising amino acids having the sequence set forth in SEQ ID NO: 223;
(xxii) a light chain variable region CDR2 comprising amino acids having the sequence set forth in SEQ ID NO:224; and
(vi) a light chain variable region CDR3 comprising amino acids having the sequence set forth in SEQ ID NO: 225.

In certain embodiments, the antibody comprises:
(i) a heavy chain variable region CDR1 comprising amino acids having the sequence set forth in SEQ ID NO: 226;
(ii) a heavy chain variable region CDR2 comprising amino acids having the sequence set forth in SEQ ID NO: 227;
(iii) a heavy chain variable region CDR3 comprising amino acids having the sequence set forth in SEQ ID NO: 228;
(iv) a light chain variable region CDR1 comprising amino acids having the sequence set forth in SEQ ID NO: 229;
(xxii) a light chain variable region CDR2 comprising amino acids having the sequence set forth in SEQ ID NO:230; and
(vi) a light chain variable region CDR3 comprising amino acids having the sequence set forth in SEQ ID NO: 231.

In certain embodiments, the antibody comprises:
(i) a heavy chain variable region CDR1 comprising amino acids having the sequence set forth in SEQ ID NO: 232;
(ii) a heavy chain variable region CDR2 comprising amino acids having the sequence set forth in SEQ ID NO: 233;
(iii) a heavy chain variable region CDR3 comprising amino acids having the sequence set forth in SEQ ID NO: 234;
(iv) a light chain variable region CDR1 comprising amino acids having the sequence set forth in SEQ ID NO: 235;
(xxii) a light chain variable region CDR2 comprising amino acids having the sequence set forth in SEQ ID NO:236; and
(vi) a light chain variable region CDR3 comprising amino acids having the sequence set forth in SEQ ID NO: 237.

In certain embodiments, the antibody comprises:
(i) a heavy chain variable region CDR1 comprising amino acids having the sequence set forth in SEQ ID NO: 238;
(ii) a heavy chain variable region CDR2 comprising amino acids having the sequence set forth in SEQ ID NO: 239;
(iii) a heavy chain variable region CDR3 comprising amino acids having the sequence set forth in SEQ ID NO: 240;
(iv) a light chain variable region CDR1 comprising amino acids having the sequence set forth in SEQ ID NO: 241;
(xxii) a light chain variable region CDR2 comprising amino acids having the sequence set forth in SEQ ID NO:242; and
(vi) a light chain variable region CDR3 comprising amino acids having the sequence set forth in SEQ ID NO: 243.

In certain embodiments, the antibody comprises:
(i) a heavy chain variable region CDR1 comprising amino acids having the sequence set forth in SEQ ID NO: 244;
(ii) a heavy chain variable region CDR2 comprising amino acids having the sequence set forth in SEQ ID NO: 245;
(iii) a heavy chain variable region CDR3 comprising amino acids having the sequence set forth in SEQ ID NO: 246;
(iv) a light chain variable region CDR1 comprising amino acids having the sequence set forth in SEQ ID NO: 247;
(xxii) a light chain variable region CDR2 comprising amino acids having the sequence set forth in SEQ ID NO:248; and
(vi) a light chain variable region CDR3 comprising amino acids having the sequence set forth in SEQ ID NO: 249.

In certain embodiments, the antibody comprises:
(i) a heavy chain variable region CDR1 comprising amino acids having the sequence set forth in SEQ ID NO: 250;
(ii) a heavy chain variable region CDR2 comprising amino acids having the sequence set forth in SEQ ID NO: 251;
(iii) a heavy chain variable region CDR3 comprising amino acids having the sequence set forth in SEQ ID NO: 252;
(iv) a light chain variable region CDR1 comprising amino acids having the sequence set forth in SEQ ID NO: 253;
(xxii) a light chain variable region CDR2 comprising amino acids having the sequence set forth in SEQ ID NO:254; and
(vi) a light chain variable region CDR3 comprising amino acids having the sequence set forth in SEQ ID NO: 255.

In certain embodiments, the antibody comprises:
(i) a heavy chain variable region CDR1 comprising amino acids having the sequence set forth in SEQ ID NO: 256;
(ii) a heavy chain variable region CDR2 comprising amino acids having the sequence set forth in SEQ ID NO: 257;
(iii) a heavy chain variable region CDR3 comprising amino acids having the sequence set forth in SEQ ID NO: 258;
(iv) a light chain variable region CDR1 comprising amino acids having the sequence set forth in SEQ ID NO: 259;
(xxii) a light chain variable region CDR2 comprising amino acids having the sequence set forth in SEQ ID NO:260; and
(vi) a light chain variable region CDR3 comprising amino acids having the sequence set forth in SEQ ID NO: 261.

In certain embodiments, the antibody comprises:
(i) a heavy chain variable region CDR1 comprising amino acids having the sequence set forth in SEQ ID NO: 262;
(ii) a heavy chain variable region CDR2 comprising amino acids having the sequence set forth in SEQ ID NO: 263;
(iii) a heavy chain variable region CDR3 comprising amino acids having the sequence set forth in SEQ ID NO: 264;
(iv) a light chain variable region CDR1 comprising amino acids having the sequence set forth in SEQ ID NO: 265;
(xxii) a light chain variable region CDR2 comprising amino acids having the sequence set forth in SEQ ID NO:266; and
(vi) a light chain variable region CDR3 comprising amino acids having the sequence set forth in SEQ ID NO: 267.

In certain embodiments, the antibody comprises:
(i) a heavy chain variable region CDR1 comprising amino acids having the sequence set forth in SEQ ID NO: 268;
(ii) a heavy chain variable region CDR2 comprising amino acids having the sequence set forth in SEQ ID NO: 269;
(iii) a heavy chain variable region CDR3 comprising amino acids having the sequence set forth in SEQ ID NO: 270;
(iv) a light chain variable region CDR1 comprising amino acids having the sequence set forth in SEQ ID NO: 271;
(xxii) a light chain variable region CDR2 comprising amino acids having the sequence set forth in SEQ ID NO:272; and
(vi) a light chain variable region CDR3 comprising amino acids having the sequence set forth in SEQ ID NO: 273.

In certain embodiments, the antibody comprises:
(i) a heavy chain variable region CDR1 comprising amino acids having the sequence set forth in SEQ ID NO: 280;
(ii) a heavy chain variable region CDR2 comprising amino acids having the sequence set forth in SEQ ID NO: 281;
(iii) a heavy chain variable region CDR3 comprising amino acids having the sequence set forth in SEQ ID NO: 282;
(iv) a light chain variable region CDR1 comprising amino acids having the sequence set forth in SEQ ID NO: 283;
(xxii) a light chain variable region CDR2 comprising amino acids having the sequence set forth in SEQ ID NO:284; and
(vi) a light chain variable region CDR3 comprising amino acids having the sequence set forth in SEQ ID NO: 285.

In certain embodiments, the antibody comprises:
(i) a heavy chain variable region CDR1 comprising amino acids having the sequence set forth in SEQ ID NO: 292;
(ii) a heavy chain variable region CDR2 comprising amino acids having the sequence set forth in SEQ ID NO: 293;
(iii) a heavy chain variable region CDR3 comprising amino acids having the sequence set forth in SEQ ID NO: 294;
(iv) a light chain variable region CDR1 comprising amino acids having the sequence set forth in SEQ ID NO: 295;
(xxii) a light chain variable region CDR2 comprising amino acids having the sequence set forth in SEQ ID NO:296; and
(vi) a light chain variable region CDR3 comprising amino acids having the sequence set forth in SEQ ID NO: 297.

In certain embodiments, the antibody comprises:
(i) a heavy chain variable region CDR1 comprising amino acids having the sequence set forth in SEQ ID NO: 303;
(ii) a heavy chain variable region CDR2 comprising amino acids having the sequence set forth in SEQ ID NO: 304;
(iii) a heavy chain variable region CDR3 comprising amino acids having the sequence set forth in SEQ ID NO: 305;
(iv) a light chain variable region CDR1 comprising amino acids having the sequence set forth in SEQ ID NO: 306;
(xxii) a light chain variable region CDR2 comprising amino acids having the sequence set forth in SEQ ID NO: 307; and
(vi) a light chain variable region CDR3 comprising amino acids having the sequence set forth in SEQ ID NO: 308.

In certain embodiments, the antibody comprises:
(i) a heavy chain variable region CDR1 comprising amino acids having the sequence set forth in SEQ ID NO: 316;
(ii) a heavy chain variable region CDR2 comprising amino acids having the sequence set forth in SEQ ID NO: 317;
(iii) a heavy chain variable region CDR3 comprising amino acids having the sequence set forth in SEQ ID NO: 318;
(iv) a light chain variable region CDR1 comprising amino acids having the sequence set forth in SEQ ID NO: 319;
(xxii) a light chain variable region CDR2 comprising amino acids having the sequence set forth in SEQ ID NO: 320; and
(vi) a light chain variable region CDR3 comprising amino acids having the sequence set forth in SEQ ID NO: 321.

In certain embodiments, the antibody comprises:
(i) a heavy chain variable region CDR1 comprising amino acids having the sequence set forth in SEQ ID NO: 328;
(ii) a heavy chain variable region CDR2 comprising amino acids having the sequence set forth in SEQ ID NO: 329;
(iii) a heavy chain variable region CDR3 comprising amino acids having the sequence set forth in SEQ ID NO: 330;
(iv) a light chain variable region CDR1 comprising amino acids having the sequence set forth in SEQ ID NO: 331;
(xxii) a light chain variable region CDR2 comprising amino acids having the sequence set forth in SEQ ID NO: 332; and
(vi) a light chain variable region CDR3 comprising amino acids having the sequence set forth in SEQ ID NO: 333.

In certain embodiments, the antibody comprises:
(i) a heavy chain variable region CDR1 comprising amino acids having the sequence set forth in SEQ ID NO: 340;
(ii) a heavy chain variable region CDR2 comprising amino acids having the sequence set forth in SEQ ID NO: 341;
(iii) a heavy chain variable region CDR3 comprising amino acids having the sequence set forth in SEQ ID NO: 342;
(iv) a light chain variable region CDR1 comprising amino acids having the sequence set forth in SEQ ID NO: 343;
(xxii) a light chain variable region CDR2 comprising amino acids having the sequence set forth in SEQ ID NO: 344; and
(vi) a light chain variable region CDR3 comprising amino acids having the sequence set forth in SEQ ID NO: 345.

In certain embodiments, the antibody comprises:
(i) a heavy chain variable region CDR1 comprising amino acids having the sequence set forth in SEQ ID NO: 352;
(ii) a heavy chain variable region CDR2 comprising amino acids having the sequence set forth in SEQ ID NO: 353;
(iii) a heavy chain variable region CDR3 comprising amino acids having the sequence set forth in SEQ ID NO: 354;
(iv) a light chain variable region CDR1 comprising amino acids having the sequence set forth in SEQ ID NO: 355;
(xxii) a light chain variable region CDR2 comprising amino acids having the sequence set forth in SEQ ID NO: 356; and
(vi) a light chain variable region CDR3 comprising amino acids having the sequence set forth in SEQ ID NO: 357.

The constant region/framework region of the anti-GPRC5D antibodies disclosed herein can be altered, for example, by amino acid substitution, to modify the properties of the antibody (e.g., to increase or decrease one or more of: antigen binding affinity, Fc receptor binding, antibody carbohydrate, for example, glycosylation, fucosylation etc, the number of cysteine residues, effector cell function, effector cell function, complement function or introduction of a conjugation site).

In certain embodiments, a presently disclosed anti-GPRC5D antibody is a fully-human antibody, e.g., any one of ET150-153, ET150-166, ET150-170, ET150-171, ET150-175, ET150-154, ET150-156, ET150-157, ET150-159, ET150-160, ET150-161, ET150-162, ET150-163, ET150-151, ET150-152, ET150-155, ET150-158, ET150-168, ET150-165, ET150-167, ET150-169, ET150-172, ET150-173, ET150-024, ET150-026, ET150-028, ET150-029, ET150-030, ET150-031, ET150-032 and ET150-033. Fully-human mAbs are preferred for therapeutic use in humans because murine antibodies cause an immunogenicity reaction, known as the HAMA (human anti-mouse antibodies) response (Azinovic I, et al. Survival benefit associated with human anti-mouse antibody (HAMA) in patients with B-cell malignancies. Cancer Immunol Immunother 2006; 55(12):1451-8; Tjandra J J, et al. Development of human anti-murine antibody (HAMA) response in patients. Immunol Cell Biol 1990; 68(6):367-76), when administered to humans, causing serious side effects, including anaphylaxis and hypersensitivity reactions. This immunogenicity reaction is triggered by the human immune system recognizing the murine antibodies as foreign because of slightly different amino acid sequences from natural human antibodies. Humanization methods known in the art (Riechmann L, et al. Reshaping human antibodies for therapy. Nature 1988; 332 (6162): 332:323; Queen C, et al. A humanized antibody that binds to the interleukin 2 receptor. Proc Natl Acad Sci USA 1989; 86 (24): 10029-33) can be employed to reduce the immunogenicity of murine-derived antibodies (Gerd R, et al. Serological Analysis of Human Anti-Human Antibody Responses in Colon Cancer Patients Treated with Repeated Doses of Humanized Monoclonal Antibody A33. Cancer Res 2001; 61, 6851-6859).

The use of phage display libraries has made it possible to select large numbers of Ab repertoires for unique and rare Abs against very defined epitopes (for more details on phage display see McCafferty et al., Phage antibodies: filamentous phage displaying antibody variable domains. Nature, 348: 552-554.) The rapid identification of human Fab or single chain Fv (ScFV) fragments highly specific for tumor antigen-derived peptide-MHC complex molecules has thus become possible (19-22). Recently, immuno-toxins, generated by fusing TCR-like Fab specific for melanoma Ag MART-1 26-35/A2 or gp100 280-288/A2 to a truncated form of Pseudomonas endotoxin, have been shown to inhibit human melanoma growth both in vitro and in vivo (Klechevsky E, et al. Antitumor activity of immunotoxins with T-cell receptor-like specificity against human melanoma xenografts. Cancer Res 2008; 68 (15): 6360-6367). In addition, by engineering full-length mAb using the Fab fragments, it is possible to directly generate a therapeutic human mAb, bypassing months of time-consuming work, normally needed for developing therapeutic mAbs. The presently disclosed subject matter involves the development of a fully human mAb that recognizes, for example, a human GPRC5D polypeptide (e.g., a polypeptide having the amino acid sequence set forth in SEQ ID NO:97) for cancer therapy.

3. Homologous Antibodies

In certain embodiments, an antibody of the presently disclosed subject matter comprises heavy and light chain variable regions comprising amino acid sequences that are homologous to the amino acid sequences of the antibodies described herein (e.g., ET150-153, ET150-166, ET150-170, ET150-171, ET150-175, ET150-154, ET150-156, ET150-157, ET150-159, ET150-160, ET150-161, ET150-162, ET150-163, ET150-151, ET150-152, ET150-155, ET150-158, ET150-168, ET150-165, ET150-167, ET150-169, ET150-172, ET150-173, ET150-024, ET150-026, ET150-028, ET150-029, ET150-030, ET150-031, ET150-032 and ET150-033 antibodies), and wherein the antibodies retain the desired functional properties of the anti-PGPRC5D antibodies of the presently disclosed subject matter.

For example, the presently disclosed subject matter provides an isolated antibody, or antigen-binding portion thereof, comprising a heavy chain variable region and a light chain variable region, wherein:
(a) the heavy chain variable region comprises an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to an amino acid sequence selected from the group consisting of SEQ ID NOs: 1, 5, 9, 13, 17, 21, 25, 29, 33, 37, 41, 45, 49, 53, 57, 61, 65, 69, 73, 77, 81, 85, 89, 93, 274, 286, 298, 310, 322, 334, 346 and 358; and
(b) the light chain variable region comprises an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to an amino acid sequence selected from the group consisting of SEQ ID NOs: 2, 6, 10, 14, 18, 22, 26, 30, 34, 38, 42, 46, 50, 54, 58, 62, 66, 70, 74, 78, 82, 86, 90, 94, 275, 287, 299, 311, 323, 335, 347 and 359; and the antibody binds to human GPRC5D with a K_{d} of 1×10⁻⁷ M or less.

In certain embodiments, the V_{H} and/or V_{L} amino acid sequences can be at least 80%, 81%, 82%, 83%, 84%, 85%, 86%, 87%, 88%, 89%, 90%, 91%, 92%, 93%, 94%, 95%, 96%, 97%, 98% or 99% homologous to the sequences set forth above. An antibody having V_{H} and V_{L} regions having high (i.e., 80% or greater) homology to the V_{H} and V_{L} regions of the sequences set forth above, can be obtained by mutagenesis (e.g., site-directed or PCR-mediated mutagenesis), followed by testing of the encoded altered antibody for retained function (i.e., the binding affinity) using the binding assays described herein.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the presently disclosed subject matter can further be used as a “query sequence” to perform a search against public databases to, for example, identify related sequences. Such searches can be performed using the)(BLAST program (version 2.0) of Altschul, et al. (1990) J. Mol. Biol. 215:403-10. BLAST protein searches can be performed with the)(BLAST program, score=50, wordlength=3 to obtain amino acid sequences homologous to the antibody molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g.,)(BLAST and NBLAST) can be used. (See www.ncbi.nlm.nih.gov).

4. Antibodies with Conservative Modifications

In certain embodiments, an antibody of the presently disclosed subject matter comprises a heavy chain variable region comprising CDR1, CDR2 and CDR3 sequences and a light chain variable region comprising CDR1, CDR2 and CDR3 sequences, wherein one or more of these CDR sequences comprise specified amino acid sequences based on the preferred antibodies described herein (e.g., ET150-153, ET150-166, ET150-170, ET150-171, ET150-175, ET150-154, ET150-156, ET150-157, ET150-159, ET150-160, ET150-161, ET150-162, ET150-163, ET150-151, ET150-152, ET150-155, ET150-158, ET150-168, ET150-165, ET150-167, ET150-169, ET150-172, ET150-173, ET150-024, ET150-026, ET150-028, ET150-029, ET150-030, ET150-031, ET150-032 and ET150-033 antibodies), or conservative modifications thereof, and wherein the antibodies retain the desired functional properties of the anti-GPRC5D antibodies of the presently disclosed subject matter.

The presently disclosed subject matter provides an isolated antibody, or antigen-binding portion thereof, comprising a heavy chain variable region comprising CDR1, CDR2, and CDR3 sequences and a light chain variable region comprising CDR1, CDR2, and CDR3 sequences, wherein:
(a) the heavy chain variable region CDR3 sequence comprises an amino acid sequence selected from the group consisting of amino acid sequences of SEQ ID NOs: 126, 132, 138, 144, 150, 156, 162, 168, 174, 180, 186, 192, 198, 204, 210, 216, 222, 228, 234, 240, 246, 252, 258, 264, 270, 282, 294, 305, 318, 330, 342 and 354, and conservative modifications thereof;
(b) the light chain variable region CDR3 sequence comprises an amino acid sequence selected from the group consisting of amino acid sequence of SEQ ID NOs: 129, 135, 141, 147, 153, 159, 165, 171, 177, 183, 189, 195, 201, 207, 213, 219, 225, 231, 237, 243, 249, 255, 261, 267, 273, 285, 297, 308, 321, 333, 345 and 357, and 431, and conservative modifications thereof; and the antibody exhibits binds to human GPRC5D with a K_{d} of 1×10⁻⁷ M or less.

In certain embodiments, the heavy chain variable region CDR2 sequence comprises an amino acid sequence selected from the group consisting of amino acid sequences of SEQ ID NOs: 125, 131, 137, 143, 149, 155, 161, 167, 173, 179, 185, 191, 197, 203, 209, 215, 221, 227, 233, 239, 245, 251, 257, 263, 269, 281, 293, 304, 317, 329, 341 and 353, and conservative modifications thereof; and the light chain variable region CDR2 sequence comprises an amino acid sequence selected from the group consisting of amino acid sequences of SEQ ID NOs: 128, 134, 140, 146, 152, 158, 164, 170, 176, 182, 188, 194, 200, 206, 212, 218, 224, 230, 236, 242, 248, 254, 260, 266, 272, 284, 296, 307, 320, 332, 344 and 356, and conservative modifications thereof.

In certain embodiments, the heavy chain variable region CDR1 sequence comprises an amino acid sequence selected from the group consisting of amino acid sequences of SEQ ID NOs: 124, 130, 136, 142, 148, 154, 160, 166, 172, 178, 184, 190, 196, 202, 208, 214, 220, 226, 232, 238, 244, 250, 256, 262, 268, 280, 292, 303, 316, 328, 340 and 352, and conservative modifications thereof; and the light chain variable region CDR1 sequence comprises an amino acid sequence selected from the group consisting of amino acid sequences of SEQ ID NOs: 127, 133, 139, 145, 151, 157, 163, 169, 175, 181, 187, 193, 199, 205, 211, 217, 223, 229, 235, 241, 247, 253, 259, 265, 271, 283, 295, 306, 319, 331, 343 and 355, and conservative modifications thereof.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis.

Conservative amino acid substitutions are ones in which the amino acid residue is replaced with an amino acid residue having a similar side chain. Families of amino acid residues having similar side chains have been defined in the art. Exemplary conservative amino acid substitutions are shown in Table 33. Amino acid substitutions may be introduced into an antibody of interest and the products screened for a desired activity, e.g., retained/improved antigen binding, decreased immunogenicity, or improved ADCC or CDC. In certain embodiments, a sequence disclosed herein, e.g., a CDR sequence, a VH sequence or a VL sequence, can have up to about one, up to about two, up to about three, up to about four, up to about five, up to about six, up to about seven, up to about eight, up to about nine or up to about ten amino acid residues that are modified and/or substituted.

TABLE 33

| Original Residue | Exemplary conservative amino acid Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe |
| Leu (xii) | Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (xxiii) | Tyr; Phe |
| Tyr (Y) | Trp; Phe; Thr; Ser |
| Val (V) | Ile; Leu; Met; Phe; Ala |

Amino acids may be grouped according to common side-chain properties:
hydrophobic: Norleucine, Met, Ala, Val, Leu, Ile;
neutral hydrophilic: Cys, Ser, Thr, Asn, Gln;
acidic: Asp, Glu;
basic: His, Lys, Arg;
residues that influence chain orientation: Gly, Pro;
aromatic: Trp, Tyr, Phe.

Non-conservative substitutions will entail exchanging a member of one of these classes for another class.

5. Anti-GPRC5D Antibodies that Cross-Compete for Binding to GPRC5D with Anti-GPRC5D Antibodies of the Invention

The presently disclosed subject matter provides antibodies that cross-compete with any of the disclosed anti-GPRC5D antibodies for binding to GPRC5D (e.g., human GPRC5D). For example, and not by way of limitation, the cross-competing antibodies can bind to the same epitope region, e.g., same epitope, adjacent epitope, or overlapping as any of the anti-GPRC5D antibodies of the presently disclosed subject matter. In certain embodiments, the reference antibody for cross-competition studies can be any one of the anti-GPRC5D antibodies disclosed herein, e.g., ET150-153, ET150-166, ET150-170, ET150-171, ET150-175, ET150-154, ET150-156, ET150-157, ET150-159, ET150-160, ET150-161, ET150-162, ET150-163, ET150-151, ET150-152, ET150-155, ET150-158, ET150-168, ET150-165, ET150-167, ET150-169, ET150-172, ET150-173, ET150-024, ET150-026, ET150-028, ET150-029, ET150-030, ET150-031, ET150-032 and ET150-033 antibodies.

In certain embodiments, the cross-competing antibody binds to an epitope region comprising amino acids 14-22 of SEQ ID NO: 97. In certain embodiments, the cross-competing antibody binds to one, two, three, four, five, six, or seven epitope regions selected from the group consisting of amino acids 5-17, 10-17, 1-27, 15-23, 16-23, 16-25, 85-93, 85-95, 145-167, 157-164, 157-167, 226-239, 230-237, 229-237, 230-243 and 227-237 of SEQ ID NO: 97.

Such cross-competing antibodies can be identified based on their ability to cross-compete with any one of the presently disclosed anti-GPRC5D antibodies in standard GPRC5D binding assays. For example, Biacore® analysis, ELISA assays or flow cytometry can be used to demonstrate cross-competition with the antibodies of the presently disclosed subject matter. The ability of a test antibody to inhibit the binding of, for example, any one of the presently disclosed GPRC5D antibodies (e.g., ET150-153, ET150-166, ET150-170, ET150-171, ET150-175, ET150-154, ET150-156, ET150-157, ET150-159, ET150-160, ET150-161, ET150-162, ET150-163, ET150-151, ET150-152, ET150-155, ET150-158, ET150-168, ET150-165, ET150-167, ET150-169, ET150-172, ET150-173, ET150-024, ET150-026, ET150-028, ET150-029, ET150-030, ET150-031, ET150-032 and ET150-033 antibodies) to human GPRC5D demonstrates that the test antibody can compete with any one of the presently disclosed anti-GPRC5D antibodies for binding to human GPRC5D and thus binds to the same epitope region on human GPRC5D as any one of the presently disclosed anti-GPRC5D antibodies. In certain embodiments, the cross-competing antibody binds to the same epitope on human GPRC5D as any one of the presently disclosed anti-GPRC5D antibodies.

6. Characterization of Antibody Binding to Antigen

Antibodies of the presently disclosed subject can be tested for binding to GPRC5D by, for example, standard ELISA. To determine if the selected anti-GPRC5D antibodies bind to unique epitopes, each antibody can be biotinylated using commercially available reagents (Pierce, Rockford, IL). Competition studies using unlabeled monoclonal antibodies and biotinylated monoclonal antibodies can be performed using GPRC5D coated-ELISA plates as described above. Biotinylated mAb binding can be detected with a strep-avidin-alkaline phosphatase probe.

To determine the isotype of purified antibodies, isotype ELISAs can be performed using reagents specific for antibodies of a particular isotype. Anti-GPRC5D human IgGs can be further tested for reactivity with GPRC5D antigen by Western blotting.

In certain embodiments, K_{d} is measured by a radiolabeled antigen binding assay (RIA). In certain embodiments, an RIA is performed with the Fab version of an antibody of interest and its antigen. For example, solution binding affinity of Fabs for antigen is measured by equilibrating Fab with a minimal concentration of (125 I)-labeled antigen in the presence of a titration series of unlabeled antigen, then capturing bound antigen with an anti-Fab antibody-coated plate (see, e.g., Chen et al., J Mol. Biol. 293:865-881(1999)).

In certain embodiments, K_{d} is measured using a BIACORE® surface plasmon resonance assay. For example, an assay using a BIACORE®-2000 or a BIACORE®-3000 (BIAcore®, Inc., Piscataway, NJ).

Epitope Mapping

In certain embodiments, the antibody or an antigen-binding fragment thereof binds to a human GPRC5D polypeptide comprising the amino acid sequence set forth in SEQ ID NO: 97. In certain embodiments, the antibody or an antigen-binding fragment thereof binds to one, two, three or four of N-terminal region (amino acids 1-27 of SEQ ID NO:97), ECL1 region (amino acids 85-93 of SEQ ID NO:97), ECL2 region (amino acids 145-167 of SEQ ID NO:97), and ECL3 region (amino acids 226-239 of SEQ ID NO:97). In certain embodiments, an antibody or an antigen-binding fragment thereof of the presently disclosed subject matter binds to an epitope region in the N-terminal region, including, but not limited to, an epitope region comprising amino acids 16-23 of SEQ ID NO:97, and an epitope region comprising amino acids 10-17 of SEQ ID NO:97. In certain embodiments, the epitope region in the N-terminal region comprises amino acids 15-23 of SEQ ID NO:97. In certain embodiments, the epitope region in the N-terminal region comprises amino acids 16-25 of SEQ ID NO:97. In certain embodiments, the epitope region in the N-terminal region comprises amino acids 10-17 of SEQ ID NO:97. In certain embodiments, the epitope region in the N-terminal region comprises amino acids 5-17 of SEQ ID NO:97.

In certain embodiments, an antibody or an antigen-binding fragment thereof of the presently disclosed subject matter binds to an epitope region in the ECL1 region, including, but not limited to, an epitope region comprising amino acids 85-95 of SEQ ID NO:97.

In certain embodiments, an antibody or an antigen-binding fragment thereof of the presently disclosed subject matter binds to an epitope region in the ECL2 region, including, but not limited to, an epitope region comprising amino acids 157-164 of SEQ ID NO:97. In certain embodiments, the epitope region in the ECL2 region comprises amino acids 157-164 of SEQ ID NO:97. In certain embodiments, the epitope region in the ECL2 region comprises amino acids 157-167 of SEQ ID NO:97.

In certain embodiments, an antibody or an antigen-binding fragment thereof of the presently disclosed subject matter binds to an epitope region in the ECL3 region, including, but not limited to, an epitope region comprising amino acids 230-237 of SEQ ID NO:97. In certain embodiments, the epitope region in the ECL3 region comprises amino acids 229-237 of SEQ ID NO:97. In certain embodiments, the epitope region in the ECL3 region comprises amino acids 230-243 of SEQ ID NO:97. In certain embodiments, the epitope region in the ECL3 region comprises amino acids 227-237 of SEQ ID NO:97.

In certain embodiments, the antibody or an antigen-binding fragment thereof binds to an epitope region comprising amino acids 16-25 of SEQ ID NO:97, an epitope region comprising amino acids 157-164 of SEQ ID NO:97, and an epitope region comprising amino acids 229-237 of SEQ ID NO:97. For example, the antibody or an antigen-binding fragment thereof comprises a heavy chain variable region comprising amino acids having the sequence set forth in SEQ ID NO:57 and a light chain variable region comprising amino acids having the sequence set forth in SEQ ID NO:58, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the antibody or an antigen-binding fragment thereof is a scFv. In certain embodiments, the antibody or an antigen-binding fragment thereof is a scFv-Fc fusion protein or full length human IgG with V_{H} and V_{L} regions or CDRs selected from Table 15. In certain embodiments, the antibody or an antigen-binding fragment thereof comprises a V_{H} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO:57. In certain embodiments, the antibody or an antigen-binding fragment thereof comprises a Vu comprising amino acids having the sequence set forth in SEQ ID NO:57. In certain embodiments, the antibody or an antigen-binding fragment thereof comprises a V_{L} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO:58. In certain embodiments, the antibody or an antigen-binding fragment thereof comprises a V_{L} comprising amino acids having the sequence set forth in SEQ ID NO:58. In certain embodiments, the antibody or an antigen-binding fragment thereof comprises a V_{H} comprising amino acids having the sequence set forth in SEQ ID NO:57 and a V_{L} comprising amino acids having the sequence set forth in SEQ ID NO:58. In certain embodiments, the antibody or an antigen-binding fragment thereof comprises a V_{H} CDR1 comprising amino acids having the sequence set forth in SEQ ID NO:208 or conservative modifications thereof, a V_{H} CDR2 comprising amino acids having the sequence set forth in SEQ ID NO: 209 or conservative modifications thereof, and a V_{H} CDR3 comprising amino acids having the sequence set forth in SEQ ID NO: 210 or conservative modifications thereof. In certain embodiments, the antibody or an antigen-binding fragment thereof comprises a V_{L} CDR1 comprising amino acids having the sequence set forth in SEQ ID NO: 211 or conservative modifications thereof, a V_{L} CDR2 comprising amino acids having the sequence set forth in SEQ ID NO: 212 or conservative modifications thereof, and a V_{L} CDR3 comprising amino acids having the sequence set forth in SEQ ID NO: 213 or conservative modifications thereof. In certain embodiments, the antibody or an antigen-binding fragment thereof comprises a V_{H} CDR1 comprising amino acids having the sequence set forth in SEQ ID NO: 208 or conservative modifications thereof, a V_{H} CDR2 comprising amino acids having the sequence set forth in SEQ ID NO: 209 or conservative modifications thereof, a V_{H} CDR3 comprising amino acids having the sequence set forth in SEQ ID NO: 210 or conservative modifications thereof, a V_{L} CDR1 comprising amino acids having the sequence set forth in SEQ ID NO: 211 or conservative modifications thereof, a V_{L} CDR2 comprising amino acids having the sequence set forth in SEQ ID NO: 212 or conservative modifications thereof, and a V_{L} CDR3 comprising amino acids having the sequence set forth in SEQ ID NO: 213 or conservative modifications thereof. In certain embodiments, the antibody or an antigen-binding fragment thereof comprises a V_{H} CDR1 comprising amino acids having the sequence set forth in SEQ ID NO: 208, a V_{H} CDR2 comprising amino acids having the sequence set forth in SEQ ID NO: 209, a V_{H} CDR3 comprising amino acids having the sequence set forth in SEQ ID NO: 210, a V_{L} CDR1 comprising amino acids having the sequence set forth in SEQ ID NO: 211, a V_{L} CDR2 comprising amino acids having the sequence set forth in SEQ ID NO: 212, and a V_{L} CDR3 comprising amino acids having the sequence set forth in SEQ ID NO: 213. In certain embodiments, the antibody or an antigen-binding fragment thereof is ET150-2 scFv (or ET150-152 scFv).

In certain embodiments, the antibody or an antigen-binding fragment thereof binds to an epitope region comprising amino acids 5-17 of SEQ ID NO:97, an epitope region comprising amino acids 85-95 of SEQ ID NO:97, and an epitope region comprising amino acids 157-164 of SEQ ID NO:97. For example, the antibody or an antigen-binding fragment thereof comprises a heavy chain variable region comprising amino acids having the sequence set forth in SEQ ID NO:61 and a light chain variable region comprising amino acids having the sequence set forth in SEQ ID NO:62, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the antibody or an antigen-binding fragment thereof is a scFv. In certain embodiments, the antibody or an antigen-binding fragment thereof is a scFv-Fc fusion protein or full length human IgG with V_{H} and V_{L} regions or CDRs selected from Table 16. In certain embodiments, the antibody or an antigen-binding fragment thereof comprises a V_{H} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO:61. In certain embodiments, the antibody or an antigen-binding fragment thereof comprises a V_{H} comprising amino acids having the sequence set forth in SEQ ID NO:61. In certain embodiments, the antibody or an antigen-binding fragment thereof comprises a V_{L} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO:62. In certain embodiments, the antibody or an antigen-binding fragment thereof comprises a V_{L} comprising amino acids having the sequence set forth in SEQ ID NO:62. In certain embodiments, the antibody or an antigen-binding fragment thereof comprises a V_{H} comprising amino acids having the sequence set forth in SEQ ID NO:61 and a V_{L} comprising amino acids having the sequence set forth in SEQ ID NO:62. In certain embodiments certain embodiments, the antibody or an antigen-binding fragment thereof comprises a V_{H} CDR1 comprising amino acids having the sequence set forth in SEQ ID NO:214 or conservative modifications thereof, a V_{H} CDR2 comprising amino acids having the sequence set forth in SEQ ID NO: 215 or conservative modifications thereof, and a V_{H} CDR3 comprising amino acids having the sequence set forth in SEQ ID NO: 216 or conservative modifications thereof. In certain embodiments, the antibody or an antigen-binding fragment thereof comprises a V_{L} CDR1 comprising amino acids having the sequence set forth in SEQ ID NO: 217 or conservative modifications thereof, a V_{L} CDR2 comprising amino acids having the sequence set forth in SEQ ID NO: 218 or conservative modifications thereof, and a V_{L} CDR3 comprising amino acids having the sequence set forth in SEQ ID NO: 219 or conservative modifications thereof. In certain embodiments, the antibody or an antigen-binding fragment thereof comprises a V_{H} CDR1 comprising amino acids having the sequence set forth in SEQ ID NO: 214 or conservative modifications thereof, a V_{H} CDR2 comprising amino acids having the sequence set forth in SEQ ID NO: 215 or conservative modifications thereof, a V_{H} CDR3 comprising amino acids having the sequence set forth in SEQ ID NO: 216 or conservative modifications thereof, a V_{L} CDR1 comprising amino acids having the sequence set forth in SEQ ID NO: 217 or conservative modifications thereof, a V_{L} CDR2 comprising amino acids having the sequence set forth in SEQ ID NO: 218 or conservative modifications thereof, and a V_{L} CDR3 comprising amino acids having the sequence set forth in SEQ ID NO: 219 or conservative modifications thereof. In certain embodiments, the antibody or an antigen-binding fragment thereof comprises a V_{H} CDR1 comprising amino acids having the sequence set forth in SEQ ID NO: 214, a V H CDR2 comprising amino acids having the sequence set forth in SEQ ID NO: 215, a V_{H} CDR3 comprising amino acids having the sequence set forth in SEQ ID NO: 216, a V_{L} CDR1 comprising amino acids having the sequence set forth in SEQ ID NO: 217, a V_{L} CDR2 comprising amino acids having the sequence set forth in SEQ ID NO: 218 and a V_{L} CDR3 comprising amino acids having the sequence set forth in SEQ ID NO: 219. In certain embodiments, the antibody or an antigen-binding fragment thereof is ET150-155 scFv (or ET150-5 scFv).

In certain embodiments, the antibody or an antigen-binding fragment thereof binds to an epitope region comprising amino acids 15-23 of SEQ ID NO:97, and an epitope region comprising amino acids 230-243 of SEQ ID NO:97. For example, the antibody or an antigen-binding fragment thereof comprises a heavy chain variable region comprising amino acids having the sequence set forth in SEQ ID NO:65 and a light chain variable region comprising amino acids having the sequence set forth in SEQ ID NO:66, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the antibody or an antigen-binding fragment thereof is a scFv. In certain embodiments, the antibody or an antigen-binding fragment thereof is a scFv-Fc fusion protein or full length human IgG with V_{H} and V_{L} regions or CDRs selected from Table 17. In certain embodiments, the antibody or an antigen-binding fragment thereof comprises a V_{H} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO:65. In certain embodiments, the antibody or an antigen-binding fragment thereof comprises a V_{H} comprising amino acids having the sequence set forth in SEQ ID NO:65. In certain embodiments, the antibody or an antigen-binding fragment thereof comprises a V_{L} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO:66, as shown in Table 17. In certain embodiments, the antibody or an antigen-binding fragment thereof comprises a V_{L} comprising amino acids having the sequence set forth in SEQ ID NO:66. In certain embodiments, the antibody or an antigen-binding fragment thereof comprises a Vu comprising amino acids having the sequence set forth in SEQ ID NO:65 and a V_{L} comprising amino acids having the sequence set forth in SEQ ID NO:66. In certain embodiments, the antibody or an antigen-binding fragment thereof comprises a V_{H} CDR1 comprising amino acids having the sequence set forth in SEQ ID NO:220 or conservative modifications thereof, a V_{H} CDR2 comprising amino acids having the sequence set forth in SEQ ID NO: 221 or conservative modifications thereof, and a V_{H} CDR3 comprising amino acids having the sequence set forth in SEQ ID NO: 222 or conservative modifications thereof. In certain embodiments, the antibody or an antigen-binding fragment thereof comprises a V_{L} CDR1 comprising amino acids having the sequence set forth in SEQ ID NO: 223 or conservative modifications thereof, a V_{L} CDR2 comprising amino acids having the sequence set forth in SEQ ID NO: 224 or conservative modifications thereof, and a V_{L} CDR3 comprising amino acids having the sequence set forth in SEQ ID NO: 225 or conservative modifications thereof. In certain embodiments, the antibody or an antigen-binding fragment thereof comprises a V_{H} CDR1 comprising amino acids having the sequence set forth in SEQ ID NO: 220 or conservative modifications thereof, a V_{H} CDR2 comprising amino acids having the sequence set forth in SEQ ID NO: 221 or conservative modifications thereof, a V_{H} CDR3 comprising amino acids having the sequence set forth in SEQ ID NO: 222 or conservative modifications thereof, a V_{L} CDR1 comprising amino acids having the sequence set forth in SEQ ID NO: 223 or conservative modifications thereof, a V_{L} CDR2 comprising amino acids having the sequence set forth in SEQ ID NO: 224 or conservative modifications thereof, and a V_{L} CDR3 comprising amino acids having the sequence set forth in SEQ ID NO: 225 or conservative modifications thereof. In certain embodiments, the antibody or an antigen-binding fragment thereof comprises a V_{H} CDR1 comprising amino acids having the sequence set forth in SEQ ID NO: 220, a V H CDR2 comprising amino acids having the sequence set forth in SEQ ID NO: 221, a V_{H} CDR3 comprising amino acids having the sequence set forth in SEQ ID NO: 222, a V_{L} CDR1 comprising amino acids having the sequence set forth in SEQ ID NO: 223, a V_{L} CDR2 comprising amino acids having the sequence set forth in SEQ ID NO: 224, and a V_{L} CDR3 comprising amino acids having the sequence set forth in SEQ ID NO: 225. In certain embodiments, the antibody or an antigen-binding fragment thereof is ET150-8 scFv (or ET150-158 scFv).

In certain embodiments, the antibody or an antigen-binding fragment thereof binds to an epitope region comprising amino acids 10-17 of SEQ ID NO:97, an epitope region comprising amino acids 157-167 of SEQ ID NO:97, and an epitope region comprising amino acids 227-237 of SEQ ID NO:97. For example, the antibody or an antigen-binding fragment thereof comprises a heavy chain variable region comprising amino acids having the sequence set forth in SEQ ID NO:69 and a light chain variable region comprising amino acids having the sequence set forth in SEQ ID NO:70, optionally with (iii) a linker sequence, for example a linker peptide, between the heavy chain variable region and the light chain variable region. In certain embodiments, the linker comprises amino acids having the sequence set forth in SEQ ID NO:98. In certain embodiments, the antibody or an antigen-binding fragment thereof is a scFv. In certain embodiments, the antibody or an antigen-binding fragment thereof is a scFv-Fc fusion protein or full length human IgG with V_{H} and V_{L} regions or CDRs selected from Table 18. In certain embodiments, the antibody or an antigen-binding fragment thereof comprises a V_{H} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO:69. In certain embodiments, the antibody or an antigen-binding fragment thereof comprises a V_{H} comprising amino acids having the sequence set forth in SEQ ID NO:69. In certain embodiments, the antibody or an antigen-binding fragment thereof comprises a V_{L} comprising an amino acid sequence that is at least 80%, 81%, 82%, 83%, 84%, 85%, 86%, 87%, 88%, 89%, 90%, 91%, 92%, 93%, 94%, 95%, 96%, 97%, 98% or 99% homologous to the amino acid sequence set forth in SEQ ID NO:70. In certain embodiments, the antibody or an antigen-binding fragment thereof comprises a V_{L} comprising amino acids having the sequence set forth in SEQ ID NO:70. In certain embodiments, the antibody or an antigen-binding fragment thereof comprises a V_{H} comprising amino acids having the sequence set forth in SEQ ID NO:69 and a V_{L} comprising amino acids having the sequence set forth in SEQ ID NO:70. In certain embodiments, the antibody or an antigen-binding fragment thereof comprises a V_{H} CDR1 comprising amino acids having the sequence set forth in SEQ ID NO:226 or conservative modifications thereof, a V_{H} CDR2 comprising amino acids having the sequence set forth in SEQ ID NO: 227 or conservative modifications thereof, and a V_{H} CDR3 comprising amino acids having the sequence set forth in SEQ ID NO: 228 or conservative modifications thereof. In certain embodiments, the antibody or an antigen-binding fragment thereof comprises a V_{L} CDR1 comprising amino acids having the sequence set forth in SEQ ID NO: 229 or conservative modifications thereof, a V_{L} CDR2 comprising amino acids having the sequence set forth in SEQ ID NO: 230 or conservative modifications thereof, and a V_{L} CDR3 comprising amino acids having the sequence set forth in SEQ ID NO: 231 or conservative modifications thereof. In certain embodiments, the antibody or an antigen-binding fragment thereof comprises a V_{H} CDR1 comprising amino acids having the sequence set forth in SEQ ID NO: 226 or conservative modifications thereof, a V_{H} CDR2 comprising amino acids having the sequence set forth in SEQ ID NO: 227 or conservative modifications thereof, a V_{H} CDR3 comprising amino acids having the sequence set forth in SEQ ID NO: 228 or conservative modifications thereof, a V_{L} CDR1 comprising amino acids having the sequence set forth in SEQ ID NO: 229 or conservative modifications thereof, a V_{L} CDR2 comprising amino acids having the sequence set forth in SEQ ID NO: 230 or conservative modifications thereof, and a V_{L} CDR3 comprising amino acids having the sequence set forth in SEQ ID NO: 231 or conservative modifications thereof. In certain embodiments, the antibody or an antigen-binding fragment thereof comprises a V_{H} CDR1 comprising amino acids having the sequence set forth in SEQ ID NO: 226, a V_{H} CDR2 comprising amino acids having the sequence set forth in SEQ ID NO: 227, a V_{H} CDR3 comprising amino acids having the sequence set forth in SEQ ID NO: 228, a V_{L} CDR1 comprising amino acids having the sequence set forth in SEQ ID NO: 229, a V_{L} CDR2 comprising amino acids having the sequence set forth in SEQ ID NO: 230, and a V_{L} CDR3 comprising amino acids having the sequence set forth in SEQ ID NO: 231. In certain embodiments, the antibody or an antigen-binding fragment thereof is ET150-18 scFv (or ET150-168 scFv).

7. Immunoconjugates

The presently disclosed subject provides an anti-GPRC5D antibody, or a fragment thereof, conjugated to a therapeutic moiety, such as a cytotoxin, a drug (e.g., an immunosuppressant) or a radiotoxin. Such conjugates are referred to herein as “immunoconjugates”. Immunoconjugates that include one or more cytotoxins are referred to as “immunotoxins.” A cytotoxin or cytotoxic agent includes any agent that is detrimental to (e.g., kills) cells. Examples include taxol (such as ricin, diphtheria, gelonin), cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, calecheamicin, aureastatin, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and ci s-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). Other examples of therapeutic cytotoxins that can be conjugated to an anti-GPRC5D antibody disclosed herein include duocarmycins, calicheamicins, maytansines and auristatins, and derivatives thereof. An example of a calicheamicin antibody conjugate is commercially available (MYLOTARG™ (Gemtuzumab ozogamicin); Wyeth-Ayerst).

Cytoxins can be conjugated to anti-GPRC5D antibody disclosed herein using linker technology available in the art. Examples of linker types that have been used to conjugate a cytotoxin to an antibody include, but are not limited to, hydrazones, thioethers, esters, disulfides and peptide-containing linkers. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D). For further discussion of types of cytotoxins, linkers and methods for conjugating therapeutic agents to antibodies, see also Saito, G. et al. (2003) Adv. Drug Deliv. Rev. 55:199-215; Trail, P. A. et al. (2003) Cancer Immunol. Immunother. 52:328-337; Payne, G. (2003) Cancer Cell 3:207-212; Allen, T. M. (2002) Nat. Rev. Cancer 2:750-763; Pastan, I. and Kreitman, R. J. (2002) Curr. Opin. Investig. Drugs 3:1089-1091; Senter, P. D. and Springer, C. J. (2001) Adv. Drug Deliv. Rev. 53:247-264.

Anti-GPRC5D antibodies of the presently disclosed subject matter also can be conjugated to a radioactive isotope to generate cytotoxic radiopharmaceuticals, also referred to as radioimmunoconjugates. Examples of radioactive isotopes that can be conjugated to antibodies for use diagnostically or therapeutically include, but are not limited to, ⁹⁰Y, ¹³¹I, ²²⁵Ac, ²¹³Bi, ²²³Ra and ²²⁷Th. Methods for preparing radio immunoconjugates are established in the art. Examples of radioimmunoconjugates are commercially available, including ZEVALIN™ (Ibritumomab tiuxetan) (DEC Pharmaceuticals) and BEXXAR™ (Tositumomab) (Corixa Pharmaceuticals), and similar methods can be used to prepare radioimmunoconjugates using the antibodies of the invention.

The antibody conjugates of the presently disclosed subject matter can be used to modify a given biological response, and the drug moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, Pseudomonas exotoxin, or diphtheria toxin; a protein such as tumor necrosis factor (TNF) or interferon-γ; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

8. Bispecific Molecules

The presently disclosed subject matter provides bispecific molecules comprising an anti-GPRC5D antibody, or a fragment thereof, disclosed herein. An antibody of the presently disclosed subject matter, or antigen-binding portions thereof, can be derivatized or linked to another functional molecule, e.g., another peptide or protein (e.g., another antibody or ligand for a receptor) to generate a bispecific molecule that binds to at least two different binding sites or target molecules. The antibody of the presently disclosed subject matter can in fact be derivatized or linked to more than one other functional molecule to generate multispecific molecules that bind to more than two different binding sites and/or target molecules; such multispecific molecules are also intended to be encompassed by the term “bispecific molecule” as used herein. To create a bispecific molecule, a presently disclosed anti-GPRC5D antibody can be functionally linked (e.g., by chemical coupling, genetic fusion, noncovalent association or otherwise) to one or more other binding molecules, such as another antibody, antibody fragment, peptide or binding mimetic, such that a bispecific molecule results.

The presently disclosed subject matter provides bispecific molecules comprising at least a first binding specificity for GPRC5D and a second binding specificity for a second target epitope. The second target epitope can be a GPRC5D epitope, or a non-GPRC5D epitope, e.g., a different antigen. In certain embodiments, the bispecific molecule is multispecific, the molecule can further include a third binding specificity. Where a first portion of a bispecific antibody binds to an antigen on a tumor cell for example and a second portion of a bispecific antibody recognizes an antigen on the surface of a human immune effector cell, the antibody is capable of recruiting the activity of that effector cell by specifically binding to the effector antigen on the human immune effector cell. In certain embodiments, bispecific antibodies, therefore, are able to form a link between effector cells, for example, T cells and tumor cells, thereby enhancing effector function. In certain embodiments, a bispecific antibody of the present disclosure comprises at least a first binding to GPRC5D and at least a second binding to an immune cell. For example, and not by way of limitation, a bispecific antibody of the present disclosure comprises at least a first binding to GPRC5D and at least a second binding to a receptor present on the surface of an immune cell, e.g., CD3.

The bispecific molecules of the presently disclosed subject matter can be prepared by conjugating the constituent binding specificities using methods known in the art. For example, each binding specificity of the bispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-S-acetyl-thioacetate (SATA), 5, 5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohaxane-1-carboxylate (sulfo-SMCC) (see e.g., Karpovsky et al. (1984) J. Exp. Med. 160:1686; Liu, M A et al. (1985) Proc. Natl. Acad. Sci. USA 82:8648). Other methods include those described in Paulus (1985) Behring Ins. Mitt. No. 78, 118-132; Brennan et al. (1985) Science 229:81-83), and Glennie et al. (1987) J. Immunol. 139: 2367-2375). Preferred conjugating agents are SATA and sulfo-SMCC, both available from Pierce Chemical Co. (Rockford, IL).

When the binding specificities are antibodies, they can be conjugated via sulfhydryl bonding of the C-terminus hinge regions of the two heavy chains. In certain embodiments, the hinge region is modified to contain an odd number of sulfhydryl residues, preferably one, prior to conjugation.

Alternatively, both binding specificities can be encoded in the same vector and expressed and assembled in the same host cell. This method is particularly useful where the bispecific molecule is a mAb×mAb, mAb×Fab, Fab×F(ab′)₂ or ligand×Fab fusion protein.

Binding of the bispecific molecules to their specific targets can be confirmed by, for example, enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest. Alternatively, the complexes can be detected using any of a variety of other immunoassays. For example, the antibody can be radioactively labeled and used in a radioimmunoassay (MA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography.

9. Selecting a High Affinity ScFv Against a GPRC5D Polypeptide

The next step is to the selection of phage that bind to the target antigen of interest with high affinity, from phage in a human phage display library that either does not bind or that binds with lower affinity. This is accomplished by iterative binding of phage to the antigen, which is bound to a solid support, for example, beads or mammalian cells followed by removal of non-bound phage and by elution of specifically bound phage. In certain embodiments, antigens are first biotinylated for immobilization to, for example, streptavidin-conjugated Dynabeads M-280. The phage library is incubated with the cells, beads or other solid support and non binding phage is removed by washing. Clones that bind are selected and tested.

Once selected, positive scFv clones are tested for their binding to GPRC5D (human GPRC5D) on live 3T3 cell surfaces by flow cytometry. Briefly, phage clones are incubated with 3T3 cells over-expressing GPRC5D. The cells are washed and then with a mouse anti-M13 coat protein mAb. Cells are washed again and labeled with a FITC-goat anti-mouse Ig prior to flow cytometry.

In other embodiments, the anti-GPRC5D antibodies can comprise one or more framework region amino acid substitutions designed to improve protein stability, antibody binding, expression levels or to introduce a site for conjugation of therapeutic agents. These scFv are then used to produce recombinant human monoclonal Igs in accordance with methods known to those of skill in the art.

10. Engineering Full Length mAb Using the Selected ScFv Fragments

Phage display technology allows for the rapid selection and production of antigen-specific scFv and Fab fragments, which are useful in and of themselves, or which can be further developed to provide complete antibodies, antigen binding proteins or antigen binding fragments thereof. Complete mAbs with Fc domains have a number of advantages over the scFv and Fab antibodies. First, only full length Abs exert immunological function such as CDC and ADCC mediated via Fc domain. Second, bivalent mAbs offer stronger antigen-binding affinity than monomeric Fab Abs. Third, plasma half-life and renal clearance will be different with the Fab and bivalent mAb. The particular features and advantages of each can be matched to the planned effector strategy. Fourth, bivalent mAb may be internalized at different rates than scFv and Fab, altering immune function or carrier function. Alpha emitters, for example, do not need to be internalized to kill the targets, but many drugs and toxins will benefit from internalization of the immune complex. In certain embodiments, therefore, once scFv clones specific for GPRC5D were obtained from phage display libraries, a full length IgG mAb using the scFv fragments was produced.

To produce recombinant human monoclonal IgG in Chinese hamster ovary (CHO) cells, a full length IgG mAb can be engineered based on a method known to those of skill in the art (Tomomatsu et al., Production of human monoclonal antibodies against FceRIa by a method combining in vitro immunization with phage display. Biosci Biotechnol Biochem 73(7): 1465-1469 2009). Briefly, antibody variable regions can be subcloned into mammalian expression vectors, with matching Lambda or Kappa light chain constant sequences and IgG1 subclass Fc (for example) (Lidija P, et al. An integrated vector system for the eukaryotic expression of antibodies or their fragments after selection from phage display libraries. Gene 1997; 187(1): 9-18; Lisa J H, et al. Crystallographic structure of an intact IgG1 monoclonal antibody. Journal of Molecular Biology 1998; 275 (5): 861-872). Kinetic binding analysis (Yasmina N A, et al. Probing the binding mechanism and affinity of tanezumab, a recombinant humanized anti-NGF monoclonal antibody, using a repertoire of biosensors. Protein Science 2008; 17(8): 1326-1335) can be used to confirm specific binding of full length IgG to GPRC5D, with a K D in nanomolar range.

Pharmaceutical Compositions and Methods of Treatment

Anti-GPRC5D antibodies of the presently disclosed subject matter can be administered for therapeutic treatments to a patient suffering from a tumor (e.g., multiple myeloma) in an amount sufficient to prevent, inhibit, or reduce the progression of the tumor. Progression includes, e.g, the growth, invasiveness, metastases and/or recurrence of the tumor. Amounts effective for this use will depend upon the severity of the disease and the general state of the patient's own immune system. Dosing schedules will also vary with the disease state and status of the patient, and will typically range from a single bolus dosage or continuous infusion to multiple administrations per day (e.g., every 4-6 hours), or as indicated by the treating physician and the patient's condition.

The identification of medical conditions treatable by anti-GPRC5D antibodies of the presently disclosed subject matter is well within the ability and knowledge of one skilled in the art. For example, human individuals who are either suffering from multiple myeloma or who are at risk of developing multiple myeloma are suitable for administration of the presently disclosed anti-GPRC5D antibodies. A clinician skilled in the art can readily determine, for example, by the use of clinical tests, physical examination and medical/family history, if an individual is a candidate for such treatment.

In certain embodiments, the presently disclosed subject matter provides a method of treating a tumor by administering a presently disclosed anti-GPRC5D antibody in combination with one or more other agents. For example, the presently disclosed subject matter provides a method of treating a tumor by administering a presently disclosed anti-GPRC5D antibody with an antineoplastic agent. The anti-GPRC5D antibody can be chemically or biosynthetically linked to one or more of the antineoplastic agents.

Non-limiting examples of suitable tumors include multiple myeloma and Waldenstrom's Macroglobulinemia. In certain embodiments, the tumor is multiple myeloma.

Any suitable method or route can be used to administer a presently disclosed anti-GPRC5D antibody, and optionally, to coadminister antineoplastic agents. Routes of administration include, for example, oral, intravenous, intraperitoneal, subcutaneous, or intramuscular administration. It should be emphasized, however, that the presently disclosed subject matter is not limited to any particular method or route of administration.

It is noted that presently disclosed anti-GPRC5D antibody can be administered as a conjugate, which binds specifically to the receptor and delivers a toxic, lethal payload following ligand-toxin internalization.

It is understood that anti-GPRC5D antibodies of the presently disclosed subject matter can be administered in the form of a composition additionally comprising a pharmaceutically acceptable carrier. Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the binding proteins. The compositions of the injection can, as is well known in the art, be formulated so as to provide quick, sustained or delayed release of the active ingredient after administration to the mammal.

The presently disclosed subject matter also provides use of antibodies and nucleic acids that encode them for treatment of a tumor (e.g., multiple myeloma), for diagnostic and prognostic applications as well as use as research tools for the detection of GPRC5D in cells and tissues. Pharmaceutical compositions comprising the disclosed antibodies and nucleic acids are encompassed by the presently disclosed subject matter. Vectors comprising the nucleic acids of the presently disclosed subject matter for antibody-based treatment by vectored immunotherapy are also contemplated by the presently disclosed subject matter. Vectors include expression vectors which enable the expression and secretion of antibodies, as well as vectors which are directed to cell surface expression of the antigen binding proteins, such as chimeric antigen receptors.

Cells comprising the nucleic acids, for example cells that have been transfected with the vectors of the invention are also encompassed by the presently disclosed subject matter.

Kits

The presently disclosed subject matter provides kits for the treatment or prevention of a tumor (e.g., multiple myeloma). In certain embodiments, the kit comprises a therapeutic composition containing an effective amount of an anti-GPRC5D antibody in unit dosage form. In certain embodiments, the kit comprises a sterile container which contains a therapeutic or prophylactic vaccine; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired, the anti-GPRC5D antibody is provided together with instructions for administering the cell to a subject having or at risk of developing a tumor (e.g., multiple myeloma). The instructions will generally include information about the use of the composition for the treatment or prevention of a tumor (e.g., multiple myeloma). In other embodiments, the instructions include at least one of the following: description of the therapeutic agent; dosage schedule and administration for treatment or prevention of a neoplasia (e.g., multiple myeloma) or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

Methods

Flow cytometry analysis. For cell surface staining, cells can be incubated with appropriate mAbs for 30 minutes on ice, washed, and incubated with secondary antibody reagents when necessary. Flow cytometry data can be collected on a FACS Calibur (Becton Dickinson) and analyzed with FlowJo V8.7.1 and 9.4.8 software.

Selection and characterization of scFv specific for GPRC5D. A human scFv antibody phage display library is used for the selection of mAb clones. In brief, biotinylated antigens can be first mixed with the human scFv phage library, then the antigen-scFv antibody complexes can be pulled down by streptavidin-conjugated Dynabeads M-280 through a magnetic rack. Bound clones can be then eluted and used to infect E. coli XL1-Blue. The scFv phage clones expressed in the bacteria can be purified (Yasmina N A, et al. Probing the binding mechanism and affinity of tanezumab, a recombinant humanized anti-NGF monoclonal antibody, using a repertoire of biosensors. Protein Science 2008; 17(8): 1326-1335; Roberts W K, et al. Vaccination with CD20 peptides induces a biologically active, specific immune response in mice. Blood 2002: 99 (10): 3748-3755). Panning can be performed for 3-4 cycles to enrich scFv phage clones binding to GPRC5D specifically. Positive clones can be determined by flow cytometry method against biotinylated single chain GPRC5D. Positive clones can be further tested for their binding to GPRC5D on live cell surfaces by flow cytometry, using a GPRC5D⁺ cell line, 3T3. The cells can be washed, and the staining can be performed in following steps.

The cells can be first stained with purified scFv phage clones, and followed by staining with a mouse anti-M13 mAb, and finally the goat anti-mouse Ig's conjugate to FITC. Each step of the staining can be done between 30-60 minutes on ice and the cells were washed twice between each step of the staining.

Engineering full length mAb using the selected ScFv fragments. Full-length human IgG of the selected phage clones can be produced in HEK293 and Chinese hamster ovary (CHO) cell lines, as described (Caron P C, Class K, Laird W, Co M S, Queen C, Scheinberg D A. Engineered humanized dimeric forms of IgG are more effective antibodies. J Exp Med 176:1 191-1 195 (1992). In brief, antibody variable regions can be subcloned into mammalian expression vectors, with matching human lambda or kappa light chain constant region and human IgG constant region sequences. Molecular weight of the purified full length IgG antibodies can be measured under both reducing and non-reducing conditions by electrophoresis.

Characterization of the full-length human IgG for GPRC5D. Initially, specificities of the fully human IgG mAbs for the GPRC5D can be determined by staining 3T3 cells transduced to overexpress GPRC5D, followed by secondary goat anti-human IgG mAb conjugate to PE or FITC. The fluorescence intensity can be measured by flow cytometry. The same method can be used to determine the binding of the mAbs to fresh tumor cells and cell lines.

Antibody-dependent cellular cytotoxicity (ADCC). Target cells used for ADCC can be 3T3 cells over-expressing GPRC5D. Anti-GPRC5D antibody or its control human IgG at various concentrations can be incubated with target cells and fresh PBMCs at different effector:target (E:T) ratio for 16 hrs. The supernatant can be harvested and the cytotoxicity can be measured by LDH release assay using Cytotox 96 nonradioactive kit from Promega following their instruction. Cytotoxicity can also be measured by standard 4 hours 51 Cr-release assay.

### EXAMPLES

The following examples are put forth so as to provide those of ordinary skill in the art with a complete disclosure and description of how to make and use the antibodies, bispecific antibodies, compositions comprising thereof, screening, and therapeutic methods of the presently disclosed subject matter, and are not intended to limit the scope of what the inventors regard as their presently disclosed subject matter. It is understood that various other embodiments may be practiced, given the general description provided above.

### Example 1—GPRC5D Expression in Various Tissues

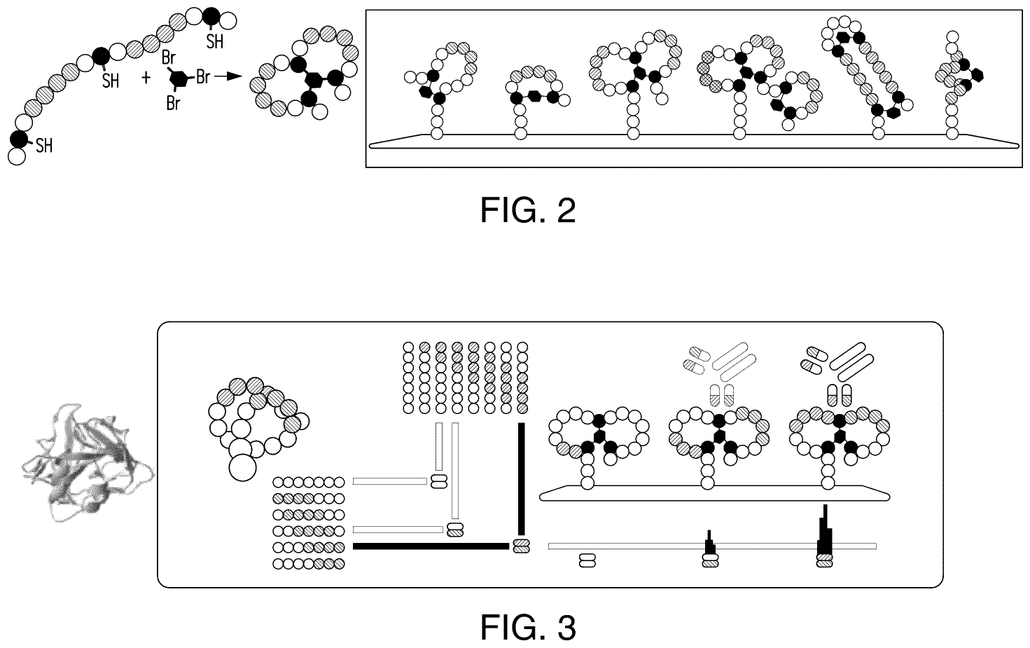

The expression of human GPRC5D was evaluated in various malignant and normal tissues by investigating gene expression profiles in databases such as the cancer cell line encyclopedia and BioGPS. As shown in FIG. 2, human GPRC5D was highly expressed in multiple myeloma, but not in other malignant tissues. Normal expression appeared limited to plasma cells. Potential GPRC5D targeted CAR T cell eradication of this normal cell type may not have significant adverse effects based on inventors' patient experience with CD19 targeted CAR T cells. Any lack of physiologic antibody production can be addressed with intravenous immunoglobulin treatment.

### Example 2—Selection of ScFv Specific for GPRC5D Using a Fully Human Phage Display Library

Phage display against GPRC5D was performed for 4 panning rounds to enrich the scFv phage clones binding to GPRC5D specifically. Four independent pannings with 12 different phage libraries were carried out against GPRC5D overexpressing 3T3 cells identifying 80 positive clones. Individual scFv phage clones positive for the GPRC5D were determined by ELISA and the clones that possessed unique DNA coding sequences were subjected to further characterization. To test if the ScFv bound to GPRC5D on live cells, the positive phage clones were tested for binding to a GPRC5D-positive cell line, 3T3. 72 positive clones were identified out of 80 clones screened FACS; the positive clone rate was 90%. After sequencing, 32 unique and GPRC5D-3T3 positive binding clones were found out of 72 sequenced positive clones; the unique clone rate was 45%.

### Example 3—Epitope Mapping of Anti-GPRC5D Antibodies

Four anti-GPRC5D antibodies: ET150-2, ET150-5, ET150-8, and ET150-18 mIgG1. “mIgG1” used in all Examples represents that the variable region is fully human and the Fc part is mouse IgG1. See Table 34.

TABLE 34

| Name | Origin | Concentration | Location | Status |
|---|---|---|---|---|
| ET150-18 mIgG1 | mouse Fc | 1.1 mg/ml | +4° C./22 | ok |
| ET150-2 mIgG1 | mouse Fc | 0.66 mg/ml | +4° C./22 | ok |
| ET150-5 mIgG1 | mouse Fc | 1.9 mg/ml | +4° C./22 | ok |
| ET150-8 mIgG1 | mouse Fc | 2.9 mg/ml | +4° C./22 | ok |

The target protein is human GPRC5D having the amino acid sequence set forth in SEQ ID NO: 97. The N-terminal region of human GPRC5D has amino acids 1-27 of SEQ ID NO:97. The extracellular loop 1 (ECL1) region of human GPRC5D has amino acids 85-93 of SEQ ID NO:97. The extracellular loop 2 (ECL2) region of human GPRC5D has amino acids 145-167 of SEQ ID NO:97. The extracellular loop 3 (ECL3) region of human GPRC5D has amino acids 226-239 of SEQ ID NO:97.

Methods

The principles of clips technology. CLIPS technology structurally fixes peptides into defined three-dimensional structures. This results in functional mimics of even the most complex binding sites. CLIPS technology is now routinely used to shape peptide libraries into single, double or triple looped structures as well as sheet- and helix-like folds (FIG. 2).

Combinatorial clips library screening in detail. CLIPS library screening starts with the conversion of the target protein into a library of up to 10,000 overlapping peptide constructs, using a combinatorial matrix design. On a solid carrier, a matrix of linear peptides is synthesized, which are subsequently shaped into spatially defined CLIPS constructs (FIG. 3). Constructs representing both parts of the discontinuous epitope in the correct conformation bind the antibody with high affinity, which is detected and quantified. Constructs presenting the incomplete epitope bind the antibody with lower affinity, whereas constructs not containing the epitope do not bind at all. Affinity information is used in iterative screens to define the sequence and conformation of epitopes in detail.

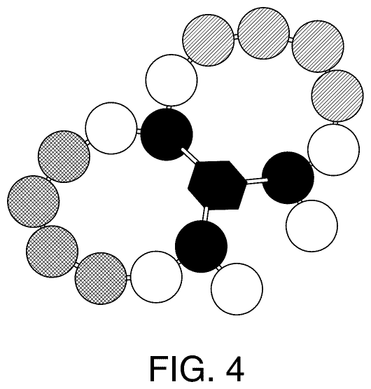

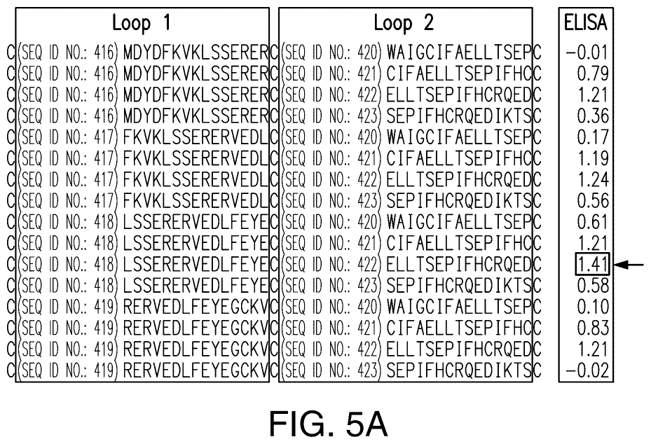

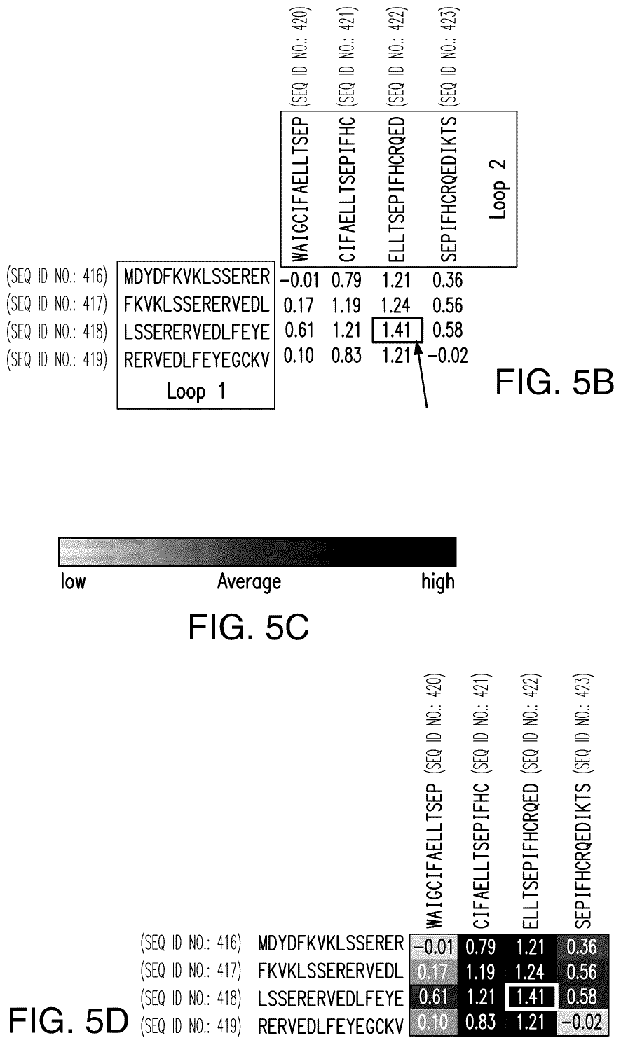

Heat map analysis. A heat map is a graphical representation of data where the values taken by a variable in a two-dimensional map are represented as colors. For double-looped CLIPS peptides, such a two-dimensional map can be derived from the independent sequences of the first and second loops. For example, the sequences of the 16 CLIPS peptides depicted in FIG. 5 are effectively permutations of 4 unique sub-sequences in loop 1 (colored in blue in FIG. 4) and 4 unique sub-sequences in loop 2 (colored in green in FIG. 4). Thus, the observed ELISA data (colored in red in FIG. 5A) can be plotted in a 4×4 matrix, where each X coordinate corresponds to the sequence of the first loop, and each Y coordinate corresponds to the sequence of the second loop. For instance, the ELISA value observed for CLIPS peptide CLSSERERVEDLFEYECELLTSEPIFHCRQEDC (SEQ ID NO: 382) indicated with an arrow in FIG. 4A) can be found at the third row, third column of FIG. 5B (indicated with an arrow and a red square). To further facilitate the visualization, ELISA values can be replaced with colors from a continuous gradient. In this case, extremely low values are colored in green, extremely high values are colored in red, and average values are colored in black (see FIG. 5C). For the aforementioned example, the average value is 0.71. When this color map is applied to the data matrix depicted in FIG. 5B, a color heat map is obtained (see FIG. 5D, the original data is still indicated for extra clarity).

Synthesis of peptides. To reconstruct epitopes of the target molecule a library of peptides was synthesized. An amino functionalized polypropylene support was obtained by grafting with a proprietary hydrophilic polymer formulation, followed by reaction with t-butyloxycarbonyl-hexamethylenediamine (BocHMDA) using dicyclohexylcarbodiimide (DCC) with Nhydroxybenzotriazole (HOBt) and subsequent cleavage of the Boc-groups using trifluoroacetic acid (TFA). Standard Fmoc-peptide synthesis was used to synthesize peptides on the amino-functionalized solid support by custom modified JANUS liquid handling stations (Perkin Elmer). Synthesis of structural mimics was done using Pepscan's proprietary Chemically Linked Peptides on Scaffolds (CLIPS) technology. CLIPS technology allows to structure peptides into single loops, doubleloops, triple loops, sheet-like folds, helix-like folds and combinations thereof. CLIPS templates are coupled to cysteine residues. The side-chains of multiple cysteines in the peptides were coupled to one or two CLIPS templates. For example, a 0.5 mM solution of the P2 CLIPS (2,6-bis(bromomethyl)pyridine) was dissolved in ammonium bicarbonate (20 mM, pH 7.8)/acetonitrile (1:3(v/v)). This solution was added onto the peptide arrays. The CLIPS template bound to side-chains of two cysteines as present in the solid-phase bound peptides of the peptide-arrays (455 wells plate with 3 μl wells). The peptide arrays were gently shaken in the solution for 30 to 60 minutes while completely covered in solution. Finally, the peptide arrays were washed extensively with excess of H2O and sonicated in disrupt-buffer containing 1% SDS/0.1% beta-mercaptoethanol in PBS (pH 7.2) at 70° C. for 30 minutes, followed by sonication in H2O for another 45 minutes. The T3 CLIPS carrying peptides were made in a similar way but now with three cysteines.

ELISA Screening. The binding of antibody to each of the synthesized peptides was tested in a PEPSCAN-based ELISA. The peptide arrays were incubated with primary antibody solution (overnight at 4° C.). After washing, the peptide arrays were incubated with a 1/1000 dilution of an appropriate antibody peroxidase conjugate (SBA) for one hour at 25° C. After washing, the peroxidase substrate 2,2′-azino-di-3-ethylbenzthiazoline sulfonate (ABTS) and 2 μl/ml of 3 percent H2O2 were added. After one hour, the color development was measured. The color development was quantified with a charge coupled device (CCD)-camera and an image processing system.

Data processing. The values obtained from the CCD camera ranged from 0 to 3000 mAU, similar to a standard 96-well plate ELISA-reader. The results were quantified and stored into the Peplab database. Occasionally a well contained an air-bubble resulting in a false-positive value, the cards were manually inspected and any values caused by an air-bubble were scored as 0.

Synthesis quality control—To verify the quality of the synthesized peptides, a separate set of positive and negative control peptides was synthesized in parallel. These were screened with antibody 57.9 (ref. Posthumus et al., J. Virology, 1990, 64:3304-3309).

Results

Screening. Antibody binding depends on a combination of factors, including concentration of the antibody and the amounts and nature of competing proteins in the ELISA buffer. Also, the pre-coat conditions (the specific treatment of the peptide arrays prior to incubation with the experimental sample) affected binding. These details are summed up in Table 35. For the Pepscan Buffer and Preconditioning (SQ), the numbers indicate the relative amount of competing protein (a combination of horse serum and ovalbumin).

TABLE 35
Screening conditions

| Label | Dilution | Sample Buffer | Pre-conditioning |
|---|---|---|---|
| ET150-18 mIgG1 | 1 μg/ml | 1% SQ | 1% SQ |
| ET150-2 mIgG1 | 1 μg/ml | 10% SQ | 10% SQ |
| ET150-5 mIgG1 | 1 μg/ml | 10% SQ | 10% SQ |
| ET150-8 mIgG1 | 3 μg/ml | 10% SQ | 10% SQ |

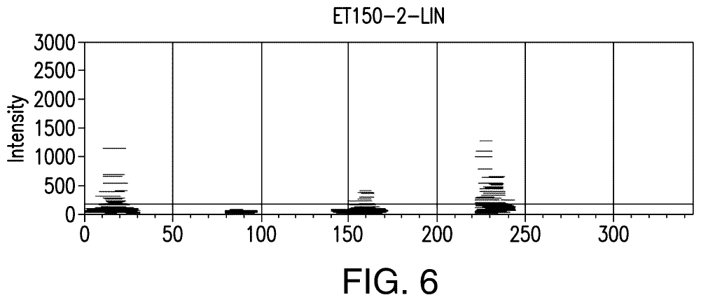

Antibody ET150-2. When tested under moderate stringency conditions antibody ET150-2 avidly bound peptides from all sets (FIG. 6). Cumulative data analysis shows that the antibody recognize a discontinuous epitope composed of peptides stretches ₁₆CDAEGPWGII₂₅ (N-term) (SEQ ID NO: 383), ₁₅₇MFVNMTPC₁₆₄ (ECL2) (SEQ ID NO: 384) and ₂₂₉PQFQRQPQW₂₃₇ (ECL3) (SEQ ID NO: 385), where peptide stretches ₁₆CDAEGPWGII₂₅ (SEQ ID NO: 383) and ₂₂₉PQFQRQPQW₂₃₇ (SEQ ID NO: 385) alone suffice for binding.

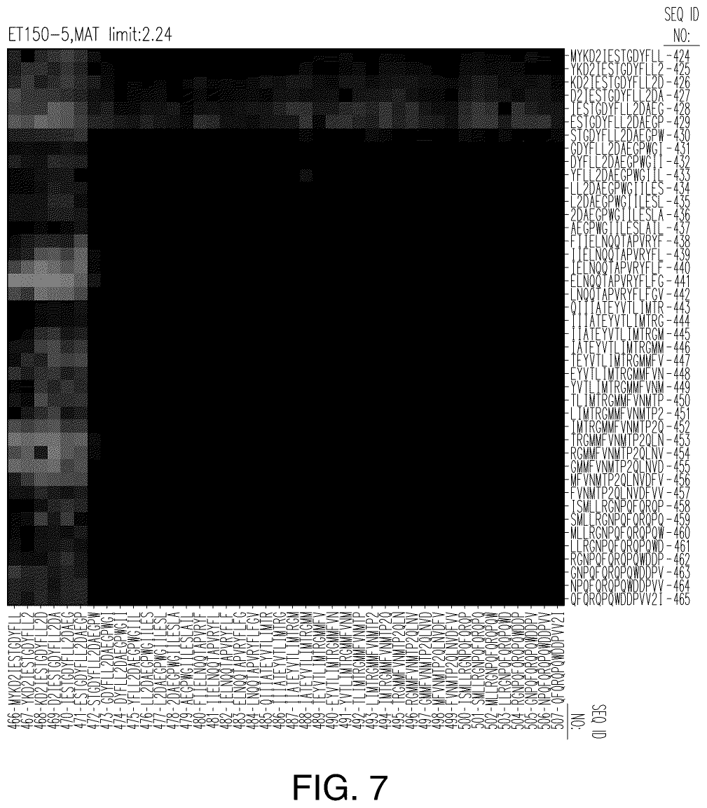

Antibody ET150-5. When tested under high stringency conditions antibody ET150-5 avidly bound peptides from all sets (FIG. 7). Cumulative data analysis shows that the antibody recognizes a discontinuous epitope composed of peptide stretches ₅CIESTGDYFLLCD₁₇ (N-term) (SEQ ID NO: 386), ₈₅NQQTAPVRYFL₉₅ (ECL1) (SEQ ID NO: 387) and ₁₅₇MFVNMTPC₁₆₄ (ECL2) (SEQ ID NO: 384), where peptide stretch ₅CIESTGDYFLLCD₁₇ (SEQ ID NO: 386) alone suffices for binding.

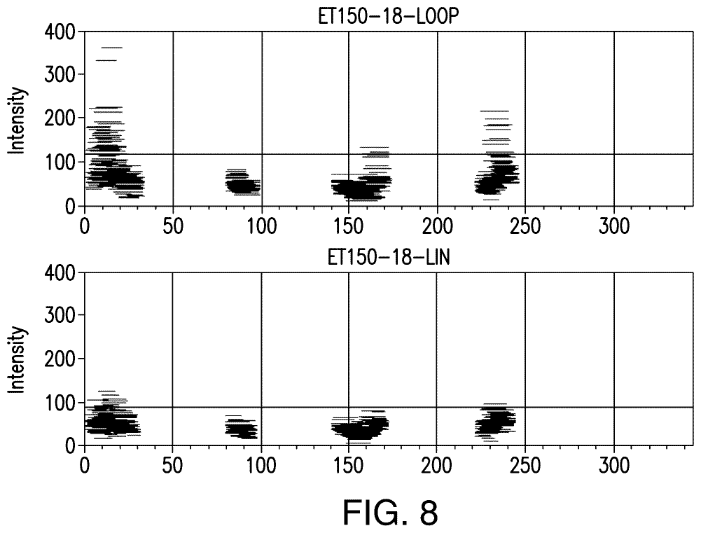

Antibody ET150-18. When tested under high stringency conditions antibody ET150-18 bound peptides from set 4 and set 7, containing structurally constrained peptides. No significant binding was recorded on sets containing linear peptides (FIG. 8). Cumulative data analysis shows that the antibody recognizes a discontinuous epitope composed of stretches ₁₀GDYFLLCD₁₇ (N-term) (SEQ ID NO: 388), ₁₅₇MFVNMTPCQLN₁₆₇ (ECL2) (SEQ ID NO: 389) and ₂₂₇GNPQFQRQPQW₂₃₇ (ECL3) (SEQ ID NO: 390). Peptide stretches ₁₀GDYFLLCD₁₇ (SEQ ID NO: 388) and ₂₂₇GNPQFQRQPQW₂₃₇ (SEQ ID NO: 390) represent the epitope's core, as both peptide stretches separately suffice for binding.

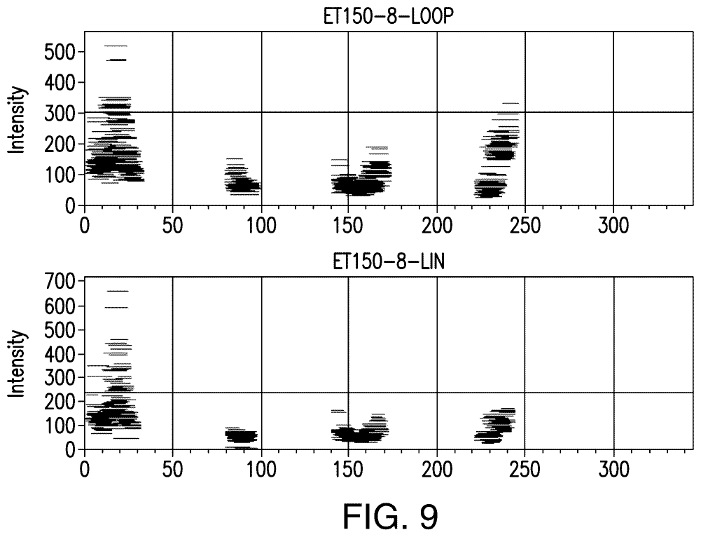

Antibody ET150-8. When tested under high stringency conditions antibody ET150-8 bound peptides from all sets, except for set 2 (FIG. 9). Cumulative data analysis shows that the antibody recognizes a discontinuous epitope composed of peptides stretches ₁₅LCDAEGPWG₂₃ (N-term) (SEQ ID NO: 391) and ₂₃₀QFQRQPQWDDPVVC₂₄₃ (ECL3) (SEQ ID NO: 392) where peptide stretch ₁₅LCDAEGPWG₂₃ (SEQ ID NO: 391) is the dominant part of the epitope, as it alone suffices for binding. Moreover, comparison of the results obtained on set 1 (linear) and set 4 (loop) shows that introduction of structural constrains to epitope mimics enhances binding of peptides, especially in case of peptides containing sequence ₂₃₀QFQRQPQWDDPVVC₂₄₃ (SEQ ID NO: 392).

Conclusions

All antibodies investigated recognized discontinuous epitopes, which were mapped using Pepscan arrays. Core tentative epitopes are listed in Table 36. All antibodies commonly recognized overlapping regions at the N-terminus of the protein in combination with regions from one or two ECLs. Two antibodies ET150-18 and ET150-8 showed a requirement for structural constraints to support antibody binding, suggesting that these two antibodies recognize not only discontinuous, but also conformational epitopes. Antibodies ET150-2 and ET150-5 did not show notable discrepancies in peptide binding between linear and looped peptides.

TABLE 36
List of epitopes

| Antibody | N-terminus | ECL1 | ECL2 | ECL3 |
|---|---|---|---|---|
| ET150-2 | ₁₆CDAEGPWGII₂₅*⁾ | — | ₁₅₇MFVNMTPC₁₆₄ | ₂₂₉PQFQRQPQW₂₃₇*⁾ |
| | (SEQ ID NO: 383) | | (SEQ ID NO: 384) | (SEQ ID NO: 385) |
| ET150-5 | ₅CIESTGDYFLLCD₁₇*⁾ | ₈₅NQQTAPVRYFL₉₅ | ₁₅₇MFVNMTPC₁₆₄ | — |
| | (SEQ ID NO: 386) | (SEQ ID NO: 387) | (SEQ ID NO: 384) | |
| ET150-8 | ₁₅LCDAEGPWG₂₃*⁾ | — | — | ₂₃₀QFQRQPQWDDPVVC₂₄₃ |
| | (SEQ ID NO: 391) | | | (SEQ ID NO: 392) |
| ET150-18 | ₁₀GDYFLLCD₁₇*⁾ | — | ₁₅₇MFVNMTPCQLN₁₆₇ | ₂₂₇GNPQFQRQPQW₂₃₇*⁾ |
| | (SEQ ID NO: 388) | | (SEQ ID NO: 389) | (SEQ ID NO: 390) |

*⁾dominant part

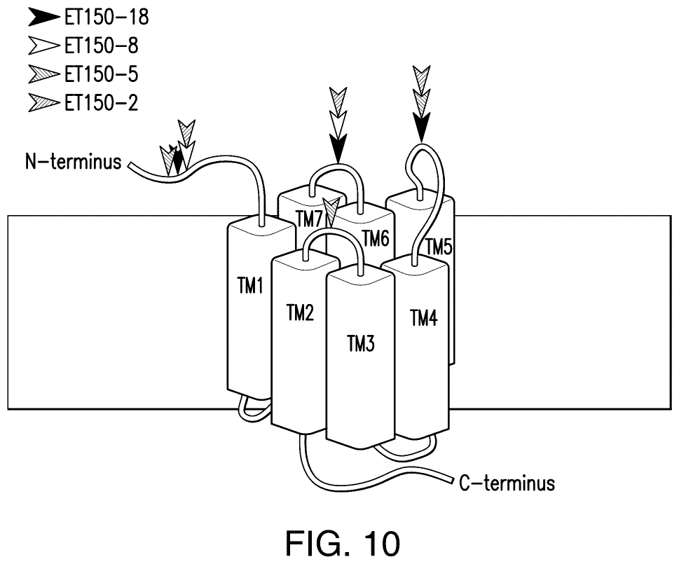

FIG. 10 is an illustration of the results of the study with respect to overall organization of GPCRs. As the N-terminus is highly flexible and unstructured, it likely transiently interacts with ECLs forming discontinuous immunodominant regions.

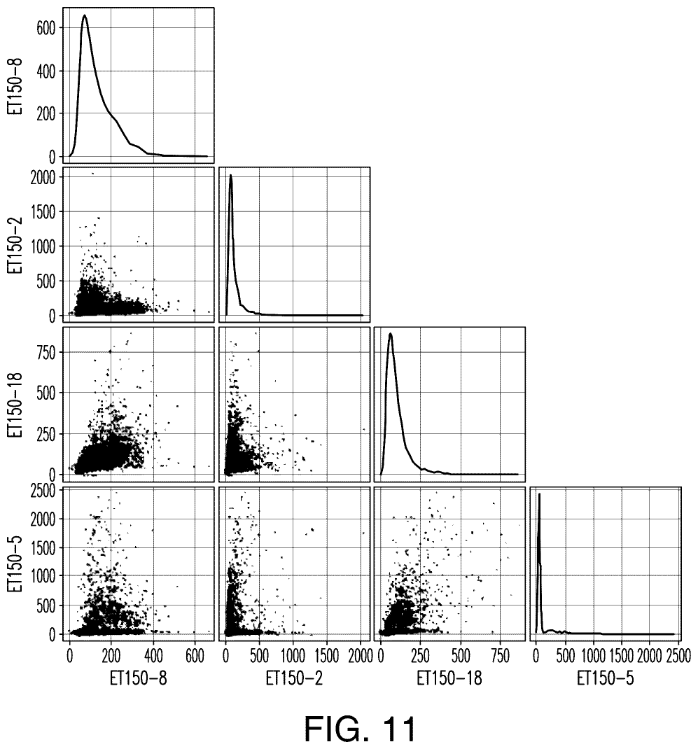

Differences and commonalities in peptide binding can be illustrated with a scatter plot analysis in FIG. 11. Data points in the top left and bottom right corners point to the differences in the binding. Despite significant epitope overlap, the fine specificities of epitopes of the individual antibodies differ to a large extent.

### Example 4—Screening Data for Anti-GPRC5D Antibodies

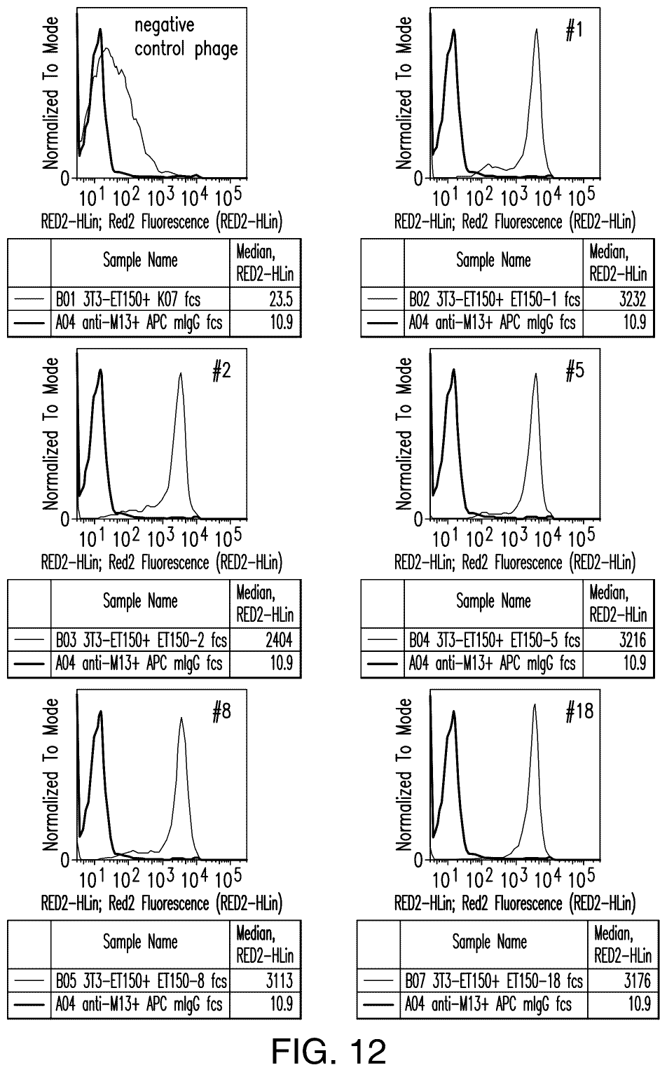

FACS Screening. FIG. 12 shows FACS analysis of the GPRC5D-specific phage antibody clones (ET150-1, ET150-2, ET150-5, ET150-8, ET150-18). Phage clones were incubated with 3T3-GPRC5D cell line, then with anti-M13 mouse antibody. Finally APC-labeled anti-mouse IgG 2nd antibody was added to the reaction after washing again. The binding was measured by FACS and expressed as mean fluorescence intensity (MFI). Cells incubated with M13 K07 helper phage and cells only were used as negative controls.

### Example 5—Binding Affinity of Anti-GPRC5D Antibodies

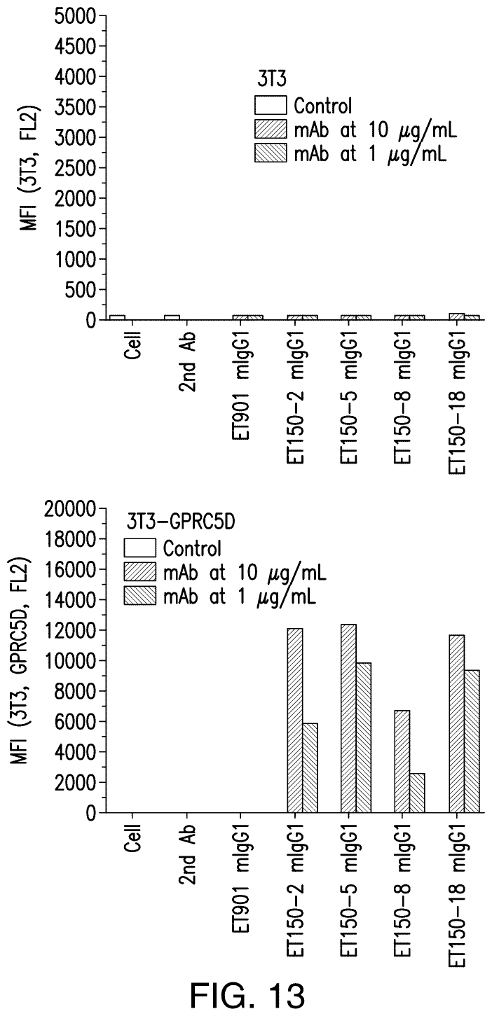

FIG. 13 shows FACS analysis of GPRC5D-specific phage antibody clones (ET150-2, ET150-5, ET150-8, ET150-18). Each antibody (ET150-1, ET150-2, ET150-5, ET150-8, ET150-18) was incubated with 3T3 or 3T3-GPRC5D cells at 10 or 1 μg/mL, then with anti-M13 mouse antibody. Finally PE-labeled anti-mouse IgG 2nd antibody was added to the reaction. The binding was measured by FACS and expressed as mean fluorescence intensity (MFI) (FIG. 13). Cells incubated with 2nd antibody alone, ET901 mIgG1 isotype control and cells only were used as negative controls.

### Example 6—Bispecific Antibodies Specific for GPRC5D and CD3

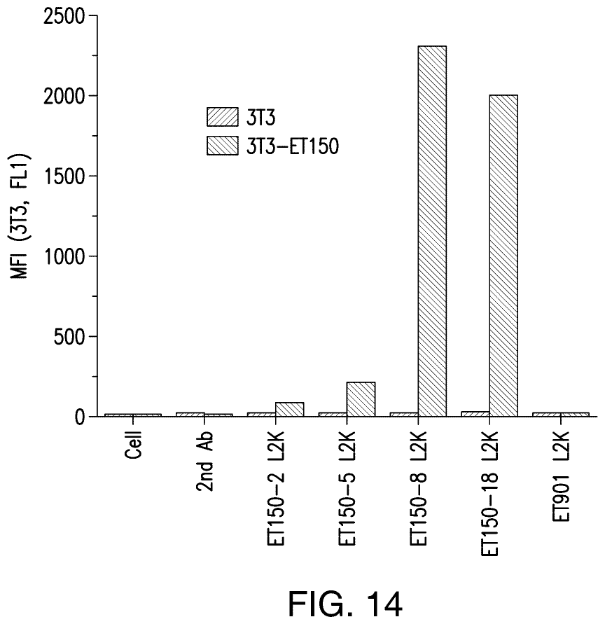

FIG. 14 shows FACS analysis of the anti-GPRC5D/anti-CD3 bispecific antibodies generated using the ET150-2, ET150-5, ET150-8, ET150-18 clones disclosed herein. Each antibody was incubated with 3T3 or 3T3-GPRC5D cells at 10 μg/ml, followed by the incubation with a FITC-conjugated anti-His tag antibody. The binding was measured by FACS and expressed as mean fluorescence intensity (MFI). Cells incubated with 2nd antibody alone, ET901 bispecific antibody control and cells only were used as negative controls. As shown in FIG. 14, the anti-GPRC5D/CD3 bispecific antibodies generated using the disclosed scFvs specifically bound to 3T3 cells expressing GPRC5D.

Although the foregoing presently disclosed subject matter has been described in some detail by way of illustration and example for purposes of clarity of understanding, the descriptions and examples should not be construed as limiting the scope of the presently disclosed subject matter. The disclosures of all patent and scientific literature cited herein are expressly incorporated in their entirety by reference.

1.-72. (canceled)

73. An antibody or antigen-binding fragment thereof comprising a heavy chain variable region that comprises CDR1, CDR2, and CDR3 domains; and a light chain variable region that comprises CDR1, CDR2, and CDR3 domains, wherein the heavy chain variable region and light chain variable region CDR3 domains are selected from the group consisting of:
(i) a heavy chain variable region CDR3 comprising amino acids having the sequence set forth in SEQ ID NO:126; and a light chain variable region CDR3 comprising amino acids having the sequence set forth in SEQ ID NO: 129;
(ii) a heavy chain variable region CDR3 comprising amino acids having the sequence set forth in SEQ ID NO: 132; and a light chain variable region CDR3 comprising amino acids having the sequence set forth in SEQ ID NO: 135;
(iii) a heavy chain variable region CDR3 comprising amino acids having the sequence set forth in SEQ ID NO: 138; and a light chain variable region CDR3 comprising amino acids having the sequence set forth in SEQ ID NO:141;
(iv) a heavy chain variable region CDR3 comprising amino acids having the sequence set forth in SEQ ID NO: 144; and a light chain variable region CDR3 comprising amino acids having the sequence set forth in SEQ ID NO: 147;
(v) a heavy chain variable region CDR3 comprising amino acids having the sequence set forth in SEQ ID NO:150; and a light chain variable region CDR3 comprising amino acids having the sequence set forth in SEQ ID NO:153;
(vi) a heavy chain variable region CDR3 comprising amino acids having the sequence set forth in SEQ ID NO: 156; and a light chain variable region CDR3 comprising amino acids having the sequence set forth in SEQ ID NO: 159;
(vii) a heavy chain variable region CDR3 comprising amino acids having the sequence set forth in SEQ ID NO: 162; and a light chain variable region CDR3 comprising amino acids having the sequence set forth in SEQ ID NO: 165;
(viii) a heavy chain variable region CDR3 comprising amino acids having the sequence set forth in SEQ ID NO: 168; and a light chain variable region CDR3 comprising amino acids having the sequence set forth in SEQ ID NO:171;
(ix) a heavy chain variable region CDR3 comprising amino acids having the sequence set forth in SEQ ID NO: 174; and a light chain variable region CDR3 comprising amino acids having the sequence set forth in SEQ ID NO: 177;
(x) a heavy chain variable region CDR3 comprising amino acids having the sequence set forth in SEQ ID NO:180; and a light chain variable region CDR3 comprising amino acids having the sequence set forth in SEQ ID NO:183;
(xi) a heavy chain variable region CDR3 comprising amino acids having the sequence set forth in SEQ ID NO: 186; and a light chain variable region CDR3 comprising amino acids having the sequence set forth in SEQ ID NO: 189;
(xii) a heavy chain variable region CDR3 comprising amino acids having the sequence set forth in SEQ ID NO: 192; and a light chain variable region CDR3 comprising amino acids having the sequence set forth in SEQ ID NO:195;
(xiii) a heavy chain variable region CDR3 comprising amino acids having the sequence set forth in SEQ ID NO: 198; and a light chain variable region CDR3 comprising amino acids having the sequence set forth in SEQ ID NO: 201;
(xiv) a heavy chain variable region CDR3 comprising amino acids having the sequence set forth in SEQ ID NO: 204; and a light chain variable region CDR3 comprising amino acids having the sequence set forth in SEQ ID NO: 207;
(xv) a heavy chain variable region CDR3 comprising amino acids having the sequence set forth in SEQ ID NO: 210; and a light chain variable region CDR3 comprising amino acids having the sequence set forth in SEQ ID NO: 213;
(xvi) a heavy chain variable region CDR3 comprising amino acids having the sequence set forth in SEQ ID NO: 216; and a light chain variable region CDR3 comprising amino acids having the sequence set forth in SEQ ID NO: 219;
(xvii) a heavy chain variable region CDR3 comprising amino acids having the sequence set forth in SEQ ID NO:222; and a light chain variable region CDR3 comprising amino acids having the sequence set forth in SEQ ID NO:225;
(xviii) a heavy chain variable region CDR3 comprising amino acids having the sequence set forth in SEQ ID NO: 228; and a light chain variable region CDR3 comprising amino acids having the sequence set forth in SEQ ID NO: 231;
(xix) a heavy chain variable region CDR3 comprising amino acids having the sequence set forth in SEQ ID NO: 234; and a light chain variable region CDR3 comprising amino acids having the sequence set forth in SEQ ID NO:237;
(xx) a heavy chain variable region CDR3 comprising amino acids having the sequence set forth in SEQ ID NO:240; and a light chain variable region CDR3 comprising amino acids having the sequence set forth in SEQ ID NO: 243;
(xxi) a heavy chain variable region CDR3 comprising amino acids having the sequence set forth in SEQ ID NO: 246; and a light chain variable region CDR3 comprising amino acids having the sequence set forth in SEQ ID NO: 249;
(xxii) a heavy chain variable region CDR3 comprising amino acids having the sequence set forth in SEQ ID NO: 252; and a light chain variable region CDR3 comprising amino acids having the sequence set forth in SEQ ID NO: 255;
(xxiii) a heavy chain variable region CDR3 comprising amino acids having the sequence set forth in SEQ ID NO: 258; and a light chain variable region CDR3 comprising amino acids having the sequence set forth in SEQ ID NO: 261;
(xxiv) a heavy chain variable region CDR3 comprising amino acids having the sequence set forth in SEQ ID NO: 264; and a light chain variable region CDR3 comprising amino acids having the sequence set forth in SEQ ID NO: 267;
(xxv) a heavy chain variable region CDR3 comprising amino acids having the sequence set forth in SEQ ID NO: 270; and a light chain variable region CDR3 comprising amino acids having the sequence set forth in SEQ ID NO: 273;
(xxvi) a heavy chain variable region CDR3 comprising amino acids having the sequence set forth in SEQ ID NO: 282; and a light chain variable region CDR3 comprising amino acids having the sequence set forth in SEQ ID NO: 285;
(xxvii) a heavy chain variable region CDR3 comprising amino acids having the sequence set forth in SEQ ID NO: 294; and a light chain variable region CDR3 comprising amino acids having the sequence set forth in SEQ ID NO: 297;
(xxviii) a heavy chain variable region CDR3 comprising amino acids having the sequence set forth in SEQ ID NO: 305; and a light chain variable region CDR3 comprising amino acids having the sequence set forth in SEQ ID NO: 308;
(xxix) a heavy chain variable region CDR3 comprising amino acids having the sequence set forth in SEQ ID NO: 318; and a light chain variable region CDR3 comprising amino acids having the sequence set forth in SEQ ID NO: 321;
(xxx) a heavy chain variable region CDR3 comprising amino acids having the sequence set forth in SEQ ID NO: 330; and a light chain variable region CDR3 comprising amino acids having the sequence set forth in SEQ ID NO: 333;
(xxxi) a heavy chain variable region CDR3 comprising amino acids having the sequence set forth in SEQ ID NO: 342; and a light chain variable region CDR3 comprising amino acids having the sequence set forth in SEQ ID NO: 345; and
(xxxii) a heavy chain variable region CDR3 comprising amino acids having the sequence set forth in SEQ ID NO: 354; and a light chain variable region CDR3 comprising amino acids having the sequence set forth in SEQ ID NO: 357,
wherein the antibody or antigen-binding portion thereof specifically binds to GPRC5D.

74. The antibody or antigen-binding portion thereof of claim 73, wherein the heavy chain variable region and light chain variable region CDR2 domains are selected from the group consisting of:
(i) a heavy chain variable region CDR2 comprising amino acids having the sequence set forth in SEQ ID NO:125; and a light chain variable region CDR2 comprising amino acids having the sequence set forth in SEQ ID NO: 128;
(ii) a heavy chain variable region CDR2 comprising amino acids having the sequence set forth in SEQ ID NO: 131; and a light chain variable region CDR2 comprising amino acids having the sequence set forth in SEQ ID NO: 134;
(iii) a heavy chain variable region CDR2 comprising amino acids having the sequence set forth in SEQ ID NO: 137; and a light chain variable region CDR2 comprising amino acids having the sequence set forth in SEQ ID NO: 140;
(iv) a heavy chain variable region CDR2 comprising amino acids having the sequence set forth in SEQ ID NO: 143; and a light chain variable region CDR2 comprising amino acids having the sequence set forth in SEQ ID NO: 146;
(v) a heavy chain variable region CDR2 comprising amino acids having the sequence set forth in SEQ ID NO: 149; and a light chain variable region CDR2 comprising amino acids having the sequence set forth in SEQ ID NO: 152;
(vi) a heavy chain variable region CDR2 comprising amino acids having the sequence set forth in SEQ ID NO: 155; and a light chain variable region CDR2 comprising amino acids having the sequence set forth in SEQ ID NO: 158;
(vii) a heavy chain variable region CDR2 comprising amino acids having the sequence set forth in SEQ ID NO: 161; and a light chain variable region CDR2 comprising amino acids having the sequence set forth in SEQ ID NO: 164;
(viii) a heavy chain variable region CDR2 comprising amino acids having the sequence set forth in SEQ ID NO: 167; and a light chain variable region CDR2 comprising amino acids having the sequence set forth in SEQ ID NO: 170;
(ix) a heavy chain variable region CDR2 comprising amino acids having the sequence set forth in SEQ ID NO: 173; and a light chain variable region CDR2 comprising amino acids having the sequence set forth in SEQ ID NO: 176;
(x) a heavy chain variable region CDR2 comprising amino acids having the sequence set forth in SEQ ID NO:179; and a light chain variable region CDR2 comprising amino acids having the sequence set forth in SEQ ID NO:182;
(xi) a heavy chain variable region CDR2 comprising amino acids having the sequence set forth in SEQ ID NO: 185; and a light chain variable region CDR2 comprising amino acids having the sequence set forth in SEQ ID NO: 188;
(xii) a heavy chain variable region CDR2 comprising amino acids having the sequence set forth in SEQ ID NO:191; and a light chain variable region CDR2 comprising amino acids having the sequence set forth in SEQ ID NO:194;
(xiii) a heavy chain variable region CDR2 comprising amino acids having the sequence set forth in SEQ ID NO:197; and a light chain variable region CDR2 comprising amino acids having the sequence set forth in SEQ ID NO:200;
(xiv) a heavy chain variable region CDR2 comprising amino acids having the sequence set forth in SEQ ID NO: 203; and a light chain variable region CDR2 comprising amino acids having the sequence set forth in SEQ ID NO: 206;
(xv) a heavy chain variable region CDR2 comprising amino acids having the sequence set forth in SEQ ID NO: 209; and a light chain variable region CDR2 comprising amino acids having the sequence set forth in SEQ ID NO: 212;
(xvi) a heavy chain variable region CDR2 comprising amino acids having the sequence set forth in SEQ ID NO: 215; and a light chain variable region CDR2 comprising amino acids having the sequence set forth in SEQ ID NO: 218;
(xvii) a heavy chain variable region CDR2 comprising amino acids having the sequence set forth in SEQ ID NO: 221; and a light chain variable region CDR2 comprising amino acids having the sequence set forth in SEQ ID NO: 224;
(xviii) a heavy chain variable region CDR2 comprising amino acids having the sequence set forth in SEQ ID NO: 227; and a light chain variable region CDR2 comprising amino acids having the sequence set forth in SEQ ID NO:230;
(xix) a heavy chain variable region CDR2 comprising amino acids having the sequence set forth in SEQ ID NO:233; and a light chain variable region CDR2 comprising amino acids having the sequence set forth in SEQ ID NO:236;
(xx) a heavy chain variable region CDR2 comprising amino acids having the sequence set forth in SEQ ID NO:239; and a light chain variable region CDR2 comprising amino acids having the sequence set forth in SEQ ID NO:242;
(xxi) a heavy chain variable region CDR2 comprising amino acids having the sequence set forth in SEQ ID NO:245; and a light chain variable region CDR2 comprising amino acids having the sequence set forth in SEQ ID NO:248;
(xxii) a heavy chain variable region CDR2 comprising amino acids having the sequence set forth in SEQ ID NO:251; and a light chain variable region CDR2 comprising amino acids having the sequence set forth in SEQ ID NO:254;
(xxiii) a heavy chain variable region CDR2 comprising amino acids having the sequence set forth in SEQ ID NO:257; and a light chain variable region CDR2 comprising amino acids having the sequence set forth in SEQ ID NO:260;
(xxiv) a heavy chain variable region CDR2 comprising amino acids having the sequence set forth in SEQ ID NO:263; and a light chain variable region CDR2 comprising amino acids having the sequence set forth in SEQ ID NO:266;
(xxv) a heavy chain variable region CDR2 comprising amino acids having the sequence set forth in SEQ ID NO:269; and a light chain variable region CDR2 comprising amino acids having the sequence set forth in SEQ ID NO:272;
(xxvi) a heavy chain variable region CDR2 comprising amino acids having the sequence set forth in SEQ ID NO:281; and a light chain variable region CDR2 comprising amino acids having the sequence set forth in SEQ ID NO:284;
(xxvii) a heavy chain variable region CDR2 comprising amino acids having the sequence set forth in SEQ ID NO:293; and a light chain variable region CDR2 comprising amino acids having the sequence set forth in SEQ ID NO:296;
(xxviii) a heavy chain variable region CDR2 comprising amino acids having the sequence set forth in SEQ ID NO:304; and a light chain variable region CDR2 comprising amino acids having the sequence set forth in SEQ ID NO:307;
(xxix) a heavy chain variable region CDR2 comprising amino acids having the sequence set forth in SEQ ID NO:317; and a light chain variable region CDR2 comprising amino acids having the sequence set forth in SEQ ID NO:320;
(xxx) a heavy chain variable region CDR2 comprising amino acids having the sequence set forth in SEQ ID NO:329; and a light chain variable region CDR2 comprising amino acids having the sequence set forth in SEQ ID NO:332;
(xxxi) a heavy chain variable region CDR2 comprising amino acids having the sequence set forth in SEQ ID NO:341; and a light chain variable region CDR2 comprising amino acids having the sequence set forth in SEQ ID NO:344; and
(xxxii) a heavy chain variable region CDR2 comprising amino acids having the sequence set forth in SEQ ID NO:353; and a light chain variable region CDR2 comprising amino acids having the sequence set forth in SEQ ID NO:356.

75. The antibody or antigen-binding portion thereof of claim 73, wherein the heavy chain variable region and light chain variable region CDR1 domains are selected from the group consisting of:
(i) a heavy chain variable region CDR1 comprising amino acids having the sequence set forth in SEQ ID NO:124; and a light chain variable region CDR1 comprising amino acids having the sequence set forth in SEQ ID NO: 127;
(ii) a heavy chain variable region CDR1 comprising amino acids having the sequence set forth in SEQ ID NO:130; and a light chain variable region CDR1 comprising amino acids having the sequence set forth in SEQ ID NO:133;
(iii) a heavy chain variable region CDR1 comprising amino acids having the sequence set forth in SEQ ID NO:136; and a light chain variable region CDR1 comprising amino acids having the sequence set forth in SEQ ID NO:139;
(iv) a heavy chain variable region CDR1 comprising amino acids having the sequence set forth in SEQ ID NO:142; and a light chain variable region CDR1 comprising amino acids having the sequence set forth in SEQ ID NO:145;
(v) a heavy chain variable region CDR1 comprising amino acids having the sequence set forth in SEQ ID NO:148; and a light chain variable region CDR1 comprising amino acids having the sequence set forth in SEQ ID NO:151;
(vi) a heavy chain variable region CDR1 comprising amino acids having the sequence set forth in SEQ ID NO:154; and a light chain variable region CDR1 comprising amino acids having the sequence set forth in SEQ ID NO:157;
(vii) a heavy chain variable region CDR1 comprising amino acids having the sequence set forth in SEQ ID NO:160; and a light chain variable region CDR1 comprising amino acids having the sequence set forth in SEQ ID NO:163;
(viii) a heavy chain variable region CDR1 comprising amino acids having the sequence set forth in SEQ ID NO:166; and a light chain variable region CDR1 comprising amino acids having the sequence set forth in SEQ ID NO:169;
(ix) a heavy chain variable region CDR1 comprising amino acids having the sequence set forth in SEQ ID NO:172; and a light chain variable region CDR1 comprising amino acids having the sequence set forth in SEQ ID NO:175;
(x) a heavy chain variable region CDR1 comprising amino acids having the sequence set forth in SEQ ID NO:178; and a light chain variable region CDR1 comprising amino acids having the sequence set forth in SEQ ID NO:181;
(xi) a heavy chain variable region CDR1 comprising amino acids having the sequence set forth in SEQ ID NO:184; and a light chain variable region CDR1 comprising amino acids having the sequence set forth in SEQ ID NO:187;
(xii) a heavy chain variable region CDR1 comprising amino acids having the sequence set forth in SEQ ID NO:190; and a light chain variable region CDR1 comprising amino acids having the sequence set forth in SEQ ID NO:193;
(xiii) a heavy chain variable region CDR1 comprising amino acids having the sequence set forth in SEQ ID NO:196; and a light chain variable region CDR1 comprising amino acids having the sequence set forth in SEQ ID NO:199;
(xiv) a heavy chain variable region CDR1 comprising amino acids having the sequence set forth in SEQ ID NO:202; and a light chain variable region CDR1 comprising amino acids having the sequence set forth in SEQ ID NO:205;
(xv) a heavy chain variable region CDR1 comprising amino acids having the sequence set forth in SEQ ID NO:208; and a light chain variable region CDR1 comprising amino acids having the sequence set forth in SEQ ID NO:211;
(xvi) a heavy chain variable region CDR1 comprising amino acids having the sequence set forth in SEQ ID NO:214; and a light chain variable region CDR1 comprising amino acids having the sequence set forth in SEQ ID NO:217;
(xvii) a heavy chain variable region CDR1 comprising amino acids having the sequence set forth in SEQ ID NO:220; and a light chain variable region CDR1 comprising amino acids having the sequence set forth in SEQ ID NO:223;
(xviii) a heavy chain variable region CDR1 comprising amino acids having the sequence set forth in SEQ ID NO:226; and a light chain variable region CDR1 comprising amino acids having the sequence set forth in SEQ ID NO:229;
(xix) a heavy chain variable region CDR1 comprising amino acids having the sequence set forth in SEQ ID NO:232; and a light chain variable region CDR1 comprising amino acids having the sequence set forth in SEQ ID NO:235;
(xx) a heavy chain variable region CDR1 comprising amino acids having the sequence set forth in SEQ ID NO:238; and a light chain variable region CDR1 comprising amino acids having the sequence set forth in SEQ ID NO:241;
(xxi) a heavy chain variable region CDR1 comprising amino acids having the sequence set forth in SEQ ID NO:244; and a light chain variable region CDR1 comprising amino acids having the sequence set forth in SEQ ID NO:247;
(xxii) a heavy chain variable region CDR1 comprising amino acids having the sequence set forth in SEQ ID NO:250; and a light chain variable region CDR1 comprising amino acids having the sequence set forth in SEQ ID NO:253;
(xxiii) a heavy chain variable region CDR1 comprising amino acids having the sequence set forth in SEQ ID NO:256; and a light chain variable region CDR1 comprising amino acids having the sequence set forth in SEQ ID NO:259;
(xxiv) a heavy chain variable region CDR1 comprising amino acids having the sequence set forth in SEQ ID NO:262; and a light chain variable region CDR1 comprising amino acids having the sequence set forth in SEQ ID NO:265;
(xxv) a heavy chain variable region CDR1 comprising amino acids having the sequence set forth in SEQ ID NO:268; and a light chain variable region CDR1 comprising amino acids having the sequence set forth in SEQ ID NO:271;
(xxvi) a heavy chain variable region CDR1 comprising amino acids having the sequence set forth in SEQ ID NO:280; and a light chain variable region CDR1 comprising amino acids having the sequence set forth in SEQ ID NO:283;
(xxvii) a heavy chain variable region CDR1 comprising amino acids having the sequence set forth in SEQ ID NO:292; and a light chain variable region CDR1 comprising amino acids having the sequence set forth in SEQ ID NO:295;
(xxviii) a heavy chain variable region CDR1 comprising amino acids having the sequence set forth in SEQ ID NO:303; and a light chain variable region CDR1 comprising amino acids having the sequence set forth in SEQ ID NO:306;
(xxix) a heavy chain variable region CDR1 comprising amino acids having the sequence set forth in SEQ ID NO:316; and a light chain variable region CDR1 comprising amino acids having the sequence set forth in SEQ ID NO:319;
(xxx) a heavy chain variable region CDR1 comprising amino acids having the sequence set forth in SEQ ID NO:328; and a light chain variable region CDR1 comprising amino acids having the sequence set forth in SEQ ID NO:331;
(xxxi) a heavy chain variable region CDR1 comprising amino acids having the sequence set forth in SEQ ID NO:340; and a light chain variable region CDR1 comprising amino acids having the sequence set forth in SEQ ID NO:343; and
(xxxii) a heavy chain variable region CDR1 comprising amino acids having the sequence set forth in SEQ ID NO:352; and a light chain variable region CDR1 comprising amino acids having the sequence set forth in SEQ ID NO:355.

76. The antibody, or an antigen-binding portion thereof of claim 75, comprising:
(i) a heavy chain variable region CDR1 comprising amino acids having the sequence set forth in SEQ ID NO: 124; a heavy chain variable region CDR2 comprising amino acids having the sequence set forth in SEQ ID NO: 125; and a heavy chain variable region CDR3 comprising amino acids having the sequence set forth in SEQ ID NO: 126;
(ii) a heavy chain variable region CDR1 comprising amino acids having the sequence set forth in SEQ ID NO: 130; a heavy chain variable region CDR2 comprising amino acids having the sequence set forth in SEQ ID NO: 131; and a heavy chain variable region CDR3 comprising amino acids having the sequence set forth in SEQ ID NO: 132;
(iii) a heavy chain variable region CDR1 comprising amino acids having the sequence set forth in SEQ ID NO: 136; a heavy chain variable region CDR2 comprising amino acids having the sequence set forth in SEQ ID NO: 137; and a heavy chain variable region CDR3 comprising amino acids having the sequence set forth in SEQ ID NO: 138;
(iv) a heavy chain variable region CDR1 comprising amino acids having the sequence set forth in SEQ ID NO: 142; a heavy chain variable region CDR2 comprising amino acids having the sequence set forth in SEQ ID NO: 143; and a heavy chain variable region CDR3 comprising amino acids having the sequence set forth in SEQ ID NO: 144;
(v) a heavy chain variable region CDR1 comprising amino acids having the sequence set forth in SEQ ID NO: 148; a heavy chain variable region CDR2 comprising amino acids having the sequence set forth in SEQ ID NO: 149; and a heavy chain variable region CDR3 comprising amino acids having the sequence set forth in SEQ ID NO: 150;
(vi) a heavy chain variable region CDR1 comprising amino acids having the sequence set forth in SEQ ID NO: 154; a heavy chain variable region CDR2 comprising amino acids having the sequence set forth in SEQ ID NO: 155; and a heavy chain variable region CDR3 comprising amino acids having the sequence set forth in SEQ ID NO: 156;
(vii) a heavy chain variable region CDR1 comprising amino acids having the sequence set forth in SEQ ID NO: 160; a heavy chain variable region CDR2 comprising amino acids having the sequence set forth in SEQ ID NO: 161; and a heavy chain variable region CDR3 comprising amino acids having the sequence set forth in SEQ ID NO: 162;
(viii) a heavy chain variable region CDR1 comprising amino acids having the sequence set forth in SEQ ID NO: 166; a heavy chain variable region CDR2 comprising amino acids having the sequence set forth in SEQ ID NO: 167; and a heavy chain variable region CDR3 comprising amino acids having the sequence set forth in SEQ ID NO: 168;
(ix) a heavy chain variable region CDR1 comprising amino acids having the sequence set forth in SEQ ID NO: 172; a heavy chain variable region CDR2 comprising amino acids having the sequence set forth in SEQ ID NO: 173; and a heavy chain variable region CDR3 comprising amino acids having the sequence set forth in SEQ ID NO: 174;
(x) a heavy chain variable region CDR1 comprising amino acids having the sequence set forth in SEQ ID NO: 178; a heavy chain variable region CDR2 comprising amino acids having the sequence set forth in SEQ ID NO: 179; and a heavy chain variable region CDR3 comprising amino acids having the sequence set forth in SEQ ID NO: 180;
(xi) a heavy chain variable region CDR1 comprising amino acids having the sequence set forth in SEQ ID NO: 184; a heavy chain variable region CDR2 comprising amino acids having the sequence set forth in SEQ ID NO: 185; and a heavy chain variable region CDR3 comprising amino acids having the sequence set forth in SEQ ID NO: 186;
(xii) a heavy chain variable region CDR1 comprising amino acids having the sequence set forth in SEQ ID NO: 190 a heavy chain variable region CDR2 comprising amino acids having the sequence set forth in SEQ ID NO: 191; and a heavy chain variable region CDR3 comprising amino acids having the sequence set forth in SEQ ID NO: 192;
(xiii) a heavy chain variable region CDR1 comprising amino acids having the sequence set forth in SEQ ID NO: 196; a heavy chain variable region CDR2 comprising amino acids having the sequence set forth in SEQ ID NO: 197; and a heavy chain variable region CDR3 comprising amino acids having the sequence set forth in SEQ ID NO: 198;
(xiv) a heavy chain variable region CDR1 comprising amino acids having the sequence set forth in SEQ ID NO: 202; a heavy chain variable region CDR2 comprising amino acids having the sequence set forth in SEQ ID NO: 203; and a heavy chain variable region CDR3 comprising amino acids having the sequence set forth in SEQ ID NO: 204;
(xv) a heavy chain variable region CDR1 comprising amino acids having the sequence set forth in SEQ ID NO: 208; a heavy chain variable region CDR2 comprising amino acids having the sequence set forth in SEQ ID NO: 209; and a heavy chain variable region CDR3 comprising amino acids having the sequence set forth in SEQ ID NO: 210;
(xvi) a heavy chain variable region CDR1 comprising amino acids having the sequence set forth in SEQ ID NO: 214; a heavy chain variable region CDR2 comprising amino acids having the sequence set forth in SEQ ID NO: 215; and a heavy chain variable region CDR3 comprising amino acids having the sequence set forth in SEQ ID NO: 216;
(xvii) a heavy chain variable region CDR1 comprising amino acids having the sequence set forth in SEQ ID NO: 220; a heavy chain variable region CDR2 comprising amino acids having the sequence set forth in SEQ ID NO: 221; and a heavy chain variable region CDR3 comprising amino acids having the sequence set forth in SEQ ID NO: 222;
(xviii) a heavy chain variable region CDR1 comprising amino acids having the sequence set forth in SEQ ID NO: 226; a heavy chain variable region CDR2 comprising amino acids having the sequence set forth in SEQ ID NO: 227; and a heavy chain variable region CDR3 comprising amino acids having the sequence set forth in SEQ ID NO: 228;
(xix) a heavy chain variable region CDR1 comprising amino acids having the sequence set forth in SEQ ID NO: 232; a heavy chain variable region CDR2 comprising amino acids having the sequence set forth in SEQ ID NO: 233; and a heavy chain variable region CDR3 comprising amino acids having the sequence set forth in SEQ ID NO: 234;
(xx) a heavy chain variable region CDR1 comprising amino acids having the sequence set forth in SEQ ID NO: 238; a heavy chain variable region CDR2 comprising amino acids having the sequence set forth in SEQ ID NO: 239; and a heavy chain variable region CDR3 comprising amino acids having the sequence set forth in SEQ ID NO: 240;
(xxi) a heavy chain variable region CDR1 comprising amino acids having the sequence set forth in SEQ ID NO: 244; a heavy chain variable region CDR2 comprising amino acids having the sequence set forth in SEQ ID NO: 245; and a heavy chain variable region CDR3 comprising amino acids having the sequence set forth in SEQ ID NO: 246;
(xxii) a heavy chain variable region CDR1 comprising amino acids having the sequence set forth in SEQ ID NO: 250; a heavy chain variable region CDR2 comprising amino acids having the sequence set forth in SEQ ID NO: 251; and a heavy chain variable region CDR3 comprising amino acids having the sequence set forth in SEQ ID NO: 252;
(xxiii) a heavy chain variable region CDR1 comprising amino acids having the sequence set forth in SEQ ID NO: 256; a heavy chain variable region CDR2 comprising amino acids having the sequence set forth in SEQ ID NO: 257; and a heavy chain variable region CDR3 comprising amino acids having the sequence set forth in SEQ ID NO: 258;
(xxiv) a heavy chain variable region CDR1 comprising amino acids having the sequence set forth in SEQ ID NO: 262; a heavy chain variable region CDR2 comprising amino acids having the sequence set forth in SEQ ID NO: 263; and a heavy chain variable region CDR3 comprising amino acids having the sequence set forth in SEQ ID NO: 264;
(xxv) a heavy chain variable region CDR1 comprising amino acids having the sequence set forth in SEQ ID NO: 268; a heavy chain variable region CDR2 comprising amino acids having the sequence set forth in SEQ ID NO: 269; and a heavy chain variable region CDR3 comprising amino acids having the sequence set forth in SEQ ID NO: 270;
(xxvi) a heavy chain variable region CDR1 comprising amino acids having the sequence set forth in SEQ ID NO: 280; a heavy chain variable region CDR2 comprising amino acids having the sequence set forth in SEQ ID NO: 281; and a heavy chain variable region CDR3 comprising amino acids having the sequence set forth in SEQ ID NO: 282;
(xxvii) a heavy chain variable region CDR1 comprising amino acids having the sequence set forth in SEQ ID NO: 292; a heavy chain variable region CDR2 comprising amino acids having the sequence set forth in SEQ ID NO: 293; and a heavy chain variable region CDR3 comprising amino acids having the sequence set forth in SEQ ID NO: 294;
(xxviii) a heavy chain variable region CDR1 comprising amino acids having the sequence set forth in SEQ ID NO: 303; a heavy chain variable region CDR2 comprising amino acids having the sequence set forth in SEQ ID NO: 304; and a heavy chain variable region CDR3 comprising amino acids having the sequence set forth in SEQ ID NO: 305;
(xxix) a heavy chain variable region CDR1 comprising amino acids having the sequence set forth in SEQ ID NO: 316; a heavy chain variable region CDR2 comprising amino acids having the sequence set forth in SEQ ID NO: 317; and a heavy chain variable region CDR3 comprising amino acids having the sequence set forth in SEQ ID NO: 318;
(xxx) a heavy chain variable region CDR1 comprising amino acids having the sequence set forth in SEQ ID NO: 328; a heavy chain variable region CDR2 comprising amino acids having the sequence set forth in SEQ ID NO: 329; and a heavy chain variable region CDR3 comprising amino acids having the sequence set forth in SEQ ID NO: 330;
(xxxi) a heavy chain variable region CDR1 comprising amino acids having the sequence set forth in SEQ ID NO: 340; a heavy chain variable region CDR2 comprising amino acids having the sequence set forth in SEQ ID NO: 341; and a heavy chain variable region CDR3 comprising amino acids having the sequence set forth in SEQ ID NO: 342; and
(xxxii) a heavy chain variable region CDR1 comprising amino acids having the sequence set forth in SEQ ID NO: 352; a heavy chain variable region CDR2 comprising amino acids having the sequence set forth in SEQ ID NO: 353; and a heavy chain variable region CDR3 comprising amino acids having the sequence set forth in SEQ ID NO: 354;
wherein the antibody or antigen-binding portion thereof specifically binds to GPRC5D.

77. The antibody, or an antigen-binding portion thereof of claim 75, comprising:
(i) a light chain variable region CDR1 comprising amino acids having the sequence set forth in SEQ ID NO: 127; a light chain variable region CDR2 comprising amino acids having the sequence set forth in SEQ ID NO:129; and a light chain variable region CDR3 comprising amino acids having the sequence set forth in SEQ ID NO: 130;
(ii) a light chain variable region CDR1 comprising amino acids having the sequence set forth in SEQ ID NO: 133; a light chain variable region CDR2 comprising amino acids having the sequence set forth in SEQ ID NO:134; and a light chain variable region CDR3 comprising SEQ ID NO: 135;
(iii) a light chain variable region CDR1 comprising amino acids having the sequence set forth in SEQ ID NO: 139; a light chain variable region CDR2 comprising amino acids having the sequence set forth in SEQ ID NO:140; and a light chain variable region CDR3 comprising amino acids having the sequence set forth in SEQ ID NO: 141;
(iv) a light chain variable region CDR1 comprising amino acids having the sequence set forth in SEQ ID NO: 145; a light chain variable region CDR2 comprising amino acids having the sequence set forth in SEQ ID NO:146; and a light chain variable region CDR3 comprising amino acids having the sequence set forth in SEQ ID NO: 147;
(v) a light chain variable region CDR1 comprising amino acids having the sequence set forth in SEQ ID NO: 151; a light chain variable region CDR2 comprising amino acids having the sequence set forth in SEQ ID NO:152; and a light chain variable region CDR3 comprising amino acids having the sequence set forth in SEQ ID NO: 153;
(vi) a light chain variable region CDR1 comprising amino acids having the sequence set forth in SEQ ID NO: 157; a light chain variable region CDR2 comprising amino acids having the sequence set forth in SEQ ID NO:158; and a light chain variable region CDR3 comprising amino acids having the sequence set forth in SEQ ID NO: 159;
(vii) a light chain variable region CDR1 comprising amino acids having the sequence set forth in SEQ ID NO: 163; a light chain variable region CDR2 comprising amino acids having the sequence set forth in SEQ ID NO:164; and a light chain variable region CDR3 comprising amino acids having the sequence set forth in SEQ ID NO: 165;
(viii) a light chain variable region CDR1 comprising amino acids having the sequence set forth in SEQ ID NO: 169; a light chain variable region CDR2 comprising amino acids having the sequence set forth in SEQ ID NO:170; and a light chain variable region CDR3 comprising SEQ ID NO: 171;
(ix) a light chain variable region CDR1 comprising amino acids having the sequence set forth in SEQ ID NO: 175; a light chain variable region CDR2 comprising amino acids having the sequence set forth in SEQ ID NO:176; and a light chain variable region CDR3 comprising amino acids having the sequence set forth in SEQ ID NO: 177;
(x) a light chain variable region CDR1 comprising amino acids having the sequence set forth in SEQ ID NO: 181; a light chain variable region CDR2 comprising amino acids having the sequence set forth in SEQ ID NO:182; and a light chain variable region CDR3 comprising amino acids having the sequence set forth in SEQ ID NO: 183;
(xi) a light chain variable region CDR1 comprising amino acids having the sequence set forth in SEQ ID NO: 187; a light chain variable region CDR2 comprising amino acids having the sequence set forth in SEQ ID NO:188; and a light chain variable region CDR3 comprising amino acids having the sequence set forth in SEQ ID NO: 189;
(xii) a light chain variable region CDR1 comprising amino acids having the sequence set forth in SEQ ID NO: 193; a light chain variable region CDR2 comprising amino acids having the sequence set forth in SEQ ID NO:194; and a light chain variable region CDR3 comprising amino acids having the sequence set forth in SEQ ID NO: 195;
(xiii) a light chain variable region CDR1 comprising amino acids having the sequence set forth in SEQ ID NO: 199; a light chain variable region CDR2 comprising amino acids having the sequence set forth in SEQ ID NO:200; and a light chain variable region CDR3 comprising amino acids having the sequence set forth in SEQ ID NO: 201;
(xiv) a light chain variable region CDR1 comprising amino acids having the sequence set forth in SEQ ID NO: 205; a light chain variable region CDR2 comprising amino acids having the sequence set forth in SEQ ID NO:206; and a light chain variable region CDR3 comprising amino acids having the sequence set forth in SEQ ID NO: 207;
(xv) a light chain variable region CDR1 comprising amino acids having the sequence set forth in SEQ ID NO: 211; a light chain variable region CDR2 comprising amino acids having the sequence set forth in SEQ ID NO:212; and a light chain variable region CDR3 comprising amino acids having the sequence set forth in SEQ ID NO: 213;
(xvi) a light chain variable region CDR1 comprising amino acids having the sequence set forth in SEQ ID NO: 217; a light chain variable region CDR2 comprising amino acids having the sequence set forth in SEQ ID NO:218; and a light chain variable region CDR3 comprising amino acids having the sequence set forth in SEQ ID NO: 219;
(xvii) a light chain variable region CDR1 comprising amino acids having the sequence set forth in SEQ ID NO: 223; a light chain variable region CDR2 comprising amino acids having the sequence set forth in SEQ ID NO:224; and a light chain variable region CDR3 comprising amino acids having the sequence set forth in SEQ ID NO: 225;
(xviii) a light chain variable region CDR1 comprising amino acids having the sequence set forth in SEQ ID NO: 229; a light chain variable region CDR2 comprising amino acids having the sequence set forth in SEQ ID NO:230; and a light chain variable region CDR3 comprising amino acids having the sequence set forth in SEQ ID NO: 231;
(xix) a light chain variable region CDR1 comprising amino acids having the sequence set forth in SEQ ID NO: 235; a light chain variable region CDR2 comprising amino acids having the sequence set forth in SEQ ID NO:236; and a light chain variable region CDR3 comprising amino acids having the sequence set forth in SEQ ID NO: 237;
(xx) a light chain variable region CDR1 comprising amino acids having the sequence set forth in SEQ ID NO: 241; a light chain variable region CDR2 comprising amino acids having the sequence set forth in SEQ ID NO:242; and a light chain variable region CDR3 comprising amino acids having the sequence set forth in SEQ ID NO: 243;
(xxi) a light chain variable region CDR1 comprising amino acids having the sequence set forth in SEQ ID NO: 247; a light chain variable region CDR2 comprising amino acids having the sequence set forth in SEQ ID NO:248; and a light chain variable region CDR3 comprising amino acids having the sequence set forth in SEQ ID NO: 249;
(xxii) a light chain variable region CDR1 comprising amino acids having the sequence set forth in SEQ ID NO: 253; a light chain variable region CDR2 comprising amino acids having the sequence set forth in SEQ ID NO:254; and a light chain variable region CDR3 comprising amino acids having the sequence set forth in SEQ ID NO: 255;
(xxiii) a light chain variable region CDR1 comprising amino acids having the sequence set forth in SEQ ID NO: 259; a light chain variable region CDR2 comprising amino acids having the sequence set forth in SEQ ID NO:250; and a light chain variable region CDR3 comprising amino acids having the sequence set forth in SEQ ID NO: 261;
(xxiv) a light chain variable region CDR1 comprising amino acids having the sequence set forth in SEQ ID NO: 265; a light chain variable region CDR2 comprising amino acids having the sequence set forth in SEQ ID NO:266; and a light chain variable region CDR3 comprising amino acids having the sequence set forth in SEQ ID NO: 267;
(xxv) a light chain variable region CDR1 comprising amino acids having the sequence set forth in SEQ ID NO: 271; a light chain variable region CDR2 comprising amino acids having the sequence set forth in SEQ ID NO:272; and a light chain variable region CDR3 comprising amino acids having the sequence set forth in SEQ ID NO: 273;
(xxvi) a light chain variable region CDR1 comprising amino acids having the sequence set forth in SEQ ID NO: 283; a light chain variable region CDR2 comprising amino acids having the sequence set forth in SEQ ID NO:284; and a light chain variable region CDR3 comprising amino acids having the sequence set forth in SEQ ID NO: 285;
(xxvii) a light chain variable region CDR1 comprising amino acids having the sequence set forth in SEQ ID NO: 295; a light chain variable region CDR2 comprising amino acids having the sequence set forth in SEQ ID NO:296; and a light chain variable region CDR3 comprising amino acids having the sequence set forth in SEQ ID NO: 297;
(xxviii) a light chain variable region CDR1 comprising amino acids having the sequence set forth in SEQ ID NO: 306; a light chain variable region CDR2 comprising amino acids having the sequence set forth in SEQ ID NO:307; and a light chain variable region CDR3 comprising amino acids having the sequence set forth in SEQ ID NO: 308;
(xxix) a light chain variable region CDR1 comprising amino acids having the sequence set forth in SEQ ID NO: 319; a light chain variable region CDR2 comprising amino acids having the sequence set forth in SEQ ID NO:320; and a light chain variable region CDR3 comprising amino acids having the sequence set forth in SEQ ID NO: 321;
(xxx) a light chain variable region CDR1 comprising amino acids having the sequence set forth in SEQ ID NO: 331; a light chain variable region CDR2 comprising amino acids having the sequence set forth in SEQ ID NO:332; and a light chain variable region CDR3 comprising amino acids having the sequence set forth in SEQ ID NO: 333;
(xxxi) a light chain variable region CDR1 comprising amino acids having the sequence set forth in SEQ ID NO: 343; a light chain variable region CDR2 comprising amino acids having the sequence set forth in SEQ ID NO:344; and a light chain variable region CDR3 comprising amino acids having the sequence set forth in SEQ ID NO: 345;
(xxxii) a light chain variable region CDR1 comprising amino acids having the sequence set forth in SEQ ID NO: 355; a light chain variable region CDR2 comprising amino acids having the sequence set forth in SEQ ID NO:356; and a light chain variable region CDR3 comprising amino acids having the sequence set forth in SEQ ID NO: 357;
wherein the antibody or antigen-binding portion thereof specifically binds to GPRC5D.

78. The antibody, or an antigen-binding portion thereof of claim 75, comprising:
(i) a heavy chain variable region CDR1 comprising amino acids having the sequence set forth in SEQ ID NO: 124; a heavy chain variable region CDR2 comprising amino acids having the sequence set forth in SEQ ID NO: 125; a heavy chain variable region CDR3 comprising amino acids having the sequence set forth in SEQ ID NO: 126; a light chain variable region CDR1 comprising amino acids having the sequence set forth in SEQ ID NO: 127; a light chain variable region CDR2 comprising amino acids having the sequence set forth in SEQ ID NO:128; and a light chain variable region CDR3 comprising amino acids having the sequence set forth in SEQ ID NO: 129;
(ii) a heavy chain variable region CDR1 comprising amino acids having the sequence set forth in SEQ ID NO: 130; a heavy chain variable region CDR2 comprising amino acids having the sequence set forth in SEQ ID NO: 131; a heavy chain variable region CDR3 comprising amino acids having the sequence set forth in SEQ ID NO: 132; a light chain variable region CDR1 comprising amino acids having the sequence set forth in SEQ ID NO: 133; a light chain variable region CDR2 comprising amino acids having the sequence set forth in SEQ ID NO:134; and a light chain variable region CDR3 comprising amino acids having the sequence set forth in SEQ ID NO: 135;
(iii) a heavy chain variable region CDR1 comprising amino acids having the sequence set forth in SEQ ID NO: 136; a heavy chain variable region CDR2 comprising amino acids having the sequence set forth in SEQ ID NO: 137; a heavy chain variable region CDR3 comprising amino acids having the sequence set forth in SEQ ID NO: 138; a light chain variable region CDR1 comprising amino acids having the sequence set forth in SEQ ID NO: 139; a light chain variable region CDR2 comprising amino acids having the sequence set forth in SEQ ID NO:140; and a light chain variable region CDR3 comprising amino acids having the sequence set forth in SEQ ID NO: 141;
(iv) a heavy chain variable region CDR1 comprising amino acids having the sequence set forth in SEQ ID NO: 142; a heavy chain variable region CDR2 comprising amino acids having the sequence set forth in SEQ ID NO: 143; a heavy chain variable region CDR3 comprising amino acids having the sequence set forth in SEQ ID NO: 144; a light chain variable region CDR1 comprising amino acids having the sequence set forth in SEQ ID NO: 145; a light chain variable region CDR2 comprising amino acids having the sequence set forth in SEQ ID NO:146; and a light chain variable region CDR3 comprising amino acids having the sequence set forth in SEQ ID NO: 147;
(v) a heavy chain variable region CDR1 comprising amino acids having the sequence set forth in SEQ ID NO: 148; a heavy chain variable region CDR2 comprising amino acids having the sequence set forth in SEQ ID NO: 149; a heavy chain variable region CDR3 comprising amino acids having the sequence set forth in SEQ ID NO: 150; a light chain variable region CDR1 comprising amino acids having the sequence set forth in SEQ ID NO: 151; a light chain variable region CDR2 comprising amino acids having the sequence set forth in SEQ ID NO:152; and a light chain variable region CDR3 comprising amino acids having the sequence set forth in SEQ ID NO: 153;
(vi) a heavy chain variable region CDR1 comprising amino acids having the sequence set forth in SEQ ID NO: 154; a heavy chain variable region CDR2 comprising amino acids having the sequence set forth in SEQ ID NO: 155; a heavy chain variable region CDR3 comprising amino acids having the sequence set forth in SEQ ID NO: 156; a light chain variable region CDR1 comprising amino acids having the sequence set forth in SEQ ID NO: 157; a light chain variable region CDR2 comprising amino acids having the sequence set forth in SEQ ID NO:158; and a light chain variable region CDR3 comprising amino acids having the sequence set forth in SEQ ID NO: 159;
(vii) a heavy chain variable region CDR1 comprising amino acids having the sequence set forth in SEQ ID NO: 160; a heavy chain variable region CDR2 comprising amino acids having the sequence set forth in SEQ ID NO: 161; a heavy chain variable region CDR3 comprising amino acids having the sequence set forth in SEQ ID NO: 162; a light chain variable region CDR1 comprising amino acids having the sequence set forth in SEQ ID NO: 163; a light chain variable region CDR2 comprising amino acids having the sequence set forth in SEQ ID NO:164; and a light chain variable region CDR3 comprising amino acids having the sequence set forth in SEQ ID NO: 165;
(viii) a heavy chain variable region CDR1 comprising amino acids having the sequence set forth in SEQ ID NO: 166; a heavy chain variable region CDR2 comprising amino acids having the sequence set forth in SEQ ID NO: 167; a heavy chain variable region CDR3 comprising amino acids having the sequence set forth in SEQ ID NO: 168; a light chain variable region CDR1 comprising amino acids having the sequence set forth in SEQ ID NO: 169; a light chain variable region CDR2 comprising amino acids having the sequence set forth in SEQ ID NO:170; and a light chain variable region CDR3 comprising amino acids having the sequence set forth in SEQ ID NO: 171;
(ix) a heavy chain variable region CDR1 comprising amino acids having the sequence set forth in SEQ ID NO: 172; a heavy chain variable region CDR2 comprising amino acids having the sequence set forth in SEQ ID NO: 173; a heavy chain variable region CDR3 comprising amino acids having the sequence set forth in SEQ ID NO: 174; a light chain variable region CDR1 comprising amino acids having the sequence set forth in SEQ ID NO: 175; a light chain variable region CDR2 comprising amino acids having the sequence set forth in SEQ ID NO:176; and a light chain variable region CDR3 comprising amino acids having the sequence set forth in SEQ ID NO: 177;
(x) a heavy chain variable region CDR1 comprising amino acids having the sequence set forth in SEQ ID NO: 178; a heavy chain variable region CDR2 comprising amino acids having the sequence set forth in SEQ ID NO: 179; a heavy chain variable region CDR3 comprising amino acids having the sequence set forth in SEQ ID NO: 180; a light chain variable region CDR1 comprising amino acids having the sequence set forth in SEQ ID NO: 181; a light chain variable region CDR2 comprising amino acids having the sequence set forth in SEQ ID NO:182; and a light chain variable region CDR3 comprising amino acids having the sequence set forth in SEQ ID NO: 183;
(xi) a heavy chain variable region CDR1 comprising amino acids having the sequence set forth in SEQ ID NO: 184; a heavy chain variable region CDR2 comprising amino acids having the sequence set forth in SEQ ID NO: 185; a heavy chain variable region CDR3 comprising amino acids having the sequence set forth in SEQ ID NO: 186; a light chain variable region CDR1 comprising amino acids having the sequence set forth in SEQ ID NO: 187; a light chain variable region CDR2 comprising amino acids having the sequence set forth in SEQ ID NO:188; and a light chain variable region CDR3 comprising amino acids having the sequence set forth in SEQ ID NO: 189;
(xii) a heavy chain variable region CDR1 comprising amino acids having the sequence set forth in SEQ ID NO: 190; a heavy chain variable region CDR2 comprising amino acids having the sequence set forth in SEQ ID NO: 191; a heavy chain variable region CDR3 comprising amino acids having the sequence set forth in SEQ ID NO: 192; a light chain variable region CDR1 comprising amino acids having the sequence set forth in SEQ ID NO: 193; a light chain variable region CDR2 comprising amino acids having the sequence set forth in SEQ ID NO:194; and a light chain variable region CDR3 comprising amino acids having the sequence set forth in SEQ ID NO: 195;
(xiii) a heavy chain variable region CDR1 comprising amino acids having the sequence set forth in SEQ ID NO: 196; a heavy chain variable region CDR2 comprising amino acids having the sequence set forth in SEQ ID NO: 197; a heavy chain variable region CDR3 comprising amino acids having the sequence set forth in SEQ ID NO: 198; a light chain variable region CDR1 comprising amino acids having the sequence set forth in SEQ ID NO: 199; a light chain variable region CDR2 comprising amino acids having the sequence set forth in SEQ ID NO:200; and a light chain variable region CDR3 comprising amino acids having the sequence set forth in SEQ ID NO: 201;
(xiv) a heavy chain variable region CDR1 comprising amino acids having the sequence set forth in SEQ ID NO: 202; a heavy chain variable region CDR2 comprising amino acids having the sequence set forth in SEQ ID NO: 203; a heavy chain variable region CDR3 comprising amino acids having the sequence set forth in SEQ ID NO: 204; a light chain variable region CDR1 comprising amino acids having the sequence set forth in SEQ ID NO: 205; a light chain variable region CDR2 comprising amino acids having the sequence set forth in SEQ ID NO:206; and a light chain variable region CDR3 comprising amino acids having the sequence set forth in SEQ ID NO: 207;
(xv) a heavy chain variable region CDR1 comprising amino acids having the sequence set forth in SEQ ID NO: 208; a heavy chain variable region CDR2 comprising amino acids having the sequence set forth in SEQ ID NO: 209; a heavy chain variable region CDR3 comprising amino acids having the sequence set forth in SEQ ID NO: 210; a light chain variable region CDR1 comprising amino acids having the sequence set forth in SEQ ID NO: 211; a light chain variable region CDR2 comprising amino acids having the sequence set forth in SEQ ID NO:212; and a light chain variable region CDR3 comprising amino acids having the sequence set forth in SEQ ID NO: 213;
(xvi) a heavy chain variable region CDR1 comprising amino acids having the sequence set forth in SEQ ID NO: 214; a heavy chain variable region CDR2 comprising amino acids having the sequence set forth in SEQ ID NO: 215; a heavy chain variable region CDR3 comprising amino acids having the sequence set forth in SEQ ID NO: 216; a light chain variable region CDR1 comprising amino acids having the sequence set forth in SEQ ID NO: 217; a light chain variable region CDR2 comprising amino acids having the sequence set forth in SEQ ID NO:218; and a light chain variable region CDR3 comprising amino acids having the sequence set forth in SEQ ID NO: 219;
(xvii) a heavy chain variable region CDR1 comprising amino acids having the sequence set forth in SEQ ID NO: 220; a heavy chain variable region CDR2 comprising amino acids having the sequence set forth in SEQ ID NO: 221; a heavy chain variable region CDR3 comprising amino acids having the sequence set forth in SEQ ID NO: 222; a light chain variable region CDR1 comprising amino acids having the sequence set forth in SEQ ID NO: 223; a light chain variable region CDR2 comprising amino acids having the sequence set forth in SEQ ID NO:224; and a light chain variable region CDR3 comprising amino acids having the sequence set forth in SEQ ID NO: 225;
(xviii) a heavy chain variable region CDR1 comprising amino acids having the sequence set forth in SEQ ID NO: 226; a heavy chain variable region CDR2 comprising amino acids having the sequence set forth in SEQ ID NO: 227; a heavy chain variable region CDR3 comprising amino acids having the sequence set forth in SEQ ID NO: 228; a light chain variable region CDR1 comprising amino acids having the sequence set forth in SEQ ID NO: 229; a light chain variable region CDR2 comprising amino acids having the sequence set forth in SEQ ID NO:230; and a light chain variable region CDR3 comprising amino acids having the sequence set forth in SEQ ID NO: 231;
(xix) a heavy chain variable region CDR1 comprising amino acids having the sequence set forth in SEQ ID NO: 232; a heavy chain variable region CDR2 comprising amino acids having the sequence set forth in SEQ ID NO: 233; a heavy chain variable region CDR3 comprising amino acids having the sequence set forth in SEQ ID NO: 234; a light chain variable region CDR1 comprising amino acids having the sequence set forth in SEQ ID NO: 235; a light chain variable region CDR2 comprising amino acids having the sequence set forth in SEQ ID NO:236; and a light chain variable region CDR3 comprising amino acids having the sequence set forth in SEQ ID NO: 237;
(xx) a heavy chain variable region CDR1 comprising amino acids having the sequence set forth in SEQ ID NO: 238; a heavy chain variable region CDR2 comprising amino acids having the sequence set forth in SEQ ID NO: 239; a heavy chain variable region CDR3 comprising amino acids having the sequence set forth in SEQ ID NO: 240; a light chain variable region CDR1 comprising amino acids having the sequence set forth in SEQ ID NO: 241; a light chain variable region CDR2 comprising amino acids having the sequence set forth in SEQ ID NO:242; and a light chain variable region CDR3 comprising amino acids having the sequence set forth in SEQ ID NO: 243;
(xxi) a heavy chain variable region CDR1 comprising amino acids having the sequence set forth in SEQ ID NO: 244; a heavy chain variable region CDR2 comprising amino acids having the sequence set forth in SEQ ID NO: 245; a heavy chain variable region CDR3 comprising amino acids having the sequence set forth in SEQ ID NO: 246; a light chain variable region CDR1 comprising amino acids having the sequence set forth in SEQ ID NO: 247; a light chain variable region CDR2 comprising amino acids having the sequence set forth in SEQ ID NO:248; and a light chain variable region CDR3 comprising amino acids having the sequence set forth in SEQ ID NO: 249;
(xxii) a heavy chain variable region CDR1 comprising amino acids having the sequence set forth in SEQ ID NO: 250; a heavy chain variable region CDR2 comprising amino acids having the sequence set forth in SEQ ID NO: 251; a heavy chain variable region CDR3 comprising amino acids having the sequence set forth in SEQ ID NO: 252; a light chain variable region CDR1 comprising amino acids having the sequence set forth in SEQ ID NO: 253; a light chain variable region CDR2 comprising amino acids having the sequence set forth in SEQ ID NO:254; and a light chain variable region CDR3 comprising amino acids having the sequence set forth in SEQ ID NO: 255;
(xxiii) a heavy chain variable region CDR1 comprising amino acids having the sequence set forth in SEQ ID NO: 256; a heavy chain variable region CDR2 comprising amino acids having the sequence set forth in SEQ ID NO: 257; a heavy chain variable region CDR3 comprising amino acids having the sequence set forth in SEQ ID NO: 258; a light chain variable region CDR1 comprising amino acids having the sequence set forth in SEQ ID NO: 259; a light chain variable region CDR2 comprising amino acids having the sequence set forth in SEQ ID NO:260; and a light chain variable region CDR3 comprising amino acids having the sequence set forth in SEQ ID NO: 261;
(xxiv) a heavy chain variable region CDR1 comprising amino acids having the sequence set forth in SEQ ID NO: 262; a heavy chain variable region CDR2 comprising amino acids having the sequence set forth in SEQ ID NO: 263; a heavy chain variable region CDR3 comprising amino acids having the sequence set forth in SEQ ID NO: 264; a light chain variable region CDR1 comprising amino acids having the sequence set forth in SEQ ID NO: 265; a light chain variable region CDR2 comprising amino acids having the sequence set forth in SEQ ID NO:266; and a light chain variable region CDR3 comprising amino acids having the sequence set forth in SEQ ID NO: 267;
(xxv) a heavy chain variable region CDR1 comprising amino acids having the sequence set forth in SEQ ID NO: 268; a heavy chain variable region CDR2 comprising amino acids having the sequence set forth in SEQ ID NO: 269; a heavy chain variable region CDR3 comprising amino acids having the sequence set forth in SEQ ID NO: 270; a light chain variable region CDR1 comprising amino acids having the sequence set forth in SEQ ID NO: 271; a light chain variable region CDR2 comprising amino acids having the sequence set forth in SEQ ID NO:272; and a light chain variable region CDR3 comprising amino acids having the sequence set forth in SEQ ID NO: 273;
(xxvi) a heavy chain variable region CDR1 comprising amino acids having the sequence set forth in SEQ ID NO: 280; a heavy chain variable region CDR2 comprising amino acids having the sequence set forth in SEQ ID NO: 281; a heavy chain variable region CDR3 comprising amino acids having the sequence set forth in SEQ ID NO: 282; a light chain variable region CDR1 comprising amino acids having the sequence set forth in SEQ ID NO: 283; a light chain variable region CDR2 comprising amino acids having the sequence set forth in SEQ ID NO:284; and a light chain variable region CDR3 comprising amino acids having the sequence set forth in SEQ ID NO: 285;
(xxvii) a heavy chain variable region CDR1 comprising amino acids having the sequence set forth in SEQ ID NO: 292; a heavy chain variable region CDR2 comprising amino acids having the sequence set forth in SEQ ID NO: 293; a heavy chain variable region CDR3 comprising amino acids having the sequence set forth in SEQ ID NO: 294; a light chain variable region CDR1 comprising amino acids having the sequence set forth in SEQ ID NO: 295; a light chain variable region CDR2 comprising amino acids having the sequence set forth in SEQ ID NO:296; and a light chain variable region CDR3 comprising amino acids having the sequence set forth in SEQ ID NO: 297;
(xxviii) a heavy chain variable region CDR1 comprising amino acids having the sequence set forth in SEQ ID NO: 303; a heavy chain variable region CDR2 comprising amino acids having the sequence set forth in SEQ ID NO: 304; a heavy chain variable region CDR3 comprising amino acids having the sequence set forth in SEQ ID NO: 305; a light chain variable region CDR1 comprising amino acids having the sequence set forth in SEQ ID NO: 306; a light chain variable region CDR2 comprising amino acids having the sequence set forth in SEQ ID NO: 307; and a light chain variable region CDR3 comprising amino acids having the sequence set forth in SEQ ID NO: 308;
(xxix) a heavy chain variable region CDR1 comprising amino acids having the sequence set forth in SEQ ID NO: 316; a heavy chain variable region CDR2 comprising amino acids having the sequence set forth in SEQ ID NO: 317; a heavy chain variable region CDR3 comprising amino acids having the sequence set forth in SEQ ID NO: 318; a light chain variable region CDR1 comprising amino acids having the sequence set forth in SEQ ID NO: 319; a light chain variable region CDR2 comprising amino acids having the sequence set forth in SEQ ID NO: 320; and a light chain variable region CDR3 comprising amino acids having the sequence set forth in SEQ ID NO: 321;
(xxx) a heavy chain variable region CDR1 comprising amino acids having the sequence set forth in SEQ ID NO: 328; a heavy chain variable region CDR2 comprising amino acids having the sequence set forth in SEQ ID NO: 329; a heavy chain variable region CDR3 comprising amino acids having the sequence set forth in SEQ ID NO: 330; a light chain variable region CDR1 comprising amino acids having the sequence set forth in SEQ ID NO: 331; a light chain variable region CDR2 comprising amino acids having the sequence set forth in SEQ ID NO: 332; and a light chain variable region CDR3 comprising amino acids having the sequence set forth in SEQ ID NO: 333;
(xxxi) a heavy chain variable region CDR1 comprising amino acids having the sequence set forth in SEQ ID NO: 340; a heavy chain variable region CDR2 comprising amino acids having the sequence set forth in SEQ ID NO: 341; a heavy chain variable region CDR3 comprising amino acids having the sequence set forth in SEQ ID NO: 342; a light chain variable region CDR1 comprising amino acids having the sequence set forth in SEQ ID NO: 343; a light chain variable region CDR2 comprising amino acids having the sequence set forth in SEQ ID NO: 344; and a light chain variable region CDR3 comprising amino acids having the sequence set forth in SEQ ID NO: 345; or
(xxxii) a heavy chain variable region CDR1 comprising amino acids having the sequence set forth in SEQ ID NO: 352; a heavy chain variable region CDR2 comprising amino acids having the sequence set forth in SEQ ID NO: 353; a heavy chain variable region CDR3 comprising amino acids having the sequence set forth in SEQ ID NO: 354; a light chain variable region CDR1 comprising amino acids having the sequence set forth in SEQ ID NO: 355; a light chain variable region CDR2 comprising amino acids having the sequence set forth in SEQ ID NO: 356; and a light chain variable region CDR3 comprising amino acids having the sequence set forth in SEQ ID NO: 357.

79. The antibody, or an antigen-binding fragment thereof of claim 75, comprising a heavy chain variable region comprising an amino acid sequence selected from the group consisting of SEQ ID NOS: 1, 5, 9, 13, 17, 21, 25, 29, 33, 37, 41, 45, 49, 53, 57, 61, 65, 69, 73, 77, 81, 85, 89, 93, 274, 286, 298, 310, 322, 334, 346 and 358.

80. The antibody, or an antigen-binding fragment thereof of claim 75, comprising a light chain variable region comprising an amino acid sequence selected from the group consisting of SEQ ID NOS: 2, 6, 10, 14, 18, 22, 26, 30, 34, 38, 42, 46, 50, 54, 58, 62, 66, 70, 74, 78, 82, 86, 90, 94, 275, 287, 299, 311, 323, 335, 347 and 359.

81. The antibody, or an antigen-binding fragment thereof of claim 75, comprising
(a) a heavy chain variable region comprising an amino acid sequence selected from the group consisting of SEQ ID NOS: 1, 5, 9, 13, 17, 21, 25, 29, 33, 37, 41, 45, 49, 53, 57, 61, 65, 69, 73, 77, 81, 85, 89, 93, 274, 286, 298, 310, 322, 334, 346 and 358; and
(b) a light chain variable region comprising an amino acid sequence selected from the group consisting of SEQ ID NOS: 2, 6, 10, 14, 18, 22, 26, 30, 34, 38, 42, 46, 50, 54, 58, 62, 66, 70, 74, 78, 82, 86, 90, 94, 275, 287, 299, 311, 323, 335, 347 and 359.

82. The antibody, or an antigen-binding fragment thereof of claim 75, comprising a heavy chain variable region and a light chain variable region, wherein the heavy chain variable region and the light chain variable region are selected from the group consisting of:
(i) a heavy chain variable region comprising amino acids having the sequence set forth in SEQ ID NO:1, and a light chain variable region comprising amino acids having the sequence set forth in SEQ ID NO:2;
(ii) a heavy chain variable region comprising amino acids having the sequence set forth in SEQ ID NO:5, and a light chain variable region comprising amino acids having the sequence set forth in SEQ ID NO:6;
(iii) a heavy chain variable region comprising amino acids having the sequence set forth in SEQ ID NO:9, and a light chain variable region comprising amino acids having the sequence set forth in SEQ ID NO:10;
(iv) a heavy chain variable region comprising amino acids having the sequence set forth in SEQ ID NO:13, and a light chain variable region comprising amino acids having the sequence set forth in SEQ ID NO:14;
(v) a heavy chain variable region comprising amino acids having the sequence set forth in SEQ ID NO:17, and a light chain variable region comprising amino acids having the sequence set forth in SEQ ID NO:18;
(vi) a heavy chain variable region comprising amino acids having the sequence set forth in SEQ ID NO:21, and a light chain variable region comprising amino acids having the sequence set forth in SEQ ID NO:22;
(vii) a heavy chain variable region comprising amino acids having the sequence set forth in SEQ ID NO:25, and a light chain variable region comprising amino acids having the sequence set forth in SEQ ID NO:26;
(viii) a heavy chain variable region comprising amino acids having the sequence set forth in SEQ ID NO:29, and a light chain variable region comprising amino acids having the sequence set forth in SEQ ID NO:30;
(ix) a heavy chain variable region comprising amino acids having the sequence set forth in SEQ ID NO:33, and a light chain variable region comprising amino acids having the sequence set forth in SEQ ID NO:34;
(x) a heavy chain variable region comprising amino acids having the sequence set forth in SEQ ID NO:37, and a light chain variable region comprising amino acids having the sequence set forth in SEQ ID NO:38;
(xi) a heavy chain variable region comprising amino acids having the sequence set forth in SEQ ID NO:41, and a light chain variable region comprising amino acids having the sequence set forth in SEQ ID NO:42;
(xii) a heavy chain variable region comprising amino acids having the sequence set forth in SEQ ID NO:45, and a light chain variable region comprising amino acids having the sequence set forth in SEQ ID NO:46;
(xiii) a heavy chain variable region comprising amino acids having the sequence set forth in SEQ ID NO:49, and a light chain variable region comprising amino acids having the sequence set forth in SEQ ID NO:50;
(xiv) a heavy chain variable region comprising amino acids having the sequence set forth in SEQ ID NO:53, and a light chain variable region comprising amino acids having the sequence set forth in SEQ ID NO:54;
(xv) a heavy chain variable region comprising amino acids having the sequence set forth in SEQ ID NO:57, and a light chain variable region comprising amino acids having the sequence set forth in SEQ ID NO:58;
(xvi) a heavy chain variable region comprising amino acids having the sequence set forth in SEQ ID NO:61, and a light chain variable region comprising amino acids having the sequence set forth in SEQ ID NO:62;
(xvii) a heavy chain variable region comprising amino acids having the sequence set forth in SEQ ID NO:65, and a light chain variable region comprising amino acids having the sequence set forth in SEQ ID NO:66;
(xviii) a heavy chain variable region comprising amino acids having the sequence set forth in SEQ ID NO:69, and a light chain variable region comprising amino acids having the sequence set forth in SEQ ID NO:70;
(xix) a heavy chain variable region comprising amino acids having the sequence set forth in SEQ ID NO:73, and a light chain variable region comprising amino acids having the sequence set forth in SEQ ID NO:74;
(xx) a heavy chain variable region comprising amino acids having the sequence set forth in SEQ ID NO:77, and a light chain variable region comprising amino acids having the sequence set forth in SEQ ID NO:78;
(xxi) a heavy chain variable region comprising amino acids having the sequence set forth in SEQ ID NO:81, and a light chain variable region comprising amino acids having the sequence set forth in SEQ ID NO:82;
(xxii) a heavy chain variable region comprising amino acids having the sequence set forth in SEQ ID NO:85, and a light chain variable region comprising amino acids having the sequence set forth in SEQ ID NO:86;
(xxiii) a heavy chain variable region comprising amino acids having the sequence set forth in SEQ ID NO:89, and a light chain variable region comprising amino acids having the sequence set forth in SEQ ID NO:90;
(xxiv) a heavy chain variable region comprising amino acids having the sequence set forth in SEQ ID NO:93, and a light chain variable region comprising amino acids having the sequence set forth in SEQ ID NO:94;
(xxv) a heavy chain variable region comprising amino acids having the sequence set forth in SEQ ID NO:274, and a light chain variable region comprising amino acids having the sequence set forth in SEQ ID NO:275;
(xxvi) a heavy chain variable region comprising amino acids having the sequence set forth in SEQ ID NO:286, and a light chain variable region comprising amino acids having the sequence set forth in SEQ ID NO:287;
(xxvii) a heavy chain variable region comprising amino acids having the sequence set forth in SEQ ID NO:298, and a light chain variable region comprising amino acids having the sequence set forth in SEQ ID NO:299;
(xxviii) a heavy chain variable region comprising amino acids having the sequence set forth in SEQ ID NO:310, and a light chain variable region comprising amino acids having the sequence set forth in SEQ ID NO:311;
(xxix) a heavy chain variable region comprising amino acids having the sequence set forth in SEQ ID NO:322, and a light chain variable region comprising amino acids having the sequence set forth in SEQ ID NO:323;
(xxx) a heavy chain variable region comprising amino acids having the sequence set forth in SEQ ID NO:334, and a light chain variable region comprising amino acids having the sequence set forth in SEQ ID NO:335;
(xxxi) a heavy chain variable region comprising amino acids having the sequence set forth in SEQ ID NO:346, and a light chain variable region comprising amino acids having the sequence set forth in SEQ ID NO:347; or
(xxxii) a heavy chain variable region comprising amino acids having the sequence set forth in SEQ ID NO:358, and a light chain variable region comprising amino acids the sequence set forth in SEQ ID NO:359.

83. The antibody or antigen-binding fragment thereof of claim 75, comprising:
(i) a heavy chain variable region comprising amino acids having a sequence set forth in SEQ ID NO:1, and a light chain variable region comprising amino acids having a sequence set forth in SEQ ID NO:2;
(ii) a heavy chain variable region comprising amino acids having a sequence set forth in SEQ ID NO:5, and a light chain variable region comprising amino acids having a sequence set forth in SEQ ID NO:6;
(iii) a heavy chain variable region comprising amino acids having a sequence set forth in SEQ ID NO:9, and a light chain variable region comprising amino acids having a sequence set forth in SEQ ID NO:10;
(iv) a heavy chain variable region comprising amino acids having a sequence set forth in SEQ ID NO:13, and a light chain variable region comprising amino acids having a sequence set forth in SEQ ID NO:14;
(v) a heavy chain variable region comprising amino acids having a sequence set forth in SEQ ID NO:17, and a light chain variable region comprising amino acids having a sequence set forth in SEQ ID NO:18;
(vi) a heavy chain variable region comprising amino acids having a sequence set forth in SEQ ID NO:21, and a light chain variable region comprising amino acids having a sequence set forth in SEQ ID NO:22;
(vii) a heavy chain variable region comprising amino acids having a sequence set forth in SEQ ID NO:25, and a light chain variable region comprising amino acids having a sequence set forth in SEQ ID NO:26;
(viii) a heavy chain variable region comprising amino acids having a sequence set forth in SEQ ID NO:29, and a light chain variable region comprising amino acids having a sequence set forth in SEQ ID NO:30;
(ix) a heavy chain variable region comprising amino acids having a sequence set forth in SEQ ID NO:33, and a light chain variable region comprising amino acids having a sequence set forth in SEQ ID NO:34;
(x) a heavy chain variable region comprising amino acids having a sequence set forth in SEQ ID NO:37, and a light chain variable region comprising amino acids having a sequence set forth in SEQ ID NO:38;
(xi) a heavy chain variable region comprising amino acids having a sequence set forth in SEQ ID NO:41, and a light chain variable region comprising amino acids having a sequence set forth in SEQ ID NO:42;
(xii) a heavy chain variable region comprising amino acids having a sequence set forth in SEQ ID NO:45, and a light chain variable region comprising amino acids having a sequence set forth in SEQ ID NO:46;
(xiii) a heavy chain variable region comprising amino acids having a sequence set forth in SEQ ID NO:49, and a light chain variable region comprising amino acids having a sequence set forth in SEQ ID NO:50;
(xiv) a heavy chain variable region comprising amino acids having a sequence set forth in SEQ ID NO:53, and a light chain variable region comprising amino acids having a sequence set forth in SEQ ID NO:54;
(xv) a heavy chain variable region comprising amino acids having a sequence set forth in SEQ ID NO:57, and a light chain variable region comprising amino acids having a sequence set forth in SEQ ID NO:58;
(xvi) a heavy chain variable region comprising amino acids having a sequence set forth in SEQ ID NO:61, and a light chain variable region comprising amino acids having a sequence set forth in SEQ ID NO:62;
(xvii) a heavy chain variable region comprising amino acids having a sequence set forth in SEQ ID NO:65, and a light chain variable region comprising amino acids having a sequence set forth in SEQ ID NO:66;
(xviii) a heavy chain variable region comprising amino acids having a sequence set forth in SEQ ID NO:69, and a light chain variable region comprising amino acids having a sequence set forth in SEQ ID NO:70;
(xix) a heavy chain variable region comprising amino acids having a sequence set forth in SEQ ID NO:73, and a light chain variable region comprising amino acids having a sequence set forth in SEQ ID NO:74;
(xx) a heavy chain variable region comprising amino acids having a sequence set forth in SEQ ID NO:77, and a light chain variable region comprising amino acids having a sequence set forth in SEQ ID NO:78;
(xxi) a heavy chain variable region comprising amino acids having a sequence set forth in SEQ ID NO:81, and a light chain variable region comprising amino acids having a sequence set forth in SEQ ID NO:82;
(xxii) a heavy chain variable region comprising amino acids having a sequence set forth in SEQ ID NO:85, and a light chain variable region comprising amino acids having a sequence set forth in SEQ ID NO:86;
(xxiii) a heavy chain variable region comprising amino acids having a sequence set forth in SEQ ID NO:89, and a light chain variable region comprising amino acids having a sequence set forth in SEQ ID NO:90;
(xxiv) a heavy chain variable region comprising amino acids having a sequence set forth in SEQ ID NO:93, and a light chain variable region comprising amino acids having a sequence set forth in SEQ ID NO:94;
(xxv) a heavy chain variable region comprising amino acids having a sequence set forth in SEQ ID NO:274, and a light chain variable region comprising amino acids having a set forth in SEQ ID NO:275;
(xxvi) a heavy chain variable region comprising amino acids having a sequence set forth in SEQ ID NO:286, and a light chain variable region comprising amino acids having a sequence set forth in SEQ ID NO:287;
(xxvii) a heavy chain variable region comprising amino acids having a sequence set forth in SEQ ID NO:298, and a light chain variable region comprising amino acids having a sequence set forth in SEQ ID NO:299;
(xxviii) a heavy chain variable region comprising amino acids having a sequence set forth in SEQ ID NO:310, and a light chain variable region comprising amino acids having a sequence set forth in SEQ ID NO:311;
(xxix) a heavy chain variable region comprising amino acids having a sequence set forth in SEQ ID NO:322, and a light chain variable region comprising amino acids having a sequence set forth in SEQ ID NO:323;
(xxx) a heavy chain variable region comprising amino acids having a sequence set forth in SEQ ID NO:334, and a light chain variable region comprising amino acids having a sequence set forth in SEQ ID NO:335;
(xxxi) a heavy chain variable region comprising amino acids having a sequence set forth in SEQ ID NO:346, and a light chain variable region comprising amino acids having a sequence set forth in SEQ ID NO:347; or
(xxxii) a heavy chain variable region comprising amino acids having a sequence set forth in SEQ ID NO:358, and a light chain variable region comprising amino acids having a sequence set forth in SEQ ID NO:359.

84. The antibody, or an antigen-binding fragment thereof of claim 75, comprising a heavy chain variable region comprising an amino acid sequence selected from the group consisting of SEQ ID NOS: 1, 5, 9, 13, 17, 21, 25, 29, 33, 37, 41, 45, 49, 53, 57, 61, 65, 69, 73, 77, 81, 85, 89, 93, 274, 286, 298, 310, 322, 334, 346 and 358.

85. The antibody, or an antigen-binding fragment thereof of claim 75, comprising a light chain variable region comprising an amino acid sequence selected from the group consisting of SEQ ID NOS: 2, 6, 10, 14, 18, 22, 26, 30, 34, 38, 42, 46, 50, 54, 58, 62, 66, 70, 74, 78, 82, 86, 90, 94, 275, 287, 299, 311, 323, 335, 347 and 359.

86. The antibody, or an antigen-binding fragment thereof of claim 75, comprising
(a) a heavy chain variable region comprising an amino acid sequence selected from the group consisting of SEQ ID NOS: 1, 5, 9, 13, 17, 21, 25, 29, 33, 37, 41, 45, 49, 53, 57, 61, 65, 69, 73, 77, 81, 85, 89, 93, 274, 286, 298, 310, 322, 334, 346 and 358; and
(b) a light chain variable region comprising an amino acid sequence selected from the group consisting of SEQ ID NOS: 2, 6, 10, 14, 18, 22, 26, 30, 34, 38, 42, 46, 50, 54, 58, 62, 66, 70, 74, 78, 82, 86, 90, 94, 275, 287, 299, 311, 323, 335, 347 and 359.

87. The antibody or antigen-binding fragment thereof of claim 73, wherein the GPRC5D comprises the amino acid sequence set forth in SEQ ID NO:97.

88. The antibody or antigen-binding fragment thereof of claim 73, wherein the antibody or antigen-binding fragment thereof binds to one, two, three or four epitope region selected from the group consisting of an epitope region in N-terminal region comprising amino acids 1-27 of SEQ ID NO:97, an epitope region in ECL1 region comprising amino acids 85-93 of SEQ ID NO:97, an epitope region in ECL2 region comprising amino acids 145-167 of SEQ ID NO:97, and an epitope region in ECL3 region comprising amino acids 226-239 of SEQ ID NO:97.

89. The antibody or antigen-binding fragment thereof of claim 73, wherein the antibody or antigen-binding fragment thereof binds to an epitope region selected from the group consisting of:
an epitope region comprising amino acids 16-23 of SEQ ID NO:97,
an epitope region comprising amino acids 15-23 of SEQ ID NO:97,
an epitope region comprising amino acids 16-25 of SEQ ID NO:97,
an epitope region comprising amino acids 10-17 of SEQ ID NO:97,
an epitope region comprising amino acids 5-17 of SEQ ID NO:97,
an epitope region comprising amino acids 85-95 of SEQ ID NO:97,
an epitope region comprising amino acids 157-164 of SEQ ID NO:97,
an epitope region comprising amino acids 157-167 of SEQ ID NO:97,
an epitope region comprising amino acids 230-237 of SEQ ID NO:97,
an epitope region comprising amino acids 229-237 of SEQ ID NO:97,
an epitope region comprising amino acids 230-243 of SEQ ID NO:97, and
an epitope region comprising amino acids 227-237 of SEQ ID NO:97.

90. The antibody, or antigen-binding fragment thereof of claim 73, comprising an amino acid sequence selected from the group consisting of SEQ ID NOS: 100-123, 276, 288, 300, 312, 324, 336, 348 and 360.

91. An anti-GPRC5D scFv antibody comprising a light chain variable region, a heavy chain variable region, and a peptide linker.

92. The antibody, or antigen-binding portion thereof of claim 73, wherein the antibody, or antigen-binding portion thereof binds to human GPRC5D with a binding affinity (K_{d}) of from about 1×10⁻⁹ M to about 1×10⁻⁸ M.

93. The antibody or antigen-binding fragment thereof of claim 73, wherein the antibody comprises a human variable region framework region.

94. The antibody or antigen-binding fragment thereof of claim 73, which is a fully human or an antigen-binding fragment thereof.

95. The antibody or antigen-binding fragment thereof of claim 73, which is a chimeric or humanized antibody or an antigen-binding fragment thereof.

96. The antibody or antigen-binding fragment thereof of claim 73, wherein the antigen-binding fragment of the antibody is an Fab, Fab′, F(ab′)2, Fv or single chain Fv (scFv).

97. A composition comprising the antibody or antigen-binding fragment thereof of claim 73, and a pharmaceutically acceptable carrier.

98. An immunoconjugate comprising the antibody or antigen-binding fragment thereof of claim 73, linked to a therapeutic agent.

99. The immunoconjugate of claim 98, wherein said therapeutic agent is a drug, cytotoxin, or a radioactive isotope.

100. A composition comprising the immunoconjugate of claim 98 and a pharmaceutically acceptable carrier.

101. A bispecific molecule comprising the antibody or antigen-binding fragment thereof of claim 73, linked to a second functional moiety.

102. The bispecific molecule of claim 101, wherein the second functional moiety has a different binding specificity than said antibody or antigen binding fragment thereof.

103. The bispecific molecule of claim 102, wherein the second functional moiety has a binding specificity for an immune cell.

104. The bispecific molecule of claim 102, wherein the second functional moiety has a binding specificity for CD3.

105. A composition comprising the bispecific molecule of claim 101 and a pharmaceutically acceptable carrier.

106. An nucleic acid that encodes an antibody or antigen-binding fragment thereof of claim 73.

107. An expression vector comprising the nucleic acid molecule of claim 106.

108. A host cell comprising the expression vector of claim 107.

109. A method for detecting GPRC5D in a whole cell or tissue, comprising:
contacting a cell or tissue with the antibody or antigen-binding fragment thereof of claim 73, wherein said antibody or antigen-binding fragment thereof comprises a detectable label; and
determining the amount of the labeled antibody or antigen-binding fragment thereof bound to said cell or tissue by measuring the amount of detectable label associated with said cell or tissue, wherein the amount of bound antibody or antigen-binding fragment thereof indicates the amount of GPRC5D in said cell or tissue.

110. A method of treating a tumor in a subject, comprising administering an effective amount of the antibody or antigen-binding fragment thereof of claim 73 to the subject, thereby inducing death of a tumor cell in the subject.

111. The method of claim 110, wherein the method reduces the number of the tumor cells, reduces the tumor size, or eradicates the tumor in the subject.

112. The method of claim 110, wherein the tumor is multiple myeloma or Waldenstrom's Macroglobulinemia.

113. The method of claim 112, wherein the tumor is multiple myeloma.

114. The method of 110, wherein the subject is a human.

115. A kit for treating a tumor, comprising the antibody or antigen-binding fragment thereof of claim 73.

116. The kit of claim 115, wherein the kit further comprises written instructions for using the antibody or antigen-binding fragment thereof for treating a subject having a tumor.

117. The kit of claim 115, wherein the tumor is multiple myeloma or Waldenstrom's Macroglobulinemia.

118. The kit of claim 115, wherein the tumor is multiple myeloma.


SEQUENCE LISTING (415 sequences)

SEQ ID NO: 1 - protein, 120 aa, synthetic construct
  REGION 1..120: note = Description of Artificial Sequence: Synthetic polypeptide
QVQLVQSGSELKKPGASVRVSCTASGYTFTSYYMHWVRQAPGQGLEWMGVINPNAGSTRYAQKFQGRVTMSTDTSTSTAYMDLSSLRSEDTAVYYCARGMYRSLLFYDPWGQGTLVTVSS

SEQ ID NO: 2 - protein, 111 aa, synthetic construct
  REGION 1..111: note = Description of Artificial Sequence: Synthetic polypeptide
QSVLTQPPSVSAAPGQKVTIPCSGSRSNVGNYYVSWYQQLPGTAPKLLIYDNNKRPSGIPDRFSGSKSGTSATLGITGLQTGDEADYFCGTWDGSLSAHVFGTGTKVTVLG

SEQ ID NO: 3 - DNA, 360 bp, synthetic construct
  misc_feature 1..360: note = Description of Artificial Sequence: Synthetic polynucleotide
  source 1..360: mol_type = other DNA
caggtgcagctggtgcagtctgggtctgagttgaagaagcctggggcctcagtcagagtctcctgcacggcttctggatacaccttcaccagttactatatgcactgggtgcgacaggcccctggacaagggcttgagtggatgggagtaatcaaccctaatgctggcagcacaagatacgcacagaaattccagggcagagtcaccatgagcactgacacgtccacgagcacagcctacatggacctgagcagtctgagatctgaggacacggccgtgtattactgtgcgcgcggtatgtaccgttctctgctgttctacgatccgtggggtcaaggtactctggtgaccgtctcctca

SEQ ID NO: 4 - DNA, 333 bp, synthetic construct
  misc_feature 1..333: note = Description of Artificial Sequence: Synthetic polynucleotide
  source 1..333: mol_type = other DNA
cagtctgtgttgacgcagccgccctcagtgtctgcggccccaggacagaaggtcaccatcccctgctctggaagccgttccaacgttgggaattattatgtgtcctggtaccagcaactcccaggaacagcccccaaactcctcatttatgacaataataagcgaccctcagggattcctgaccgattctctggctccaagtctggcacgtcagccaccctgggcatcaccggactccagactggggacgaggccgattatttctgcggaacatgggatggcagcctgagtgcccatgtcttcggaactgggaccaaggtcaccgtcctaggt

SEQ ID NO: 5 - protein, 116 aa, synthetic construct
  REGION 1..116: note = Description of Artificial Sequence: Synthetic polypeptide
EVQLVESGGGLVQPGGSLRLSCAASGFTFSNYAMSWVRQAPGKGLEWVSAISGSGNTYYADSVKGRFTISRDNSKNTLYLQMNSLRAEDTAVYYCARGSVRYTDIWGQGTLVTVSS

SEQ ID NO: 6 - protein, 113 aa, synthetic construct
  REGION 1..113: note = Description of Artificial Sequence: Synthetic polypeptide
NFMLTQPHSVSESPGKTVSISCTRTSGAIAGAYVQWFQQRPGSAPTTVIYDDNKRPSGVPDRFSGSIDKSSNSASLTISGLKTEDEADYYCQSYDYDSSNVLFGGGTKLTVLG

SEQ ID NO: 7 - DNA, 348 bp, synthetic construct
  misc_feature 1..348: note = Description of Artificial Sequence: Synthetic polynucleotide
  source 1..348: mol_type = other DNA
gaggtgcagctggtggagtctgggggaggcttggtacagcctggggggtccctgagactctcctgtgcagcctctggattcacctttagcaactatgccatgagttgggtccgccaggctccagggaagggactggagtgggtctcagctattagtggtagtggtaacacatactacgcagactccgtgaagggccggttcaccatctccagagacaattccaagaacacgctgtatctgcaaatgaacagcctgagagccgaggacacggccgtatattactgtgcgcgcggttctgttcgttacactgatatctggggtcaaggtactctggtgaccgtctcctca

SEQ ID NO: 8 - DNA, 339 bp, synthetic construct
  misc_feature 1..339: note = Description of Artificial Sequence: Synthetic polynucleotide
  source 1..339: mol_type = other DNA
aattttatgctgactcagccccactcagtgtcggagtctccggggaagacggtaagcatctcctgcacccgcaccagtggcgccattgccggcgcctatgtgcagtggttccagcagcgcccgggcagtgcccccaccactgtgatctatgacgataacaaaagaccctctggggtccctgatcggttctctgggtccatcgacaagtcctccaactctgcctccctcaccatctctggactgaagactgaggacgaggctgactattattgtcagtcttatgattatgatagcagcaatgtgctattcggcggagggaccaagctgaccgtcctaggt

SEQ ID NO: 9 - protein, 119 aa, synthetic construct
  REGION 1..119: note = Description of Artificial Sequence: Synthetic polypeptide
EVQLVQSGGGLVQPGGSLRLSCATSGFTFNNYWMSWVRQAPGKGLEWVANIKQDGSEKYYADSVRGRFTISRDNAKNSLSLQLNNLRAEDTAVYYCARSMSTAWGYDEWGQGTLVTVSS

SEQ ID NO: 10 - protein, 108 aa, synthetic construct
  REGION 1..108: note = Description of Artificial Sequence: Synthetic polypeptide
DIQLTQSPSSLSASVGDRVTITCRASQSISSYLNWYQQKPGKAPKLLIYAASSLQSGVPSRFSGSGSGTDFTLTISSLQPADFATYYCQQSYSVPYTFGQGTKLEIKR

SEQ ID NO: 11 - DNA, 357 bp, synthetic construct
  misc_feature 1..357: note = Description of Artificial Sequence: Synthetic polynucleotide
  source 1..357: mol_type = other DNA
gaggtgcagctggtgcagtctgggggaggcttggtccagcctggggggtccctgagactctcctgtgcaacctctggattcacctttaataactattggatgagttgggtccgccaggctccagggaaggggctggagtgggtggccaacataaagcaagatggaagtgagaaatactacgcggactctgtgaggggccgattcaccatctccagagacaacgccaagaactcactgtctctgcaattgaacaacctgagagccgaggacacggccgtgtattactgtgcgcgctctatgtctactgcttggggttacgatgaatggggtcaaggtactctggtgaccgtctcctca

SEQ ID NO: 12 - DNA, 324 bp, synthetic construct
  misc_feature 1..324: note = Description of Artificial Sequence: Synthetic polynucleotide
  source 1..324: mol_type = other DNA
gacatccagttgacccagtctccatcctccctgtctgcatctgtcggagacagagtcaccatcacttgccgggcaagtcagagcattagcagctatttaaattggtatcaacagaaaccagggaaagcccctaagctcctgatctatgctgcatccagtttgcaaagtggggtcccatcaaggttcagtggcagtggatctgggacagatttcactctcaccatcagcagtctgcaacctgcagattttgcaacttactactgtcaacagagttacagtgtcccgtacacttttggccaggggaccaagctggagatcaaacgt

SEQ ID NO: 13 - protein, 121 aa, synthetic construct
  REGION 1..121: note = Description of Artificial Sequence: Synthetic polypeptide
QVQLVQSGAEVKKPGASVKVSCKASGYTFTSYYMHWVRQAPGQGLEWMGIINPSGGSTRYAQKFQGRVTMTRDTSTSTVYMELSSLRSEDTAVYYCARGSSRWGGWTGDYWGQGTLVTVSS

SEQ ID NO: 14 - protein, 111 aa, synthetic construct
  REGION 1..111: note = Description of Artificial Sequence: Synthetic polypeptide
QSALTQPASVSGSPGQSITISCTGTSSDVGGYNFVSWYQQHPGKAPKVMIYDVSKRPSGISNRFSGSKSGNTASLTISGLQVEDEAEYYCSSYTSTRTVIFAGGTKVTVLG

SEQ ID NO: 15 - DNA, 363 bp, synthetic construct
  misc_feature 1..363: note = Description of Artificial Sequence: Synthetic polynucleotide
  source 1..363: mol_type = other DNA
caggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaaggtacgcacagaagttccagggcagagtcaccatgaccagggacacgtcaacgagcacagtctacatggagctgagcagcctgagatctgaggacacggccgtgtattactgtgcgcgcggttcttctcgctggggtggttggactggtgattactggggtcaaggtactctggtgaccgtctcctca

SEQ ID NO: 16 - DNA, 333 bp, synthetic construct
  misc_feature 1..333: note = Description of Artificial Sequence: Synthetic polynucleotide
  source 1..333: mol_type = other DNA
caatctgccctgactcagcctgcctccgtgtctgggtctcctggacagtcgatcaccatctcctgcactggaaccagcagtgacgttggtggttataactttgtctcctggtaccaacagcacccaggcaaagcccccaaagtcatgatttatgatgtcagtaagcggccctcagggatttctaatcgcttctctggctccaagtctggcaacacggcctccctgaccatctctgggctccaggttgaggacgaggctgaatattactgcagctcatatacaagcactagaactgtgatattcgccggagggaccaaggtcaccgtcctaggt

SEQ ID NO: 17 - protein, 117 aa, synthetic construct
  REGION 1..117: note = Description of Artificial Sequence: Synthetic polypeptide
EVQLVETGGGLVQPGGSLRLSCAASGSTFSSYAMSWVRQAPGKGLEWVSAISGRGRSTYYADSVKGRFTISRDNSKNTLYLQMNSLRAEDTAVYYCARYYKSSKDHWGQGTLVTVSS

SEQ ID NO: 18 - protein, 111 aa, synthetic construct
  REGION 1..111: note = Description of Artificial Sequence: Synthetic polypeptide
  MOD_RES 37: note = Any amino acid
QSVLTQPPSLSGAPGQRVTISCSGSRSNIGTNYVSWXQQLPGTAPKLLIYRNHQWPSGVPDRFTGSKSGTSASLAISGLRSEDEADYYCAAWDDNLSGVVFGGGTKLTVLG

SEQ ID NO: 19 - DNA, 351 bp, synthetic construct
  misc_feature 1..351: note = Description of Artificial Sequence: Synthetic polynucleotide
  source 1..351: mol_type = other DNA
gaggtgcagctggtggagactgggggaggcttggtacagcctggggggtccctgagactctcctgtgcagcctctggatccacctttagcagctatgccatgagctgggtccgccaggctccagggaaggggctggagtgggtctcagctattagtggtcgtggtcgtagcacatactacgcagactccgtgaagggccggttcaccatctccagagacaattccaagaacacgctgtatctgcaaatgaacagcctgagagccgaggacacggccgtatattactgtgcgcgctactacaaatcttctaaagatcattggggtcaaggtactctggtgaccgtctcctca

SEQ ID NO: 20 - DNA, 333 bp, synthetic construct
  misc_feature 1..333: note = Description of Artificial Sequence: Synthetic polynucleotide
  modified_base 109: mod_base = x; note = a, c, t, g, unknown or other
  source 1..333: mol_type = other DNA
cagtctgtgttgacgcagccgccctcactgtctggggccccagggcagagggtcaccatctcttgttccggaagcaggtccaacatcggaactaattatgtatcctggnaccagcaactcccaggaacggcccccaaactcctcatctataggaatcatcagtggccctcaggggtccctgaccgattcactggctccaagtctggcacctcagcctccctggccatcagtgggctccggtccgaggatgaggctgattactactgtgcagcatgggatgacaatttgagtggtgtggtgttcggcggagggaccaagctgaccgtcctaggt

SEQ ID NO: 21 - protein, 121 aa, synthetic construct
  REGION 1..121: note = Description of Artificial Sequence: Synthetic polypeptide
QVQLVQSGAEVQRPGASVRVSCKAIAYTFTDYYIHWVRQAPGQGPEWMGWINPKSGRTQYAPKFQDRVTLARETPISTASMELRGLTSDDTAVYYCARVYGYSRWSGFDLWGQGTLVTVSS

SEQ ID NO: 22 - protein, 111 aa, synthetic construct
  REGION 1..111: note = Description of Artificial Sequence: Synthetic polypeptide
QAVLTQPPSASGTPGQRVTISCSGSSSNIGSNYVYWYQQLPGTAPKLLIYRNNQRPSGVPDRFSGSKSGTSASLAISGLRSEDEADYYCAAWDDSLSGYVFGTGTKVTVLG

SEQ ID NO: 23 - DNA, 363 bp, synthetic construct
  misc_feature 1..363: note = Description of Artificial Sequence: Synthetic polynucleotide
  source 1..363: mol_type = other DNA
caggtccagctggtgcagtctggggctgaggtgcagaggcctggggcctcagtgagggtctcctgcaaggctattgcgtacaccttcaccgactactatatccactgggtgcgacaggcccctggacaagggcctgagtggatggggtggatcaaccctaaaagtggtcgcacacagtatgcaccgaagtttcaagacagggtcaccctggccagggagacgcccatcagcacagcctccatggagctgcgcggactgacatctgacgacacggccgtgtattactgtgcgcgcgtttacggttactctcgttggtctggtttcgatctgtggggtcaaggtactctggtgaccgtctcctca

SEQ ID NO: 24 - DNA, 333 bp, synthetic construct
  misc_feature 1..333: note = Description of Artificial Sequence: Synthetic polynucleotide
  source 1..333: mol_type = other DNA
caggctgtgctgactcagccaccctcagcgtctgggacccccgggcagagggtcaccatctcttgttctggaagcagctccaacatcggaagtaattatgtatactggtaccagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgagtggttatgtcttcggaactgggaccaaggtcaccgtcctaggt

SEQ ID NO: 25 - protein, 124 aa, synthetic construct
  REGION 1..124: note = Description of Artificial Sequence: Synthetic polypeptide
QVQLVQSGAEVKQPGASVKVSCQASGYTFTTYYMHWVRQAPGQGLEWMGIINPNGGGTFYAQKFQDRVTMTRDTSTGTVYMELSSLRSDDTAVYYCARGHKVYKSHPTGGYDRWGQGTLVTVSS

SEQ ID NO: 26 - protein, 111 aa, synthetic construct
  REGION 1..111: note = Description of Artificial Sequence: Synthetic polypeptide
QSALTQPASVSGSPGQSITISCTGTSRDVGGYNYVSWYQQYPGKAPKLMIYEVSKRPSGVSNRFSGSKSGNTASLTISGLQAEDEADYYCSSYTSSSTLDFGTGTKVTVLG

SEQ ID NO: 27 - DNA, 372 bp, synthetic construct
  misc_feature 1..372: note = Description of Artificial Sequence: Synthetic polynucleotide
  source 1..372: mol_type = other DNA
caggtgcagctggtgcaatctggggctgaggtgaagcagcctggggcctcagtgaaggtttcctgccaggcatctggatacaccttcaccacttattatatgcactgggtgcgacaggcccctggacaagggcttgagtggatgggaataatcaaccctaatggtggtggcacattctacgcacagaagttccaggacagagtcaccatgaccagggacacgtccacgggcacagtctacatggaactgagcagcctgagatctgacgacactgccgtgtattactgtgcgcgcggtcataaagtttacaaatctcatccgactggtggttacgatcgttggggtcaaggtactctggtgaccgtctcctca

SEQ ID NO: 28 - DNA, 333 bp, synthetic construct
  misc_feature 1..333: note = Description of Artificial Sequence: Synthetic polynucleotide
  source 1..333: mol_type = other DNA
caatctgccctgactcagcctgcctccgtgtctgggtctcctggacagtcgatcaccatctcctgcactggaaccagccgtgacgttggtggttataactatgtctcctggtaccaacagtacccaggcaaagcccccaaactcatgatttatgaggtcagtaagcggccctcaggggtttctaatcgcttctctggctccaagtctggcaacacggcctccctgaccatctctgggctccaggctgaggacgaggctgattattactgcagctcatataccagtagcagcactttagacttcggaactgggaccaaggtcaccgtcctaggt

SEQ ID NO: 29 - protein, 119 aa, synthetic construct
  REGION 1..119: note = Description of Artificial Sequence: Synthetic polypeptide
EVQLVQSGAEVKKPGSSVKVSCKASGGTFSSYAISWVRQAPGQGLEWMGGIIPIFGTAKYAQKFQGRVTITADESTSTAYMELSSLRSEDTAVYYCARSHVAWSLLDYWGQGTLVTVSS

SEQ ID NO: 30 - protein, 111 aa, synthetic construct
  REGION 1..111: note = Description of Artificial Sequence: Synthetic polypeptide
SYELTQPPSASGTPGQRVTISCSGSSSNIGSNYVSWYQQLPGTAPKLLIYRNNQRPSGVPDRFSGSKSGTSASLAISGLRSEDEADYYCAAWDDSLSGVVFGGGTKLTVLG

SEQ ID NO: 31 - DNA, 357 bp, synthetic construct
  misc_feature 1..357: note = Description of Artificial Sequence: Synthetic polynucleotide
  source 1..357: mol_type = other DNA
gaggtccagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggattatccctatctttggtacagcaaaatatgcacagaagttccagggcagagtcacgattaccgcggacgaatccacgagcacagcctacatggagctgagcagcctgagatctgaggacacggccgtgtattactgtgcgcgctctcatgttgcttggtctctgctggattactggggtcaaggtactctggtgaccgtctcctca

SEQ ID NO: 32 - DNA, 333 bp, synthetic construct
  misc_feature 1..333: note = Description of Artificial Sequence: Synthetic polynucleotide
  source 1..333: mol_type = other DNA
tcctatgagctgactcagccaccctcagcgtctgggacccccgggcagagggtcaccatctcttgttctggaagcagctccaacatcggaagtaattatgtatcctggtaccagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgagtggtgtggtattcggcggagggaccaagctgaccgtcctaggt

SEQ ID NO: 33 - protein, 120 aa, synthetic construct
  REGION 1..120: note = Description of Artificial Sequence: Synthetic polypeptide
QVQLVQSGAEVKKPGSSVKVSCKASGGTFSSYAISWVRQAPGQGLEWMGWMNPNSGNTGYAQKFQGRVTMTRNTSISTAYMELSSLRSEDTAVYYCARYQSYKGSQSDSWGQGTLVTVSS

SEQ ID NO: 34 - protein, 111 aa, synthetic construct
  REGION 1..111: note = Description of Artificial Sequence: Synthetic polypeptide
QSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLSGWVFGGGTKLTVLG

SEQ ID NO: 35 - DNA, 360 bp, synthetic construct
  misc_feature 1..360: note = Description of Artificial Sequence: Synthetic polynucleotide
  source 1..360: mol_type = other DNA
caggtgcagctggtgcagtctggggctgaggtgaagaagcctgggtcctcagtgaaggtctcctgcaaggcttctggaggcaccttcagcagctatgctatcagctgggtgcgacaggcccctggacaagggcttgagtggatgggatggatgaaccctaacagtggtaacacaggctatgcacagaagttccagggcagagtcaccatgaccaggaacacctccataagcacagcctacatggagctgagcagcctgagatctgaggacacggccgtgtattactgtgcgcgctaccagtcttacaaaggttctcagtctgattcttggggtcaaggtactctggtgaccgtctcctca

SEQ ID NO: 36 - DNA, 333 bp, synthetic construct
  misc_feature 1..333: note = Description of Artificial Sequence: Synthetic polynucleotide
  source 1..333: mol_type = other DNA
cagtctgtgttgacgcagccaccctcagcgtctgggacccccgggcagagggtcaccatctcttgttctggaagcagctccaacatcggaagtaattatgtatactggtaccagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgagtggttgggtgttcggcggagggaccaagctgaccgtcctaggt

SEQ ID NO: 37 - protein, 127 aa, synthetic construct
  REGION 1..127: note = Description of Artificial Sequence: Synthetic polypeptide
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGGSKKWSGEKWRRENFDYWGQGTLVTVSS

SEQ ID NO: 38 - protein, 111 aa, synthetic construct
  REGION 1..111: note = Description of Artificial Sequence: Synthetic polypeptide
QSALTQPASVSGSPGQSITISCTGTSSDVGGYNYVSWYQQHPGKAPKLMIYDVSKRPSGVSNRFSGSKSGNTASLTISGLQAEDEADYYCSSYTRSSTEVFGGGTKLTVLG

SEQ ID NO: 39 - DNA, 381 bp, synthetic construct
  misc_feature 1..381: note = Description of Artificial Sequence: Synthetic polynucleotide
  source 1..381: mol_type = other DNA
gaggtccagctggtacagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgcgcggtggttctaaaaaatggtctggtgaaaaatggcgtcgtgaaaacttcgattactggggtcaaggtactctggtgaccgtctcctca

SEQ ID NO: 40 - DNA, 333 bp, synthetic construct
  misc_feature 1..333: note = Description of Artificial Sequence: Synthetic polynucleotide
  source 1..333: mol_type = other DNA
caatctgccctgactcagcctgcctccgtgtctgggtctcctggacagtcgatcaccatctcctgcactggaaccagcagtgacgttggtggttataactatgtctcctggtaccaacagcacccaggcaaagcccccaaactcatgatttatgatgtcagtaagcggccctcaggggtttctaatcgcttctctggctccaagtctggcaacacggcctccctgaccatctctgggctccaggctgaggacgaggctgattattactgcagctcatatacaagaagcagcactgaggtattcggcggagggaccaagctgaccgtcctaggt

SEQ ID NO: 41 - protein, 113 aa, synthetic construct
  REGION 1..113: note = Description of Artificial Sequence: Synthetic polypeptide
QMQLVQSGAEVKKPGASVKVSCKASEYTFTRHILHWVRQAPGQSLEWMGWINPGNGNTKYSQKFQVRVTFTRDTSASTVYMELSSLRSEDTAVYYCARLPDQWGQGTLVTVSS

SEQ ID NO: 42 - protein, 110 aa, synthetic construct
  REGION 1..110: note = Description of Artificial Sequence: Synthetic polypeptide
SYVLTQPPSASGTPGQRVTISCSGSSSNIGSNTVNWYQQLPGTAPKLLIYRNNQRPSGVPDRFSGSKSGTSASLAISGLRSEDEADYYCAAWDDSLSGLFGTGTKVTVLG

SEQ ID NO: 43 - DNA, 339 bp, synthetic construct
  misc_feature 1..339: note = Description of Artificial Sequence: Synthetic polynucleotide
  source 1..339: mol_type = other DNA
cagatgcagctggtgcagtctggggctgaggtgaagaagcctggggcctcagtgaaggtttcctgcaaggcttctgaatacaccttcactaggcatattctacattgggtgcgccaggctcccggacaaagccttgagtggatgggatggatcaacccaggcaatggtaatacaaaatattcacagaagttccaggtcagagtcacctttaccagggacacatccgcgagcacagtctatatggagctgagcagcctgagatctgaagacacggccgtgtattactgtgcgcgcctgccggatcagtggggtcaaggtactctggtgaccgtctcctca

SEQ ID NO: 44 - DNA, 330 bp, synthetic construct
  misc_feature 1..330: note = Description of Artificial Sequence: Synthetic polynucleotide
  source 1..330: mol_type = other DNA
tcctatgtgctgactcagccaccctcagcgtctgggacccccgggcagagggtcaccatctcttgttctggaagcagctccaacatcggaagtaatactgtaaactggtaccagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgagtggtctcttcggaactgggaccaaggtcaccgtcctaggt

SEQ ID NO: 45 - protein, 114 aa, synthetic construct
  REGION 1..114: note = Description of Artificial Sequence: Synthetic polypeptide
EVQLVESGGGVVRPGGSLRLSCAASGFTFGDYGMSWVRQAPGKGLEWVSGINWNGGSTGYADSVKGRFTISRDNAKNSLYLQMNSLRAEDTAVYYCARSKQDYWGQGTLVTVSS

SEQ ID NO: 46 - protein, 112 aa, synthetic construct
  REGION 1..112: note = Description of Artificial Sequence: Synthetic polypeptide
QSALTQPPSASGSPGQSVTISCTGTSRDAGGYNYFSWYQQHPGKAPKLLIYEVTKRPSGVPDRFSGSKSGKTASLTVSGLQADDEAVYYCSSYGGSNNFRVFGGGTKLTVLG

SEQ ID NO: 47 - DNA, 342 bp, synthetic construct
  misc_feature 1..342: note = Description of Artificial Sequence: Synthetic polynucleotide
  source 1..342: mol_type = other DNA
gaggtgcagctggtggagtctgggggaggtgtggtacggcctggggggtccctgagactctcctgtgcagcctctggattcacctttggtgattatggcatgagctgggtccgccaagctccagggaaggggctggagtgggtctctggtattaattggaatggtggtagcacaggttatgcagactctgtgaagggccgattcaccatctccagagacaacgccaagaactccctgtatctgcaaatgaacagtctgagagccgaggacacggccgtatattactgtgcgcgctctaaacaggattactggggtcaaggtactctggtgaccgtctcctca

SEQ ID NO: 48 - DNA, 336 bp, synthetic construct
  misc_feature 1..336: note = Description of Artificial Sequence: Synthetic polynucleotide
  source 1..336: mol_type = other DNA
cagtctgccctgactcagcctccctccgcgtccgggtctcctggacagtcagtcaccatctcctgcactggaaccagcagggacgctggtggttataattatttctcctggtaccaacaacacccaggcaaagcccccaaactcctgatttatgaggtcactaagcggccctcaggggtccctgatcgcttctctggctccaagtctggcaagacggcctccctgaccgtctctgggctccaggctgacgatgaggctgtatattactgcagctcatatggaggcagcaacaactttcgggtgttcggcggagggaccaagctgaccgtcctaggt

SEQ ID NO: 49 - protein, 114 aa, synthetic construct
  REGION 1..114: note = Description of Artificial Sequence: Synthetic polypeptide
EVQLVETGGNLVQPGASLRLSCAASGFSFSGTAMHWVRQAPGKGLEWVSTISSTGRSTYYRDSVKGRFTISRDNSKNTLYLQMNSLRGEDTAVYYCARVSFDYWGQGTLVTVSS

SEQ ID NO: 50 - protein, 113 aa, synthetic construct
  REGION 1..113: note = Description of Artificial Sequence: Synthetic polypeptide
QSVVTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSYVFGTGTKLTVLG

SEQ ID NO: 51 - DNA, 342 bp, synthetic construct
  misc_feature 1..342: note = Description of Artificial Sequence: Synthetic polynucleotide
  source 1..342: mol_type = other DNA
gaggtgcagctggtggagactgggggaaacttggtacagccgggggcgtccctgagactctcctgtgcagcctctggattcagctttagtggcactgccatgcactgggtccgccaggctccagggaaggggctggaatgggtctcgactattagtagtactgggcgtagcacatactacagagactccgtgaagggccggttcaccatctccagagacaattccaagaacacgctgtatctgcaaatgaacagcctgagaggcgaggacacggccgtatattactgtgcgcgcgtttctttcgattactggggtcaaggtactctggtgaccgtctcctca

SEQ ID NO: 52 - DNA, 339 bp, synthetic construct
  misc_feature 1..339: note = Description of Artificial Sequence: Synthetic polynucleotide
  source 1..339: mol_type = other DNA
cagtctgtcgtgacgcagccgccctcagtgtctggggccccagggcagagggtcaccatctcctgcactgggagcagctccaacatcggggcaggttatgatgtacactggtaccagcagcttccaggaacagcccccaaactcctcatctatggtaacagcaatcggccctcaggggtccctgaccgattctctggctccaagtctggcacctcagcctccctggccatcactgggctccaggctgaggatgaggctgattattactgccagtcctatgacagcagcctgagtggctcctacgtcttcggaactgggaccaagctgaccgtcctaggt

SEQ ID NO: 53 - protein, 118 aa, synthetic construct
  REGION 1..118: note = Description of Artificial Sequence: Synthetic polypeptide
EVQLVESGGAFVQPGGSLRLSCAASGFTFSSYAMTWVRQAPGKGLEWVSTISGRGRSTFYADSVKGRFTISRDNSKNTLYLQMNSLRAEDTAVYYCARYYHAGAFDLWGQGTLVTVSS

SEQ ID NO: 54 - protein, 111 aa, synthetic construct
  REGION 1..111: note = Description of Artificial Sequence: Synthetic polypeptide
QSVVTQPASVSGSPGQSITISCTGTSSDVGGYNYVSWYQQHPGKAPKLMIYDVSKRPSGVSNRFSGSKSGNTASLTISGLQAEDEADYYCSSYTSSSTLVFGGGTKLTVLG

SEQ ID NO: 55 - DNA, 354 bp, synthetic construct
  misc_feature 1..354: note = Description of Artificial Sequence: Synthetic polynucleotide
  source 1..354: mol_type = other DNA
gaggtgcagctggtggagtctgggggagcctttgtacagcctggggggtccctgagactctcctgtgcagcctctggattcacctttagcagctatgccatgacctgggtccgccaggctccagggaagggcctggaatgggtctcgactattagtggtcgtggtcgtagcacattctacgcagactccgtgaagggccggtttaccatctccagagacaattccaagaacacgctatatctgcaaatgaacagtctgagagccgaggacacggccgtatattactgtgcgcgctactaccatgctggtgctttcgatctgtggggtcaaggtactctggtgaccgtctcctca

SEQ ID NO: 56 - DNA, 333 bp, synthetic construct
  misc_feature 1..333: note = Description of Artificial Sequence: Synthetic polynucleotide
  source 1..333: mol_type = other DNA
cagtctgtcgtgacgcagcctgcctccgtgtctgggtctcctggacagtcgatcaccatctcctgcactggaaccagcagtgacgttggtggttataactatgtctcctggtaccaacagcacccaggcaaagcccccaaactcatgatttatgatgtcagtaagcggccctcaggggtttctaatcgcttctctggctccaagtctggcaacacggcctccctgaccatctctgggctccaggctgaggacgaggctgattattactgcagctcatatacaagcagcagcactttggtattcggcggagggaccaagctgaccgtcctaggt

SEQ ID NO: 57 - protein, 114 aa, synthetic construct
  REGION 1..114: note = Description of Artificial Sequence: Synthetic polypeptide
QMQLVQSGAEVKKPGASVKVSCKASGYTFNRYAITWVRQAPGQGLEWMGWISAYNGNSHYAQKLQGRVTMTTDTSTGTAYMELRRLRSDDTAVYYCARMAYDSWGQGTLVTVSS

SEQ ID NO: 58 - protein, 111 aa, synthetic construct
  REGION 1..111: note = Description of Artificial Sequence: Synthetic polypeptide
QSVLTQPASVSGSPGQSLTISCTGTSNDVGAYKYVSWYQQYPGKAPKLILYDVFKRPSGVSNRFSGSKSDNTASLTISGLQAEDEADYYCFSLTSSNTYVFGTGTKVTVLG

SEQ ID NO: 59 - DNA, 342 bp, synthetic construct
  misc_feature 1..342: note = Description of Artificial Sequence: Synthetic polynucleotide
  source 1..342: mol_type = other DNA
cagatgcagctggtgcagtctggagctgaggtgaagaagcctggggcctcagtgaaggtctcctgcaaggcttctggttacacctttaacagatatgctatcacctgggtgcgacaggcccctggacaaggccttgagtggatgggatggatcagcgcttacaatggtaattcacactatgcacagaagctccagggcagagtcaccatgaccacagacacatccacgggcacagcctatatggagctgaggaggctgagatctgacgacacggccgtgtattactgtgcgcgcatggcttacgattcttggggtcaaggtactctggtgaccgtctcctca

SEQ ID NO: 60 - DNA, 333 bp, synthetic construct
  misc_feature 1..333: note = Description of Artificial Sequence: Synthetic polynucleotide
  source 1..333: mol_type = other DNA
cagtctgtgttgacgcagcctgcctccgtgtctgggtctcctggacagtcgctcaccatctcctgcactggaaccagcaatgacgttggtgcttataagtatgtctcctggtatcaacagtacccaggcaaagcccccaaactcatactttatgatgtctttaagcggccctcaggggtctctaatcgcttctctggctccaagtctgacaacacggcctccctgaccatctctgggctccaggctgaggacgaggctgattattactgcttctcacttacaagcagtaacacttatgtcttcggaactgggaccaaggtcaccgtcctaggt

SEQ ID NO: 61 - protein, 117 aa, synthetic construct
  REGION 1..117: note = Description of Artificial Sequence: Synthetic polypeptide
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGYGKAYDQWGQGTLVTVSS

SEQ ID NO: 62 - protein, 111 aa, synthetic construct
  REGION 1..111: note = Description of Artificial Sequence: Synthetic polypeptide
QSVLTQPPSASGTPGQRVTISCSGSRSNVGGNYVFWYQQVPGATPKLLIYRSNQRPSGVPDRFAGSKSGSSASLAISGLRSEDEADYYCATWDDSLSGFVFGTGTKVTVLG

SEQ ID NO: 63 - DNA, 351 bp, synthetic construct
  misc_feature 1..351: note = Description of Artificial Sequence: Synthetic polynucleotide
  source 1..351: mol_type = other DNA
gaggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaactcactgtatctgcaaatgaacagcctgagagccgaggacacggccgtatattactgtgcgcgcggttacggtaaagcttacgatcagtggggtcaaggtactctggtgaccgtctcctca

SEQ ID NO: 64 - DNA, 333 bp, synthetic construct
  misc_feature 1..333: note = Description of Artificial Sequence: Synthetic polynucleotide
  source 1..333: mol_type = other DNA
cagtctgtgttgactcagccaccctcagcgtctgggacccccggacagagggtcaccatctcttgttctggaagcaggtccaacgtaggaggtaattatgtattttggtaccagcaagtccccggagcgacccccaaactcctcatctataggagtaatcagcggccctcgggggtccctgaccgattcgctggctccaagtctggctcctcagcctccctggccatcagtggactccggtccgaggatgaggctgattattactgtgcaacatgggatgacagcctgagtggttttgtcttcggaactgggaccaaggtcaccgtcctaggt

SEQ ID NO: 65 - protein, 119 aa, synthetic construct
  REGION 1..119: note = Description of Artificial Sequence: Synthetic polypeptide
QVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSS

SEQ ID NO: 66 - protein, 110 aa, synthetic construct
  REGION 1..110: note = Description of Artificial Sequence: Synthetic polypeptide
SSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLG

SEQ ID NO: 67 - DNA, 357 bp, synthetic construct
  misc_feature 1..357: note = Description of Artificial Sequence: Synthetic polynucleotide
  source 1..357: mol_type = other DNA
caggtgcagctggtggagtctgggggaggcctggtccaccctggggggtccctgagactctcctgtgcagcctctggattcaccttcagaagccatagcatgaactgggtccgccaggctccagggaaggggctggagtgggtctcatccattagtagtgatagtacttacacatactacgcagactcagtgaagggccgattcaccatctccagagacaacgccaagaactcactgtatctgcaaatgaacagcctgagagccgaggacacggccgtatattactgtgcgcgctctggtggtcagtggaaatactacgattactggggtcaaggtactctggtgaccgtctcctca

SEQ ID NO: 68 - DNA, 330 bp, synthetic construct
  misc_feature 1..330: note = Description of Artificial Sequence: Synthetic polynucleotide
  source 1..330: mol_type = other DNA
tcttctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcccgggacagcagtggtaacccccctgtggtattcggcggagggaccaagctgaccgtcctaggt

SEQ ID NO: 69 - protein, 116 aa, synthetic construct
  REGION 1..116: note = Description of Artificial Sequence: Synthetic polypeptide
EVQLVESGGGLIQPGGSLRLSCAASGFTFSNYAMNWVRQAPGKGLEWVSTINGRGSSTIYADSVKGRFTISRDNSKNTLYLQMNSLRAEDTATYYCARYISRGLGDSWGQGTLVTV

SEQ ID NO: 70 - protein, 111 aa, synthetic construct
  REGION 1..111: note = Description of Artificial Sequence: Synthetic polypeptide
QSVVTQPPSMSAAPGQQVTISCSGGNSNIERNYVSWYLQLPGTAPKLVIFDNDRRPSGIPDRFSGSKSGTSATLGITGLQTGDEADYYCGTWDSSLRGWVFGGGTKLTVLG

SEQ ID NO: 71 - DNA, 354 bp, synthetic construct
  misc_feature 1..354: note = Description of Artificial Sequence: Synthetic polynucleotide
  source 1..354: mol_type = other DNA
gaggtgcagctggtggagtccgggggaggcttgatacagcctggggggtccctgagactctcctgtgcagcctctggattcacctttagcaactatgccatgaactgggtccgccaggctccagggaaggggctggagtgggtctcaactattaatggtcgtggtagtagtacaatctacgcagactccgtgaagggccggttcaccatctccagagacaattccaagaacacgctgtatctgcaaatgaacagcctgagagccgaggacacagccacgtattactgtgcgcgctacatctctcgtggtctgggtgattcttggggtcaaggtactctggtgaccgtctcctca

SEQ ID NO: 72 - DNA, 333 bp, synthetic construct
  misc_feature 1..333: note = Description of Artificial Sequence: Synthetic polynucleotide
  source 1..333: mol_type = other DNA
cagtctgtcgtgacgcagccgccctcaatgtctgcggccccaggacagcaagtcaccatctcctgctctggaggcaactccaacattgagagaaattatgtatcctggtacctccagctccctggaacagcccccaaactcgtcatttttgacaatgataggcgaccctcagggattcctgaccgattctctggctccaagtctggcacgtcagccaccctgggcatcaccggactccagactggggacgaggccgattattactgcggaacatgggatagcagcctgagaggttgggtgttcggcggagggaccaagctgaccgtcctaggt

SEQ ID NO: 73 - protein, 116 aa, synthetic construct
  REGION 1..116: note = Description of Artificial Sequence: Synthetic polypeptide
QMQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARAGMGMDTWGQGTLVTVSS

SEQ ID NO: 74 - protein, 111 aa, synthetic construct
  REGION 1..111: note = Description of Artificial Sequence: Synthetic polypeptide
QSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNTLVFGGGTKLTVLG

SEQ ID NO: 75 - DNA, 348 bp, synthetic construct
  misc_feature 1..348: note = Description of Artificial Sequence: Synthetic polynucleotide
  source 1..348: mol_type = other DNA
cagatgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgcgcgctggtatgggtatggatacttggggtcaaggtactctggtgaccgtctcctca

SEQ ID NO: 76 - DNA, 333 bp, synthetic construct
  misc_feature 1..333: note = Description of Artificial Sequence: Synthetic polynucleotide
  source 1..333: mol_type = other DNA
cagtctgccctgactcagcctccctccgcgtccgggtctcctggacagtcagtcaccatctcctgcactggaaccagcagtgacgttggtggttataactatgtctcctggtaccaacagcacccaggcaaagcccccaaactcatgatttatgaggtcagtaagcggccctcaggggtccctgatcgcttctctggctccaagtctggcaacacggcctccctgaccgtctctgggctccaggctgaggatgaggctgattattactgcagctcatatgcaggcagcaacaccttggtgttcggcggagggaccaagctgaccgtcctaggt

SEQ ID NO: 77 - protein, 117 aa, synthetic construct
  REGION 1..117: note = Description of Artificial Sequence: Synthetic polypeptide
QVQLVQSGAEVKKPGASVKVSCRASGYTFTAYSLHWVRQAPGQGLEWMGWINPSSGGAVYAQKFQGRVTMTRDTSISTAYMELSGLRSDDTAVYYCARNVGGQADDWGQGTLVTVSS

SEQ ID NO: 78 - protein, 111 aa, synthetic construct
  REGION 1..111: note = Description of Artificial Sequence: Synthetic polypeptide
QSALTQPPSASGSPGQSVTISCTGTSSDIGGYNYVSWYQQHPGKAPKLMIYEVNKRPSGVPDRFSGSKSGNTASLTVSGLQAEDEADYYCASFAGRKTLVFGGGTKLTVLG

SEQ ID NO: 79 - DNA, 351 bp, synthetic construct
  misc_feature 1..351: note = Description of Artificial Sequence: Synthetic polynucleotide
  source 1..351: mol_type = other DNA
caggtgcagctggtgcagtctggggctgaggtgaagaagcctggggcctcagtgaaggtctcctgcagggcttctggatacaccttcaccgcctactctttacactgggtgcgacaggcccctggacaagggcttgagtggatgggatggatcaaccctagcagtggtggcgcagtttatgcacagaaatttcagggtagggtcaccatgaccagggacacgtccatcagcacagcctacatggagctgagtggcctgagatctgacgacacggccgtgtattactgtgcgcgcaacgttggtggtcaggctgatgactggggtcaaggtactctggtgaccgtctcctca

SEQ ID NO: 80 - DNA, 333 bp, synthetic construct
  misc_feature 1..333: note = Description of Artificial Sequence: Synthetic polynucleotide
  source 1..333: mol_type = other DNA
caatctgccctgactcagcctccctccgcgtccgggtctcctggacagtcagtcaccatctcctgcactggaaccagcagtgacattggtggttataactatgtctcctggtaccaacagcacccaggcaaagcccccaaactcatgatttatgaggtcaataagcggccctcaggggtccctgatcgcttctcgggctccaagtctggcaacacggcctccctgaccgtctctgggctccaggctgaggatgaggctgattattactgcgcctcatttgcgggcaggaagacattggtcttcggcggagggaccaagctgaccgtcctaggt

SEQ ID NO: 81 - protein, 117 aa, synthetic construct
  REGION 1..117: note = Description of Artificial Sequence: Synthetic polypeptide
QVQLVQSGAEVKKPGASVKVSCRASGYTFTAYSLHWVRQAPGQGLEWMGWINPSSGGAVYAQKFQGRVTMTRDTSISTAYMELSGLRSDDTAVYYCARNVGGHADDWGQGTLVTVSS

SEQ ID NO: 82 - protein, 111 aa, synthetic construct
  REGION 1..111: note = Description of Artificial Sequence: Synthetic polypeptide
QSALTQPPSASGSPGQSVTISCTGTSTDIGGYNYVSWYQHHPSKAPKLMIYEVNKRPSGVPDRFSGSKSGNTASLTVSGLQAEDEADYYCASFAGRKTLVFGGGTKLTVLG

SEQ ID NO: 83 - DNA, 351 bp, synthetic construct
  misc_feature 1..351: note = Description of Artificial Sequence: Synthetic polynucleotide
  source 1..351: mol_type = other DNA
caggtgcagctggtgcagtctggggctgaggtgaaaaagcctggggcctcagtgaaagtctcctgcagggcttctggatacaccttcaccgcctactctttacactgggtgcgacaggcccctggacaagggcttgagtggatgggatggatcaaccctagcagtggtggcgcagtttatgcacagaaatttcagggtagggtcaccatgaccagggacacgtccatcagcacagcctacatggagctgagtggcctgagatctgacgacacggccgtgtattactgtgcgcgcaacgttggtggtcacgctgatgactggggtcaaggtactctggtgaccgtctcctca

SEQ ID NO: 84 - DNA, 333 bp, synthetic construct
  misc_feature 1..333: note = Description of Artificial Sequence: Synthetic polynucleotide
  source 1..333: mol_type = other DNA
caatctgccctgactcagcctccctccgcgtccgggtctcctggacagtcagtcaccatctcctgcactggaaccagcactgacattggtggttataactatgtctcctggtaccaacaccacccaagcaaagcccccaaactcatgatttatgaggtcaataagcggccctcaggggtccctgatcgcttctcgggctccaagtctggcaacacggcctccctgaccgtctctgggctccaggctgaggatgaggctgattattactgcgcctcatttgcgggcaggaagacattggtcttcggcggagggaccaagctgaccgtcctaggt

SEQ ID NO: 85 - protein, 129 aa, synthetic construct
  REGION 1..129: note = Description of Artificial Sequence: Synthetic polypeptide
QVQLVQSGGEVKKPGASVKVSCKASGFTFNTYGISWVRQAPGQGLEWMGWISANNGHTKSAQRFQDRVAMATDTSTSTAYMELRSLKFDDTAVYYCARGGYHHQMQRYYKATSVYSDYWGQGTLVTVSS

SEQ ID NO: 86 - protein, 111 aa, synthetic construct
  REGION 1..111: note = Description of Artificial Sequence: Synthetic polypeptide
QSVVTQPPSVSAAPGQKVTISCSGSSSNIGNNYVSWYQQLPGTAPKLLIYDNNKRPSGIPDRFSGSKSGTSATLGITGLQTGDEADYYCGTWDSSLSGVVFGGGTKLTVLG

SEQ ID NO: 87 - DNA, 387 bp, synthetic construct
  misc_feature 1..387: note = Description of Artificial Sequence: Synthetic polynucleotide
  source 1..387: mol_type = other DNA
caggtccagctggtgcagtctggaggtgaggtgaagaagcctggggcctcagtgaaggtctcctgcaaggcttctggtttcacctttaacacctatggcatcagttgggtgcgacaggcccctggacaagggcttgagtggatgggatggatcagcgctaacaatggtcacacaaagtctgcacagaggttccaggacagagtcgccatggccacagacacatccacgagcacggcctacatggagctgaggagcctgaaatttgacgacacggccgtgtattactgtgcgcgcggtggttaccatcatcagatgcagcggtactacaaagctacttctgtttactctgattactggggtcaaggtactctggtgaccgtctcctca

SEQ ID NO: 88 - DNA, 333 bp, synthetic construct
  misc_feature 1..333: note = Description of Artificial Sequence: Synthetic polynucleotide
  source 1..333: mol_type = other DNA
cagtctgtcgtgacgcagccgccctcagtgtctgcggccccaggacagaaggtcaccatctcctgctctggaagcagctccaacattgggaataattatgtatcctggtaccagcaactcccaggaacagcccccaaactcctcatttatgacaataataagcgaccctcagggattcctgaccgattctctggctccaagtctggcacgtctgccaccctgggcatcaccggactccagactggggacgaggccgattattactgcggaacatgggatagcagcctgagtggtgtggtattcggcggagggaccaagctgaccgtcctaggt

SEQ ID NO: 89 - protein, 116 aa, synthetic construct
  REGION 1..116: note = Description of Artificial Sequence: Synthetic polypeptide
QMQLVQSGAEVKKPGASVKVSCKASGYTFTSYYMHWVRQAPGQGLEWMGIINPSGGSSSYAQKFQGRVTMTRDTSTSTVYMELSSLRSEDTAVYYCARAGMGMDTWGQGTLVTVSS

SEQ ID NO: 90 - protein, 111 aa, synthetic construct; identical to SEQ ID NO: 74
  REGION 1..111: note = Description of Artificial Sequence: Synthetic polypeptide

SEQ ID NO: 91 - DNA, 348 bp, synthetic construct
  misc_feature 1..348: note = Description of Artificial Sequence: Synthetic polynucleotide
  source 1..348: mol_type = other DNA
cagatgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagctcaagctacgcacagaagttccagggcagagtcaccatgaccagggacacgtccacgagcacagtctacatggagctgagcagcctgagatctgaggacacggccgtgtattactgtgcgcgcgctggtatgggtatggatacttggggtcaaggtactctggtgaccgtctcctca

SEQ ID NO: 92 - DNA, 333 bp, synthetic construct; identical to SEQ ID NO: 76
  misc_feature 1..333: note = Description of Artificial Sequence: Synthetic polynucleotide
  source 1..333: mol_type = other DNA

SEQ ID NO: 93 - protein, 117 aa, synthetic construct
  REGION 1..117: note = Description of Artificial Sequence: Synthetic polypeptide
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDVISGFDSWGQGTLVTVSS

SEQ ID NO: 94 - protein, 111 aa, synthetic construct
  REGION 1..111: note = Description of Artificial Sequence: Synthetic polypeptide
QSALTQPASVSGSPGQSITISCTGTSSDVGGYNYVSWYQQSPGKAPRLMIYGVSKRPSGVPDRFSGSKSGNTASLTVSGLQAEDEADYYCSSYAGVNNLMFGGGTKLTVLG

SEQ ID NO: 95 - DNA, 351 bp, synthetic construct
  misc_feature 1..351: note = Description of Artificial Sequence: Synthetic polynucleotide
  source 1..351: mol_type = other DNA
caggtgcagctggtgcaatctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacactgccgtgtattactgtgcgcgcgacgttatctctggtttcgattcttggggtcaaggtactctggtgaccgtctcctca

SEQ ID NO: 96 - DNA, 333 bp, synthetic construct
  misc_feature 1..333: note = Description of Artificial Sequence: Synthetic polynucleotide
  source 1..333: mol_type = other DNA
cagtctgccctgactcagcctgcctccgtgtctgggtctcctggacagtcgatcaccatctcctgcactggaaccagcagtgacgttggtggttataactatgtctcctggtaccaacaatccccaggcaaagcccccagactcatgatttatggggtcagtaagcggccctctggggtccctgatcgcttctctggctccaagtctggcaacacggcctccctgaccgtctctgggctccaggctgaagatgaggctgattattactgcagctcatatgcaggcgtcaacaatttaatgttcggcggagggaccaagctgaccgtcctaggt

SEQ ID NO: 97 - protein, 345 aa, Homo sapiens
MYKDCIESTGDYFLLCDAEGPWGIILESLAILGIVVTILLLLAFLFLMRKIQDCSQWNVLPTQLLFLLSVLGLFGLAFAFIIELNQQTAPVRYFLFGVLFALCFSCLLAHASNLVKLVRGCVSFSWTTILCIAIGCSLLQIIIATEYVTLIMTRGMMFVNMTPCQLNVDFVVLLVYVLFLMALTFFVSKATFCGPCENWKQHGRLIFITVLFSIIIWVVWISMLLRGNPQFQRQPQWDDPVVCIALVTNAWVFLLLYIVPELCILYRSCRQECPLQGNACPVTAYQHSFQVENQELSRARDSDGAEEDVALTSYGTPIQPQTVDPTQECFIPQAKLSPQQDAGGV

SEQ ID NO: 98 - protein, 21 aa, synthetic construct
  REGION 1..21: note = Description of Artificial Sequence: Synthetic peptide
SRGGGGSGGGGSGGGGSLEMA

SEQ ID NO: 99 - DNA, 63 bp, synthetic construct
  misc_feature 1..63: note = Description of Artificial Sequence: Synthetic oligonucleotide
  source 1..63: mol_type = other DNA
tctagaggtggtggtggtagcggcggcggcggctctggtggtggtggatccctcgagatggcc

SEQ ID NO: 100 - protein, 252 aa, synthetic construct
  REGION 1..252: note = Description of Artificial Sequence: Synthetic polypeptide
QSVLTQPPSVSAAPGQKVTIPCSGSRSNVGNYYVSWYQQLPGTAPKLLIYDNNKRPSGIPDRFSGSKSGTSATLGITGLQTGDEADYFCGTWDGSLSAHVFGTGTKVTVLGSRGGGGSGGGGSGGGGSLEMAQVQLVQSGSELKKPGASVRVSCTASGYTFTSYYMHWVRQAPGQGLEWMGVINPNAGSTRYAQKFQGRVTMSTDTSTSTAYMDLSSLRSEDTAVYYCARGMYRSLLFYDPWGQGTLVTVSS

SEQ ID NO: 101 - protein, 250 aa, synthetic construct
  REGION 1..250: note = Description of Artificial Sequence: Synthetic polypeptide
NFMLTQPHSVSESPGKTVSISCTRTSGAIAGAYVQWFQQRPGSAPTTVIYDDNKRPSGVPDRFSGSIDKSSNSASLTISGLKTEDEADYYCQSYDYDSSNVLFGGGTKLTVLGSRGGGGSGGGGSGGGGSLEMAEVQLVESGGGLVQPGGSLRLSCAASGFTFSNYAMSWVRQAPGKGLEWVSAISGSGNTYYADSVKGRFTISRDNSKNTLYLQMNSLRAEDTAVYYCARGSVRYTDIWGQGTLVTVSS

SEQ ID NO: 102 - protein, 248 aa, synthetic construct
  REGION 1..248: note = Description of Artificial Sequence: Synthetic polypeptide
DIQLTQSPSSLSASVGDRVTITCRASQSISSYLNWYQQKPGKAPKLLIYAASSLQSGVPSRFSGSGSGTDFTLTISSLQPADFATYYCQQSYSVPYTFGQGTKLEIKRSRGGGGSGGGGSGGGGSLEMAEVQLVQSGGGLVQPGGSLRLSCATSGFTFNNYWMSWVRQAPGKGLEWVANIKQDGSEKYYADSVRGRFTISRDNAKNSLSLQLNNLRAEDTAVYYCARSMSTAWGYDEWGQGTLVTVSS

SEQ ID NO: 103 - protein, 253 aa, synthetic construct
  REGION 1..253: note = Description of Artificial Sequence: Synthetic polypeptide
QSALTQPASVSGSPGQSITISCTGTSSDVGGYNFVSWYQQHPGKAPKVMIYDVSKRPSGISNRFSGSKSGNTASLTISGLQVEDEAEYYCSSYTSTRTVIFAGGTKVTVLGSRGGGGSGGGGSGGGGSLEMAQVQLVQSGAEVKKPGASVKVSCKASGYTFTSYYMHWVRQAPGQGLEWMGIINPSGGSTRYAQKFQGRVTMTRDTSTSTVYMELSSLRSEDTAVYYCARGSSRWGGWTGDYWGQGTLVTVSS

SEQ ID NO: 104 - protein, 249 aa, synthetic construct
  REGION 1..249: note = Description of Artificial Sequence: Synthetic polypeptide
  MOD_RES 37: note = Any amino acid
QSVLTQPPSLSGAPGQRVTISCSGSRSNIGTNYVSWXQQLPGTAPKLLIYRNHQWPSGVPDRFTGSKSGTSASLAISGLRSEDEADYYCAAWDDNLSGVVFGGGTKLTVLGSRGGGGSGGGGSGGGGSLEMAEVQLVETGGGLVQPGGSLRLSCAASGSTFSSYAMSWVRQAPGKGLEWVSAISGRGRSTYYADSVKGRFTISRDNSKNTLYLQMNSLRAEDTAVYYCARYYKSSKDHWGQGTLVTVSS

SEQ ID NO: 105 - protein, 253 aa, synthetic construct
  REGION 1..253: note = Description of Artificial Sequence: Synthetic polypeptide
QAVLTQPPSASGTPGQRVTISCSGSSSNIGSNYVYWYQQLPGTAPKLLIYRNNQRPSGVPDRFSGSKSGTSASLAISGLRSEDEADYYCAAWDDSLSGYVFGTGTKVTVLGSRGGGGSGGGGSGGGGSLEMAQVQLVQSGAEVQRPGASVRVSCKAIAYTFTDYYIHWVRQAPGQGPEWMGWINPKSGRTQYAPKFQDRVTLARETPISTASMELRGLTSDDTAVYYCARVYGYSRWSGFDLWGQGTLVTVSS

SEQ ID NO: 106 - protein, 256 aa, synthetic construct
  REGION 1..256: note = Description of Artificial Sequence: Synthetic polypeptide
QSALTQPASVSGSPGQSITISCTGTSRDVGGYNYVSWYQQYPGKAPKLMIYEVSKRPSGVSNRFSGSKSGNTASLTISGLQAEDEADYYCSSYTSSSTLDFGTGTKVTVLGSRGGGGSGGGGSGGGGSLEMAQVQLVQSGAEVKQPGASVKVSCQASGYTFTTYYMHWVRQAPGQGLEWMGIINPNGGGTFYAQKFQDRVTMTRDTSTGTVYMELSSLRSDDTAVYYCARGHKVYKSHPTGGYDRWGQGTLVTVSS

SEQ ID NO: 107 - protein, 251 aa, synthetic construct
  REGION 1..251: note = Description of Artificial Sequence: Synthetic polypeptide
SYELTQPPSASGTPGQRVTISCSGSSSNIGSNYVSWYQQLPGTAPKLLIYRNNQRPSGVPDRFSGSKSGTSASLAISGLRSEDEADYYCAAWDDSLSGVVFGGGTKLTVLGSRGGGGSGGGGSGGGGSLEMAEVQLVQSGAEVKKPGSSVKVSCKASGGTFSSYAISWVRQAPGQGLEWMGGIIPIFGTAKYAQKFQGRVTITADESTSTAYMELSSLRSEDTAVYYCARSHVAWSLLDYWGQGTLVTVSS

SEQ ID NO: 108 - protein, 252 aa, synthetic construct
  REGION 1..252: note = Description of Artificial Sequence: Synthetic polypeptide
QSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLSGWVFGGGTKLTVLGSRGGGGSGGGGSGGGGSLEMAQVQLVQSGAEVKKPGSSVKVSCKASGGTFSSYAISWVRQAPGQGLEWMGWMNPNSGNTGYAQKFQGRVTMTRNTSISTAYMELSSLRSEDTAVYYCARYQSYKGSQSDSWGQGTLVTVSS

SEQ ID NO: 109 - protein, 259 aa, synthetic construct
  REGION 1..259: note = Description of Artificial Sequence: Synthetic polypeptide
QSALTQPASVSGSPGQSITISCTGTSSDVGGYNYVSWYQQHPGKAPKLMIYDVSKRPSGVSNRFSGSKSGNTASLTISGLQAEDEADYYCSSYTRSSTEVFGGGTKLTVLGSRGGGGSGGGGSGGGGSLEMAEVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGGSKKWSGEKWRRENFDYWGQGTLVTVSS

SEQ ID NO: 110 - protein, 244 aa, synthetic construct
  REGION 1..244: note = Description of Artificial Sequence: Synthetic polypeptide
SYVLTQPPSASGTPGQRVTISCSGSSSNIGSNTVNWYQQLPGTAPKLLIYRNNQRPSGVPDRFSGSKSGTSASLAISGLRSEDEADYYCAAWDDSLSGLFGTGTKVTVLGSRGGGGSGGGGSGGGGSLEMAQMQLVQSGAEVKKPGASVKVSCKASEYTFTRHILHWVRQAPGQSLEWMGWINPGNGNTKYSQKFQVRVTFTRDTSASTVYMELSSLRSEDTAVYYCARLPDQWGQGTLVTVSS

SEQ ID NO: 111 - protein, 247 aa, synthetic construct
  REGION 1..247: note = Description of Artificial Sequence: Synthetic polypeptide
QSALTQPPSASGSPGQSVTISCTGTSRDAGGYNYFSWYQQHPGKAPKLLIYEVTKRPSGVPDRFSGSKSGKTASLTVSGLQADDEAVYYCSSYGGSNNFRVFGGGTKLTVLGSRGGGGSGGGGSGGGGSLEMAEVQLVESGGGVVRPGGSLRLSCAASGFTFGDYGMSWVRQAPGKGLEWVSGINWNGGSTGYADSVKGRFTISRDNAKNSLYLQMNSLRAEDTAVYYCARSKQDYWGQGTLVTVSS

SEQ ID NO: 112 - protein, 248 aa, synthetic construct
  REGION 1..248: note = Description of Artificial Sequence: Synthetic polypeptide
QSVVTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSYVFGTGTKLTVLGSRGGGGSGGGGSGGGGSLEMAEVQLVETGGNLVQPGASLRLSCAASGFSFSGTAMHWVRQAPGKGLEWVSTISSTGRSTYYRDSVKGRFTISRDNSKNTLYLQMNSLRGEDTAVYYCARVSFDYWGQGTLVTVSS

SEQ ID NO: 113 - protein, 250 aa, synthetic construct
  REGION 1..250: note = Description of Artificial Sequence: Synthetic polypeptide
QSVVTQPASVSGSPGQSITISCTGTSSDVGGYNYVSWYQQHPGKAPKLMIYDVSKRPSGVSNRFSGSKSGNTASLTISGLQAEDEADYYCSSYTSSSTLVFGGGTKLTVLGSRGGGGSGGGGSGGGGSLEMAEVQLVESGGAFVQPGGSLRLSCAASGFTFSSYAMTWVRQAPGKGLEWVSTISGRGRSTFYADSVKGRFTISRDNSKNTLYLQMNSLRAEDTAVYYCARYYHAGAFDLWGQGTLVTVSS

SEQ ID NO: 114 - protein, 246 aa, synthetic construct
  REGION 1..246: note = Description of Artificial Sequence: Synthetic polypeptide
QSVLTQPASVSGSPGQSLTISCTGTSNDVGAYKYVSWYQQYPGKAPKLILYDVFKRPSGVSNRFSGSKSDNTASLTISGLQAEDEADYYCFSLTSSNTYVFGTGTKVTVLGSRGGGGSGGGGSGGGGSLEMAQMQLVQSGAEVKKPGASVKVSCKASGYTFNRYAITWVRQAPGQGLEWMGWISAYNGNSHYAQKLQGRVTMTTDTSTGTAYMELRRLRSDDTAVYYCARMAYDSWGQGTLVTVSS

SEQ ID NO: 115 - protein, 249 aa, synthetic construct
  REGION 1..249: note = Description of Artificial Sequence: Synthetic polypeptide
QSVLTQPPSASGTPGQRVTISCSGSRSNVGGNYVFWYQQVPGATPKLLIYRSNQRPSGVPDRFAGSKSGSSASLAISGLRSEDEADYYCATWDDSLSGFVFGTGTKVTVLGSRGGGGSGGGGSGGGGSLEMAEVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGYGKAYDQWGQGTLVTVSS

SEQ ID NO: 116 - protein, 250 aa, synthetic construct
  REGION 1..250: note = Description of Artificial Sequence: Synthetic polypeptide
SSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLGSRGGGGSGGGGSGGGGSLEMAQVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSS

SEQ ID NO: 117 - protein, 248 aa, synthetic construct
  REGION 1..248: note = Description of Artificial Sequence: Synthetic polypeptide
QSVVTQPPSMSAAPGQQVTISCSGGNSNIERNYVSWYLQLPGTAPKLVIFDNDRRPSGIPDRFSGSKSGTSATLGITGLQTGDEADYYCGTWDSSLRGWVFGGGTKLTVLGSRGGGGSGGGGSGGGGSLEMAEVQLVESGGGLIQPGGSLRLSCAASGFTFSNYAMNWVRQAPGKGLEWVSTINGRGSSTIYADSVKGRFTISRDNSKNTLYLQMNSLRAEDTATYYCARYISRGLGDSWGQGTLVTV

SEQ ID NO: 118 - protein, 248 aa, synthetic construct
  REGION 1..248: note = Description of Artificial Sequence: Synthetic polypeptide
QSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNTLVFGGGTKLTVLGSRGGGGSGGGGSGGGGSLEMAQMQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARAGMGMDTWGQGTLVTVSS

SEQ ID NO: 119 - protein, 249 aa, synthetic construct
  REGION 1..249: note = Description of Artificial Sequence: Synthetic polypeptide
QSALTQPPSASGSPGQSVTISCTGTSSDIGGYNYVSWYQQHPGKAPKLMIYEVNKRPSGVPDRFSGSKSGNTASLTVSGLQAEDEADYYCASFAGRKTLVFGGGTKLTVLGSRGGGGSGGGGSGGGGSLEMAQVQLVQSGAEVKKPGASVKVSCRASGYTFTAYSLHWVRQAPGQGLEWMGWINPSSGGAVYAQKFQGRVTMTRDTSISTAYMELSGLRSDDTAVYYCARNVGGQADDWGQGTLVTVSS

SEQ ID NO: 120 - protein, 249 aa, synthetic construct
  REGION 1..249: note = Description of Artificial Sequence: Synthetic polypeptide
QSALTQPPSASGSPGQSVTISCTGTSTDIGGYNYVSWYQHHPSKAPKLMIYEVNKRPSGVPDRFSGSKSGNTASLTVSGLQAEDEADYYCASFAGRKTLVFGGGTKLTVLGSRGGGGSGGGGSGGGGSLEMAQVQLVQSGAEVKKPGASVKVSCRASGYTFTAYSLHWVRQAPGQGLEWMGWINPSSGGAVYAQKFQGRVTMTRDTSISTAYMELSGLRSDDTAVYYCARNVGGHADDWGQGTLVTVSS

SEQ ID NO: 121 - protein, 261 aa, synthetic construct
  REGION 1..261: note = Description of Artificial Sequence: Synthetic polypeptide
QSVVTQPPSVSAAPGQKVTISCSGSSSNIGNNYVSWYQQLPGTAPKLLIYDNNKRPSGIPDRFSGSKSGTSATLGITGLQTGDEADYYCGTWDSSLSGVVFGGGTKLTVLGSRGGGGSGGGGSGGGGSLEMAQVQLVQSGGEVKKPGASVKVSCKASGFTFNTYGISWVRQAPGQGLEWMGWISANNGHTKSAQRFQDRVAMATDTSTSTAYMELRSLKFDDTAVYYCARGGYHHQMQRYYKATSVYSDYWGQGTLVTVSS

SEQ ID NO: 122 - protein, 248 aa, synthetic construct
  REGION 1..248: note = Description of Artificial Sequence: Synthetic polypeptide
QSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNTLVFGGGTKLTVLGSRGGGGSGGGGSGGGGSLEMAQMQLVQSGAEVKKPGASVKVSCKASGYTFTSYYMHWVRQAPGQGLEWMGIINPSGGSSSYAQKFQGRVTMTRDTSTSTVYMELSSLRSEDTAVYYCARAGMGMDTWGQGTLVTVSS

SEQ ID NO: 123 - protein, 249 aa, synthetic construct
  REGION 1..249: note = Description of Artificial Sequence: Synthetic polypeptide
QSALTQPASVSGSPGQSITISCTGTSSDVGGYNYVSWYQQSPGKAPRLMIYGVSKRPSGVPDRFSGSKSGNTASLTVSGLQAEDEADYYCSSYAGVNNLMFGGGTKLTVLGSRGGGGSGGGGSGGGGSLEMAQVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDVISGFDSWGQGTLVTVSS

SEQ ID NO: 124 - protein, 8 aa, synthetic construct
  REGION 1..8: note = Description of Artificial Sequence: Synthetic peptide
GYTFTSYY

SEQ ID NO: 125 - protein, 8 aa, synthetic construct
  REGION 1..8: note = Description of Artificial Sequence: Synthetic peptide
GYTFTSYY

SEQ ID NO: 126 - protein, 13 aa, synthetic construct
  REGION 1..13: note = Description of Artificial Sequence: Synthetic peptide
ARGMYRSLLFYDP

SEQ ID NO: 127 - protein, 8 aa, synthetic construct
  REGION 1..8: note = Description of Artificial Sequence: Synthetic peptide
RSNVGNYY

SEQ ID NO: 128 - skipped

SEQ ID NO: 129 - protein, 11 aa, synthetic construct
  REGION 1..11: note = Description of Artificial Sequence: Synthetic peptide
GTWDGSLSAHV

SEQ ID NO: 130 - protein, 8 aa, synthetic construct
  REGION 1..8: note = Description of Artificial Sequence: Synthetic peptide
GFTFSNYA

SEQ ID NO: 131 - protein, 7 aa, synthetic construct
  REGION 1..7: note = Description of Artificial Sequence: Synthetic peptide
ISGSGNT

SEQ ID NO: 132 - protein, 10 aa, synthetic construct
  REGION 1..10: note = Description of Artificial Sequence: Synthetic peptide
ARGSVRYTDI

SEQ ID NO: 133 - protein, 8 aa, synthetic construct
  REGION 1..8: note = Description of Artificial Sequence: Synthetic peptide
SGAIAGAY

SEQ ID NO: 134 - skipped

SEQ ID NO: 135 - protein, 11 aa, synthetic construct
  REGION 1..11: note = Description of Artificial Sequence: Synthetic peptide
QSYDYDSSNVL

SEQ ID NO: 136 - protein, 8 aa, synthetic construct
  REGION 1..8: note = Description of Artificial Sequence: Synthetic peptide
GFTFNNYW

SEQ ID NO: 137 - protein, 8 aa, synthetic construct
  REGION 1..8: note = Description of Artificial Sequence: Synthetic peptide
IKQDGSEK

SEQ ID NO: 138 - protein, 8 aa, synthetic construct
  REGION 1..8: note = Description of Artificial Sequence: Synthetic peptide
ARSMSTAV

SEQ ID NO: 139 - protein, 6 aa, synthetic construct
  REGION 1..6: note = Description of Artificial Sequence: Synthetic peptide
QSISSY

SEQ ID NO: 140 - skipped

SEQ ID NO: 141 - protein, 9 aa, synthetic construct
  REGION 1..9: note = Description of Artificial Sequence: Synthetic peptide
QQSYSVPYT

SEQ ID NO: 142 - protein, 8 aa, synthetic construct
  REGION 1..8: note = Description of Artificial Sequence: Synthetic peptide
GYTFTSYY

SEQ ID NO: 143 - protein, 8 aa, synthetic construct
  REGION 1..8: note = Description of Artificial Sequence: Synthetic peptide
INPSGGST

SEQ ID NO: 144 - protein, 14 aa, synthetic construct
  REGION 1..14: note = Description of Artificial Sequence: Synthetic peptide
ARGSSRWGGWTGDY

SEQ ID NO: 145 - protein, 9 aa, synthetic construct
  REGION 1..9: note = Description of Artificial Sequence: Synthetic peptide
SSDVGGYNF

SEQ ID NO: 146 - skipped

SEQ ID NO: 147 - protein, 20 aa, synthetic construct
  REGION 1..20: note = Description of Artificial Sequence: Synthetic peptide
SSYTSTRTVIFAGGTKVTVL

SEQ ID NO: 148 - protein, 8 aa, synthetic construct
  REGION 1..8: note = Description of Artificial Sequence: Synthetic peptide
GSTFSSYA

SEQ ID NO: 149 - protein, 8 aa, synthetic construct
  REGION 1..8: note = Description of Artificial Sequence: Synthetic peptide
ISGRGRST

SEQ ID NO: 150 - protein, 9 aa, synthetic construct
  REGION 1..9: note = Description of Artificial Sequence: Synthetic peptide
ARYYKSKDH

SEQ ID NO: 151 - protein, 8 aa, synthetic construct
  REGION 1..8: note = Description of Artificial Sequence: Synthetic peptide
RSNIGTNY

SEQ ID NO: 152 - skipped

SEQ ID NO: 153 - protein, 11 aa, synthetic construct
  REGION 1..11: note = Description of Artificial Sequence: Synthetic peptide
AAWDDNLSGVV

SEQ ID NO: 154 - protein, 8 aa, synthetic construct
  REGION 1..8: note = Description of Artificial Sequence: Synthetic peptide
AYTFTDYY

SEQ ID NO: 155 - protein, 8 aa, synthetic construct
  REGION 1..8: note = Description of Artificial Sequence: Synthetic peptide
INPKSGRT

SEQ ID NO: 156 - protein, 14 aa, synthetic construct
  REGION 1..14: note = Description of Artificial Sequence: Synthetic peptide
ARVYGYSRWSGFDL

SEQ ID NO: 157 - protein, 8 aa, synthetic construct
  REGION 1..8: note = Description of Artificial Sequence: Synthetic peptide
SSNIGSNY

SEQ ID NO: 158 - skipped

SEQ ID NO: 159 - protein, 11 aa, synthetic construct
  REGION 1..11: note = Description of Artificial Sequence: Synthetic peptide
AAWDDSLSGYV

SEQ ID NO: 160 - protein, 8 aa, synthetic construct
  REGION 1..8: note = Description of Artificial Sequence: Synthetic peptide
GYTFTTYY

SEQ ID NO: 161 - protein, 8 aa, synthetic construct
  REGION 1..8: note = Description of Artificial Sequence: Synthetic peptide
INPNGGGT

SEQ ID NO: 162 - protein, 17 aa, synthetic construct
  REGION 1..17: note = Description of Artificial Sequence: Synthetic peptide
ARGHKVYKSHPTGGYDR

SEQ ID NO: 163 - protein, 9 aa, synthetic construct
  REGION 1..9: note = Description of Artificial Sequence: Synthetic peptide
SRDVGGYNY

SEQ ID NO: 164 - skipped

SEQ ID NO: 165 - protein, 10 aa, synthetic construct
  REGION 1..10: note = Description of Artificial Sequence: Synthetic peptide
SSYTSSSTLD

SEQ ID NO: 166 - protein, 8 aa, synthetic construct
  REGION 1..8: note = Description of Artificial Sequence: Synthetic peptide
GGTFSSYA

SEQ ID NO: 167 - protein, 8 aa, synthetic construct
  REGION 1..8: note = Description of Artificial Sequence: Synthetic peptide
IIPIFGTA

SEQ ID NO: 168 - protein, 12 aa, synthetic construct
  REGION 1..12: note = Description of Artificial Sequence: Synthetic peptide
ARSHVAWSLLDY

SEQ ID NO: 169 - protein, 8 aa, synthetic construct
  REGION 1..8: note = Description of Artificial Sequence: Synthetic peptide
SSNIGSNY

SEQ ID NO: 170 - skipped

SEQ ID NO: 171 - protein, 11 aa, synthetic construct
  REGION 1..11: note = Description of Artificial Sequence: Synthetic peptide
AAWDDSLSGVV

SEQ ID NO: 172 - protein, 8 aa, synthetic construct
  REGION 1..8: note = Description of Artificial Sequence: Synthetic peptide
GGTFSSYA

SEQ ID NO: 173 - protein, 8 aa, synthetic construct
  REGION 1..8: note = Description of Artificial Sequence: Synthetic peptide
MNPNSGNT

SEQ ID NO: 174 - protein, 13 aa, synthetic construct
  REGION 1..13: note = Description of Artificial Sequence: Synthetic peptide
ARYQSYKGSQSDS

SEQ ID NO: 175 - protein, 8 aa, synthetic construct
  REGION 1..8: note = Description of Artificial Sequence: Synthetic peptide
SSNIGSNY

SEQ ID NO: 176 - skipped

SEQ ID NO: 177 - protein, 11 aa, synthetic construct
  REGION 1..11: note = Description of Artificial Sequence: Synthetic peptide
AAWDDSLSGWV

SEQ ID NO: 178 - protein, 8 aa, synthetic construct
  REGION 1..8: note = Description of Artificial Sequence: Synthetic peptide
GYTFTSYY

SEQ ID NO: 179 - protein, 8 aa, synthetic construct
  REGION 1..8: note = Description of Artificial Sequence: Synthetic peptide
INPSGGST

SEQ ID NO: 180 - protein, 20 aa, synthetic construct
  REGION 1..20: note = Description of Artificial Sequence: Synthetic peptide
ARGGSKKWSGEKWRRENFDY

SEQ ID NO: 181 - protein, 9 aa, synthetic construct
  REGION 1..9: note = Description of Artificial Sequence: Synthetic peptide
SSDVGGYNY

SEQ ID NO: 182 - skipped

SEQ ID NO: 183 - protein, 10 aa, synthetic construct
  REGION 1..10: note = Description of Artificial Sequence: Synthetic peptide
SSYTRSSTEV

SEQ ID NO: 184 - protein, 8 aa, synthetic construct
  REGION 1..8: note = Description of Artificial Sequence: Synthetic peptide
EYTFTRHI

SEQ ID NO: 185 - protein, 8 aa, synthetic construct
  REGION 1..8: note = Description of Artificial Sequence: Synthetic peptide
INPGNGNT

SEQ ID NO: 186 - protein, 6 aa, synthetic construct
  REGION 1..6: note = Description of Artificial Sequence: Synthetic peptide
ARLPDQ

SEQ ID NO: 187 - protein, 8 aa, synthetic construct
  REGION 1..8: note = Description of Artificial Sequence: Synthetic peptide
SSNIGSNT

SEQ ID NO: 188 - skipped

SEQ ID NO: 189 - protein, 10 aa, synthetic construct
  REGION 1..10: note = Description of Artificial Sequence: Synthetic peptide
AAWDDSLSGL

SEQ ID NO: 190 - protein, 8 aa, synthetic construct
  REGION 1..8: note = Description of Artificial Sequence: Synthetic peptide
GFTFGDYG

SEQ ID NO: 191 - protein, 8 aa, synthetic construct
  REGION 1..8: note = Description of Artificial Sequence: Synthetic peptide
INWNGGST

SEQ ID NO: 192 - protein, 7 aa, synthetic construct
  REGION 1..7: note = Description of Artificial Sequence: Synthetic peptide
ARSKQDY

SEQ ID NO: 193 - protein, 9 aa, synthetic construct
  REGION 1..9: note = Description of Artificial Sequence: Synthetic peptide
SRDAGGYNY

SEQ ID NO: 194 - skipped

SEQ ID NO: 195 - protein, 11 aa, synthetic construct
  REGION 1..11: note = Description of Artificial Sequence: Synthetic peptide
SSYGGSNNFRV

SEQ ID NO: 196 - protein, 8 aa, synthetic construct
  REGION 1..8: note = Description of Artificial Sequence: Synthetic peptide
GFSFSGTA

SEQ ID NO: 197 - protein, 8 aa, synthetic construct
  REGION 1..8: note = Description of Artificial Sequence: Synthetic peptide
ISSTGRST

SEQ ID NO: 198 - protein, 7 aa, synthetic construct
  REGION 1..7: note = Description of Artificial Sequence: Synthetic peptide
ARVSFDY

SEQ ID NO: 199 - protein, 9 aa, synthetic construct
  REGION 1..9: note = Description of Artificial Sequence: Synthetic peptide
SSNIGAGYD

SEQ ID NO: 200 - skipped

SEQ ID NO: 201 - protein, 12 aa, synthetic construct
  REGION 1..12: note = Description of Artificial Sequence: Synthetic peptide
QSYDSSLSGSYV

SEQ ID NO: 202 - protein, 8 aa, synthetic construct
  REGION 1..8: note = Description of Artificial Sequence: Synthetic peptide
GFTFSSYA

SEQ ID NO: 203 - protein, 8 aa, synthetic construct
  REGION 1..8: note = Description of Artificial Sequence: Synthetic peptide
ISGRGRST

SEQ ID NO: 204 - protein, 11 aa, synthetic construct
  REGION 1..11: note = Description of Artificial Sequence: Synthetic peptide
ARYYHAGAFDL

SEQ ID NO: 205 - protein, 9 aa, synthetic construct
  REGION 1..9: note = Description of Artificial Sequence: Synthetic peptide
SSDVGGYNY

SEQ ID NO: 206 - skipped

SEQ ID NO: 207 - protein, 10 aa, synthetic construct
  REGION 1..10: note = Description of Artificial Sequence: Synthetic peptide
SSYTSSSTLV

SEQ ID NO: 208 - protein, 8 aa, synthetic construct
  REGION 1..8: note = Description of Artificial Sequence: Synthetic peptide
GYTFNRYA

SEQ ID NO: 209 - protein, 8 aa, synthetic construct
  REGION 1..8: note = Description of Artificial Sequence: Synthetic peptide
ISAYNGNS

SEQ ID NO: 210 - protein, 7 aa, synthetic construct
  REGION 1..7: note = Description of Artificial Sequence: Synthetic peptide
ARMAYDS

SEQ ID NO: 211 - protein, 9 aa, synthetic construct
  REGION 1..9: note = Description of Artificial Sequence: Synthetic peptide
SNDVGAYKY

SEQ ID NO: 212 - skipped

SEQ ID NO: 213 - protein, 10 aa, synthetic construct
  REGION 1..10: note = Description of Artificial Sequence: Synthetic peptide
FSLTSSNTYV

SEQ ID NO: 214 - protein, 8 aa, synthetic construct
  REGION 1..8: note = Description of Artificial Sequence: Synthetic peptide
GFTFSDYY

SEQ ID NO: 215 - protein, 8 aa, synthetic construct
  REGION 1..8: note = Description of Artificial Sequence: Synthetic peptide
ISSSGSTI

SEQ ID NO: 216 - protein, 10 aa, synthetic construct
  REGION 1..10: note = Description of Artificial Sequence: Synthetic peptide
ARGYGKAYDQ

SEQ ID NO: 217 - protein, 8 aa, synthetic construct
  REGION 1..8: note = Description of Artificial Sequence: Synthetic peptide
RSNVGGNY

SEQ ID NO: 218 - skipped

SEQ ID NO: 219 - protein, 11 aa, synthetic construct
  REGION 1..11: note = Description of Artificial Sequence: Synthetic peptide
ATWDDSLSGFV

SEQ ID NO: 220 - protein, 8 aa, synthetic construct
  REGION 1..8: note = Description of Artificial Sequence: Synthetic peptide
GFTFRSHS

SEQ ID NO: 221 - protein, 8 aa, synthetic construct
  REGION 1..8: note = Description of Artificial Sequence: Synthetic peptide
ISSDSTYT

SEQ ID NO: 222 - protein, 12 aa, synthetic construct
  REGION 1..12: note = Description of Artificial Sequence: Synthetic peptide
ARSGGQWKYYDY

SEQ ID NO: 223 - protein, 6 aa, synthetic construct
  REGION 1..6: note = Description of Artificial Sequence: Synthetic peptide
SLRSYY

SEQ ID NO: 224 - skipped

SEQ ID NO: 225 - protein, 12 aa, synthetic construct
  REGION 1..12: note = Description of Artificial Sequence: Synthetic peptide
NSRDSSGNPPVV

SEQ ID NO: 226 - protein, 8 aa, synthetic construct
  REGION 1..8: note = Description of Artificial Sequence: Synthetic peptide
GFTFSNYA

SEQ ID NO: 227 - protein, 8 aa, synthetic construct
  REGION 1..8: note = Description of Artificial Sequence: Synthetic peptide
INGRGSST

SEQ ID NO: 228 - protein, 11 aa, synthetic construct
  REGION 1..11: note = Description of Artificial Sequence: Synthetic peptide
ARYISRGLGDS

SEQ ID NO: 229 - protein, 8 aa, synthetic construct
  REGION 1..8: note = Description of Artificial Sequence: Synthetic peptide
NSNIERNY

SEQ ID NO: 230 - skipped

SEQ ID NO: 231 - protein, 11 aa, synthetic construct
  REGION 1..11: note = Description of Artificial Sequence: Synthetic peptide
GTWDSSLRGWV

SEQ ID NO: 232 - protein, 8 aa, synthetic construct
  REGION 1..8: note = Description of Artificial Sequence: Synthetic peptide
GYTFTSYY

SEQ ID NO: 233 - protein, 8 aa, synthetic construct
  REGION 1..8: note = Description of Artificial Sequence: Synthetic peptide
INPSGGST

SEQ ID NO: 234 - protein, 9 aa, synthetic construct
  REGION 1..9: note = Description of Artificial Sequence: Synthetic peptide
ARAGMGMDT

SEQ ID NO: 235 - protein, 9 aa, synthetic construct
  REGION 1..9: note = Description of Artificial Sequence: Synthetic peptide
SSDVGGYNY

SEQ ID NO: 236 - skipped

SEQ ID NO: 237 - protein, 10 aa, synthetic construct
  REGION 1..10: note = Description of Artificial Sequence: Synthetic peptide
SSYAGSNTLV

SEQ ID NO: 238 - protein, 8 aa, synthetic construct
  REGION 1..8: note = Description of Artificial Sequence: Synthetic peptide
GYTFTAYS

SEQ ID NO: 239 - protein, 8 aa, synthetic construct
  REGION 1..8: note = Description of Artificial Sequence: Synthetic peptide
INPSSGGA

SEQ ID NO: 240 - protein, 10 aa, synthetic construct
  REGION 1..10: note = Description of Artificial Sequence: Synthetic peptide
ARNVGGQADD

SEQ ID NO: 241 - protein, 9 aa, synthetic construct
  REGION 1..9: note = Description of Artificial Sequence: Synthetic peptide
SSDIGGYNY

SEQ ID NO: 242 - skipped

SEQ ID NO: 243 - protein, 10 aa, synthetic construct
  REGION 1..10: note = Description of Artificial Sequence: Synthetic peptide
ASFAGRKTLV

SEQ ID NO: 244 - protein, 8 aa, synthetic construct
  REGION 1..8: note = Description of Artificial Sequence: Synthetic peptide
GYTFTAYS

SEQ ID NO: 245 - protein, 8 aa, synthetic construct
  REGION 1..8: note = Description of Artificial Sequence: Synthetic peptide
INPSSGGA

SEQ ID NO: 246 - protein, 10 aa, synthetic construct
  REGION 1..10: note = Description of Artificial Sequence: Synthetic peptide
ARNVGGHADD

SEQ ID NO: 247 - protein, 9 aa, synthetic construct
  REGION 1..9: note = Description of Artificial Sequence: Synthetic peptide
STDIGGYNY

SEQ ID NO: 248 - skipped

SEQ ID NO: 249 - protein, 10 aa, synthetic construct
  REGION 1..10: note = Description of Artificial Sequence: Synthetic peptide
ASFAGRKTLV

SEQ ID NO: 250 - protein, 8 aa, synthetic construct
  REGION 1..8: note = Description of Artificial Sequence: Synthetic peptide
GFTFNTYG

SEQ ID NO: 251 - protein, 8 aa, synthetic construct
  REGION 1..8: note = Description of Artificial Sequence: Synthetic peptide
ISANNGHT

SEQ ID NO: 252 - protein, 22 aa, synthetic construct
  REGION 1..22: note = Description of Artificial Sequence: Synthetic peptide
ARGGYHHQMQRYYKATSVYSDY

SEQ ID NO: 253 - protein, 8 aa, synthetic construct
  REGION 1..8: note = Description of Artificial Sequence: Synthetic peptide
SSNIGNNY

SEQ ID NO: 254 - skipped

SEQ ID NO: 255 - protein, 11 aa, synthetic construct
  REGION 1..11: note = Description of Artificial Sequence: Synthetic peptide
GTWDSSLSGVV

SEQ ID NO: 256 - protein, 8 aa, synthetic construct
  REGION 1..8: note = Description of Artificial Sequence: Synthetic peptide
GYTFTSYY

SEQ ID NO: 257 - protein, 8 aa, synthetic construct
  REGION 1..8: note = Description of Artificial Sequence: Synthetic peptide
INPSGGSS

SEQ ID NO: 258 - protein, 9 aa, synthetic construct
  REGION 1..9: note = Description of Artificial Sequence: Synthetic peptide
ARAGMGMDT

SEQ ID NO: 259 - protein, 9 aa, synthetic construct
  REGION 1..9: note = Description of Artificial Sequence: Synthetic peptide
SSDVGGYNY

SEQ ID NO: 260 - skipped

SEQ ID NO: 261 - protein, 10 aa, synthetic construct
  REGION 1..10: note = Description of Artificial Sequence: Synthetic peptide
SSYAGSNTLV

SEQ ID NO: 262 - protein, 8 aa, synthetic construct
  REGION 1..8: note = Description of Artificial Sequence: Synthetic peptide
GYTFTSYY

SEQ ID NO: 263 - protein, 8 aa, synthetic construct
  REGION 1..8: note = Description of Artificial Sequence: Synthetic peptide
INPSGGST

SEQ ID NO: 264 - protein, 10 aa, synthetic construct
  REGION 1..10: note = Description of Artificial Sequence: Synthetic peptide
ARDVISGFDS

SEQ ID NO: 265 - protein, 9 aa, synthetic construct
  REGION 1..9: note = Description of Artificial Sequence: Synthetic peptide
SSDVGGYNY

SEQ ID NO: 266 - skipped

SEQ ID NO: 267 - protein, 10 aa, synthetic construct
  REGION 1..10: note = Description of Artificial Sequence: Synthetic peptide
SSYAGVNNLM

SEQ ID NO: 268 - protein, 8 aa, synthetic construct
  REGION 1..8: note = Description of Artificial Sequence: Synthetic peptide
GFTFGDYG

SEQ ID NO: 269 - protein, 8 aa, synthetic construct
  REGION 1..8: note = Description of Artificial Sequence: Synthetic peptide
INWNGGST

SEQ ID NO: 270 - protein, 7 aa, synthetic construct
  REGION 1..7: note = Description of Artificial Sequence: Synthetic peptide
ARSKQGY

SEQ ID NO: 271 - protein, 9 aa, synthetic construct
  REGION 1..9: note = Description of Artificial Sequence: Synthetic peptide
SRDAGGYNY

SEQ ID NO: 272 - skipped

SEQ ID NO: 273 - protein, 11 aa, synthetic construct
  REGION 1..11: note = Description of Artificial Sequence: Synthetic peptide
SSYGGSNNFRV

SEQ ID NO: 274 - protein, 114 aa, synthetic construct; identical to SEQ ID NO: 45
  REGION 1..114: note = Description of Artificial Sequence: Synthetic polypeptide

SEQ ID NO: 275 - protein, 136 aa, synthetic construct
  REGION 1..136: note = Description of Artificial Sequence: Synthetic polypeptide
MKKTAIAIAVALAGFATVAQAAELQSALTQPPSASGSPGQSVTISCTGTSRDAGGYNYFSWYQQHPGKAPKLLIYEVTKRPSGVPDRFSGSKSGKTASLTVSGLQADDEAVYYCSSYGGSNNFRVFGGGTKLTVLG

SEQ ID NO: 276 - protein, 271 aa, synthetic construct
  REGION 1..271: note = Description of Artificial Sequence: Synthetic polypeptide
MKKTAIAIAVALAGFATVAQAAELQSALTQPPSASGSPGQSVTISCTGTSRDAGGYNYFSWYQQHPGKAPKLLIYEVTKRPSGVPDRFSGSKSGKTASLTVSGLQADDEAVYYCSSYGGSNNFRVFGGGTKLTVLGSRGGGGSGGGGSGGGGSLEMAEVQLVESGGGVVRPGGSLRLSCAASGFTFGDYGMSWVRQAPGKGLEWVSGINWNGGSTGYADSVKGRFTISRDNAKNSLYLQMNSLRAEDTAVYYCARSKQDYWGQGTLVTVSS

SEQ ID NO: 277 - DNA, 342 bp, synthetic construct; identical to SEQ ID NO: 47
  misc_feature 1..342: note = Description of Artificial Sequence: Synthetic polynucleotide
  source 1..342: mol_type = other DNA

SEQ ID NO: 278 - DNA, 408 bp, synthetic construct
  misc_feature 1..408: note = Description of Artificial Sequence: Synthetic polynucleotide
  source 1..408: mol_type = other DNA
atgaaaaagacagctatcgcgattgcagtggcactggctggtttcgctaccgtggcccaggcggccgagctccagtctgccctgactcagcctccctccgcgtccgggtctcctggacagtcagtcaccatctcctgcactggaaccagcagggacgctggtggttataattatttctcctggtaccaacaacacccaggcaaagcccccaaactcctgatttatgaggtcactaagcggccctcaggggtccctgatcgcttctctggctccaagtctggcaagacggcctccctgaccgtctctgggctccaggctgacgatgaggctgtatattactgcagctcatatggaggcagcaacaactttcgggtgttcggcggagggaccaagctgaccgtcctaggt

SEQ ID NO: 279 - DNA, 813 bp, synthetic construct
  misc_feature 1..813: note = Description of Artificial Sequence: Synthetic polynucleotide
  source 1..813: mol_type = other DNA
atgaaaaagacagctatcgcgattgcagtggcactggctggtttcgctaccgtggcccaggcggccgagctccagtctgccctgactcagcctccctccgcgtccgggtctcctggacagtcagtcaccatctcctgcactggaaccagcagggacgctggtggttataattatttctcctggtaccaacaacacccaggcaaagcccccaaactcctgatttatgaggtcactaagcggccctcaggggtccctgatcgcttctctggctccaagtctggcaagacggcctccctgaccgtctctgggctccaggctgacgatgaggctgtatattactgcagctcatatggaggcagcaacaactttcgggtgttcggcggagggaccaagctgaccgtcctaggttctagaggtggtggtggtagcggcggcggcggctctggtggtggtggatccctcgagatggccgaggtgcagctggtggagtctgggggaggtgtggtacggcctggggggtccctgagactctcctgtgcagcctctggattcacctttggtgattatggcatgagctgggtccgccaagctccagggaaggggctggagtgggtctctggtattaattggaatggtggtagcacaggttatgcagactctgtgaagggccgattcaccatctccagagacaacgccaagaactccctgtatctgcaaatgaacagtctgagagccgaggacacggccgtatattactgtgcgcgctctaaacaggattactggggtcaaggtactctggtgaccgtctcctca

SEQ ID NO: 280 - protein, 8 aa, synthetic construct
  REGION 1..8: note = Description of Artificial Sequence: Synthetic peptide
GFTFSNYA

SEQ ID NO: 281 - protein, 8 aa, synthetic construct
  REGION 1..8: note = Description of Artificial Sequence: Synthetic peptide
ITNSGRST

SEQ ID NO: 282 - protein, 14 aa, synthetic construct
  REGION 1..14: note = Description of Artificial Sequence: Synthetic peptide
ARVTHRRYGSTFDS

SEQ ID NO: 283 - protein, 8 aa, synthetic construct
  REGION 1..8: note = Description of Artificial Sequence: Synthetic peptide
SSNIGSNT

SEQ ID NO: 284 - skipped

SEQ ID NO: 285 - protein, 11 aa, synthetic construct
  REGION 1..11: note = Description of Artificial Sequence: Synthetic peptide
AAWDDSVNGYV

SEQ ID NO: 286 - protein, 121 aa, synthetic construct
  REGION 1..121: note = Description of Artificial Sequence: Synthetic polypeptide
QLQLQESGGGSVQPGGSLRLSCAASGFTFSNYAMSWVRQAPGKGLEWVSAITNSGRSTYYADSVKGRFTISRDNSKNTLSLQMSSLRAEDTAVYYCARVTHRRYGSTFDSRGQGTLVTVSS

SEQ ID NO: 287 - protein, 135 aa, synthetic construct
  REGION 1..135: note = Description of Artificial Sequence: Synthetic polypeptide
MKKTAIAIAVALAGFATVAQAAELSYELTQPPSASGTPGQRVSISCSGSSSNIGSNTVNWYQQFPGTAPKLLIHSNNQRPSGVPDRFSGSKSGTSASLAISGPQSEDEADYYCAAWDDSVNGYVFGTGTKVTVLG

SEQ ID NO: 288 - protein, 277 aa, synthetic construct
  REGION 1..277: note = Description of Artificial Sequence: Synthetic polypeptide
MKKTAIAIAVALAGFATVAQAAELSYELTQPPSASGTPGQRVSISCSGSSSNIGSNTVNWYQQFPGTAPKLLIHSNNQRPSGVPDRFSGSKSGTSASLAISGPQSEDEADYYCAAWDDSVNGYVFGTGTKVTVLGSRGGGGSGGGGSGGGGSLEMAQLQLQESGGGSVQPGGSLRLSCAASGFTFSNYAMSWVRQAPGKGLEWVSAITNSGRSTYYADSVKGRFTISRDNSKNTLSLQMSSLRAEDTAVYYCARVTHRRYGSTFDSRGQGTLVTVSS

SEQ ID NO: 289 - DNA, 385 bp, synthetic construct
  misc_feature 1..385: note = Description of Artificial Sequence: Synthetic polynucleotide
  source 1..385: mol_type = other DNA
cagctgcagctgcaggagtcggggggaggctcggtacagccgggggggtctctgagactgtcctgtgcagcctctggattcacctttagcaactatgccatgagctgggtccgccaggctccagggaaggggctggagtgggtctcagctatcactaatagtggtcgtagtacatactacgcagactccgtgaagggccggttcaccatctccagagacaattccaagaacacgctgtctttgcaaatgagcagcctgagagccgaagacacggccgtgtattactgtgcgcgcgttactcatcgtcgttacggttctactttcgattctcggggtcaaggtactctggtgaccgtctcctcaactagtggccaggccggccagc

SEQ ID NO: 290 - DNA, 405 bp, synthetic construct
  misc_feature 1..405: note = Description of Artificial Sequence: Synthetic polynucleotide
  source 1..405: mol_type = other DNA
atgaaaaagacagctatcgcgattgcagtggcactggctggtttcgctaccgtggcccaggcggccgagctctcctatgagctgactcagccaccctcagcgtctgggacccccgggcagagggtcagcatctcttgttctggaagcagctccaacatcgggagtaatactgtaaactggtaccaacagttccccggaacggcccccaaactcctcatccatagtaataatcagcggccctcaggggtccctgaccgattctctggctccaagtctggcacctcagcctccctggccatcagtgggccccagtctgaggatgaggctgattattactgtgcagcttgggatgacagtgtgaatggttatgtcttcggaactgggaccaaggtcaccgtcctaggt

SEQ ID NO: 291 - DNA, 831 bp, synthetic construct
  misc_feature 1..831: note = Description of Artificial Sequence: Synthetic polynucleotide
  source 1..831: mol_type = other DNA
atgaaaaagacagctatcgcgattgcagtggcactggctggtttcgctaccgtggcccaggcggccgagctctcctatgagctgactcagccaccctcagcgtctgggacccccgggcagagggtcagcatctcttgttctggaagcagctccaacatcgggagtaatactgtaaactggtaccaacagttccccggaacggcccccaaactcctcatccatagtaataatcagcggccctcaggggtccctgaccgattctctggctccaagtctggcacctcagcctccctggccatcagtgggccccagtctgaggatgaggctgattattactgtgcagcttgggatgacagtgtgaatggttatgtcttcggaactgggaccaaggtcaccgtcctaggttctagaggtggtggtggtagcggcggcggcggctctggtggtggtggatccctcgagatggcccagctgcagctgcaggagtcggggggaggctcggtacagccgggggggtctctgagactgtcctgtgcagcctctggattcacctttagcaactatgccatgagctgggtccgccaggctccagggaaggggctggagtgggtctcagctatcactaatagtggtcgtagtacatactacgcagactccgtgaagggccggttcaccatctccagagacaattccaagaacacgctgtctttgcaaatgagcagcctgagagccgaagacacggccgtgtattactgtgcgcgcgttactcatcgtcgttacggttctactttcgattctcggggtcaaggtactctggtgaccgtctcctca

SEQ ID NO: 292 - protein, 8 aa, synthetic construct
  REGION 1..8: note = Description of Artificial Sequence: Synthetic peptide
GGTFRSYA

SEQ ID NO: 293 - protein, 8 aa, synthetic construct
  REGION 1..8: note = Description of Artificial Sequence: Synthetic peptide
IIPMLDIT

SEQ ID NO: 294 - protein, 14 aa, synthetic construct
  REGION 1..14: note = Description of Artificial Sequence: Synthetic peptide
ARTYSRSPFHMEDF

SEQ ID NO: 295 - protein, 8 aa, synthetic construct
  REGION 1..8: note = Description of Artificial Sequence: Synthetic peptide
SSNIGGNT

SEQ ID NO: 296 - skipped

SEQ ID NO: 297 - protein, 10 aa, synthetic construct
  REGION 1..10: note = Description of Artificial Sequence: Synthetic peptide
AAWDASRQGV

SEQ ID NO: 298 - protein, 121 aa, synthetic construct
  REGION 1..121: note = Description of Artificial Sequence: Synthetic polypeptide
QVQLVQSGAEVKKPGSSVKVSCKASGGTFRSYAITWVRQAPGQGLEWMGRIIPMLDITNYAQKFQGRVTITADKSTSTAYMELSSLRSEDTAVYYCARTYSRSPFHMEDFWGQGTLVTVSS

SEQ ID NO: 299 - protein, 134 aa, synthetic construct
  REGION 1..134: note = Description of Artificial Sequence: Synthetic polypeptide
MKKTAIAIAVALAGFATVAQAAELQPVLTQPPSASGTPGQRVTISCSGSSSNIGGNTVSWYQQVPGTAPRLLIFRNNQRPPGVPDRFSGSKSGTSASLAISGLRSEDEADYYCAAWDASRQGVFGGGTKLTVLG

SEQ ID NO: 300 - protein, 276 aa, synthetic construct
  REGION 1..276: note = Description of Artificial Sequence: Synthetic polypeptide
MKKTAIAIAVALAGFATVAQAAELQPVLTQPPSASGTPGQRVTISCSGSSSNIGGNTVSWYQQVPGTAPRLLIFRNNQRPPGVPDRFSGSKSGTSASLAISGLRSEDEADYYCAAWDASRQGVFGGGTKLTVLGSRGGGGSGGGGSGGGGSLEMAQVQLVQSGAEVKKPGSSVKVSCKASGGTFRSYAITWVRQAPGQGLEWMGRIIPMLDITNYAQKFQGRVTITADKSTSTAYMELSSLRSEDTAVYYCARTYSRSPFHMEDFWGQGTLVTVSS

SEQ ID NO: 301 - DNA, 402 bp, synthetic construct
  misc_feature 1..402: note = Description of Artificial Sequence: Synthetic polynucleotide
  source 1..402: mol_type = other DNA
atgaaaaagacagctatcgcgattgcagtggcactggctggtttcgctaccgtggcccaggcggccgagctccagcctgtgctgactcagccaccctcagcgtctgggacccccgggcagagggtcaccatctcttgttctggaagcagctccaatatcggaggtaacactgtcagctggtaccagcaggtcccaggaacggcccccagactcctcatttttaggaataatcaacggcccccaggggtccctgaccgattctctggctccaagtctggcacctcagcctccctggccatcagtgggctccggtctgaggatgaggctgattattactgtgcagcatgggacgccagtcgacaaggggtgttcggcggagggaccaagctgaccgtcctaggt

SEQ ID NO: 302 - DNA, 828 bp, synthetic construct
  misc_feature 1..828: note = Description of Artificial Sequence: Synthetic polynucleotide
  source 1..828: mol_type = other DNA
atgaaaaagacagctatcgcgattgcagtggcactggctggtttcgctaccgtggcccaggcggccgagctccagcctgtgctgactcagccaccctcagcgtctgggacccccgggcagagggtcaccatctcttgttctggaagcagctccaatatcggaggtaacactgtcagctggtaccagcaggtcccaggaacggcccccagactcctcatttttaggaataatcaacggcccccaggggtccctgaccgattctctggctccaagtctggcacctcagcctccctggccatcagtgggctccggtctgaggatgaggctgattattactgtgcagcatgggacgccagtcgacaaggggtgttcggcggagggaccaagctgaccgtcctaggttctagaggtggtggtggtagcggcggcggcggctctggtggtggtggatccctcgagatggcccaggtgcagctggtgcagtctggggctgaggtgaagaagcctgggtcctcggtgaaggtctcctgcaaggcttctggaggcaccttccgcagctatgctatcacctgggtgcgacaggcccctggacaagggcttgagtggatgggaaggatcatccctatgcttgatataacaaactacgcacagaagttccagggcagagtcacgattaccgcggacaaatccacgagcacagcctacatggagctgagcagcctgagatctgaggacacggccgtgtattactgtgcgcgcacttactctcgttctccgttccatatggaagatttctggggtcaaggtactctggtgaccgtctcctca

SEQ ID NO: 303 - protein, 8 aa, synthetic construct
  REGION 1..8: note = Description of Artificial Sequence: Synthetic peptide
GFTFSSYA

SEQ ID NO: 304 - protein, 8 aa, synthetic construct
  REGION 1..8: note = Description of Artificial Sequence: Synthetic peptide
ISGSGGST

SEQ ID NO: 305 - protein, 7 aa, synthetic construct
  REGION 1..7: note = Description of Artificial Sequence: Synthetic peptide
ARKYQDV

SEQ ID NO: 306 - protein, 8 aa, synthetic construct
  REGION 1..8: note = Description of Artificial Sequence: Synthetic peptide
SSNIGSNT

SEQ ID NO: 307 - skipped

SEQ ID NO: 308 - protein, 11 aa, synthetic construct
  REGION 1..11: note = Description of Artificial Sequence: Synthetic peptide
AAWDDSLSGRV

SEQ ID NO: 309 - protein, 15 aa, synthetic construct
  REGION 1..15: note = Description of Artificial Sequence: Synthetic peptide
GGGGSGGGGSGGGGS

SEQ ID NO: 310 - protein, 114 aa, synthetic construct
  REGION 1..114: note = Description of Artificial Sequence: Synthetic polypeptide
EVQLVESGGGLVQPGGSLRLSCAASGFTFSSYAMSWVRQAPGKGLEWVSAISGSGGSTYYADSVKGRFTISRDNAKNTLYLQMNSLRAEDTAVYYCARKYQDVWGQGTLVTVSS

SEQ ID NO: 311 - protein, 135 aa, synthetic construct
  REGION 1..135: note = Description of Artificial Sequence: Synthetic polypeptide
MKKTAIAIAVALAGFATVAQAAELQSVLTQPPSASGTPGQRVTISCSGSSSNIGSNTVNWYQQLPGTAPKLLIYRNNQRPSGVPDRFSGSKSGTSASLAISGLRSEDEADYYCAAWDDSLSGRVFGGGTKLTVLG

SEQ ID NO: 312 - protein, 270 aa, synthetic construct
  REGION 1..270: note = Description of Artificial Sequence: Synthetic polypeptide
MKKTAIAIAVALAGFATVAQAAELQSVLTQPPSASGTPGQRVTISCSGSSSNIGSNTVNWYQQLPGTAPKLLIYRNNQRPSGVPDRFSGSKSGTSASLAISGLRSEDEADYYCAAWDDSLSGRVFGGGTKLTVLGSRGGGGSGGGGSGGGGSLEMAEVQLVESGGGLVQPGGSLRLSCAASGFTFSSYAMSWVRQAPGKGLEWVSAISGSGGSTYYADSVKGRFTISRDNAKNTLYLQMNSLRAEDTAVYYCARKYQDVWGQGTLVTVSS

SEQ ID NO: 313 - DNA, 342 bp, synthetic construct
  misc_feature 1..342: note = Description of Artificial Sequence: Synthetic polynucleotide
  source 1..342: mol_type = other DNA
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaatgccaagaacacgctgtatctgcaaatgaacagcctgagagccgaggacacggccgtatattactgtgcgcgcaaataccaggatgtttggggtcaaggtactctggtgaccgtctcctca

SEQ ID NO: 314 - DNA, 405 bp, synthetic construct
  misc_feature 1..405: note = Description of Artificial Sequence: Synthetic polynucleotide
  source 1..405: mol_type = other DNA
atgaaaaagacagctatcgcgattgcagtggcactggctggtttcgctaccgtggcccaggcggccgagctccagtctgtgctgacgcagccgccctcagcgtctgggacccccgggcagagggtcaccatctcttgttctggaagcagctccaacatcggaagtaatactgtaaactggtaccagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgagtggtagggtgttcggcggagggaccaagctgaccgtcctaggt

SEQ ID NO: 315 - DNA, 810 bp, synthetic construct
  misc_feature 1..810: note = Description of Artificial Sequence: Synthetic polynucleotide
  source 1..810: mol_type = other DNA
atgaaaaagacagctatcgcgattgcagtggcactggctggtttcgctaccgtggcccaggcggccgagctccagtctgtgctgacgcagccgccctcagcgtctgggacccccgggcagagggtcaccatctcttgttctggaagcagctccaacatcggaagtaatactgtaaactggtaccagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgagtggtagggtgttcggcggagggaccaagctgaccgtcctaggttctagaggtggtggtggtagcggcggcggcggctctggtggtggtggatccctcgagatggccgaggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaatgccaagaacacgctgtatctgcaaatgaacagcctgagagccgaggacacggccgtatattactgtgcgcgcaaataccaggatgtttggggtcaaggtactctggtgaccgtctcctca

SEQ ID NO: 316 - protein, 8 aa, synthetic construct
  REGION 1..8: note = Description of Artificial Sequence: Synthetic peptide
GFSFSGTA

SEQ ID NO: 317 - protein, 8 aa, synthetic construct
  REGION 1..8: note = Description of Artificial Sequence: Synthetic peptide
ISSTGRST

SEQ ID NO: 318 - protein, 15 aa, synthetic construct
  REGION 1..15: note = Description of Artificial Sequence: Synthetic peptide
ARPVSSMTLSIQSDG

SEQ ID NO: 319 - protein, 9 aa, synthetic construct
  REGION 1..9: note = Description of Artificial Sequence: Synthetic peptide
SSNIGAGYD

SEQ ID NO: 320 - skipped

SEQ ID NO: 321 - protein, 11 aa, synthetic construct
  REGION 1..11: note = Description of Artificial Sequence: Synthetic peptide
QSYDSSLRGYV

SEQ ID NO: 322 - protein, 122 aa, synthetic construct
  REGION 1..122: note = Description of Artificial Sequence: Synthetic polypeptide
QVQLVQSGGGVVQPGRSLRLSCAASGFSFSGTAMHWVRQAPGKGLEWVSTISSTGRSTYYRDSVKGRFTISRDNSKNTLYLQMNSLRGEDTAVYYCARPVSSMTLSIQSDGWGQGTLVTVSS

SEQ ID NO: 323 - protein, 136 aa, synthetic construct
  REGION 1..136: note = Description of Artificial Sequence: Synthetic polypeptide
MKKTAIAIAVALAGFATVAQAAELQSVLTQPPSVSGAPGQRVTISCTGSSSNIGAGYDVHWYQQLPGRAPKLLIYGNSNRPSGVPDRFSGSKSGTSASLAITGLQAEDEADYYCQSYDSSLRGYVFGTGTKVTVLG

SEQ ID NO: 324 - protein, 279 aa, synthetic construct
  REGION 1..279: note = Description of Artificial Sequence: Synthetic polypeptide
MKKTAIAIAVALAGFATVAQAAELQSVLTQPPSVSGAPGQRVTISCTGSSSNIGAGYDVHWYQQLPGRAPKLLIYGNSNRPSGVPDRFSGSKSGTSASLAITGLQAEDEADYYCQSYDSSLRGYVFGTGTKVTVLGSRGGGGSGGGGSGGGGSLEMAQVQLVQSGGGVVQPGRSLRLSCAASGFSFSGTAMHWVRQAPGKGLEWVSTISSTGRSTYYRDSVKGRFTISRDNSKNTLYLQMNSLRGEDTAVYYCARPVSSMTLSIQSDGWGQGTLVTVSS

SEQ ID NO: 325 - DNA, 366 bp, synthetic construct
  misc_feature 1..366: note = Description of Artificial Sequence: Synthetic polynucleotide
  source 1..366: mol_type = other DNA
caggtgcagctggtgcagtctgggggaggcgtggtccagcctgggaggtccctgagactctcctgtgcagcctctggattcagctttagtggcactgccatgcactgggtccgccaggctccagggaaggggctggaatgggtctcgactattagtagtactgggcgtagcacatactacagagactccgtgaagggccggttcaccatctccagagacaattccaagaacacgctgtatctgcaaatgaacagcctgagaggcgaggacacggccgtatattactgtgcgcgcccggtttcttctatgactctgtctatccagtctgatggttggggtcaaggtactctggtgaccgtctcctca

SEQ ID NO: 326 - DNA, 408 bp, synthetic construct
  misc_feature 1..408: note = Description of Artificial Sequence: Synthetic polynucleotide
  source 1..408: mol_type = other DNA
atgaaaaagacagctatcgcgattgcagtggcactggctggtttcgctaccgtggcccaggcggccgagctccagtctgtgttgacgcagccgccctcagtgtctggggccccagggcagagggtcaccatctcctgcactgggagcagctccaacatcggggcaggttatgatgtacactggtaccagcagcttccaggaagagcccccaaactcctcatctatggtaacagcaatcggccctcaggggtccctgaccgattctctggctccaagtctggcacctcagcctccctggccatcactgggctccaggctgaggatgaggctgattattactgccagtcctatgacagcagcctgagaggttatgtcttcggaactgggaccaaggtcaccgtcctaggt

SEQ ID NO: 327 - DNA, 837 bp, synthetic construct
  misc_feature 1..837: note = Description of Artificial Sequence: Synthetic polynucleotide
  source 1..837: mol_type = other DNA
atgaaaaagacagctatcgcgattgcagtggcactggctggtttcgctaccgtggcccaggcggccgagctccagtctgtgttgacgcagccgccctcagtgtctggggccccagggcagagggtcaccatctcctgcactgggagcagctccaacatcggggcaggttatgatgtacactggtaccagcagcttccaggaagagcccccaaactcctcatctatggtaacagcaatcggccctcaggggtccctgaccgattctctggctccaagtctggcacctcagcctccctggccatcactgggctccaggctgaggatgaggctgattattactgccagtcctatgacagcagcctgagaggttatgtcttcggaactgggaccaaggtcaccgtcctaggttctagaggtggtggtggtagcggcggcggcggctctggtggtggtggatccctcgagatggcccaggtgcagctggtgcagtctgggggaggcgtggtccagcctgggaggtccctgagactctcctgtgcagcctctggattcagctttagtggcactgccatgcactgggtccgccaggctccagggaaggggctggaatgggtctcgactattagtagtactgggcgtagcacatactacagagactccgtgaagggccggttcaccatctccagagacaattccaagaacacgctgtatctgcaaatgaacagcctgagaggcgaggacacggccgtatattactgtgcgcgcccggtttcttctatgactctgtctatccagtctgatggttggggtcaaggtactctggtgaccgtctcctca

SEQ ID NO: 328 - protein, 8 aa, synthetic construct
  REGION 1..8: note = Description of Artificial Sequence: Synthetic peptide
GYTFTSYY

SEQ ID NO: 329 - protein, 8 aa, synthetic construct
  REGION 1..8: note = Description of Artificial Sequence: Synthetic peptide
INPSGGST

SEQ ID NO: 330 - protein, 23 aa, synthetic construct
  REGION 1..23: note = Description of Artificial Sequence: Synthetic peptide
ARGQKYHSQYSRGGTGGGMTQDM

SEQ ID NO: 331 - protein, 8 aa, synthetic construct
  REGION 1..8: note = Description of Artificial Sequence: Synthetic peptide
SSNIGNNY

SEQ ID NO: 332 - skipped

SEQ ID NO: 333 - protein, 11 aa, synthetic construct
  REGION 1..11: note = Description of Artificial Sequence: Synthetic peptide
GTWDSSLRNWV

SEQ ID NO: 334 - protein, 130 aa, synthetic construct
  REGION 1..130: note = Description of Artificial Sequence: Synthetic polypeptide
QMQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGQKYHSQYSRGGTGGGMTQDMWGQGTLVTVSS

SEQ ID NO: 335 - protein, 135 aa, synthetic construct
  REGION 1..135: note = Description of Artificial Sequence: Synthetic polypeptide
MKKTAIAIAVALAGFATVAQAAELQSVVTQPPSVSAAPGQRVTISCSGGSSNIGNNYVSWFQQLPRTAPKLLIYDNNKRPSGIPDRFSGSKSGTSAALDITVLQTGDEADYYCGTWDSSLRNWVFGGGTKLTVLG

SEQ ID NO: 336 - protein, 286 aa, synthetic construct
  REGION 1..286: note = Description of Artificial Sequence: Synthetic polypeptide
MKKTAIAIAVALAGFATVAQAAELQSVVTQPPSVSAAPGQRVTISCSGGSSNIGNNYVSWFQQLPRTAPKLLIYDNNKRPSGIPDRFSGSKSGTSAALDITVLQTGDEADYYCGTWDSSLRNWVFGGGTKLTVLGSRGGGGSGGGGSGGGGSLEMAQMQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGQKYHSQYSRGGTGGGMTQDMWGQGTLVTVSS

SEQ ID NO: 337 - DNA, 390 bp, synthetic construct
  misc_feature 1..390: note = Description of Artificial Sequence: Synthetic polynucleotide
  source 1..390: mol_type = other DNA
cagatgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacaaaagttccagggcagagtcaccatgaccagggacacgtccacgagcacagtctacatggagctgagcagcctgagatctgaggacacggccgtgtattactgtgcgcgcggtcagaaataccattctcagtactctcgtggtggtactggtggtggtatgactcaggatatgtggggtcaaggtactctggtgaccgtctcctca

SEQ ID NO: 338 - DNA, 405 bp, synthetic construct
  misc_feature 1..405: note = Description of Artificial Sequence: Synthetic polynucleotide
  source 1..405: mol_type = other DNA
atgaaaaagacagctatcgcgattgcagtggcactggctggtttcgctaccgtggcccaggcggccgagctccagtctgtcgtgacgcagccgccctctgtgtctgcggccccaggacagagggtcaccatctcctgctctggaggtagttccaacattgggaataattatgtttcctggttccaacaactcccacgaacagcccccaaactcctcatttatgacaataataagcgaccctcagggattcctgaccgattctctggctccaagtctggcacgtcagccgccctggacatcaccgttctccagactggggacgaggccgattattactgcggaacttgggatagcagcctgagaaattgggtgttcggcggagggaccaagctgaccgtcctaggt

SEQ ID NO: 339 - DNA, 858 bp, synthetic construct
  misc_feature 1..858: note = Description of Artificial Sequence: Synthetic polynucleotide
  source 1..858: mol_type = other DNA
atgaaaaagacagctatcgcgattgcagtggcactggctggtttcgctaccgtggcccaggcggccgagctccagtctgtcgtgacgcagccgccctctgtgtctgcggccccaggacagagggtcaccatctcctgctctggaggtagttccaacattgggaataattatgtttcctggttccaacaactcccacgaacagcccccaaactcctcatttatgacaataataagcgaccctcagggattcctgaccgattctctggctccaagtctggcacgtcagccgccctggacatcaccgttctccagactggggacgaggccgattattactgcggaacttgggatagcagcctgagaaattgggtgttcggcggagggaccaagctgaccgtcctaggttctagaggtggtggtggtagcggcggcggcggctctggtggtggtggatccctcgagatggcccagatgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacaaaagttccagggcagagtcaccatgaccagggacacgtccacgagcacagtctacatggagctgagcagcctgagatctgaggacacggccgtgtattactgtgcgcgcggtcagaaataccattctcagtactctcgtggtggtactggtggtggtatgactcaggatatgtggggtcaaggtactctggtgaccgtctcctca

SEQ ID NO: 340 - protein, 8 aa, synthetic construct
  REGION 1..8: note = Description of Artificial Sequence: Synthetic peptide
GYTFSRYY

SEQ ID NO: 341 - protein, 8 aa, synthetic construct
  REGION 1..8: note = Description of Artificial Sequence: Synthetic peptide
MNPNSGNT

SEQ ID NO: 342 - protein, 10 aa, synthetic construct
  REGION 1..10: note = Description of Artificial Sequence: Synthetic peptide
ARGRYHVIDY

SEQ ID NO: 343 - protein, 9 aa, synthetic construct
  REGION 1..9: note = Description of Artificial Sequence: Synthetic peptide
SSDVGGYNH

SEQ ID NO: 344 - skipped

SEQ ID NO: 345 - protein, 10 aa, synthetic construct
  REGION 1..10: note = Description of Artificial Sequence: Synthetic peptide
SSYAGSAHWV

SEQ ID NO: 346 - protein, 117 aa, synthetic construct
  REGION 1..117: note = Description of Artificial Sequence: Synthetic polypeptide
EVQLVQSGAEVKKPGASVKVSCKASGYTFSRYYIHWVRQAPGQGLEWMGWMNPNSGNTGYAQKFQGRVTMTRNTSISTAYMELSSLRSEDTAVYYCARGRYHVIDYWGQGTLVTVSS

SEQ ID NO: 347 - protein, 135 aa, synthetic construct
  REGION 1..135: note = Description of Artificial Sequence: Synthetic polypeptide
MKKTAIAIAVALAGFATVAQAAELQSVLTQPPSASGSPGQSLTISCTGTSSDVGGYNHVSWYQQYPGKAPKLMIYEVTKRPSGVPDRFSGSKSGNTASLTVSGLQAEDEADYYCSSYAGSAHWVFGGGTKLTVLG

SEQ ID NO: 348 - protein, 273 aa, synthetic construct
  REGION 1..273: note = Description of Artificial Sequence: Synthetic polypeptide
MKKTAIAIAVALAGFATVAQAAELQSVLTQPPSASGSPGQSLTISCTGTSSDVGGYNHVSWYQQYPGKAPKLMIYEVTKRPSGVPDRFSGSKSGNTASLTVSGLQAEDEADYYCSSYAGSAHWVFGGGTKLTVLGSRGGGGSGGGGSGGGGSLEMAEVQLVQSGAEVKKPGASVKVSCKASGYTFSRYYIHWVRQAPGQGLEWMGWMNPNSGNTGYAQKFQGRVTMTRNTSISTAYMELSSLRSEDTAVYYCARGRYHVIDYWGQGTLVTVSS

SEQ ID NO: 349 - DNA, 351 bp, synthetic construct
  misc_feature 1..351: note = Description of Artificial Sequence: Synthetic polynucleotide
  source 1..351: mol_type = other DNA
gaggtccagctggtgcagtctggggctgaggtgaagaagcctggggcctcagtgaaggtttcctgcaaggcatctggatacaccttcagcaggtactatatacactgggtgcgacaggcccctggacaagggcttgagtggatgggatggatgaaccctaacagtggtaacacaggctatgcacagaagttccagggcagagtcaccatgaccaggaacacctccataagcacagcctacatggagctgagcagcctgagatctgaggacacggccgtgtattactgtgcgcgcggtcgttaccatgttatcgattactggggtcaaggtactctggtgaccgtctcctca

SEQ ID NO: 350 - DNA, 405 bp, synthetic construct
  misc_feature 1..405: note = Description of Artificial Sequence: Synthetic polynucleotide
  source 1..405: mol_type = other DNA
atgaaaaagacagctatcgcgattgcagtggcactggctggtttcgctaccgtggcccaggcggccgagctccagtctgtgttgactcagccaccctccgcgtccgggtctcctggacagtcactcaccatctcctgcactggaaccagcagtgacgttggtggttataaccatgtctcctggtaccaacagtacccaggcaaagcccccaaactcatgatttatgaggtcactaagcggccctcaggggtccctgatcgcttctctggctccaagtctggcaacacggcctccctgaccgtctctgggctccaggctgaggatgaggctgattattactgcagctcatatgcaggcagcgcccattgggtgttcggcggagggaccaagctgaccgtcctaggt

SEQ ID NO: 351 - DNA, 819 bp, synthetic construct
  misc_feature 1..819: note = Description of Artificial Sequence: Synthetic polynucleotide
  source 1..819: mol_type = other DNA
atgaaaaagacagctatcgcgattgcagtggcactggctggtttcgctaccgtggcccaggcggccgagctccagtctgtgttgactcagccaccctccgcgtccgggtctcctggacagtcactcaccatctcctgcactggaaccagcagtgacgttggtggttataaccatgtctcctggtaccaacagtacccaggcaaagcccccaaactcatgatttatgaggtcactaagcggccctcaggggtccctgatcgcttctctggctccaagtctggcaacacggcctccctgaccgtctctgggctccaggctgaggatgaggctgattattactgcagctcatatgcaggcagcgcccattgggtgttcggcggagggaccaagctgaccgtcctaggttctagaggtggtggtggtagcggcggcggcggctctggtggtggtggatccctcgagatggccgaggtccagctggtgcagtctggggctgaggtgaagaagcctggggcctcagtgaaggtttcctgcaaggcatctggatacaccttcagcaggtactatatacactgggtgcgacaggcccctggacaagggcttgagtggatgggatggatgaaccctaacagtggtaacacaggctatgcacagaagttccagggcagagtcaccatgaccaggaacacctccataagcacagcctacatggagctgagcagcctgagatctgaggacacggccgtgtattactgtgcgcgcggtcgttaccatgttatcgattactggggtcaaggtactctggtgaccgtctcctca

SEQ ID NO: 352 - protein, 8 aa, synthetic construct
  REGION 1..8: note = Description of Artificial Sequence: Synthetic peptide
GYTFNTYY

SEQ ID NO: 353 - protein, 8 aa, synthetic construct
  REGION 1..8: note = Description of Artificial Sequence: Synthetic peptide
INPNNGGT

SEQ ID NO: 354 - protein, 6 aa, synthetic construct
  REGION 1..6: note = Description of Artificial Sequence: Synthetic peptide
ARSYDY

SEQ ID NO: 355 - protein, 8 aa, synthetic construct
  REGION 1..8: note = Description of Artificial Sequence: Synthetic peptide
SSNIGSNY

SEQ ID NO: 356 - skipped

SEQ ID NO: 357 - protein, 11 aa, synthetic construct
  REGION 1..11: note = Description of Artificial Sequence: Synthetic peptide
AAWDDSLSGRV

SEQ ID NO: 358 - protein, 113 aa, synthetic construct
  REGION 1..113: note = Description of Artificial Sequence: Synthetic polypeptide
QLQLVQSGAEVKKPGSSVKVSCKASGYTFNTYYLHWVRQAPGQGLEWMGRINPNNGGTNYAQKFQGRVTMTRDTSINTAYMELSRLRSDDTAVYYCARSYDYWGQGTLVTVSS

SEQ ID NO: 359 - protein, 135 aa, synthetic construct
  REGION 1..135: note = Description of Artificial Sequence: Synthetic polypeptide
MKKTAIAIAVALAGFATVAQAAELQAVLTQPPSASGTPGQRVTISCSGSSSNIGSNYVYWYQQLPGTAPKLLIYRNNQRPSGVPDRFSGSKSGTSASLAISGLRSEDEADYYCAAWDDSLSGRVFGTGTKVTVLG

SEQ ID NO: 360 - protein, 269 aa, synthetic construct
  REGION 1..269: note = Description of Artificial Sequence: Synthetic polypeptide
MKKTAIAIAVALAGFATVAQAAELQAVLTQPPSASGTPGQRVTISCSGSSSNIGSNYVYWYQQLPGTAPKLLIYRNNQRPSGVPDRFSGSKSGTSASLAISGLRSEDEADYYCAAWDDSLSGRVFGTGTKVTVLGSRGGGGSGGGGSGGGGSLEMAQLQLVQSGAEVKKPGSSVKVSCKASGYTFNTYYLHWVRQAPGQGLEWMGRINPNNGGTNYAQKFQGRVTMTRDTSINTAYMELSRLRSDDTAVYYCARSYDYWGQGTLVTVSS

SEQ ID NO: 361 - DNA, 339 bp, synthetic construct
  misc_feature 1..339: note = Description of Artificial Sequence: Synthetic polynucleotide
  source 1..339: mol_type = other DNA
cagctgcagctggtgcaatctggggctgaggtgaagaagcctgggtcctcggtgaaggtctcctgcaaggcttctggatacaccttcaacacctactatctgcactgggtacgacaggcccctggacaagggcttgagtggatgggacggatcaaccctaacaatggtggcacaaactatgcacagaagtttcagggcagggtcaccatgaccagggacacgtccatcaacacagcctacatggagctgagcaggctgagatctgacgacacggccgtgtattactgtgcgcgctcttacgattactggggtcaaggtactctggtgaccgtctcctca

SEQ ID NO: 362 - DNA, 405 bp, synthetic construct
  misc_feature 1..405: note = Description of Artificial Sequence: Synthetic polynucleotide
  source 1..405: mol_type = other DNA
atgaaaaagacagctatcgcgattgcagtggcactggctggtttcgctaccgtggcccaggcggccgagctccaggctgtgctgactcagccaccctcagcgtctgggacccccgggcagagggtcaccatctcttgttctggaagcagctccaacatcggaagtaattatgtatactggtaccagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgagtggtcgggtcttcggaactgggaccaaggtcaccgtcctaggt

SEQ ID NO: 363 - DNA, 807 bp, synthetic construct
  misc_feature 1..807: note = Description of Artificial Sequence: Synthetic polynucleotide
  source 1..807: mol_type = other DNA
atgaaaaagacagctatcgcgattgcagtggcactggctggtttcgctaccgtggcccaggcggccgagctccaggctgtgctgactcagccaccctcagcgtctgggacccccgggcagagggtcaccatctcttgttctggaagcagctccaacatcggaagtaattatgtatactggtaccagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgagtggtcgggtcttcggaactgggaccaaggtcaccgtcctaggttctagaggtggtggtggtagcggcggcggcggctctggtggtggtggatccctcgagatggcccagctgcagctggtgcaatctggggctgaggtgaagaagcctgggtcctcggtgaaggtctcctgcaaggcttctggatacaccttcaacacctactatctgcactgggtacgacaggcccctggacaagggcttgagtggatgggacggatcaaccctaacaatggtggcacaaactatgcacagaagtttcagggcagggtcaccatgaccagggacacgtccatcaacacagcctacatggagctgagcaggctgagatctgacgacacggccgtgtattactgtgcgcgctcttacgattactggggtcaaggtactctggtgaccgtctcctca

SEQ ID NO: 364 - DNA, 45 bp, synthetic construct
  misc_feature 1..45: note = Description of Artificial Sequence: Synthetic oligonucleotide
  source 1..45: mol_type = other DNA
ggtggaggtggatcaggtggaggtggatctggtggaggtggatct

SEQ ID NO: 365 - protein, 5 aa, synthetic construct
  REGION 1..5: note = Description of Artificial Sequence: Synthetic peptide
GGGGS

SEQ ID NO: 366 - protein, 7 aa, synthetic construct
  REGION 1..7: note = Description of Artificial Sequence: Synthetic peptide
SGGSGGS

SEQ ID NO: 367 - protein, 9 aa, synthetic construct
  REGION 1..9: note = Description of Artificial Sequence: Synthetic peptide
GGGGSGGGS

SEQ ID NO: 368 - protein, 10 aa, synthetic construct
  REGION 1..10: note = Description of Artificial Sequence: Synthetic peptide
GGGGSGGGGS

SEQ ID NO: 369 - protein, 18 aa, synthetic construct
  REGION 1..18: note = Description of Artificial Sequence: Synthetic peptide
GGGGSGGGGSGGGGGGGS

SEQ ID NO: 370 - protein, 20 aa, synthetic construct
  REGION 1..20: note = Description of Artificial Sequence: Synthetic peptide
GGGGSGGGGSGGGGSGGGGS

SEQ ID NO: 371 - protein, 25 aa, synthetic construct
  REGION 1..25: note = Description of Artificial Sequence: Synthetic peptide
GGGGSGGGGSGGGGSGGGGSGGGGS

SEQ ID NO: 372 - protein, 30 aa, synthetic construct
  REGION 1..30: note = Description of Artificial Sequence: Synthetic polypeptide
GGGGSGGGGSGGGGSGGGGSGGGGSGGGGS

SEQ ID NO: 373 - protein, 35 aa, synthetic construct
  REGION 1..35: note = Description of Artificial Sequence: Synthetic polypeptide
GGGGSGGGGSGGGGSGGGGSGGGGSGGGGSGGGGS

SEQ ID NO: 374 - protein, 15 aa, synthetic construct
  REGION 1..15: note = Description of Artificial Sequence: Synthetic peptide
EPKSCDKTHTCPPCP

SEQ ID NO: 375 - protein, 24 aa, synthetic construct
  REGION 1..24: note = Description of Artificial Sequence: Synthetic peptide
GGGGSGGGSEPKSCDKTHTCPPCP

SEQ ID NO: 376 - protein, 62 aa, synthetic construct
  REGION 1..62: note = Description of Artificial Sequence: Synthetic polypeptide
ELKTPLGDTTHTCPRCPEPKSCDTPPPCPRCPEPKSCDTPPPCPRCPEPKSCDTPPPCPRCP

SEQ ID NO: 377 - protein, 6 aa, synthetic construct
  REGION 1..6: note = Description of Artificial Sequence: Synthetic peptide
GSGSGS

SEQ ID NO: 378 - skipped

SEQ ID NO: 379 - protein, 25 aa, synthetic construct
  REGION 1..25: note = Description of Artificial Sequence: Synthetic peptide
TSGQAGQHHHHHHGAYPYDVPDYAS

SEQ ID NO: 380 - DNA, 75 bp, synthetic construct
  misc_feature 1..75: note = Description of Artificial Sequence: Synthetic oligonucleotide
  source 1..75: mol_type = other DNA
actagtggccaggccggccagcaccatcaccatcaccatggcgcatacccgtacgacgttccggactacgcttct

SEQ ID NO: 381 - DNA, 363 bp, synthetic construct
  misc_feature 1..363: note = Description of Artificial Sequence: Synthetic polynucleotide
  source 1..363: mol_type = other DNA
caggtgcagctggtgcagtctggggctgaggtgaagaagcctgggtcctcggtgaaggtctcctgcaaggcttctggaggcaccttccgcagctatgctatcacctgggtgcgacaggcccctggacaagggcttgagtggatgggaaggatcatccctatgcttgatataacaaactacgcacagaagttccagggcagagtcacgattaccgcggacaaatccacgagcacagcctacatggagctgagcagcctgagatctgaggacacggccgtgtattactgtgcgcgcacttactctcgttctccgttccatatggaagatttctggggtcaaggtactctggtgaccgtctcctca

SEQ ID NO: 382 - protein, 33 aa, synthetic construct
  REGION 1..33: note = Description of Artificial Sequence: Synthetic polypeptide
CLSSERERVEDLFEYECELLTSEPIFHCRQEDC

SEQ ID NO: 383 - protein, 10 aa, Homo sapiens
CDAEGPWGII

SEQ ID NO: 384 - protein, 8 aa, Homo sapiens
MFVNMTPC

SEQ ID NO: 385 - protein, 9 aa, Homo sapiens
PQFQRQPQW

SEQ ID NO: 386 - protein, 13 aa, Homo sapiens
CIESTGDYFLLCD

SEQ ID NO: 387 - protein, 11 aa, Homo sapiens
NQQTAPVRYFL

SEQ ID NO: 388 - protein, 8 aa, Homo sapiens
GDYFLLCD

SEQ ID NO: 389 - protein, 11 aa, Homo sapiens
MFVNMTPCQLN

SEQ ID NO: 390 - protein, 11 aa, Homo sapiens
GNPQFQRQPQW

SEQ ID NO: 391 - protein, 9 aa, Homo sapiens
LCDAEGPWG

SEQ ID NO: 392 - protein, 14 aa, Homo sapiens
QFQRQPQWDDPVVC

SEQ ID NO: 393 - protein, 33 aa, synthetic construct
  REGION 1..33: note = Description of Artificial Sequence: Synthetic polypeptide
CMDYDFKVKLSSERERCWAIGCIFAELLTSEPC

SEQ ID NO: 394 - protein, 33 aa, synthetic construct
  REGION 1..33: note = Description of Artificial Sequence: Synthetic polypeptide
CMDYDFKVKLSSERERCCIFAELLTSEPIFHCC

SEQ ID NO: 395 - protein, 33 aa, synthetic construct
  REGION 1..33: note = Description of Artificial Sequence: Synthetic polypeptide
CMDYDFKVKLSSERERCELLTSEPIFHCRQEDC

SEQ ID NO: 396 - protein, 33 aa, synthetic construct
  REGION 1..33: note = Description of Artificial Sequence: Synthetic polypeptide
CMDYDFKVKLSSERERCSEPIFHCRQEDIKTSC

SEQ ID NO: 397 - protein, 33 aa, synthetic construct
  REGION 1..33: note = Description of Artificial Sequence: Synthetic polypeptide
CFKVKLSSERERVEDLCWAIGCIFAELLTSEPC

SEQ ID NO: 398 - protein, 33 aa, synthetic construct
  REGION 1..33: note = Description of Artificial Sequence: Synthetic polypeptide
CFKVKLSSERERVEDLCCIFAELLTSEPIFHCC

SEQ ID NO: 399 - protein, 33 aa, synthetic construct
  REGION 1..33: note = Description of Artificial Sequence: Synthetic polypeptide
CFKVKLSSERERVEDLCELLTSEPIFHCRQEDC

SEQ ID NO: 400 - protein, 33 aa, synthetic construct
  REGION 1..33: note = Description of Artificial Sequence: Synthetic polypeptide
CFKVKLSSERERVEDLCSEPIFHCRQEDIKTSC

SEQ ID NO: 401 - protein, 33 aa, synthetic construct
  REGION 1..33: note = Description of Artificial Sequence: Synthetic polypeptide
CLSSERERVEDLFEYECWAIGCIFAELLTSEPC

SEQ ID NO: 402 - protein, 33 aa, synthetic construct
  REGION 1..33: note = Description of Artificial Sequence: Synthetic polypeptide
CLSSERERVEDLFEYECCIFAELLTSEPIFHCC

SEQ ID NO: 403 - protein, 33 aa, synthetic construct
  REGION 1..33: note = Description of Artificial Sequence: Synthetic polypeptide
CLSSERERVEDLFEYECSEPIFHCRQEDIKTSC

SEQ ID NO: 404 - protein, 33 aa, synthetic construct
  REGION 1..33: note = Description of Artificial Sequence: Synthetic polypeptide
CRERVEDLFEYEGCKVCWAIGCIFAELLTSEPC

SEQ ID NO: 405 - protein, 33 aa, synthetic construct
  REGION 1..33: note = Description of Artificial Sequence: Synthetic polypeptide
CRERVEDLFEYEGCKVCCIFAELLTSEPIFHCC

SEQ ID NO: 406 - protein, 33 aa, synthetic construct
  REGION 1..33: note = Description of Artificial Sequence: Synthetic polypeptide
CRERVEDLFEYEGCKVCELLTSEPIFHCRQEDC

SEQ ID NO: 407 - protein, 33 aa, synthetic construct
  REGION 1..33: note = Description of Artificial Sequence: Synthetic polypeptide
CRERVEDLFEYEGCKVCSEPIFHCRQEDIKTSC

SEQ ID NO: 408 - protein, 15 aa, synthetic construct
  REGION 1..15: note = Description of Artificial Sequence: Synthetic peptide
MDYDFKVKLSSERER

SEQ ID NO: 409 - protein, 15 aa, synthetic construct
  REGION 1..15: note = Description of Artificial Sequence: Synthetic peptide
FKVKLSSERERVEDL

SEQ ID NO: 410 - protein, 15 aa, synthetic construct
  REGION 1..15: note = Description of Artificial Sequence: Synthetic peptide
LSSERERVEDLFEYE

SEQ ID NO: 411 - protein, 15 aa, synthetic construct
  REGION 1..15: note = Description of Artificial Sequence: Synthetic peptide
RERVEDLFEYEGCKV

SEQ ID NO: 412 - protein, 15 aa, synthetic construct
  REGION 1..15: note = Description of Artificial Sequence: Synthetic peptide
WAIGCIFAELLTSEP

SEQ ID NO: 413 - protein, 15 aa, synthetic construct
  REGION 1..15: note = Description of Artificial Sequence: Synthetic peptide
CIFAELLTSEPIFHC

SEQ ID NO: 414 - protein, 15 aa, synthetic construct
  REGION 1..15: note = Description of Artificial Sequence: Synthetic peptide
ELLTSEPIFHCRQED

SEQ ID NO: 415 - protein, 15 aa, synthetic construct
  REGION 1..15: note = Description of Artificial Sequence: Synthetic peptide
SEPIFHCRQEDIKTS